(19) United States
(12) Patent Application Publication
(10) Pub. No.: US 2018/0092866 A1
(43) Pub. Date: Apr. 5, 2018

(54) SUBSTITUTED N-([1,1'-BIPHENYL]-3-YL)-[1,1'-BIPHENYL]-3-CARBOXAMIDE ANALOGS AS INHIBITORS FOR BETA-CATENIN/B-CELL LYMPHOMA 9 INTERACTIONS

(71) Applicant: University of Utah Research Foundation, Salt Lake City, UT (US)

(72) Inventors: Haitao Ji, Salt Lake City, UT (US); Logan R. Hoggard, Logan, UT (US); Yongqiang Zhang, Salt Lake City, UT (US); John A. Wisniewski, Salt Lake City, UT (US)

(21) Appl. No.: 15/566,568

(22) PCT Filed: Apr. 14, 2016
(86) PCT No.: PCT/US16/27640

§ 371 Date: Oct. 13, 2017

Related U.S. Application Data
(60) Provisional application No. 62/147,901, filed on Apr. 15, 2015.

(51) Int. Cl. A61K 31/167; A61K 31/40; A61K 31/4402; A61K 31/41; C07D 403/14; C07D 403/04; A61K 47/10; A61K 47/34; A61K 47/36; A61K 9/00; A61K 9/10; A61K 9/20; A61P 43/00

(52) U.S. Cl. CPC A61K 31/167; A61P 43/00; A61K 31/4402; A61K 31/41; C07D 403/14; C07D 403/04; A61K 47/10; A61K 47/34; A61K 47/36; A61K 9/0019; A61K 9/10; A61K 9/2018; A61K 9/2027; A61K 9/2059; A61K 31/40

(57) ABSTRACT

In one aspect, the invention relates to substituted N-([1,1′-biphenyl]-3-yl)-[1,1′-biphenyl]-3-carboxamide analogues, derivatives thereof, and related compound; synthetic methods for making the compounds; pharmaceutical compositions comprising the compounds; and methods of treating disorders, e.g., various tumors and cancers, associated with β-Catenin/BCL9 protein-protein interaction dysfunction using the compounds and compositions. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 62/147,901, filed on Apr. 15, 2015, which is incorporated herein by reference in its entirety.

### STATEMENT REGARDING FEDERALLY SPONSORED RESEARCH

This invention was made with government support under Grant No. 11491162 awarded by the Department of Defense. The United States government has certain rights in the invention.

### REFERENCE TO SEQUENCE LISTING

The Sequence Listing submitted Apr. 15, 2016 as a text file named “21101_0300P1.txt,” created on Apr. 8, 2016, and having a size of 4,319 bytes is hereby incorporated by reference pursuant to 37 C.F.R. § 1.52(e)(5).

### BACKGROUND

The canonical Wnt/β-catenin pathway is of particular importance in regulating cell proliferation, differentiation and cell-cell communication. The aberrant activation of Wnt/β-catenin signaling leads to the initiation and progression of many cancers such as colorectal cancers (P. Morin, et al. Science 275 (1997) 1787-1790), hepatocellular carcinoma (A., de La Coste, et al. Proc. Natl. Acad. Sci. U.S.A 95 (1998) 8847-8851), breast cancers (C. Scheel, E. N. Eaton, S. H. Li, et al. Cell 145 (2011) 926-940), leukaemia (D. Lu, et al. Proc. Natl. Acad. Sci. U.S.A 101 (2004) 3118-3123), and multiple myeloma (K. Sukhdeo, et al. Proc. Natl. Acad. Sci. U.S.A 104 (2007) 7516-7521). Moreover, cancer stem cells, which are resistant to conventional chemotherapies and are especially virulent, are controlled by the overactivated Wnt//β-catenin signaling (L. Vermeulen, E. De Sousa, F. Melo, et al. Nat. Cell Biol. 2010, 12 (5), 468-476; C. Scheel, E. N. Eaton, S. Li, et al. Cell 2011, 145 (6), 926-940). In addition, dysfunction in the Wnt/β-catenin signaling pathway can lead to fibrotic diseases, e.g., pulmonary fibrosis (W. R. Henderson Jr., et al. Proc. Natl. Acad. Sci. U.S.A 107 (2010) 14309-14314), liver fibrosis (J. H. Cheng, et al. Am. J. Physiol. Gastrointest. Liver Physiol. 294 (2008) G39-G49) and cystic kidney disease (M. A. Lancaster, et al. Nat. Med. 15 (2009) 1046-1054).

β-Catenin is the key mediator of the canonical Wnt pathway. The hyperactivation of canonical Wnt signaling leads to an accumulation of β-catenin in the cell nucleus. Nucleus β-catenin forms a supercomplex with T-cell factor/lymphoid enhancer-binding factor (LEF), B-cell lymphoma 9 (BCL9)/B9L (a BCL9 paralogue), and CREB (cAMP response element-binding protein)-binding protein (CBP)/p300, etc. to activate transcription of a number of β-catenin target genes including AXIN2, LGR5, cyclin D1, c-myc, LEF1, survivin, and multidrug resistance 1 (MDR1), which further promote cancer cell growth, migration, resistance to current drugs, and evasion from apoptosis. Canonical Wnt signaling is also aberrantly overactivated in cancer stem cells, which drives cancer growth, seeds metastases, and causes cancer recurrence after remission (Anastas and Moon Nat. Rev. Cancer 13 (2013) 11-26). The penultimate step of this signaling pathway is the formation of the β-catenin/BCL9 complex in the cell nucleus (S. Adachi et al. Cancer Res. 64 (2004) 8496-8501).

Despite advances in research directed to identifying inhibitors the Wnt signaling pathway generally, and specifically inhibitors of β-catenin/BCL9 interactions, there remains a scarcity of compounds that are both potent, efficacious, and selective inhibitors of β-catenin/BCL9 interactions and also effective in the treatment of cancers and other diseases associated with uncontrolled cellular proliferation, e.g., fibrotic diseases, associated with β-catenin/BCL9 dysfunction. These needs and other needs are satisfied by the present invention.

### SUMMARY

In accordance with the purpose(s) of the invention, as embodied and broadly described herein, the invention, in one aspect, relates to compounds useful useful as inhibitors of β-catenin/B-cell lymphoma 9 protein-protein interactions, methods of making same, pharmaceutical compositions comprising same, and methods of treating disorders, e.g., various tumors and cancers, associated with a β-catenin/B-cell lymphoma 9 protein-protein interaction dysfunction or a Wnt pathway dysregulation using same.

Disclosed are compounds having a structure represented by a formula:

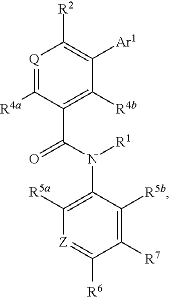

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; wherein each occurrence of R²⁰, when present, is independently selected from C1-C3 alkyl, C1-C3 monohaloalkyl, C1-C3 polyhaloalkyl, and cyclopropyl; wherein each occurrence of R^{21a} and R^{21b}, when present, is independently selected from hydrogen, C1-C3 alkyl, C1-C3 monohaloalkyl, C1-C3 polyhaloalkyl, and cyclopropyl; wherein R⁷ is selected from Ar², -A¹-A²-Ar², and

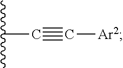

wherein each of A¹ and A², when present, is independently selected from O, NH, and CH₂, provided that each of A¹ and A² is simultaneously O; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; or a pharmaceutically acceptable salt thereof.

Also disclosed are compounds having a structure represented by a formula:

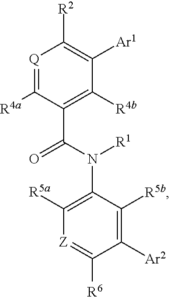

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

Also disclosed are pharmaceutical compositions comprising a therapeutically effective amount of a disclosed compound, or a pharmaceutically acceptable salt thereof, and a pharmaceutically acceptable carrier.

Also disclosed are methods for the treatment of a disorder of uncontrolled cellular proliferation associated with a β-catenin/BCL9 dysfunction in a mammal comprising the step of administering to the mammal an effective amount of at least one disclosed compound, or a pharmaceutically acceptable salt thereof.

Also disclosed are methods for inhibiting protein-protein interactions of β-catenin and BCL9 in a mammal comprising the step of administering to the mammal a therapeutically effective amount of at least one disclosed compound, or a pharmaceutically acceptable salt thereof.

Also disclosed are methods for inhibiting protein-protein interactions of β-catenin and BCL9 in at least one cell, comprising the step of contacting the at least one cell with an effective amount of at least one disclosed compound, or a pharmaceutically acceptable salt thereof.

Also disclosed are uses of at least one disclosed compound for inhibiting β-catenin/BCL9 activity.

Also disclosed are uses of at least one disclosed compound for administration to a subject; wherein the subject has a disorder of uncontrolled cellular proliferation.

Also disclosed are kits comprising at least one disclosed compound, or a pharmaceutically acceptable salt thereof; and one or more of:
(a) at least one agent known to increase BCL9 activity;
(b) at least one agent known to increase β-catenin activity;
(c) at least one agent known to decrease BCL9 activity;
(d) at least one agent known to decrease β-catenin activity;
(e) at least one agent known to treat a disease of uncontrolled cellular proliferation;
(f) instructions for treating a disorder associated uncontrolled cellular proliferation; or
(g) instructions for treating a disorder associated with a β-catenin/BCL9 dysfunction.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A and FIG. 1B show representative images of a surface model (FIG. 1A) and a stick model (FIG. 1B) from a hydrophobic SiteMap analysis.

FIG. 2A and FIG. 2B show representative images of a H-bond donor map (FIG. 2A) and a H-bond acceptory map (FIG. 2B) from a H-bond SiteMap analysis.

FIG. 3A-C show representative images of a conformational analysis of 4′-fluoro-N-phenyl-[1,1′-biphenyl]-3-carboxamide.

FIG. 4A-F show representative data related to the design of a scaffold to mimc the sides chains of residues i, i+3, and i+7 of an α-helix.

FIG. 5 shows representative data from an AlphaScreen competitive inhibition assay of compounds 1-15 for the inhibition of β-catenin/BCL9 protein-protein interactions.

FIG. 6 shows a representative image of a stick model of the AutoDock predicted binding conformation of compound 9 in β-catenin.

FIG. 7A-E show representative data related to the optimization of β-catenin/BCL9 inhibitors.

FIG. 8 shows a representative image of a stick model of the AutoDock predicted binding conformation of compound 21 in β-catenin.

FIG. 9 shows representative data from an AlphaScreen competitive inhibition assay of compounds 16-29 for the inhibition of β-catenin/BCL9 protein-protein interactions.

FIG. 10 shows representative data from an AlphaScreen competitive inhibition assay of compounds 17, 20-23, 26-29, and carnosic acid for the inhibition of β-catenin/E-cadherin protein-protein interactions.

FIG. 11A and FIG. 11B shows representative data from an isothermal titration calorimetry (ITC) study to determine the binding affinity of compound 21 with human β-catenin (residues 138-686, FIG. 11A) and human BCL9 (residues 350-375, FIG. 11B).

FIG. 12A and FIG. 12B shows representative data from an isothermal titration calorimetry (ITC) study to determine the binding affinity of compound 21 with wild-type (FIG. 12A) and D145A/E155A mutant (FIG. 12B) β-catenin (residues 138-686) proteins.

FIG. 13A and FIG. 13B shows representative data from an isothermal titration calorimetry (ITC) study to determine the binding affinity of compound 21 with L159S mutant (FIG. 13A) and L156S/L178S mutant (FIG. 13B) β-catenin (residues 138-686) proteins.

FIG. 14 shows representative data from an AlphaScreen competitive inhibition assay of compounds 17, 20, and 21 with wild-type and mutant β-catenin proteins for the inhibition of β-catenin/BCL9 interactions.

FIG. 15 shows representative data from an AlphaScreen competitive binding assay to determine the apparent K_{d} values for the wild-type β-catenin/wild-type BCL9 interaction and the mutant β-catenin/wild-type BCL9 interactions.

FIG. 16 shows representative data from FP saturation binding experiments to determine the apparent K_{d} values for the wild-type β-catenin/wild-type BCL9 interaction and the mutant β-catenin/wild-type BCL9 interactions.

FIG. 17A and FIG. 17B show representative images of the structural superimposition of the crystal structures of β-catenin in complexes with BCL9 (PDB id, 2GL7) and region V of E-cadherin (PDB id, 1I7W).

FIG. 18A and FIG. 18B show representative images of the AutoDock predicted binding conformation of compound 29 with β-catenin (PDB id, 2GL7).

FIG. 19A-E show representative data related to cell-based studies of β-catenin/BCL9 inhibitors. Specifically, data from a Wnt-responsive luciferase reporter assay (FIG. 19A), a quantitative real-time PCR study (FIG. 19B), a Western blot analysis (FIG. 19C), co-immunoprecipitation experiments (FIG. 19D), and MTs assay (FIG. 19E) are shown.

FIG. 20 shows representative data related to the effects of compounds 20, 21, and carnosic acid on the transactivation of the canonical Wnt signaling pathway as determined by a luciferase reporter assay.

FIG. 21 shows representative data related to the quantitative real-time PCR results of compound 21 in colorectal cancer cells SW480.

FIG. 22 shows representative data related to the inhibitory effect of compound 21 on HCT116 colony formation activity.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, brosylate, and halides.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

It is also appreciated that certain compounds described herein can be present as an equilibrium mixture of tautomers. For example, ketones with an α-hydrogen can exist in an equilibrium mixture of the keto form and the enol form.

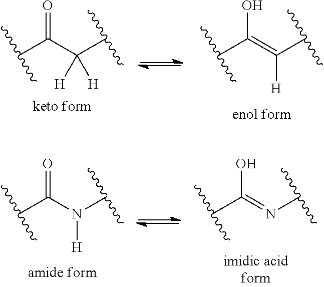 [SMILES: *CC(*)=O ; */C=C(/*)O ; *NC(*)=O ; */N=C(/*)O]

Likewise, amides with an N-hydrogen can exist in an equilibrium mixture of the amide form and the imidic acid form. As another example, tetrazoles can exist in two tautomeric forms, N¹-unsubstituted and N²-unsubstituted, as shown below.

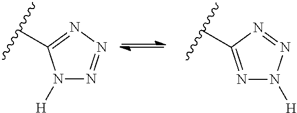 [SMILES: *c1nnn[nH]1 ; *c1nn[nH]n1]

Unless stated to the contrary, the invention includes all such possible tautomers.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

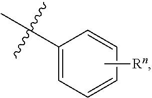

which is understood to be equivalent to a formula:

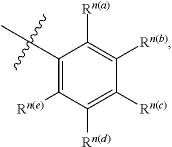

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to compounds useful as inhibitors of β-catenin/BCL9 protein-protein interactions, and thus down-regulating Wnt signaling. In a further aspect, the compound selectively inhibits β-catenin/BCL9 interactions compared to β-catenin/cadherin interactions. In a still further aspect, the compound inhibits Wnt signaling. In yet a further aspect, the compound inhibits transcription of at least one β-catenin target gene.

In a further aspect, the compound inhibits cell viability. In a still further aspect, the compound inhibits cell migration. In yet a further aspect, the compound inhibits angiogenesis. In an even further aspect, the compound inhibits tumor metastasis. In a still further aspect, the compound inhibits tumor progression.

In a further aspect, the compound exhibits inhibition with a Kᵢ of less than about 1.0×10⁻⁴ M when determined in competitive inhibition assay. In a still further aspect, the compound exhibits inhibition with a Kᵢ of less than about 7.0×10⁻⁵ M when determined in competitive inhibition assay. In yet a further aspect, the compound exhibits inhibition with a Kᵢ of less than about 5.0×10⁻⁵ M when determined in competitive inhibition assay. In an even further aspect, the compound exhibits inhibition with a Kᵢ of less than about 2.5×10⁻⁵ M when determined in competitive inhibition assay. In a still further aspect, the compound exhibits inhibition with a Kᵢ of less than about 1.0×10⁻⁵ M when determined in competitive inhibition assay. In yet a further aspect, the compound exhibits inhibition with a Kᵢ of less than about 5.0×10⁻⁶ M when determined in competitive inhibition assay.

In one aspect, the compounds of the invention are useful in the treatment of disorders, e.g., various tumors and cancers, associated with a β-catenin/BCL9 protein-protein interaction dysfunction or a Wnt pathway dysregulation using same, and other diseases in which β-catenin/BCL9 or the Wnt signaling pathway are involved, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, the invention relates to a compound having a structure represented by a formula:

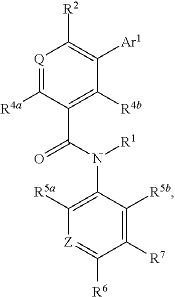

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; wherein each occurrence of R²⁰, when present, is independently selected from C1-C3 alkyl, C1-C3 monohaloalkyl, C1-C3 polyhaloalkyl, and cyclopropyl; wherein each occurrence of R^{21a} and R^{21b}, when present, is independently selected from hydrogen, C1-C3 alkyl, C1-C3 monohaloalkyl, C1-C3 polyhaloalkyl, and cyclopropyl; wherein R⁷ is selected from Ar², -A¹-A²-Ar², and

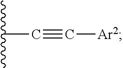

wherein each of A¹ and A², when present, is independently selected from O, NH, and CH₂, provided that each of A¹ and A² is simultaneously O; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; or a pharmaceutically acceptable salt thereof.

In one aspect, the invention relates to a compound having a structure represented by a formula:

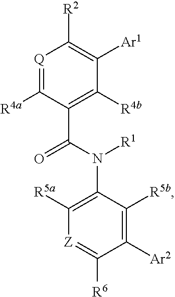

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

In a further aspect, the compound has a structure represented by a formula listed below:

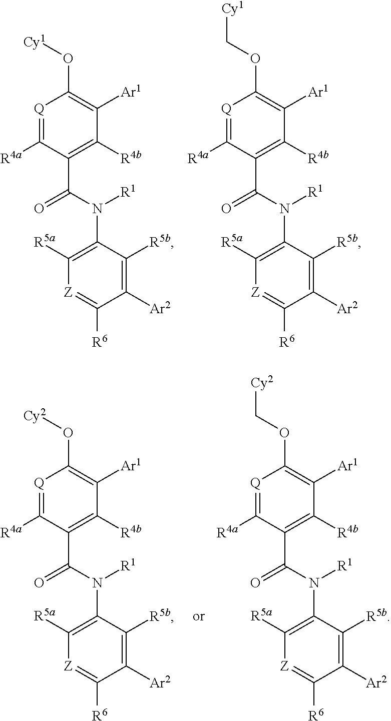

In a further aspect, the compound has a structure represented by a formula listed below:

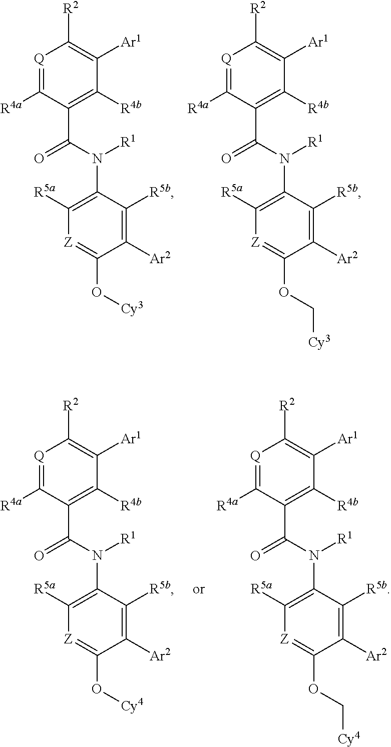

In a further aspect, the compound has a structure represented by a formula listed below:

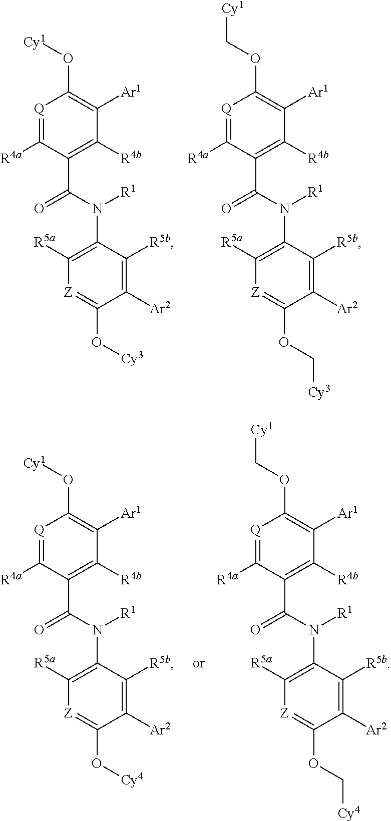

In a further aspect, the compound has a structure represented by a formula listed below:

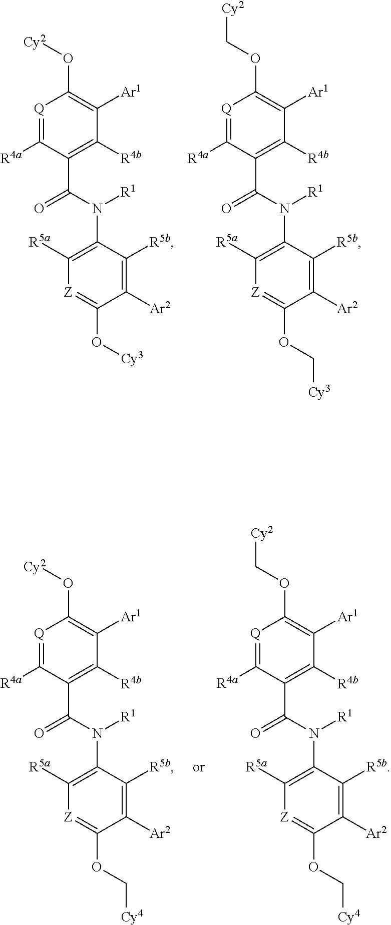

In a further aspect, the compound has a structure represented by a formula listed below:

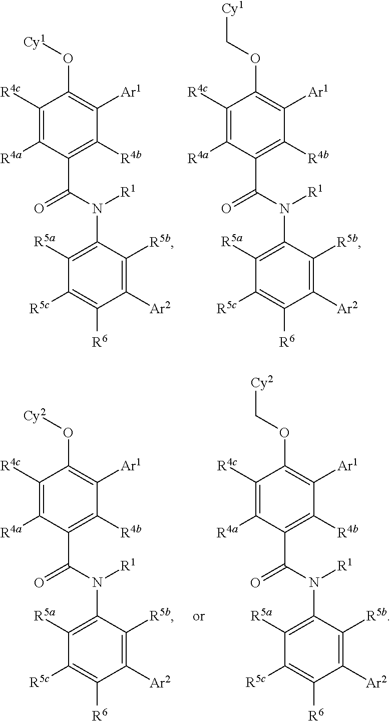

In a further aspect, the compound has a structure represented by a formula listed below:

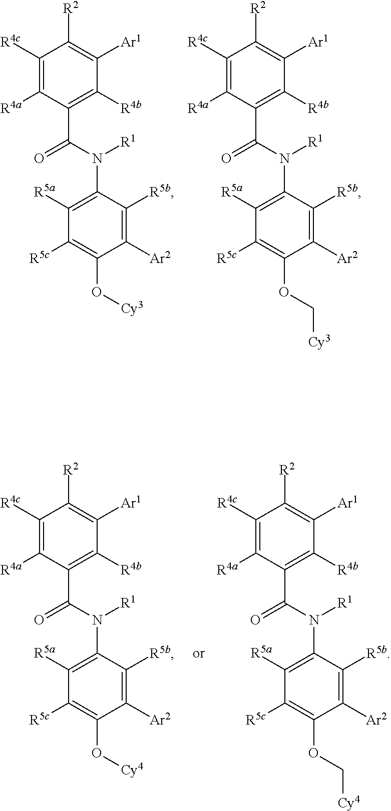

In a further aspect, the compound has a structure represented by a formula listed below:

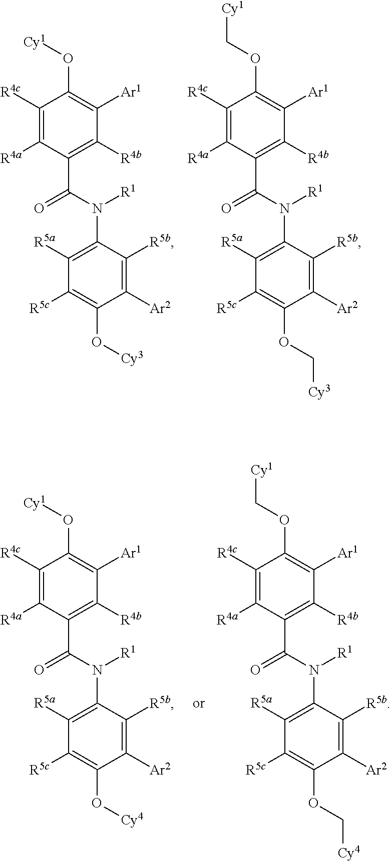

In a further aspect, the compound has a structure represented by a formula listed below:

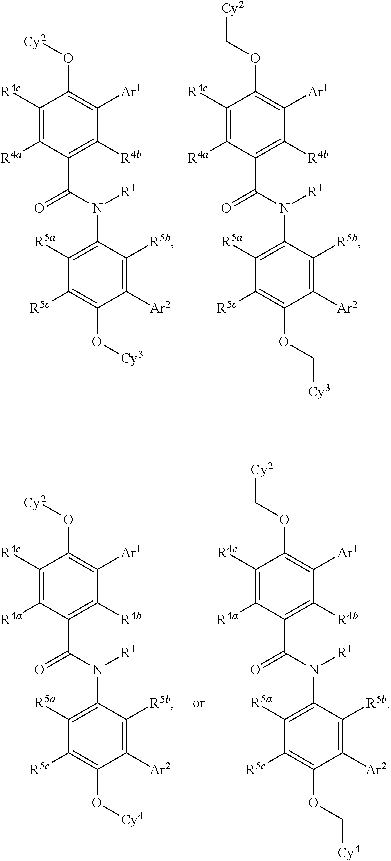

In a further aspect, the compound has a structure represented by a formula listed below:

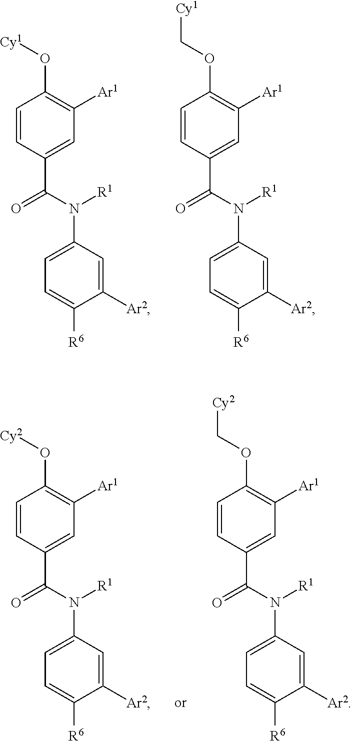

In a further aspect, the compound has a structure represented by a formula listed below:

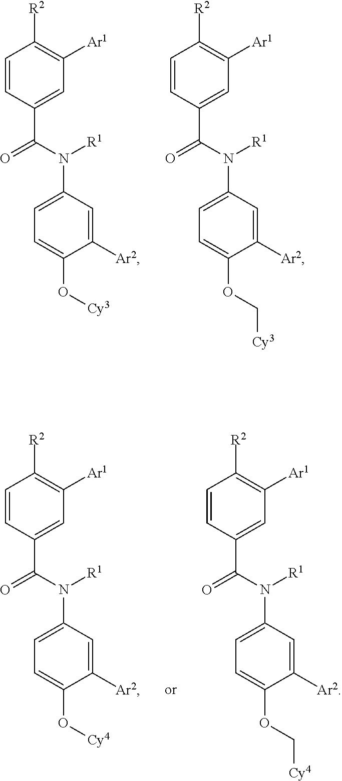

In a further aspect, the compound has a structure represented by a formula listed below:

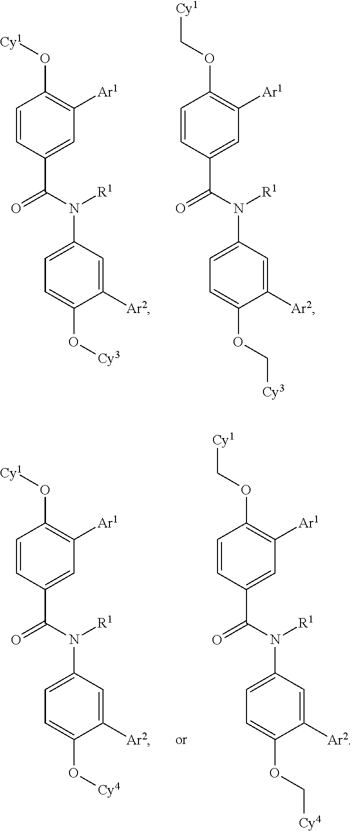

In a further aspect, the compound has a structure represented by a formula listed below:

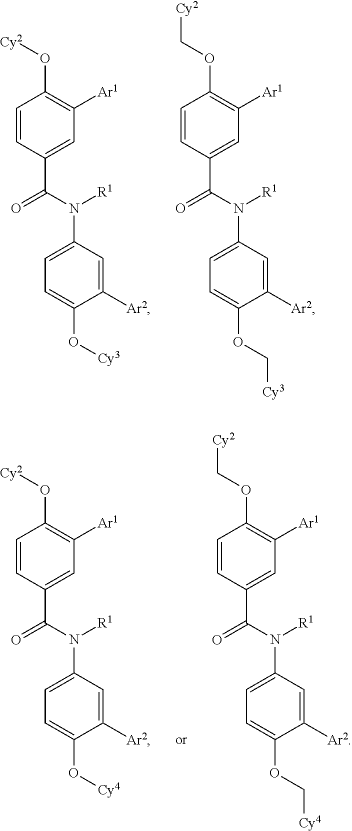

In a further aspect, the compound has a structure represented by a formula listed below:

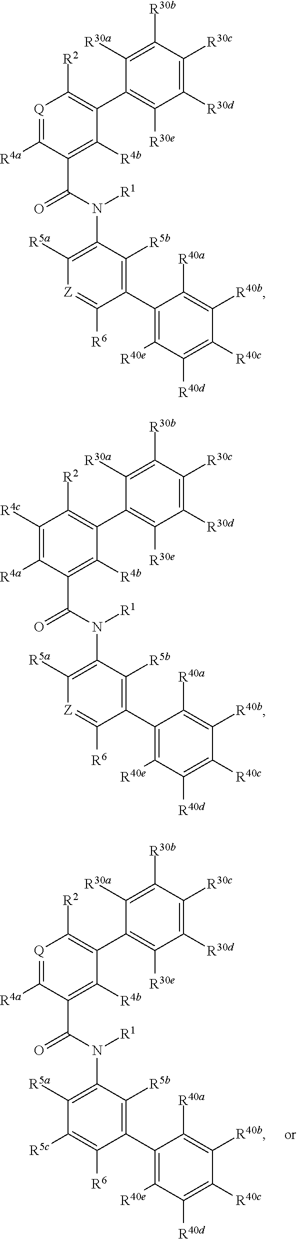

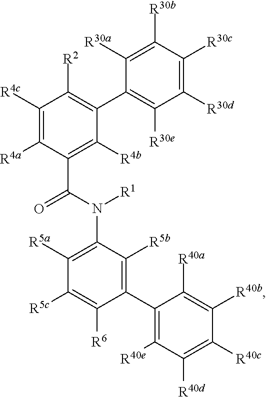

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

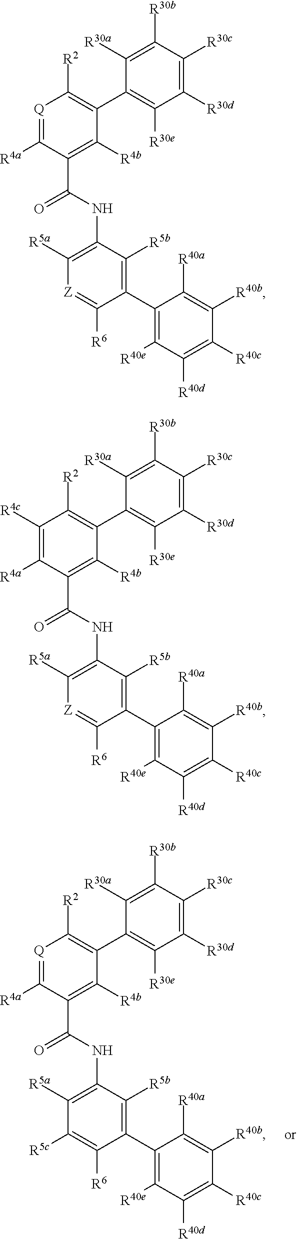

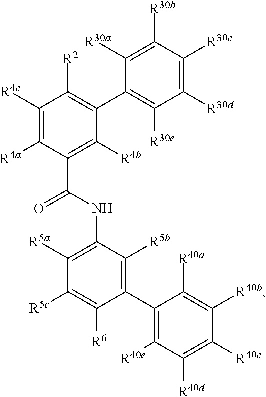

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

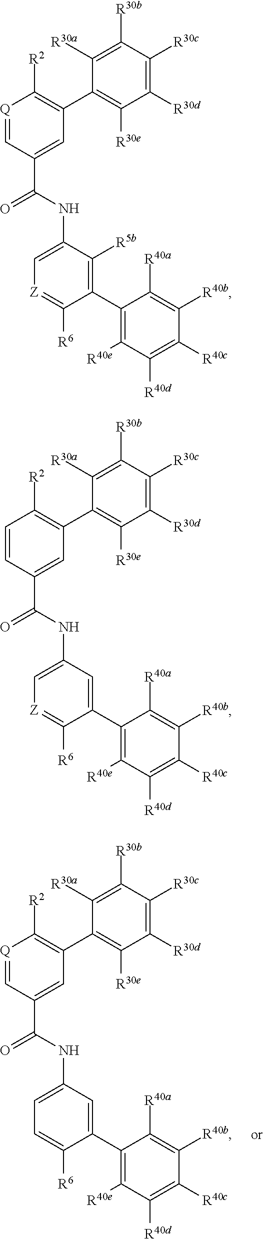

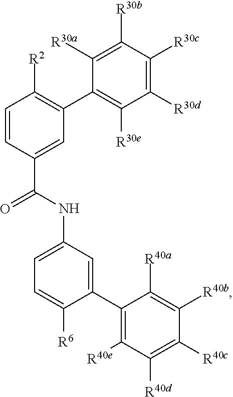

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

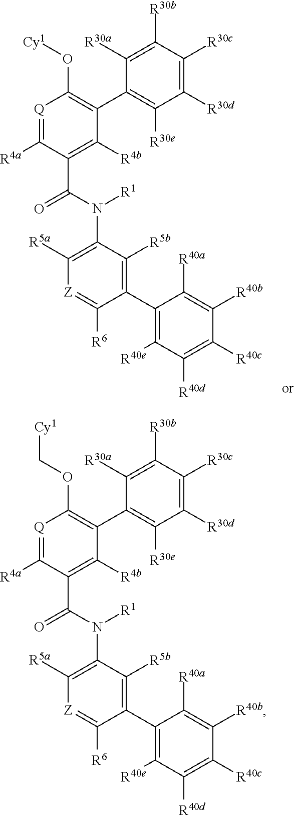

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

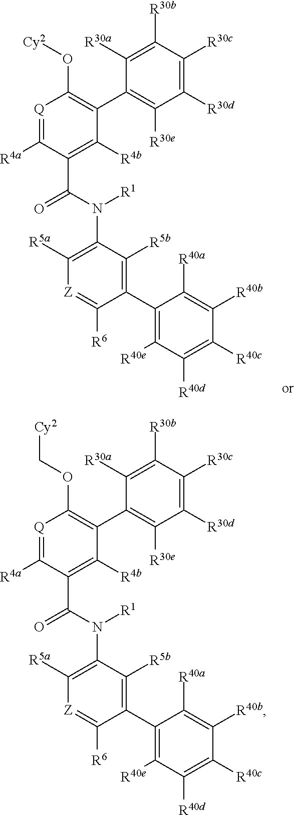

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

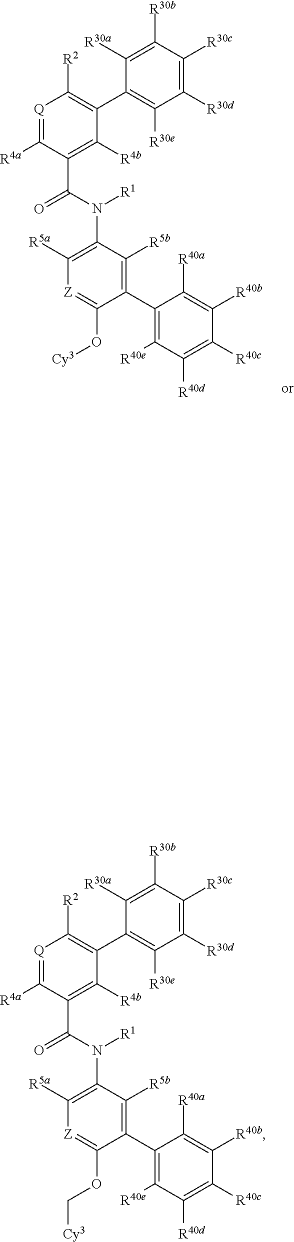

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

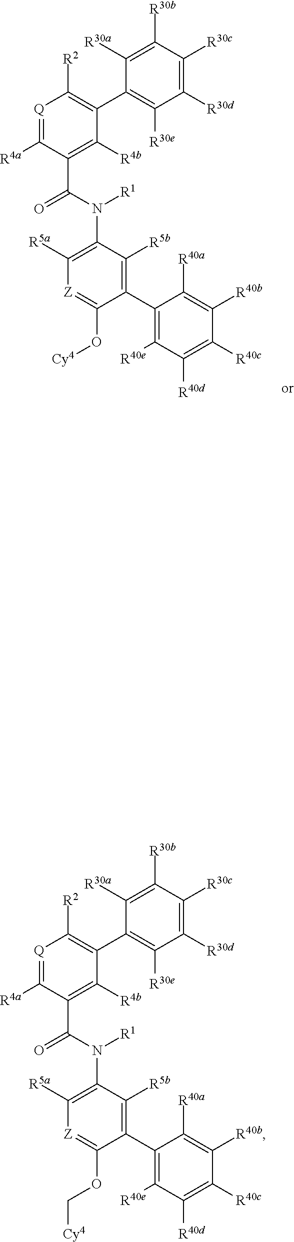

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

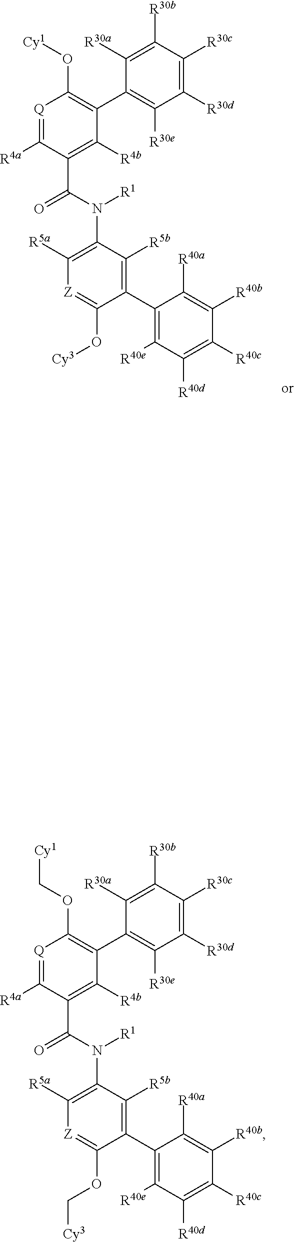

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

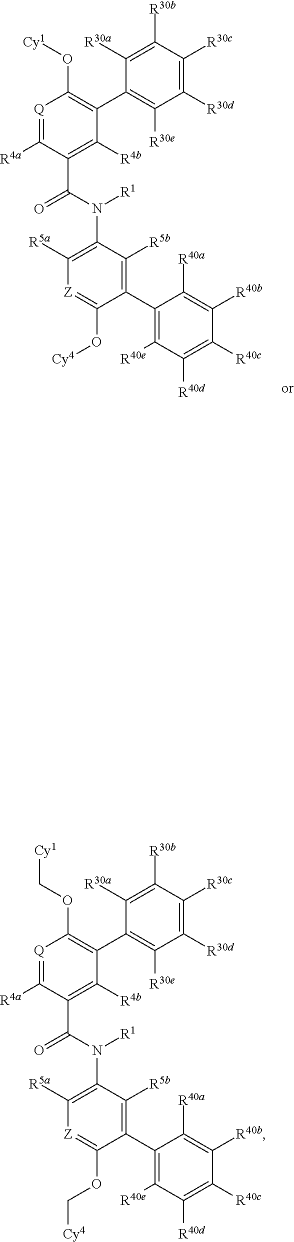

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

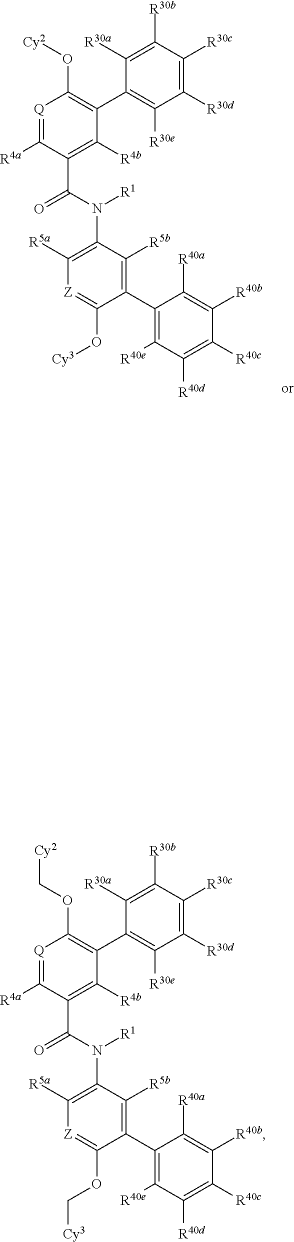

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

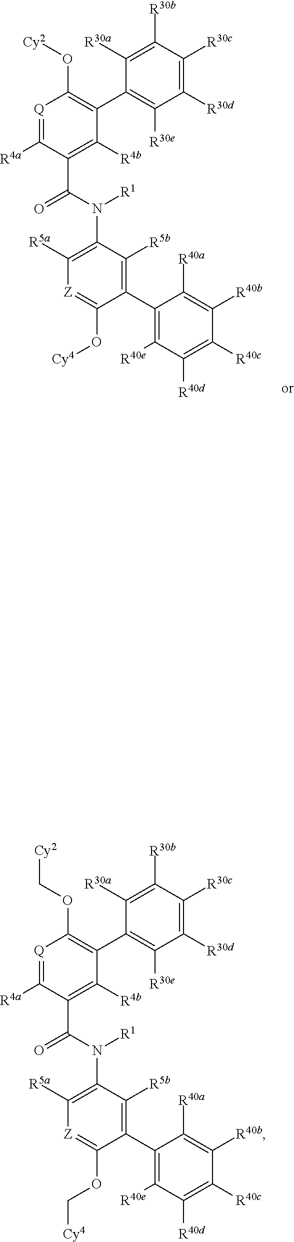

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

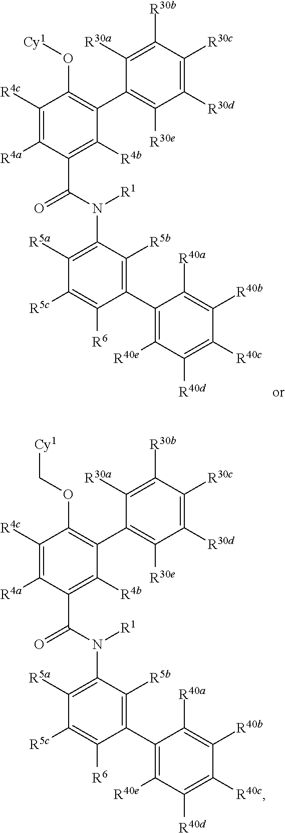

wherein each of R^{30a}, R^{30b}, R^{30c}, R^{3d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

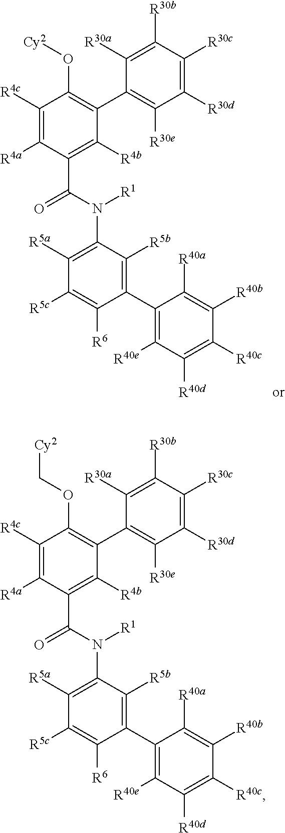

wherein each of R^{30a}, R^{30b}, Roc, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

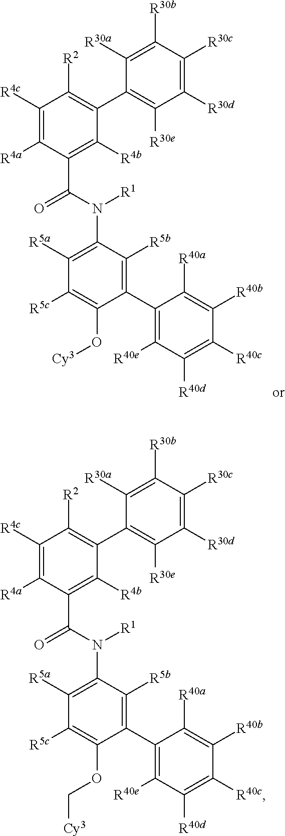

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

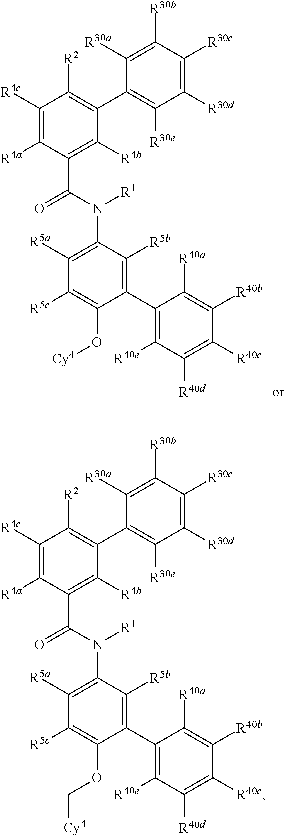

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

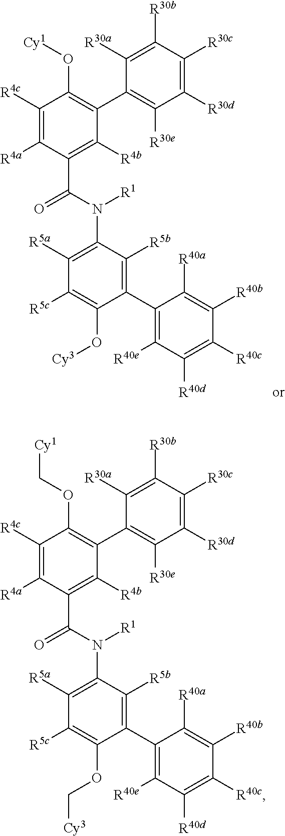

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

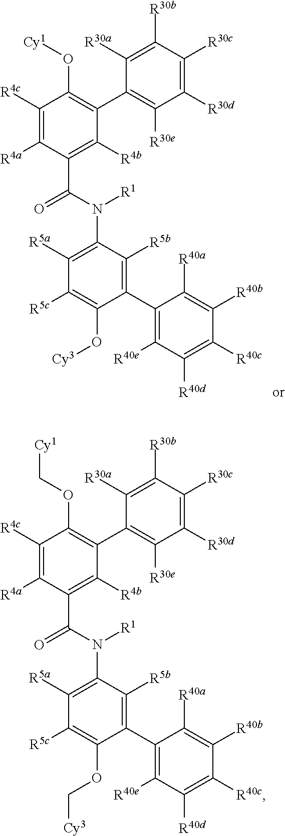

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

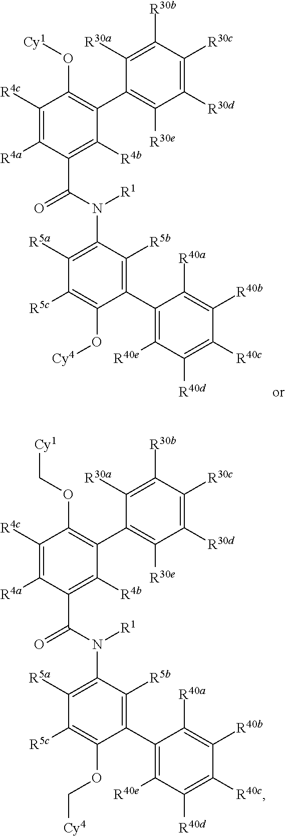

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

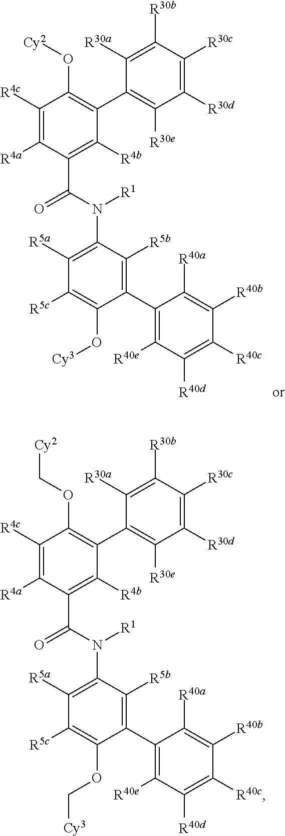

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

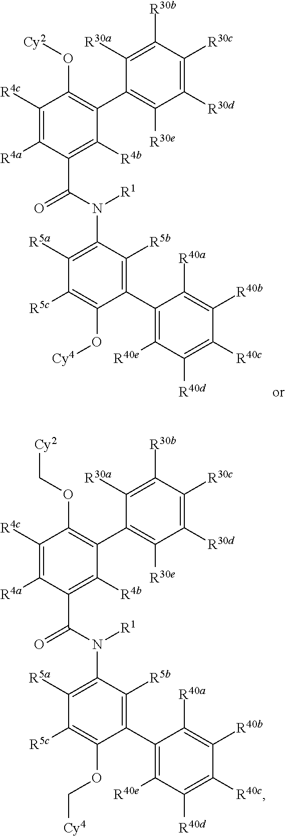

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

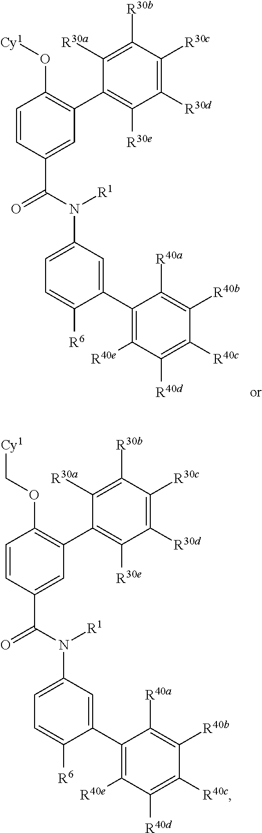

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

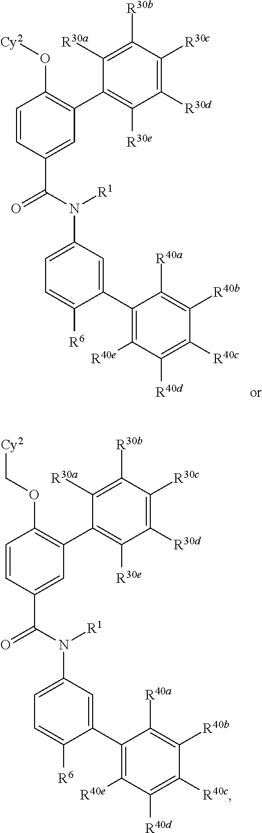

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

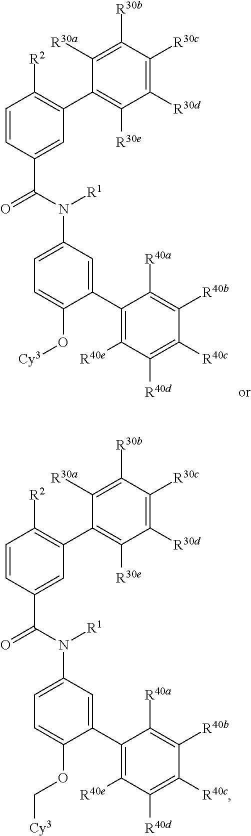

or
wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, The compound of claim 1, having a structure represented by a formula:

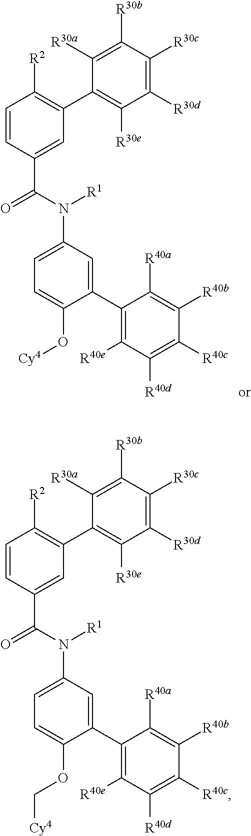

or
wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

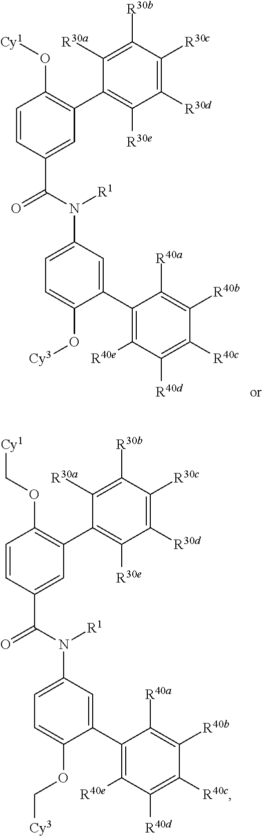

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

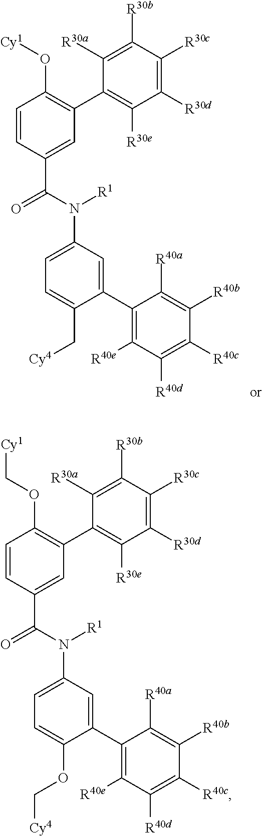

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

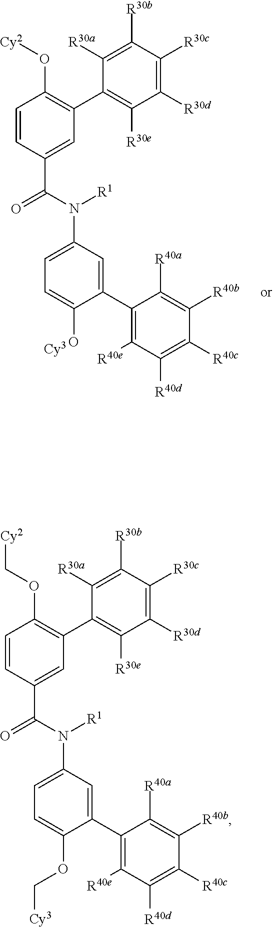

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

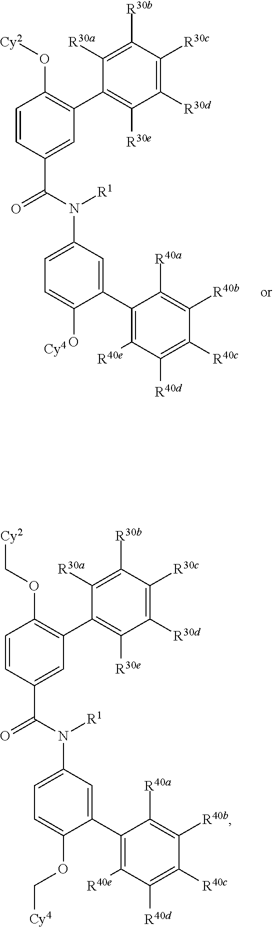

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula:

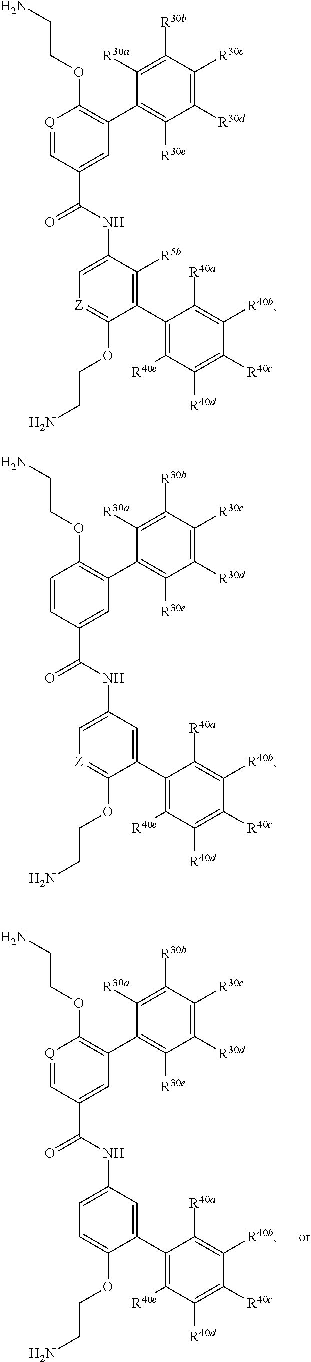

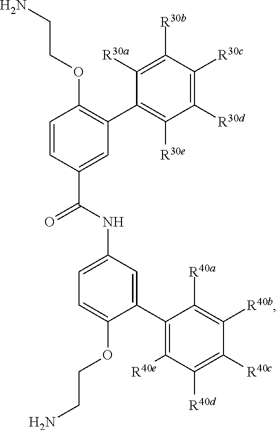

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

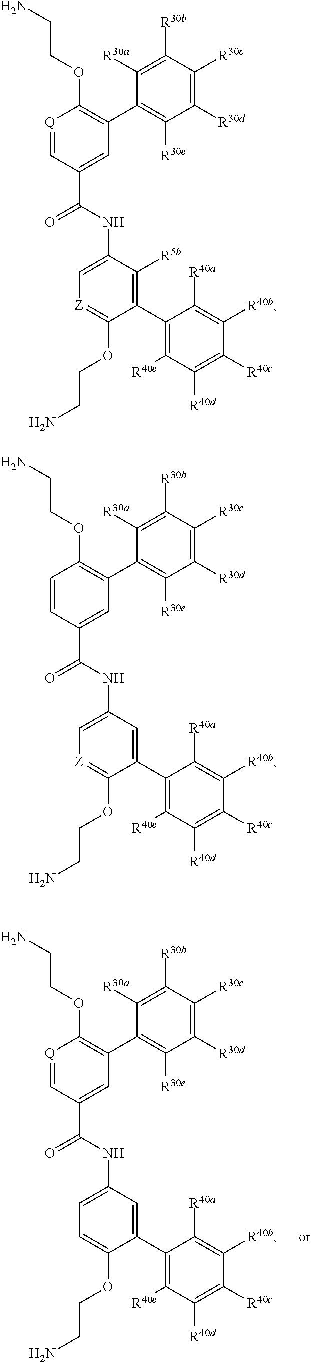

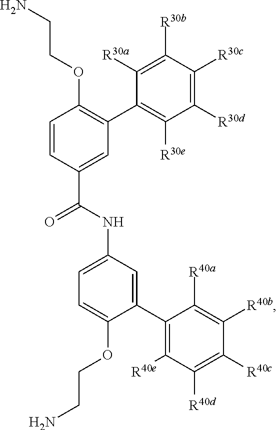

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

In a further aspect, the compound has a structure represented by a formula listed below:

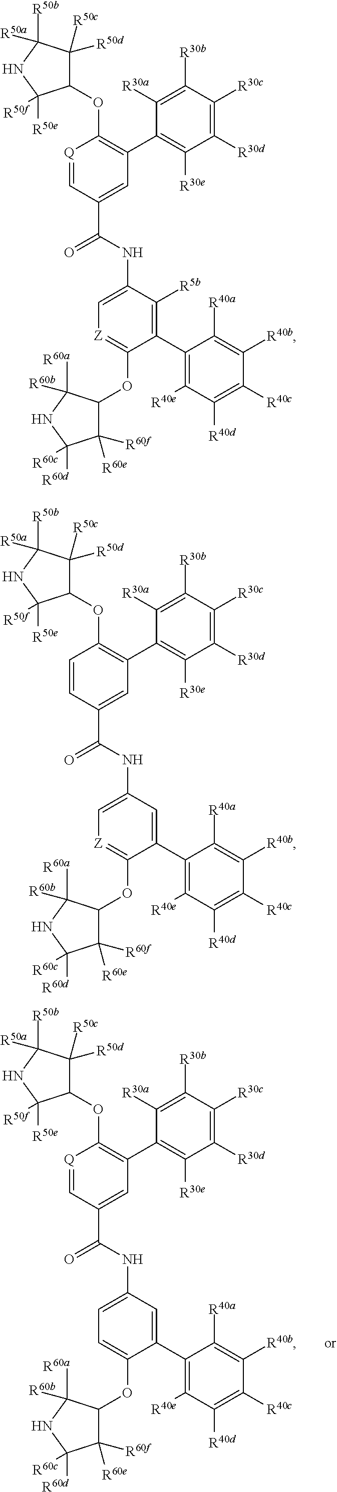

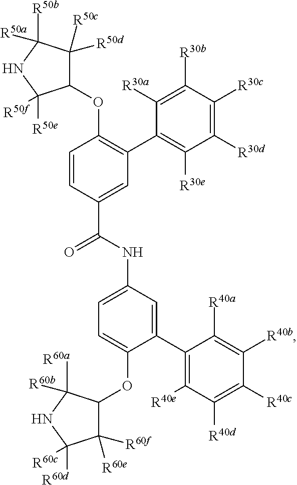

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen; wherein each of R^{50a}R^{50b}, R^{50c}, R^{50d}, R^{50e}, and R^{50f} is independently selected from hydrogen, halogen, C1-C4 alkyl, C1-C4 monohaloalkyl, and C1-C4 polyhaloalkyl; and wherein at least three of R^{50a}, R^{50b}, R^{50c}, R^{50d}, R^{50e}, and R^{50f} are hydrogen; and wherein each of R^{60a}, R^{60b}, R^{60c}, R^{60d}, R^{60e}, and R^{60f} is independently selected from hydrogen, halogen, C1-C4 alkyl, C1-C4 monohaloalkyl, and C1-C4 polyhaloalkyl; and wherein at least three of R^{60a}, R^{60b}, R^{60c}, R^{60d}R^{60e}, and R^{60f} are hydrogen.

In a further aspect, the compound has a structure represented by a formula listed below:

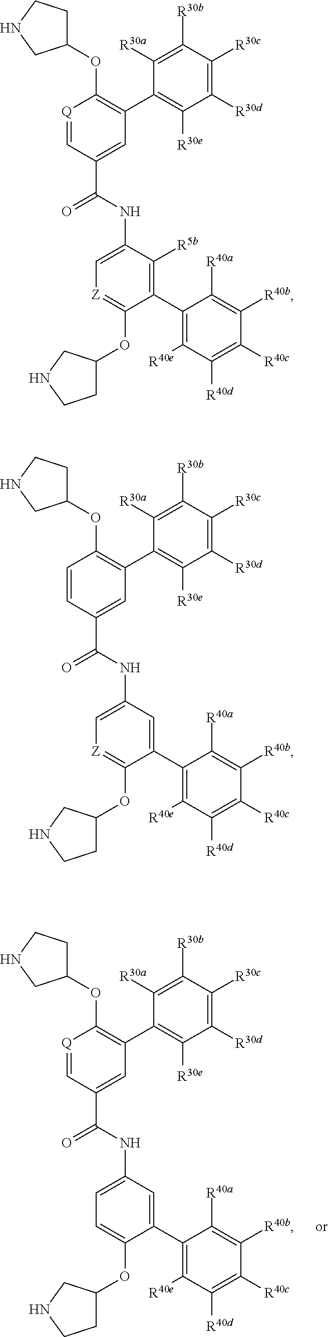

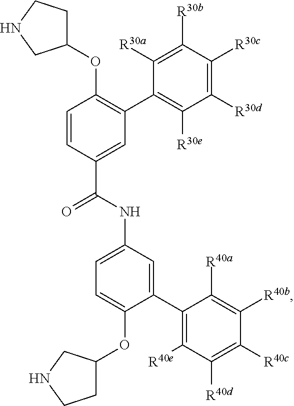

wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

a. A¹ and A²

In one aspect, each of A¹ and A², when present, is independently selected from O, NH, and CH₂, provided that each of A¹ and A² is simultaneously O. In a further aspect, each of A¹ and A², when present, is independently selected from O and NH. In a still further aspect, each of A¹ and A², when present, is independently selected from O and CH₂. In yet a further aspect, each of A¹ and A², when present, is independently selected from NH and CH₂. In a still further aspect, each of A¹ and A², when present, is NH. In yet a further aspect, each of A¹ and A², when present, is CH₂.

b. Q

In one aspect, Q is selected from N and CR^{4c}. In a further aspect, Q is N. In a still further aspect, Q is CR^{4c}.

c. Z

In one aspect, Z is selected from N and CR^{5c}. In a further aspect, Z is N. In a still further aspect, Z is CR^{5c}.

d. R¹ Groups

In one aspect, R¹ is selected from hydrogen and C1-C4 alkyl. In a still further aspect, R¹ is hydrogen.

In a further aspect, R¹ is C1-C4 alkyl. In a still further aspect, R¹ is selected from methyl, ethyl, propyl, and isopropyl. In yet a further aspect, R¹ is selected from methyl and ethyl. In an even further aspect, R¹ is methyl. In a still further aspect, R¹ is ethyl.

In a further aspect, R¹ is selected from hydrogen, methyl, ethyl, propyl, and isopropyl. In a still further aspect, R¹ is selected from hydrogen, methyl, and ethyl. In yet a further aspect, R¹ is selected from hydrogen and methyl.

e. R² Groups

In one aspect, R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy². In a further aspect, R² is selected from —(C2-C4 alkyl)-OH, —(C2-C4 alkyl)-NH₂, —O—(C2-C4 alkyl)-OH, —O—(C2-C4 alkyl)-NH₂, —NH—(C2-C4 alkyl)-OH, —NH—(C2-C4 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy².

In a further aspect, R² is selected from —(C2-C8 alkyl)-OH, —O—(C2-C8 alkyl)-OH, —O—(C2-C8 alkyl)-NH₂, —O-Cy¹, —O-Cy², —OCH₂-Cy¹, and —OCH₂-Cy². In a still further aspect, R² is selected from —(C2-C8 alkyl)-OH, —O—(C2-C8 alkyl)-OH, and —O—(C2-C8 alkyl)-NH₂. In yet a further aspect, R² is —(C2-C8 alkyl)-OH. In an even further aspect, —O—(C2-C8 alkyl)-OH. In a still further aspect, R² is —O—(C2-C8 alkyl)-NH₂.

In a further aspect, R² is selected from —O-Cy¹, —O-Cy², —OCH₂-Cy¹, and —OCH₂-Cy². In a still further aspect, R² is selected from —O-Cy¹ and —O-Cy². In yet a further aspect, R² is selected from —OCH₂-Cy¹ and —OCH₂-Cy². In an even further aspect, R² is —O-Cy¹. In a still further aspect, R² is —O-Cy². In yet a further aspect, R² is —OCH₂-Cy¹. In an even further aspect, R² is —OCH₂-Cy².

In a further aspect, R² is selected from —(C2-C8 alkyl)-NH₂, —NH—(C2-C8 alkyl)-OH, —NH—(C2-C8 alkyl)-NH₂, —NH-Cy¹, —NH-Cy², —NHCH₂-Cy¹, and —NHCH₂-Cy². In a still further aspect, R² is selected from —(C2-C8 alkyl)-NH₂, —NH—(C2-C8 alkyl)-OH, and —NH—(C2-C8 alkyl)-NH₂. In yet a further aspect, R² is —(C2-C8 alkyl)-NH₂. In an even further aspect, R² is —NH—(C2-C8 alkyl)-OH. In a still further aspect, R² is —NH—(C2-C8 alkyl)-NH₂.

In a further aspect, R² is selected from —NH-Cy¹, —NH-Cy², —NHCH₂-Cy¹, and —NHCH₂-Cy². In a still further aspect, R² is selected from —NH-Cy¹ and —NH-Cy². In yet a further aspect, R² is —NH-Cy¹. In an even further aspect, R² is —NH-Cy². In a still further aspect, R² is selected —NHCH₂-Cy¹ and —NHCH₂-Cy². In yet a further aspect, R² is —NHCH₂-Cy¹. In an even further aspect, R² is —NHCH₂-Cy².

In a further aspect, R² is selected from —NH-Cy¹, —NH-Cy², —O-Cy¹, —O-Cy², —NHCH₂-Cy¹, —NHCH₂-Cy²; —OCH₂-Cy¹, and —OCH₂-Cy².

In a further aspect, R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂.

f. R^{4a}, R^{4b}, and R^{4c} Groups

In one aspect, each of R^{4a}, R^{4b}, and R^{4c}, when present, is independently selected from hydrogen, C1-C4 alkyl, C1-C4 monohaloalkyl, and C1-C4 polyhaloalkyl. In a further aspect, each of R^{4a}, R^{4b}, and R^{4c}, when present, is independently selected from hydrogen and C1-C4 alkyl. In a still further aspect, each of R^{4a}, R^{4b}, and R^{4c}, when present, is independently selected from hydrogen and C1-C4 monohaloalkyl. In yet a further aspect, each of R^{4a}, R^{4b}, and R^{4c}, when present, is independently selected from hydrogen and C1-C4 polyhaloalkyl. In an even further aspect, each of R^{4a}, R^{4b}, and R^{4c}, when present, is hydrogen.

In a further aspect, each of R^{4a}, R^{4b}, and R^{4c}, when present, is independently selected from hydrogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, each of R^{4a}, R^{4b}, and R^{4c}, when present, is independently selected from hydrogen, methyl, —CH₂F, —CHF₂, —CF₃, —CHCl₂, and —CCl₃. In yet a further aspect, each of R^{4a}, R^{4b}, and R^{4c}, when present, is independently selected from hydrogen, methyl, ethyl, —CH₂F, —CH₂CH₂F, —CHF₂, —CF₃, —CH₂CHF₂, and —CH₂CF₃. In an even further aspect, each of R^{4a}, R^{4b}, and R^{4c}, when present, is independently selected from hydrogen, methyl, —CH₂F, —CHF₂, and —CF₃.

g. R^{5a}, R^{5b}, and R^{5c} Groups

In one aspect, each of R^{5a}, R^{5b}, and R^{5c}, when present, is independently selected from hydrogen, C1-C4 alkyl, C1-C4 monohaloalkyl, and C1-C4 polyhaloalkyl. In a further aspect, each of R^{5a}, R^{5b}, and R^{5c}, when present, is independently selected from hydrogen and C1-C4 alkyl. In a still further aspect, each of R^{5a}, R^{5b}, and R^{5c}, when present, is independently selected from hydrogen and C1-C4 monohaloalkyl. In yet a further aspect, each of R^{5a}, R^{5b}, and R^{5c}, when present, is independently selected from hydrogen and C1-C4 polyhaloalkyl. In an even further aspect, each of R^{5a}, R^{5b}, and R^{5c}, when present, is hydrogen.

In a further aspect, each of R^{5a}, R^{5b}, and R^{5c}, when present, is independently selected from hydrogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, each of R^{5a}, R^{5b}, and R^{5c}, when present, is independently selected from hydrogen, methyl, —CH₂F, —CHF₂, —CF₃, —CHCl₂, and —CCl₃. In yet a further aspect, each of R^{5a}, R^{5b}, and R^{5c}, when present, is independently selected from hydrogen, methyl, ethyl, —CH₂F, —CH₂CH₂F, —CHF₂, —CF₃, —CH₂CHF₂, and —CH₂CF₃. In an even further aspect, each of R^{5a}, R^{5b}, and R^{5c}, when present, is independently selected from hydrogen, methyl, —CH₂F, —CHF₂, and —CF₃.

h. R⁶ Groups

In one aspect, R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴. In a further aspect, R⁶ is selected from hydrogen, —(C2-C4 alkyl)-OH, —(C2-C4 alkyl)-NH₂, —O—(C2-C4 alkyl)-OH, —O—(C2-C4 alkyl)-NH₂, —NH—(C2-C4 alkyl)-OH, and —NH—(C2-C4 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴.

In a further aspect, R⁶ is selected from —(C2-C8 alkyl)-OH, —O—(C2-C8 alkyl)-OH, —O—(C2-C8 alkyl)-NH₂, —O-Cy³, —O-Cy⁴, —OCH₂-Cy³, and —OCH₂-Cy⁴. In a still further aspect, R⁶ is selected from —(C2-C8 alkyl)-OH, —O—(C2-C8 alkyl)-OH, and —O—(C2-C8 alkyl)-NH₂. In yet a further aspect, R⁶ is —(C2-C8 alkyl)-OH. In an even further aspect, R⁶ is —O—(C2-C8 alkyl)-OH. In a still further aspect, R⁶ is —O—(C2-C8 alkyl)-NH₂.

In a further aspect, R⁶ is selected from —O-Cy³, —O-Cy⁴, —OCH₂-Cy³, and —OCH₂-Cy⁴. In a still further aspect, R⁶ is selected from —O-Cy³ and —O-Cy⁴. In yet a further aspect, R⁶ is selected from —OCH₂-Cy³ and —OCH₂-Cy⁴. In an even further aspect, R⁶ is —O-Cy³. In a still further aspect, R⁶ is —O-Cy⁴. In yet a further aspect, R⁶ is —OCH₂-Cy³. In an even further aspect, R⁶ is —OCH₂-Cy⁴.

In a further aspect, R⁶ is selected from —(C2-C8 alkyl)-NH₂, —NH—(C2-C8 alkyl)-OH, —NH—(C2-C8 alkyl)-NH₂, —NH-Cy³, —NH-Cy⁴, —NHCH₂-Cy³, and —NHCH₂-Cy⁴. In a still further aspect, R⁶ is selected from —(C2-C8 alkyl)-NH₂, —NH—(C2-C8 alkyl)-OH, and —NH—(C2-C8 alkyl)-NH₂. In yet a further aspect, R⁶ is —(C2-C8 alkyl)-NH₂. In an even further aspect, R⁶ is —NH—(C2-C8 alkyl)-OH. In a still further aspect, R⁶ is —NH—(C2-C8 alkyl)-NH₂.

In a further aspect, R⁶ is selected from —NH-Cy³, —NH-Cy⁴, —NHCH₂-Cy³, and —NHCH₂-Cy⁴. In a still further aspect, R⁶ is selected from —NH-Cy³ and —NH-Cy⁴. In yet a further aspect, R⁶ is —NH-Cy³. In an even further aspect, R⁶ is —NH-Cy⁴. In a still further aspect, R⁶ is selected from —NHCH₂-Cy³ and —NHCH₂-Cy⁴. In yet a further aspect, R⁶ is —NHCH₂-Cy³. In an even further aspect, R⁶ is —NHCH₂-Cy⁴.

In a further aspect, R⁶ is selected from —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴.

In a further aspect, R⁶ is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂.

i. R⁷ Groups

In one aspect, R⁷ is selected from Ar², -A¹-A²-Ar², and

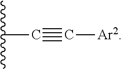

In a still further aspect, R⁷ is selected from Ar² and

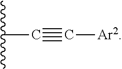

In yet a further aspect, R⁷ is selected from Ar² and -A¹-A²-Ar². In an even further aspect, R⁷ is selected from -A¹-A²-Ar² and

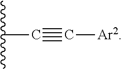

In a still further aspect, R⁷ is

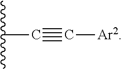

In yet a further aspect, R⁷ is -A¹-A²-Ar². In an even further aspect, R⁷ is Ar².

j. R²⁰ Groups

In one aspect, each occurrence of R²⁰, when present, is independently selected from C1-C3 alkyl, C1-C3 monohaloalkyl, C1-C3 polyhaloalkyl, and cyclopropyl. In a further aspect, each occurrence of R²⁰, when present, is independently selected from C1-C3 alkyl. In a still further aspect, each occurrence of R²⁰, when present, is independently selected from C1-C3 monohaloalkyl. In yet a further aspect, each occurrence of R²⁰, when present, is independently selected from C1-C3 polyhaloalkyl.

In a further aspect, each occurrence of R²⁰, when present, is independently selected from methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, each occurrence of R²⁰, when present, is independently selected from methyl, —CH₂F, —CHF₂, —CF₃, —CHCl₂, and —CCl₃. In yet a further aspect, each occurrence of R²⁰, when present, is independently selected from methyl, ethyl, —CH₂F, —CH₂CH₂F, —CHF₂, —CF₃, —CH₂CHF₂, and —CH₂CF₃. In an even further aspect, each occurrence of R²⁰, when present, is independently selected from methyl, —CH₂F, —CHF₂, and —CF₃.

k. R^{21a} and R^{21b} Groups

In one aspect, each occurrence of R^{21a} and R^{21b}, when present, is independently selected from hydrogen, C1-C3 alkyl, C1-C3 monohaloalkyl, C1-C3 polyhaloalkyl, and cyclopropyl. In a further aspect, each occurrence of R^{21a} and R^{21b}, when present, is independently selected from hydrogen and C1-C3 alkyl. In a still further aspect, each occurrence of R^{21a} and R^{21b}, when present, is independently selected from hydrogen and C1-C3 monohaloalkyl. In yet a further aspect, each occurrence of R^{21a} and R^{21b}, when present, is independently selected from hydrogen and C1-C4 polyhaloalkyl. In an even further aspect, each occurrence of R^{21a} and R^{21b}, when present, is hydrogen.

In a further aspect, each occurrence of R^{21a} and R^{21b}, when present, is independently selected from hydrogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, each occurrence of R^{21a} and R^{21b}, when present, is independently selected from hydrogen, methyl, —CH₂F, —CHF₂, —CF₃, —CHCl₂, and —CCl₃. In yet a further aspect, each occurrence of R^{21a} and R^{21b}, when present, is independently selected from hydrogen, methyl, ethyl, —CH₂F, —CH₂CH₂F, —CHF₂, —CF₃, —CH₂CHF₂, and —CH₂CF₃. In an even further aspect, each occurrence of R^{21a} and R^{21b}, when present, is independently selected from hydrogen, methyl, —CH₂F, —CHF₂, and —CF₃.

l. R^{30a}, R^{30b}, R^{30c}, R^{30d}, and R^{30e} Groups

In one aspect, each of R^{3a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{3a}, R^{30b}, R^{30c}, R^{30d}, and R^{30e} are hydrogen.

m. R^{40a}, R^{40b}, R^{40c}, R^{40d}, and R^{40e} Groups

In one aspect, each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

n. R^{50a}, R^{50b}, R^{50c}, R^{50d}, R^{50e}, and R^{50f} Groups

In one aspect, each of R^{50a}, R^{50b}, R^{50c}, R^{50d}, R^{50e}, and R^{50f} is independently selected from hydrogen, halogen, C1-C4 alkyl, C1-C4 monohaloalkyl, and C1-C4 polyhaloalkyl; and wherein at least three of R^{50a}, R^{50b}, R^{50c}, R^{50d}, R^{50e}, and R^{50f} are hydrogen.

o. R^{60a}, R^{60b}, R^{60c}, R^{60d}, R^{60e} and R^{60f} Groups

In one aspect, each of R^{60a}, R^{60b}, R^{60c}, R^{60d}, R^{60e}, and R^{60f} is independently selected from hydrogen, halogen, C1-C4 alkyl, C1-C4 monohaloalkyl, and C1-C4 polyhaloalkyl; and wherein at least three of R^{60a}, R^{60b}, R^{60c}, R^{60d}, R^{60e}, and R^{60f} are hydrogen.

p. Cy¹ Groups

In one aspect, Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl.

q. Cy² Groups

In one aspect, Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl. In a further aspect, Cy², when present, is a C2-C7 heterocycloalkyl comprising at least one oxygen or nitrogen atom, and wherein Cy² is substituted with 0, 1, or 2 groups independently selected from halogen, C1-C4 alkyl, C1-C4 monohaloalkyl, and C1-C4 polyhaloalkyl. In a still further aspect, Cy², when present, is a C2-C7 heterocycloalkyl comprising at least one oxygen or nitrogen atom, and wherein Cy² is substituted with 0 or 1 group selected from halogen, C1-C4 alkyl, C1-C4 monohaloalkyl, and C1-C4 polyhaloalkyl. In yet a further aspect, Cy², when present, is a C2-C7 heterocycloalkyl comprising at least one oxygen or nitrogen atom, and wherein Cy² is monosubstituted with a group selected from halogen, C1-C4 alkyl, C1-C4 monohaloalkyl, and C1-C4 polyhaloalkyl. In an even further aspect, Cy², when present, is an unsubstituted C2-C7 heterocycloalkyl comprising at least one oxygen or nitrogen atom.

In a further aspect, Cy², when present, is a C2-C6 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl. In a still further aspect, Cy², when present, is a C2-C6 heterocycloalkyl comprising at least one oxygen or nitrogen atom, and wherein Cy² is substituted with 0, 1, or 2 groups independently selected from halogen, C1-C4 alkyl, C1-C4 monohaloalkyl, and C1-C4 polyhaloalkyl. In yet a further aspect, Cy², when present, is a C2-C6 heterocycloalkyl comprising at least one oxygen or nitrogen atom, and wherein Cy² is substituted with 0 or 1 group selected from halogen, C1-C4 alkyl, C1-C4 monohaloalkyl, and C1-C4 polyhaloalkyl. In an even further aspect, Cy², when present, is a C2-C6 heterocycloalkyl comprising at least one oxygen or nitrogen atom, and wherein Cy² is monosubstituted with a group selected from halogen, C1-C4 alkyl, C1-C4 monohaloalkyl, and C1-C4 polyhaloalkyl. In a still further aspect, Cy², when present, is an unsubstituted C2-C6 heterocycloalkyl comprising at least one oxygen or nitrogen atom.

In a further aspect, Cy², when present, is a C2-C5 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl. In a still further aspect, Cy², when present, is a C2-C5 heterocycloalkyl comprising at least one oxygen or nitrogen atom, and wherein Cy² is substituted with 0, 1, or 2 groups independently selected from halogen, C1-C4 alkyl, C1-C4 monohaloalkyl, and C1-C4 polyhaloalkyl. In yet a further aspect, Cy², when present, is a C2-C5 heterocycloalkyl comprising at least one oxygen or nitrogen atom, and wherein Cy² is substituted with 0 or 1 group selected from halogen, C1-C4 alkyl, C1-C4 monohaloalkyl, and C1-C4 polyhaloalkyl. In an even further aspect, Cy², when present, is a C2-C5 heterocycloalkyl comprising at least one oxygen or nitrogen atom, and wherein Cy² is monosubstituted with a group selected from halogen, C1-C4 alkyl, C1-C4 monohaloalkyl, and C1-C4 polyhaloalkyl. In a still further aspect, Cy², when present, is an unsubstituted C2-C5 heterocycloalkyl comprising at least one oxygen or nitrogen atom.

In a further aspect, Cy², when present, is a C2-C4 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl. In a still further aspect, Cy², when present, is a C2-C4 heterocycloalkyl comprising at least one oxygen or nitrogen atom, and wherein Cy² is substituted with 0, 1, or 2 groups independently selected from halogen, C1-C4 alkyl, C1-C4 monohaloalkyl, and C1-C4 polyhaloalkyl. In yet a further aspect, Cy², when present, is a C2-C4 heterocycloalkyl comprising at least one oxygen or nitrogen atom, and wherein Cy² is substituted with 0 or 1 group selected from halogen, C1-C4 alkyl, C1-C4 monohaloalkyl, and C1-C4 polyhaloalkyl. In an even further aspect, Cy², when present, is a C2-C4 heterocycloalkyl comprising at least one oxygen or nitrogen atom, and wherein Cy² is monosubstituted with a group selected from halogen, C1-C4 alkyl, C1-C4 monohaloalkyl, and C1-C4 polyhaloalkyl. In a still further aspect, Cy², when present, is an unsubstituted C2-C4 heterocycloalkyl comprising at least one oxygen or nitrogen atom.

In a further aspect, Cy², when present, is a C4 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl. In a still further aspect, Cy², when present, is a C4 heterocycloalkyl comprising at least one oxygen or nitrogen atom, and wherein Cy² is substituted with 0, 1, or 2 groups independently selected from halogen, C1-C4 alkyl, C1-C4 monohaloalkyl, and C1-C4 polyhaloalkyl. In yet a further aspect, Cy², when present, is a C4 heterocycloalkyl comprising at least one oxygen or nitrogen atom, and wherein Cy² is substituted with 0 or 1 group selected from halogen, C1-C4 alkyl, C1-C4 monohaloalkyl, and C1-C4 polyhaloalkyl. In an even further aspect, Cy², when present, is a C4 heterocycloalkyl comprising at least one oxygen or nitrogen atom, and wherein Cy² is monosubstituted with a group selected from halogen, C1-C4 alkyl, C1-C4 monohaloalkyl, and C1-C4 polyhaloalkyl. In a still further aspect, Cy², when present, is an unsubstituted C4 heterocycloalkyl comprising at least one oxygen or nitrogen atom.

In a further aspect, Cy², when present, is pyrrolidinyl substituted with 0, 1, 2, or 3 groups independently selected from halogen, C1-C4 alkyl, C1-C4 monohaloalkyl, and C1-C4 polyhaloalkyl. In a still further aspect, Cy², when present, is pyrrolidinyl substituted with 0, 1, or 2 groups independently selected from halogen, C1-C4 alkyl, C1-C4 monohaloalkyl, and C1-C4 polyhaloalkyl. In yet a further aspect, Cy², when present, is pyrrolidinyl substituted with 0 or 1 group selected from halogen, C1-C4 alkyl, C1-C4 monohaloalkyl, and C1-C4 polyhaloalkyl. In an even further aspect, Cy², when present, is pyrrolidinyl monosubstituted with a group selected from halogen, C1-C4 alkyl, C1-C4 monohaloalkyl, and C1-C4 polyhaloalkyl. In a still further aspect, Cy², when present, is an unsubstituted pyrrolidinyl.

In a further aspect, Cy², when present, is pyrrolidinyl substituted with 0, 1, 2, or 3 groups independently selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, Cy², when present, is pyrrolidinyl substituted with 0, 1, or 2 groups independently selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In yet a further aspect, Cy², when present, is pyrrolidinyl substituted with 0 or 1 group selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In an even further aspect, Cy², when present, is pyrrolidinyl monosubstituted with a group selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃.

In a further aspect, Cy², when present, is pyrrolidinyl substituted with 0, 1, 2, or 3 groups independently selected from —F, —Cl, methyl, —CH₂F, —CH₂Cl, —CHF₂, —CF₃, —CHCl₂, and —CCl₃. In a still further aspect, Cy², when present, is pyrrolidinyl substituted with 0, 1, or 2 groups independently selected from —F, —Cl, methyl, —CH₂F, —CH₂Cl, —CHF₂, —CF₃, —CHCl₂, and —CCl₃. In yet a further aspect, Cy², when present, is pyrrolidinyl substituted with 0 or 1 group selected from —F, —Cl, methyl, —CH₂F, —CH₂Cl, —CHF₂, —CF₃, —CHCl₂, and —CCl₃. In an even further aspect, Cy², when present, is pyrrolidinyl monosubstituted with a group selected from —F, —Cl, methyl, —CH₂F, —CH₂Cl, —CHF₂, —CF₃, —CHCl₂, and —CCl₃.

In a further aspect, Cy², when present, is pyrrolidinyl substituted with 0, 1, 2, or 3 groups independently selected from —F, methyl, —CH₂F, —CHF₂, and —CF₃. In a still further aspect, Cy², when present, is pyrrolidinyl substituted with 0, 1, or 2 groups independently selected from —F, methyl, —CH₂F, —CHF₂, and —CF₃. In yet a further aspect, Cy², when present, is pyrrolidinyl substituted with 0 or 1 group selected from —F, methyl, —CH₂F, —CHF₂, and —CF₃. In an even further aspect, Cy², when present, is pyrrolidinyl monosubstituted with a group selected from —F, methyl, —CH₂F, —CHF₂, and —CF₃.

r. Cy³ Groups

In one aspect, Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl.

s. Cy⁴ Groups

In one aspect, Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl. In a further aspect, Cy⁴, when present, is a C2-C7 heterocycloalkyl comprising at least one oxygen or nitrogen atom, and wherein Cy⁴ is substituted with 0, 1, or 2 groups independently selected from halogen, C1-C4 alkyl, C1-C4 monohaloalkyl, and C1-C4 polyhaloalkyl. In a still further aspect, Cy⁴, when present, is a C2-C7 heterocycloalkyl comprising at least one oxygen or nitrogen atom, and wherein Cy⁴ is substituted with 0 or 1 group selected from halogen, C1-C4 alkyl, C1-C4 monohaloalkyl, and C1-C4 polyhaloalkyl. In yet a further aspect, Cy⁴, when present, is a C2-C7 heterocycloalkyl comprising at least one oxygen or nitrogen atom, and wherein Cy⁴ is monosubstituted with a group selected from halogen, C1-C4 alkyl, C1-C4 monohaloalkyl, and C1-C4 polyhaloalkyl. In an even further aspect, Cy⁴, when present, is an unsubstituted C2-C7 heterocycloalkyl comprising at least one oxygen or nitrogen atom.

In a further aspect, Cy⁴, when present, is a C2-C6 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl. In a still further aspect, Cy⁴, when present, is a C2-C6 heterocycloalkyl comprising at least one oxygen or nitrogen atom, and wherein Cy⁴ is substituted with 0, 1, or 2 groups independently selected from halogen, C1-C4 alkyl, C1-C4 monohaloalkyl, and C1-C4 polyhaloalkyl. In yet a further aspect, Cy⁴, when present, is a C2-C6 heterocycloalkyl comprising at least one oxygen or nitrogen atom, and wherein Cy⁴ is substituted with 0 or 1 group selected from halogen, C1-C4 alkyl, C1-C4 monohaloalkyl, and C1-C4 polyhaloalkyl. In an even further aspect, Cy⁴, when present, is a C2-C6 heterocycloalkyl comprising at least one oxygen or nitrogen atom, and wherein Cy⁴ is monosubstituted with a group selected from halogen, C1-C4 alkyl, C1-C4 monohaloalkyl, and C1-C4 polyhaloalkyl. In a still further aspect, Cy⁴, when present, is an unsubstituted C2-C6 heterocycloalkyl comprising at least one oxygen or nitrogen atom.

In a further aspect, Cy⁴, when present, is a C2-C5 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl. In a still further aspect, Cy⁴, when present, is a C2-C5 heterocycloalkyl comprising at least one oxygen or nitrogen atom, and wherein Cy⁴ is substituted with 0, 1, or 2 groups independently selected from halogen, C1-C4 alkyl, C1-C4 monohaloalkyl, and C1-C4 polyhaloalkyl. In yet a further aspect, Cy⁴, when present, is a C2-C5 heterocycloalkyl comprising at least one oxygen or nitrogen atom, and wherein Cy⁴ is substituted with 0 or 1 group selected from halogen, C1-C4 alkyl, C1-C4 monohaloalkyl, and C1-C4 polyhaloalkyl. In an even further aspect, Cy⁴, when present, is a C2-C5 heterocycloalkyl comprising at least one oxygen or nitrogen atom, and wherein Cy⁴ is monosubstituted with a group selected from halogen, C1-C4 alkyl, C1-C4 monohaloalkyl, and C1-C4 polyhaloalkyl. In a still further aspect, Cy⁴, when present, is an unsubstituted C2-C5 heterocycloalkyl comprising at least one oxygen or nitrogen atom.

In a further aspect, Cy⁴, when present, is a C2-C4 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl. In a still further aspect, Cy⁴, when present, is a C2-C4 heterocycloalkyl comprising at least one oxygen or nitrogen atom, and wherein Cy⁴ is substituted with 0, 1, or 2 groups independently selected from halogen, C1-C4 alkyl, C1-C4 monohaloalkyl, and C1-C4 polyhaloalkyl. In yet a further aspect, Cy⁴, when present, is a C2-C4 heterocycloalkyl comprising at least one oxygen or nitrogen atom, and wherein Cy⁴ is substituted with 0 or 1 group selected from halogen, C1-C4 alkyl, C1-C4 monohaloalkyl, and C1-C4 polyhaloalkyl. In an even further aspect, Cy⁴, when present, is a C2-C4 heterocycloalkyl comprising at least one oxygen or nitrogen atom, and wherein Cy⁴ is monosubstituted with a group selected from halogen, C1-C4 alkyl, C1-C4 monohaloalkyl, and C1-C4 polyhaloalkyl. In a still further aspect, Cy⁴, when present, is an unsubstituted C2-C4 heterocycloalkyl comprising at least one oxygen or nitrogen atom.

In a further aspect, Cy⁴, when present, is a C4 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl. In a still further aspect, Cy⁴, when present, is a C4 heterocycloalkyl comprising at least one oxygen or nitrogen atom, and wherein Cy⁴ is substituted with 0, 1, or 2 groups independently selected from halogen, C1-C4 alkyl, C1-C4 monohaloalkyl, and C1-C4 polyhaloalkyl. In yet a further aspect, Cy⁴, when present, is a C4 heterocycloalkyl comprising at least one oxygen or nitrogen atom, and wherein Cy⁴ is substituted with 0 or 1 group selected from halogen, C1-C4 alkyl, C1-C4 monohaloalkyl, and C1-C4 polyhaloalkyl. In an even further aspect, Cy⁴, when present, is a C4 heterocycloalkyl comprising at least one oxygen or nitrogen atom, and wherein Cy⁴ is monosubstituted with a group selected from halogen, C1-C4 alkyl, C1-C4 monohaloalkyl, and C1-C4 polyhaloalkyl. In a still further aspect, Cy⁴, when present, is an unsubstituted C4 heterocycloalkyl comprising at least one oxygen or nitrogen atom.

In a further aspect, Cy⁴, when present, is pyrrolidinyl substituted with 0, 1, 2, or 3 groups independently selected from halogen, C1-C4 alkyl, C1-C4 monohaloalkyl, and C1-C4 polyhaloalkyl. In a still further aspect, Cy⁴, when present, is pyrrolidinyl substituted with 0, 1, or 2 groups independently selected from halogen, C1-C4 alkyl, C1-C4 monohaloalkyl, and C1-C4 polyhaloalkyl. In yet a further aspect, Cy⁴, when present, is pyrrolidinyl substituted with 0 or 1 group selected from halogen, C1-C4 alkyl, C1-C4 monohaloalkyl, and C1-C4 polyhaloalkyl. In an even further aspect, Cy⁴, when present, is pyrrolidinyl monosubstituted with a group selected from halogen, C1-C4 alkyl, C1-C4 monohaloalkyl, and C1-C4 polyhaloalkyl. In a still further aspect, Cy⁴, when present, is an unsubstituted pyrrolidinyl.

In a further aspect, Cy⁴, when present, is pyrrolidinyl substituted with 0, 1, 2, or 3 groups independently selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In a still further aspect, Cy⁴, when present, is pyrrolidinyl substituted with 0, 1, or 2 groups independently selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In yet a further aspect, Cy⁴, when present, is pyrrolidinyl substituted with 0 or 1 group selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃. In an even further aspect, Cy⁴, when present, is pyrrolidinyl monosubstituted with a group selected from halogen, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, and —CH₂CCl₃.

In a further aspect, Cy⁴, when present, is pyrrolidinyl substituted with 0, 1, 2, or 3 groups independently selected from —F, —Cl, methyl, —CH₂F, —CH₂Cl, —CHF₂, —CF₃, —CHCl₂, and —CCl₃. In a still further aspect, Cy⁴, when present, is pyrrolidinyl substituted with 0, 1, or 2 groups independently selected from —F, —Cl, methyl, —CH₂F, —CH₂Cl, —CHF₂, —CF₃, —CHCl₂, and —CCl₃. In yet a further aspect, Cy⁴, when present, is pyrrolidinyl substituted with 0 or 1 group selected from —F, —Cl, methyl, —CH₂F, —CH₂Cl, —CHF₂, —CF₃, —CHCl₂, and —CCl₃. In an even further aspect, Cy⁴, when present, is pyrrolidinyl monosubstituted with a group selected from —F, —Cl, methyl, —CH₂F, —CH₂Cl, —CHF₂, —CF₃, —CHCl₂, and —CCl₃.

In a further aspect, Cy⁴, when present, is pyrrolidinyl substituted with 0, 1, 2, or 3 groups independently selected from —F, methyl, —CH₂F, —CHF₂, and —CF₃. In a still further aspect, Cy⁴, when present, is pyrrolidinyl substituted with 0, 1, or 2 groups independently selected from —F, methyl, —CH₂F, —CHF₂, and —CF₃. In yet a further aspect, Cy⁴, when present, is pyrrolidinyl substituted with 0 or 1 group selected from —F, methyl, —CH₂F, —CHF₂, and —CF₃. In an even further aspect, Cy⁴, when present, is pyrrolidinyl monosubstituted with a group selected from —F, methyl, —CH₂F, —CHF₂, and —CF₃.

t. Ar¹ Groups

In one aspect, Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a further aspect, Ar¹ is selected from aryl and heteroaryl, and wherein Ar¹ is substituted with 0, 1, or 2 groups independently selected from halogen, —CN, C1-C3 alkyl, C1-C3 monohaloalkyl, C1-C3 polyhaloalkyl, cyclopropyl, and —CO₂H. In a still further aspect, Ar¹ is selected from aryl and heteroaryl, and wherein Ar¹ is substituted with 0 or 1 group selected from halogen, —CN, C1-C3 alkyl, C1-C3 monohaloalkyl, C1-C3 polyhaloalkyl, cyclopropyl, and —CO₂H. In yet a further aspect, Ar¹ is selected from aryl and heteroaryl, and wherein Ar¹ is monosubstituted with a group selected from halogen, —CN, C1-C3 alkyl, C1-C3 monohaloalkyl, C1-C3 polyhaloalkyl, cyclopropyl, and —CO₂H. In an even further aspect, Ar¹ is selected from aryl and heteroaryl, and wherein Ar¹ is unsubstituted.

In a further aspect, Ar¹ is selected from phenyl, naphthyl, pyridinyl, pyrimidinyl, pyridazinyl, and pyrazinyl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar¹ is selected from phenyl, naphthyl, pyridinyl, pyrimidinyl, pyridazinyl, and pyrazinyl, and wherein Ar¹ is substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar¹ is selected from phenyl, naphthyl, pyridinyl, pyrimidinyl, pyridazinyl, and pyrazinyl, and wherein Ar¹ is substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar¹ is selected from phenyl, naphthyl, pyridinyl, pyrimidinyl, pyridazinyl, and pyrazinyl, and wherein Ar¹ is monosubstituted with a group selected from halogen, —CN, C1-C3 alkyl, C1-C3 monohaloalkyl, C1-C3 polyhaloalkyl, cyclopropyl, and —CO₂H. In a still further aspect, Ar¹ is selected from phenyl, naphthyl, pyridinyl, pyrimidinyl, pyridazinyl, and pyrazinyl, and wherein Ar¹ is unsubstituted.

In a further aspect, Ar¹ is selected from phenyl and pyridinyl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar¹ is selected from phenyl and pyridinyl, and wherein Ar¹ is substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar¹ is selected from phenyl and pyridinyl, and wherein Ar¹ is substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar¹ is selected from phenyl and pyridinyl, and wherein Ar¹ is monosubstituted with a group selected from halogen, —CN, C1-C3 alkyl, C1-C3 monohaloalkyl, C1-C3 polyhaloalkyl, cyclopropyl, and —CO₂H. In a still further aspect, Ar¹ is selected from phenyl and pyridinyl, and wherein Ar¹ is unsubstituted.

In a further aspect, Ar¹ is phenyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar¹ is phenyl substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar¹ is phenyl substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar¹ is phenyl monosubstituted with a group selected from halogen, —CN, C1-C3 alkyl, C1-C3 monohaloalkyl, C1-C3 polyhaloalkyl, cyclopropyl, and —CO₂H. In a still further aspect, Ar¹ is unsubstituted phenyl.

In a further aspect, Ar¹ is pyridinyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar¹ is pyridinyl substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar¹ is pyridinyl substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar¹ is pyridinyl monosubstituted with a group selected from halogen, —CN, C1-C3 alkyl, C1-C3 monohaloalkyl, C1-C3 polyhaloalkyl, cyclopropyl, and —CO₂H. In a still further aspect, Ar¹ is unsubstituted pyridinyl.

In a further aspect, Ar¹ is selected from phenyl and pyridinyl, and wherein Ar¹ is substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar¹ is selected from phenyl and pyridinyl, and wherein Ar¹ is substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar¹ is selected from phenyl and pyridinyl, and wherein Ar¹ is substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar¹ is selected from phenyl and pyridinyl, and wherein Ar¹ is monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

In a further aspect, Ar¹ is phenyl substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar¹ is phenyl substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar¹ is phenyl substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar¹ is phenyl monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

In a further aspect, Ar¹ is pyridinyl substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar¹ is pyridinyl substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar¹ is pyridinyl substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar¹ is pyridinyl monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

u. Ar² Groups

In one aspect, Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a further aspect, Ar² is selected from aryl and heteroaryl, and wherein Ar² is substituted with 0, 1, or 2 groups independently selected from halogen, —CN, C1-C3 alkyl, C1-C3 monohaloalkyl, C1-C3 polyhaloalkyl, cyclopropyl, and —CO₂H. In a still further aspect, Ar² is selected from aryl and heteroaryl, and wherein Ar² is substituted with 0 or 1 group selected from halogen, —CN, C1-C3 alkyl, C1-C3 monohaloalkyl, C1-C3 polyhaloalkyl, cyclopropyl, and —CO₂H. In yet a further aspect, Ar² is selected from aryl and heteroaryl, and wherein Ar² is monosubstituted with a group selected from halogen, —CN, C1-C3 alkyl, C1-C3 monohaloalkyl, C1-C3 polyhaloalkyl, cyclopropyl, and —CO₂H. In an even further aspect, Ar² is selected from aryl and heteroaryl, and wherein Ar² is unsubstituted.

In a further aspect, Ar² is selected from phenyl, naphthyl, pyridinyl, pyrimidinyl, pyridazinyl, and pyrazinyl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar² is selected from phenyl, naphthyl, pyridinyl, pyrimidinyl, pyridazinyl, and pyrazinyl, and wherein Ar² is substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar² is selected from phenyl, naphthyl, pyridinyl, pyrimidinyl, pyridazinyl, and pyrazinyl, and wherein Ar² is substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar² is selected from phenyl, naphthyl, pyridinyl, pyrimidinyl, pyridazinyl, and pyrazinyl, and wherein Ar² is monosubstituted with a group selected from halogen, —CN, C1-C3 alkyl, C1-C3 monohaloalkyl, C1-C3 polyhaloalkyl, cyclopropyl, and —CO₂H. In a still further aspect, Ar² is selected from phenyl, naphthyl, pyridinyl, pyrimidinyl, pyridazinyl, and pyrazinyl, and wherein Ar² is unsubstituted.

In a further aspect, Ar² is selected from phenyl and pyridinyl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar² is selected from phenyl and pyridinyl, and wherein Ar² is substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar² is selected from phenyl and pyridinyl, and wherein Ar² is substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar² is selected from phenyl and pyridinyl, and wherein Ar² is monosubstituted with a group selected from halogen, —CN, C1-C3 alkyl, C1-C3 monohaloalkyl, C1-C3 polyhaloalkyl, cyclopropyl, and —CO₂H. In a still further aspect, Ar² is selected from phenyl and pyridinyl, and wherein Ar² is unsubstituted.

In a further aspect, Ar² is phenyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar² is phenyl substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar² is phenyl substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar² is phenyl monosubstituted with a group selected from halogen, —CN, C1-C3 alkyl, C1-C3 monohaloalkyl, C1-C3 polyhaloalkyl, cyclopropyl, and —CO₂H. In a still further aspect, Ar² is unsubstituted phenyl.

In a further aspect, Ar² is pyridinyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H. In a still further aspect, Ar² is pyridinyl substituted with 0, 1, or 2 groups independently selected from halogen, —CN, C1-C3 alkyl, C1-C3 monohaloalkyl, C1-C3 polyhaloalkyl, cyclopropyl, and —CO₂H. In yet a further aspect, Ar² is pyridinyl substituted with 0 or 1 group selected from halogen, —CN, C1-C3 alkyl, C1-C3 monohaloalkyl, C1-C3 polyhaloalkyl, cyclopropyl, and —CO₂H. In an even further aspect, Ar² is pyridinyl monosubstituted with a group selected from halogen, —CN, C1-C3 alkyl, C1-C3 monohaloalkyl, C1-C3 polyhaloalkyl, cyclopropyl, and —CO₂H. In a still further aspect, Ar² is unsubstituted pyridinyl.

In a further aspect, Ar² is selected from phenyl and pyridinyl, and wherein Ar² is substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar² is selected from phenyl and pyridinyl, and wherein Ar² is substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar² is selected from phenyl and pyridinyl, and wherein Ar² is substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar² is selected from phenyl and pyridinyl, and wherein Ar² is monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

In a further aspect, Ar² is phenyl substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar² is phenyl substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar² is phenyl substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar² is phenyl monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

In a further aspect, Ar² is pyridinyl substituted with 0, 1, 2, or 3 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In a still further aspect, Ar² is pyridinyl substituted with 0, 1, or 2 groups independently selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In yet a further aspect, Ar² is pyridinyl substituted with 0 or 1 group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H. In an even further aspect, Ar² is pyridinyl monosubstituted with a group selected from halogen, —CN, methyl, ethyl, —CH₂F, —CH₂Cl, —CH₂CH₂F, —CH₂CH₂Cl, —CHF₂, —CF₃, —CHCl₂, —CCl₃, —CH₂CHF₂, —CH₂CF₃, —CH₂CHCl₂, —CH₂CCl₃. and —CO₂H.

### 2. Example Compounds

In one aspect, a compound can be present as:

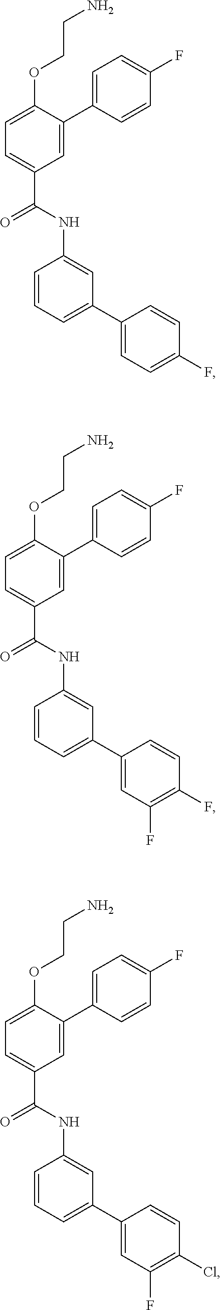 [SMILES: NCCOc1ccc(C(=O)Nc2cccc(-c3ccc(F)cc3)c2)cc1-c1ccc(F)cc1 ; NCCOc1ccc(C(=O)Nc2cccc(-c3ccc(F)c(F)c3)c2)cc1-c1ccc(F)cc1 ; NCCOc1ccc(C(=O)Nc2cccc(-c3ccc(Cl)c(F)c3)c2)cc1-c1ccc(F)cc1]

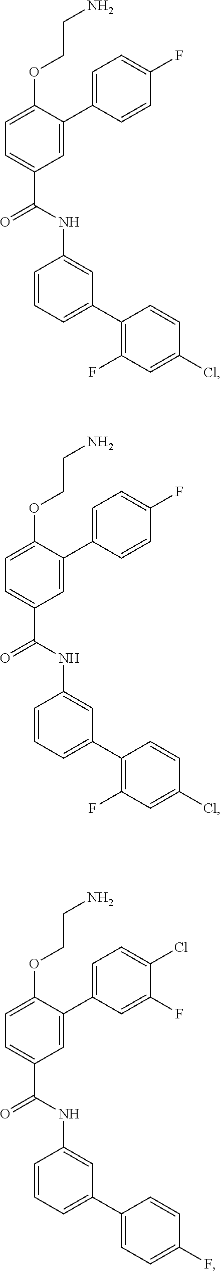 [SMILES: NCCOc1ccc(C(=O)Nc2cccc(-c3ccc(Cl)cc3F)c2)cc1-c1ccc(F)cc1 ; NCCOc1ccc(C(=O)Nc2cccc(-c3ccc(Cl)cc3F)c2)cc1-c1ccc(F)cc1 ; NCCOc1ccc(C(=O)Nc2cccc(-c3ccc(F)cc3)c2)cc1-c1ccc(Cl)c(F)c1]

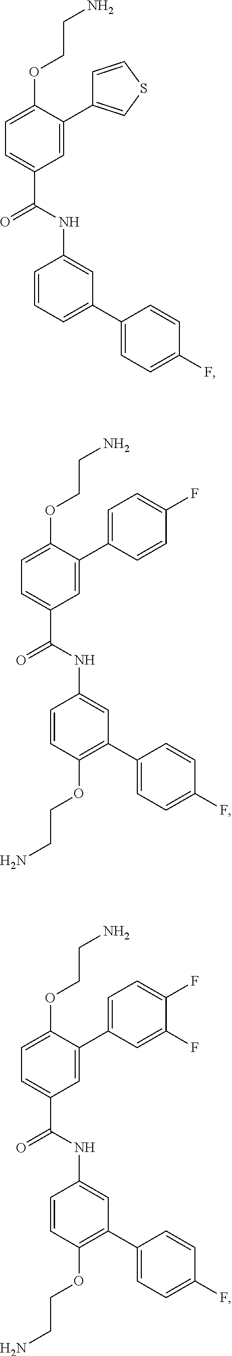 [SMILES: NCCOc1ccc(C(=O)Nc2cccc(-c3ccc(F)cc3)c2)cc1-c1ccsc1 ; NCCOc1ccc(NC(=O)c2ccc(OCCN)c(-c3ccc(F)cc3)c2)cc1-c1ccc(F)cc1 ; NCCOc1ccc(NC(=O)c2ccc(OCCN)c(-c3ccc(F)c(F)c3)c2)cc1-c1ccc(F)cc1]

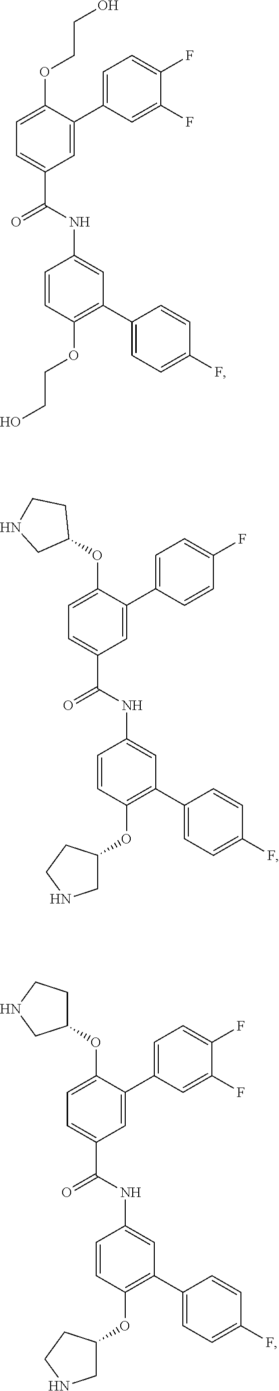 [SMILES: O=C(Nc1ccc(OCCO)c(-c2ccc(F)cc2)c1)c1ccc(OCCO)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc(F)cc2)c1 ; O=C(Nc1ccc(O[C@H]2CCNC2)c(-c2ccc(F)cc2)c1)c1ccc(O[C@H]2CCNC2)c(-c2ccc(F)c(F)c2)c1]

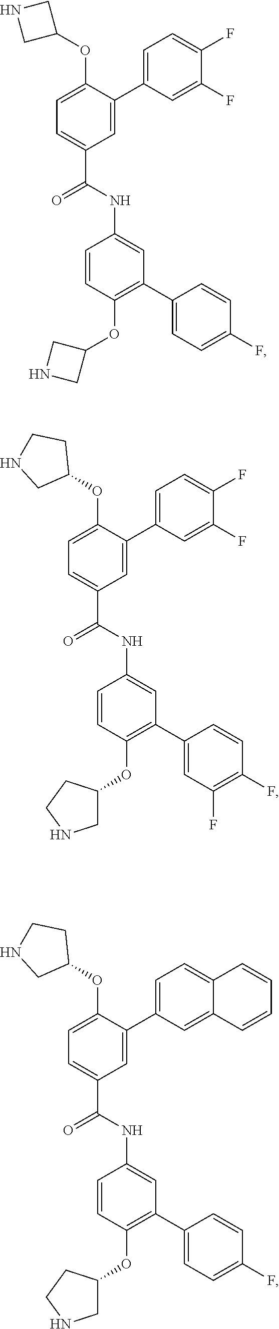 [SMILES: O=C(Nc1ccc(OC2CNC2)c(-c2ccc(F)cc2)c1)c1ccc(OC2CNC2)c(-c2ccc(F)c(F)c2)c1 ; O=C(Nc1ccc(O[C@H]2CCNC2)c(-c2ccc(F)c(F)c2)c1)c1ccc(O[C@H]2CCNC2)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc3ccccc3c2)c1]

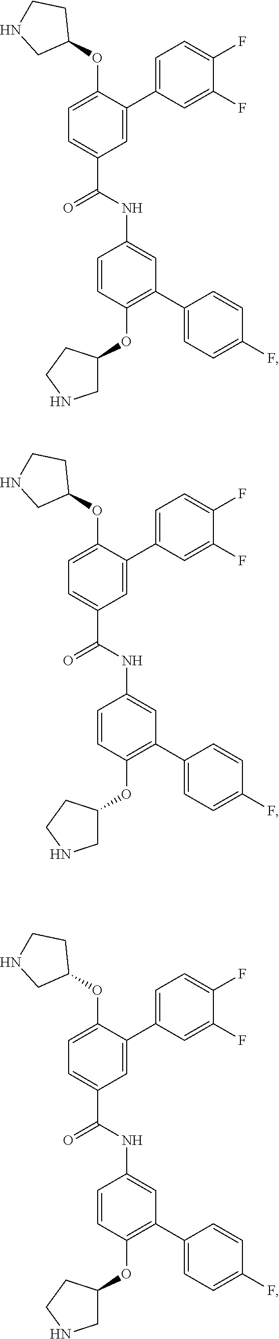 [SMILES: O=C(Nc1ccc(O[C@@H]2CCNC2)c(-c2ccc(F)cc2)c1)c1ccc(O[C@@H]2CCNC2)c(-c2ccc(F)c(F)c2)c1 ; O=C(Nc1ccc(O[C@H]2CCNC2)c(-c2ccc(F)cc2)c1)c1ccc(O[C@@H]2CCNC2)c(-c2ccc(F)c(F)c2)c1 ; O=C(Nc1ccc(O[C@@H]2CCNC2)c(-c2ccc(F)cc2)c1)c1ccc(O[C@H]2CCNC2)c(-c2ccc(F)c(F)c2)c1]

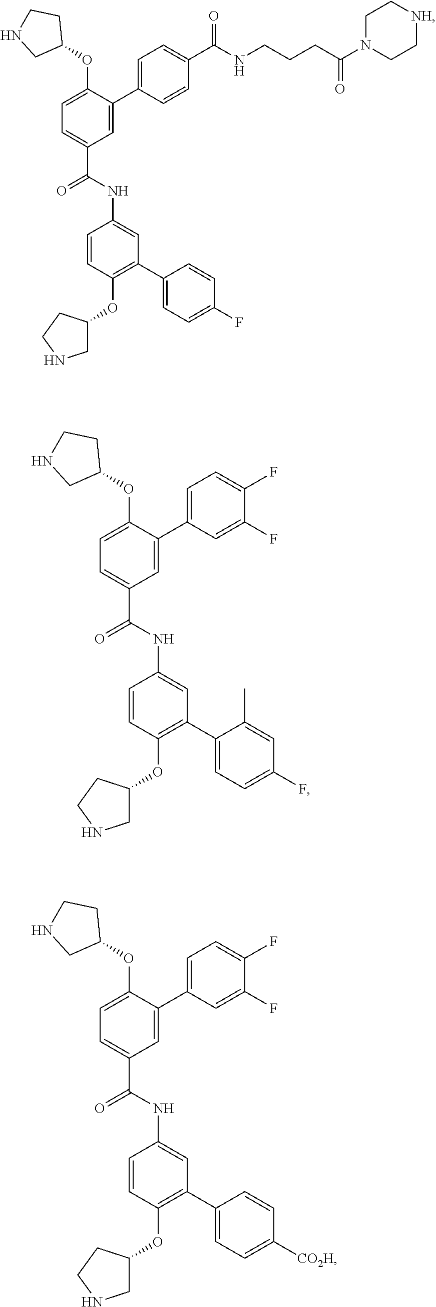 [SMILES: O=C(NCCCC(=O)N1CCNCC1)c1ccc(-c2cc(C(=O)Nc3ccc(O[C@H]4CCNC4)c(-c4ccc(F)cc4)c3)ccc2O[C@H]2CCNC2)cc1 ; Cc1cc(F)ccc1-c1cc(NC(=O)c2ccc(O[C@H]3CCNC3)c(-c3ccc(F)c(F)c3)c2)ccc1O[C@H]1CCNC1 ; O=C(O)c1ccc(-c2cc(NC(=O)c3ccc(O[C@H]4CCNC4)c(-c4ccc(F)c(F)c4)c3)ccc2O[C@H]2CCNC2)cc1]

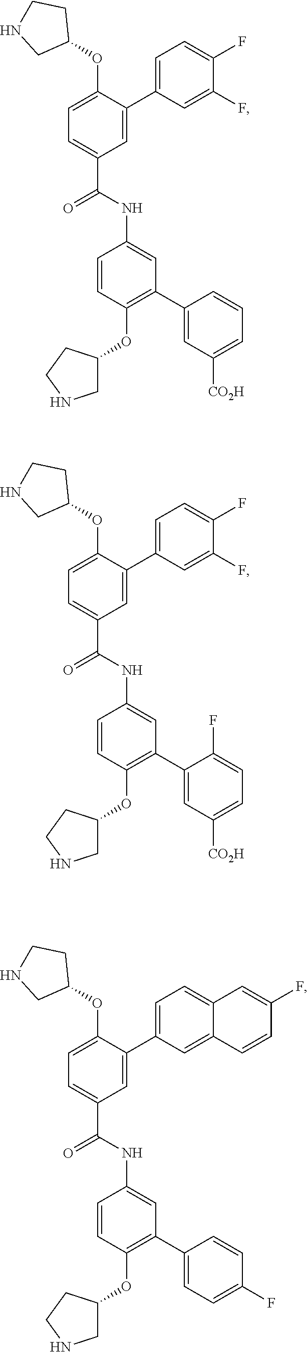 [SMILES: O=C(O)c1cccc(-c2cc(NC(=O)c3ccc(O[C@H]4CCNC4)c(-c4ccc(F)c(F)c4)c3)ccc2O[C@H]2CCNC2)c1 ; O=C(O)c1ccc(F)c(-c2cc(NC(=O)c3ccc(O[C@H]4CCNC4)c(-c4ccc(F)c(F)c4)c3)ccc2O[C@H]2CCNC2)c1 ; O=C(Nc1ccc(O[C@H]2CCNC2)c(-c2ccc(F)cc2)c1)c1ccc(O[C@H]2CCNC2)c(-c2ccc3cc(F)ccc3c2)c1]

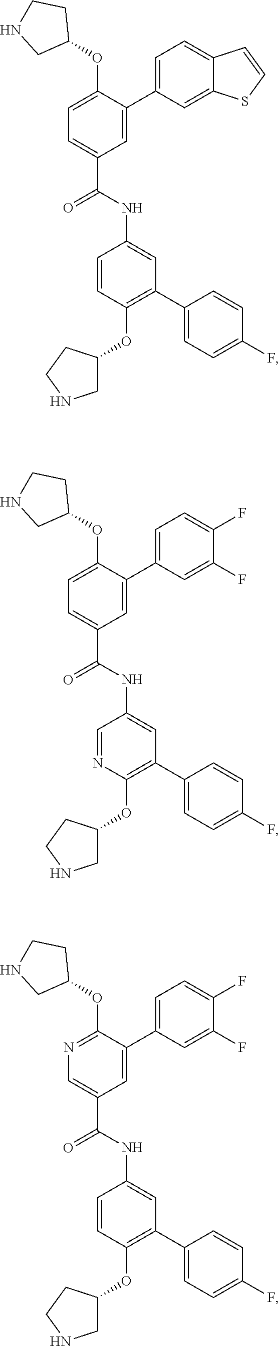 [SMILES: O=C(Nc1ccc(O[C@H]2CCNC2)c(-c2ccc(F)cc2)c1)c1ccc(O[C@H]2CCNC2)c(-c2ccc3ccsc3c2)c1 ; O=C(Nc1cnc(O[C@H]2CCNC2)c(-c2ccc(F)cc2)c1)c1ccc(O[C@H]2CCNC2)c(-c2ccc(F)c(F)c2)c1 ; O=C(Nc1ccc(O[C@H]2CCNC2)c(-c2ccc(F)cc2)c1)c1cnc(O[C@H]2CCNC2)c(-c2ccc(F)c(F)c2)c1]

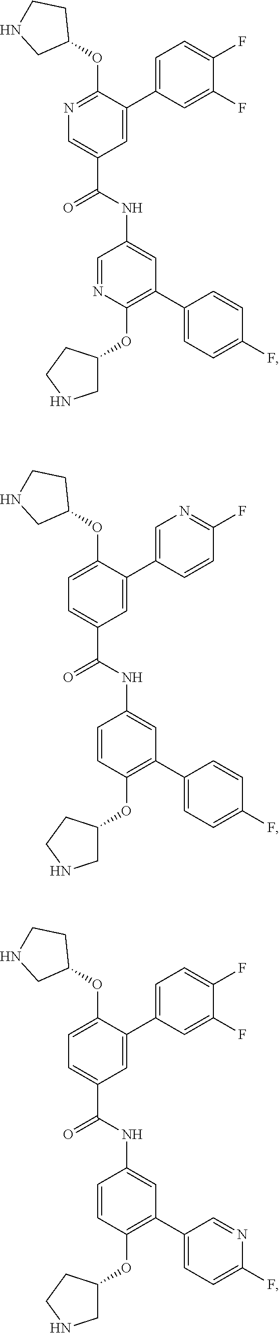 [SMILES: O=C(Nc1cnc(O[C@H]2CCNC2)c(-c2ccc(F)cc2)c1)c1cnc(O[C@H]2CCNC2)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc(F)nc2)c1 ; O=C(Nc1ccc(O[C@H]2CCNC2)c(-c2ccc(F)nc2)c1)c1ccc(O[C@H]2CCNC2)c(-c2ccc(F)c(F)c2)c1]

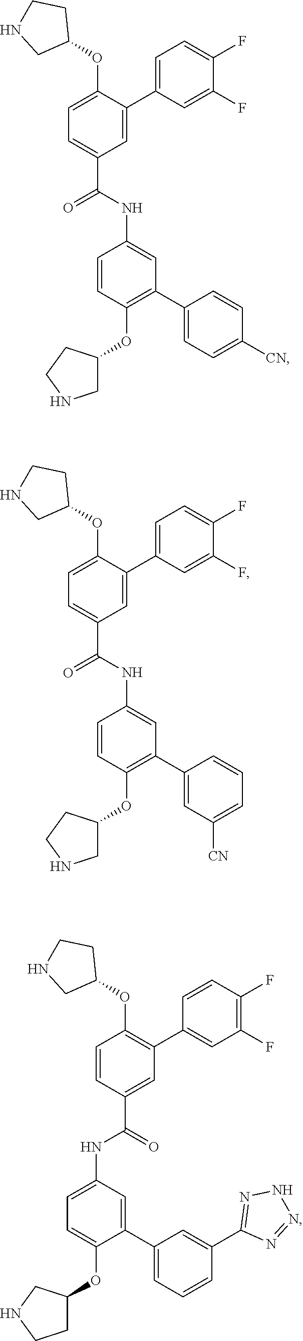 [SMILES: N#Cc1ccc(-c2cc(NC(=O)c3ccc(O[C@H]4CCNC4)c(-c4ccc(F)c(F)c4)c3)ccc2O[C@H]2CCNC2)cc1 ; N#Cc1cccc(-c2cc(NC(=O)c3ccc(O[C@H]4CCNC4)c(-c4ccc(F)c(F)c4)c3)ccc2O[C@H]2CCNC2)c1 ; O=C(Nc1ccc(O[C@H]2CCNC2)c(-c2cccc(-c3nn[nH]n3)c2)c1)c1ccc(O[C@H]2CCNC2)c(-c2ccc(F)c(F)c2)c1]

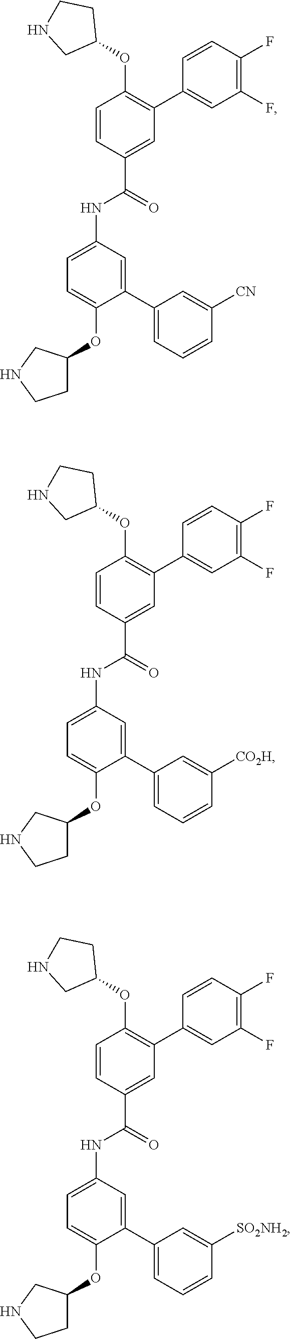 [SMILES: N#Cc1cccc(-c2cc(NC(=O)c3ccc(O[C@H]4CCNC4)c(-c4ccc(F)c(F)c4)c3)ccc2O[C@H]2CCNC2)c1 ; O=C(O)c1cccc(-c2cc(NC(=O)c3ccc(O[C@H]4CCNC4)c(-c4ccc(F)c(F)c4)c3)ccc2O[C@H]2CCNC2)c1 ; NS(=O)(=O)c1cccc(-c2cc(NC(=O)c3ccc(O[C@H]4CCNC4)c(-c4ccc(F)c(F)c4)c3)ccc2O[C@H]2CCNC2)c1]

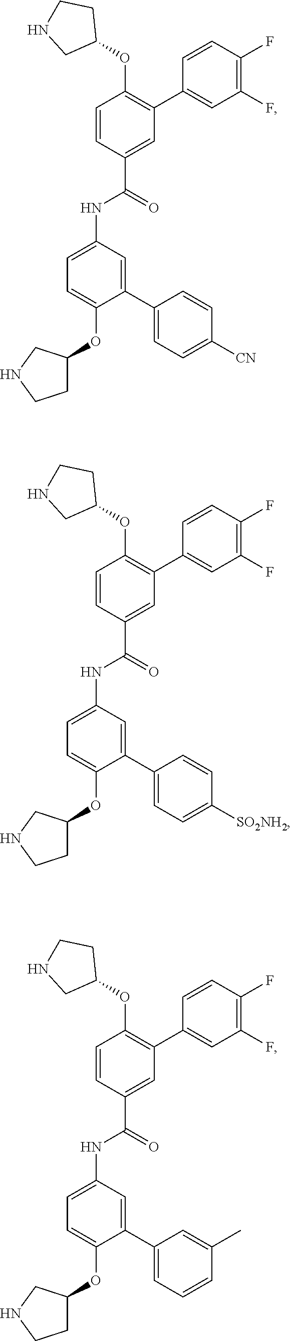 [SMILES: N#Cc1ccc(-c2cc(NC(=O)c3ccc(O[C@H]4CCNC4)c(-c4ccc(F)c(F)c4)c3)ccc2O[C@H]2CCNC2)cc1 ; NS(=O)(=O)c1ccc(-c2cc(NC(=O)c3ccc(O[C@H]4CCNC4)c(-c4ccc(F)c(F)c4)c3)ccc2O[C@H]2CCNC2)cc1 ; Cc1cccc(-c2cc(NC(=O)c3ccc(O[C@H]4CCNC4)c(-c4ccc(F)c(F)c4)c3)ccc2O[C@H]2CCNC2)c1]

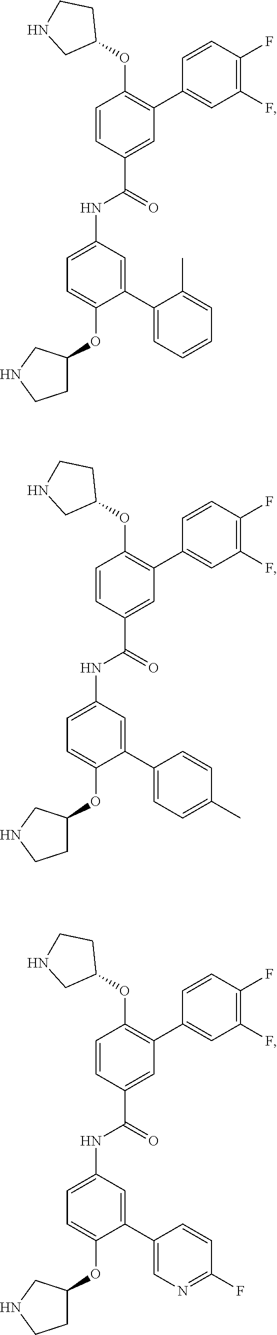 [SMILES: Cc1ccccc1-c1cc(NC(=O)c2ccc(O[C@H]3CCNC3)c(-c3ccc(F)c(F)c3)c2)ccc1O[C@H]1CCNC1 ; Cc1ccc(-c2cc(NC(=O)c3ccc(O[C@H]4CCNC4)c(-c4ccc(F)c(F)c4)c3)ccc2O[C@H]2CCNC2)cc1 ; O=C(Nc1ccc(O[C@H]2CCNC2)c(-c2ccc(F)nc2)c1)c1ccc(O[C@H]2CCNC2)c(-c2ccc(F)c(F)c2)c1]

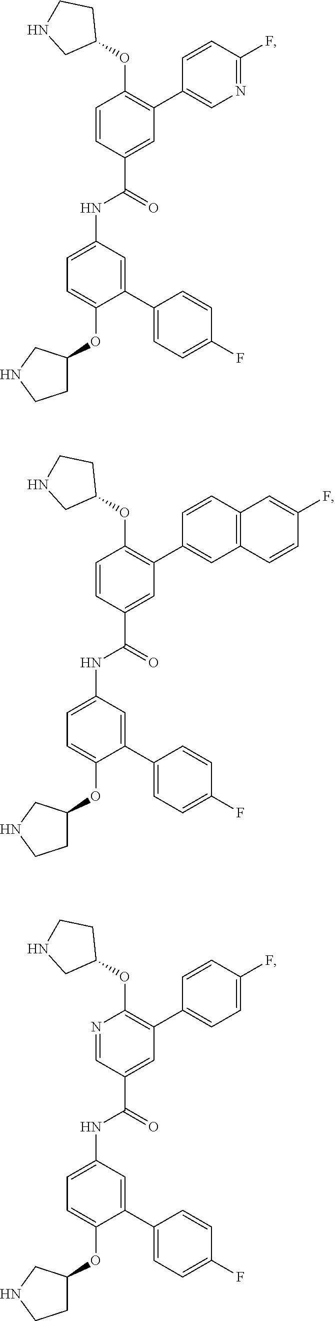 [SMILES: O=C(Nc1ccc(O[C@H]2CCNC2)c(-c2ccc(F)cc2)c1)c1ccc(O[C@H]2CCNC2)c(-c2ccc(F)nc2)c1 ; O=C(Nc1ccc(O[C@H]2CCNC2)c(-c2ccc(F)cc2)c1)c1ccc(O[C@H]2CCNC2)c(-c2ccc3cc(F)ccc3c2)c1 ; O=C(Nc1ccc(O[C@H]2CCNC2)c(-c2ccc(F)cc2)c1)c1cnc(O[C@H]2CCNC2)c(-c2ccc(F)cc2)c1]

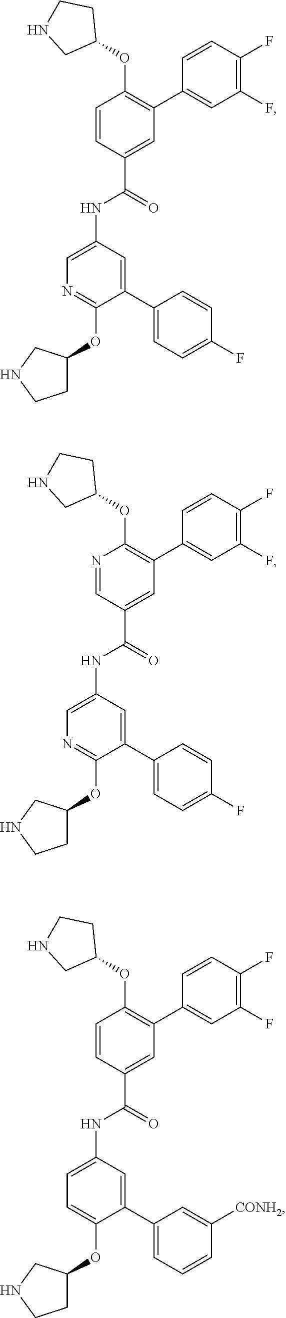 [SMILES: O=C(Nc1cnc(O[C@H]2CCNC2)c(-c2ccc(F)cc2)c1)c1ccc(O[C@H]2CCNC2)c(-c2ccc(F)c(F)c2)c1 ; O=C(Nc1cnc(O[C@H]2CCNC2)c(-c2ccc(F)cc2)c1)c1cnc(O[C@H]2CCNC2)c(-c2ccc(F)c(F)c2)c1 ; NC(=O)c1cccc(-c2cc(NC(=O)c3ccc(O[C@H]4CCNC4)c(-c4ccc(F)c(F)c4)c3)ccc2O[C@H]2CCNC2)c1]

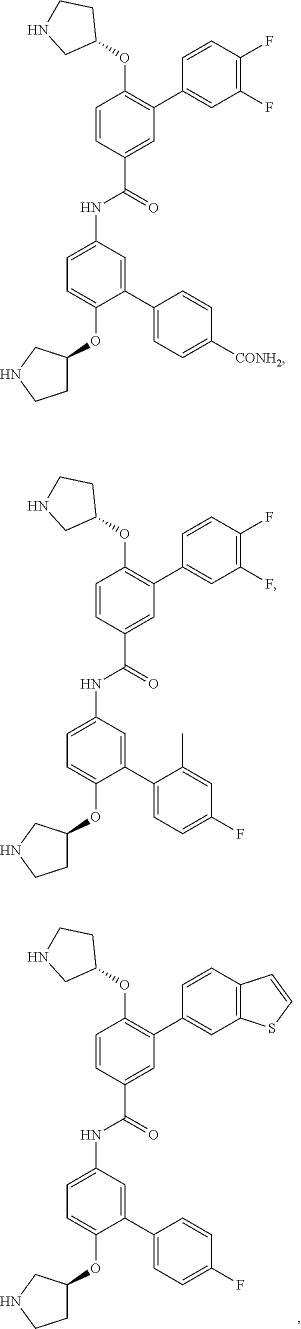 [SMILES: NC(=O)c1ccc(-c2cc(NC(=O)c3ccc(O[C@H]4CCNC4)c(-c4ccc(F)c(F)c4)c3)ccc2O[C@H]2CCNC2)cc1 ; Cc1cc(F)ccc1-c1cc(NC(=O)c2ccc(O[C@H]3CCNC3)c(-c3ccc(F)c(F)c3)c2)ccc1O[C@H]1CCNC1 ; O=C(Nc1ccc(O[C@H]2CCNC2)c(-c2ccc(F)cc2)c1)c1ccc(O[C@H]2CCNC2)c(-c2ccc3ccsc3c2)c1]

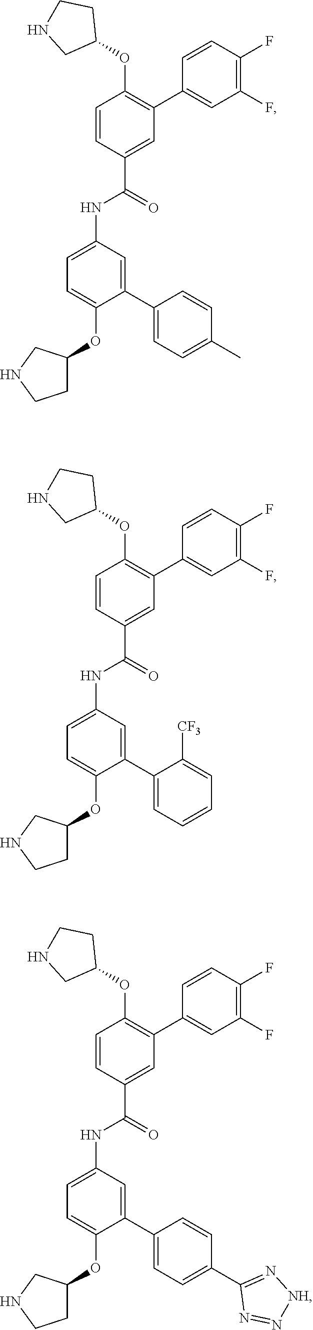 [SMILES: Cc1ccc(-c2cc(NC(=O)c3ccc(O[C@H]4CCNC4)c(-c4ccc(F)c(F)c4)c3)ccc2O[C@H]2CCNC2)cc1 ; O=C(Nc1ccc(O[C@H]2CCNC2)c(-c2ccccc2C(F)(F)F)c1)c1ccc(O[C@H]2CCNC2)c(-c2ccc(F)c(F)c2)c1 ; O=C(Nc1ccc(O[C@H]2CCNC2)c(-c2ccc(-c3nn[nH]n3)cc2)c1)c1ccc(O[C@H]2CCNC2)c(-c2ccc(F)c(F)c2)c1]

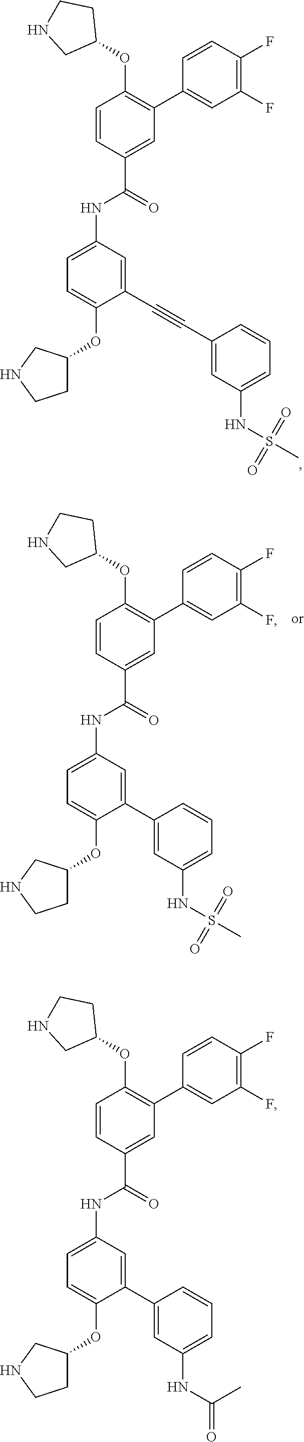 [SMILES: CS(=O)(=O)Nc1cccc(C#Cc2cc(NC(=O)c3ccc(O[C@H]4CCNC4)c(-c4ccc(F)c(F)c4)c3)ccc2O[C@@H]2CCNC2)c1 ; CS(=O)(=O)Nc1cccc(-c2cc(NC(=O)c3ccc(O[C@H]4CCNC4)c(-c4ccc(F)c(F)c4)c3)ccc2O[C@@H]2CCNC2)c1 ; "or" ; CC(=O)Nc1cccc(-c2cc(NC(=O)c3ccc(O[C@H]4CCNC4)c(-c4ccc(F)c(F)c4)c3)ccc2O[C@@H]2CCNC2)c1]

or a subgroup thereof.

### C. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound, or a pharmaceutically acceptable salt thereof, or at least one product of a disclosed method, and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require inhibition of β-catenin/BCL9 protein-protein interactions an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for inhibiting β-catenin/BCL9 protein-protein interactions (e.g., treatment of one or more disorders of uncontrolled cellular proliferation associated with β-catenin/BCL9 protein-protein interaction dysfunction or Wnt dysregulation) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

In a further aspect, the pharmaceutical composition further comprises a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the pharmaceutical composition further comprises a chemotherapeutic agent. In a still further aspect, the chemotherapeutic agent is selected from one or more of the group consisting of an alkylating agent, an antimetabolite agent, an antineoplastic antibiotic agent, a mitotic inhibitor agent, a mTor inhibitor agent or other chemotherapeutic agent, or a pharmaceutically acceptable salt thereof. In yet a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof. In an even further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof. In a still further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof. In yet a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof. In an even further aspect, the mTor inhibitor agent is selected from one or more of the group consisting of everolimus, siroliumus, and temsirolimus, or a pharmaceutically acceptable salt thereof.

It is understood that the disclosed pharmaceutical compositions can be prepared from the disclosed compounds. It is also understood that the disclosed pharmaceutical compositions can be employed in the disclosed methods of using.

### D. METHODS OF USING THE COMPOUNDS AND COMPOSITIONS

The pharmaceutical compositions and methods of the present invention can further comprise other therapeutically active compounds as noted herein which are usually applied in the treatment of the above mentioned pathological conditions.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

1. Treatment Methods

The compounds disclosed herein are useful for treating, preventing, ameliorating, controlling or reducing the risk of a variety of disorders wherein the patient or subject would benefit from inhibition or negative modulation of β-catenin/BCL9 protein-protein interaction. In one aspect, a treatment can include selective inhibition of β-catenin/BCL9 protein-protein interaction to an extent effective to effect down-regulation of Wnt pathway signaling activity. In one aspect, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which f-catenin/BCL9 protein-protein interaction inhibition is predicted to be beneficial, in a subject comprising the step of administering to the subject at least one disclosed compound; at least one disclosed pharmaceutical composition; and/or at least one disclosed product in a dosage and amount effective to treat the disorder in the subject.

In one aspect, provided is a method for treating a disorder of uncontrolled cellular proliferation, comprising: administering to a subject at least one disclosed compound; at least one disclosed pharmaceutical composition; and/or at least one disclosed product in a dosage and amount effective to treat the disorder in the subject. In a further aspect, provided is a method for treating or preventing a disorder characterized by fibrosis, comprising: administering to a subject at least one disclosed compound; at least one disclosed pharmaceutical composition; and/or at least one disclosed product in a dosage and amount effective to treat the disorder in the subject. Also provided is a method for the treatment of a disorder in a mammal comprising the step of administering to the mammal at least one disclosed compound, composition, or medicament.

The invention is directed at the use of described chemical compositions to treat diseases or disorders in patients (preferably human) wherein wherein β-catenin/BCL9 protein-protein interaction inhibition would be predicted to have a therapeutic effect, such as disorders of uncontrolled cellular proliferation (e.g. tumors and cancers) and disorders characterized by fibrosis (e.g. polycystic kidney disease), by administering one or more disclosed compounds or products.

The compounds disclosed herein are useful for treating, preventing, ameliorating, controlling or reducing the risk of a variety of disorders of uncontrolled cellular proliferation. In one aspect, the disorder of uncontrolled cellular proliferation is associated with dysregulation of the Wnt signaling pathway. In a further aspect, the Wnt signaling pathway dysregulation is associated with a β-catenin/BCL9 protein-protein interaction dysfunction.

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

Examples of disorders associated with β-catenin/BCL9 protein-protein interaction dysfunction include a disorder of uncontrolled cellular proliferation. In a still further aspect, the disorder is cancer. In yet a further aspect, the cancer is a sarcoma. In an even further aspect, the cancer is a carcinoma. In a still further aspect, the cancer is a hematological cancer. In a yet further aspect, the cancer is a solid tumor.

It is understood that cancer refers to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

In a further aspect, the cancer is selected from a cancer of the breast, cervix, gastrointestinal tract, colorectal tract, brain, skin, prostate, ovary, thyroid, testes, genitourinary tract, pancreas, and endometrias. In a still further aspect, the cancer is a cancer of the breast. In yet a further aspect, the cancer of the breast is a hormone resistant cancer. In an even further aspect, the cancer of the breast is a hormone resistant cancer. In a still further aspect, the cancer is a cancer of the cervix. In yet a further aspect, the cancer is a cancer of the ovary. In an even further aspect, the cancer is a cancer of the endometrias. In a still further aspect, the cancer is a cancer of the genitourinary tract. In yet a further aspect, the cancer is a cancer of the colorectal tract. In an even further aspect, the cancer of the colorectal tract is a colorectal carcinoma. In a still further aspect, the cancer is a cancer of the gastrointestinal tract. In yet a further aspect, the cancer of the gastrointestinal tract is a gastrointestinal stromal tumor. In an even further aspect, the cancer is a cancer of the skin. In a still further aspect, the cancer of the skin is a melanoma. In yet a further aspect, the cancer is a cancer of the brain. In an even further aspect, the cancer of the brain is a glioma. In a still further aspect, the glioma is glioblastoma multiforme. In yet a further aspect, glioma is selected from is selected from a ependymoma, astrocytoma, oligodendroglioma, and oligoastrocytoma. In an even further aspect, the cancer of the brain is selected from acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, and hemangiopercytoma. In a still further aspect, the hematological cancer is selected from a leukemia, lymphoma, chronic myeloproliferative disorder, myelodysplastic syndrome, myeloproliferative neoplasm, and plasma cell neoplasm (myeloma). In yet a further aspect, the hematological cancer is leukemia. In an even further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia. In a still further aspect, the leukemia is acute lymphocytic leukemia. In yet a further aspect, the hematological cancer is lymphoma. In an even further aspect, the hematological cancer is myeloma. In a still further aspect, the myeloma is multiple myeloma.

In a further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

In a further aspect, the cancer is treatment-resistant. In a still further aspect, the cancer is resistant to treatment with the at least one chemotherapeutic agent. In yet a further aspect, the cancer is resistant to treatment with the at least one hormone therapy agent.

In various aspects, disorders associated with a β-catenin/BCL9 protein-protein interaction dysfunction include disorders characterized by fibrosis. In a further aspect, the fibrotic disease is selected from pulmonary fibrosis, liver fibrosis, and polycystic kidney disease.

The compounds are further useful in a method for the prevention, treatment, control, amelioration, or reduction of risk of the diseases, disorders and conditions noted herein. The compounds are further useful in a method for the prevention, treatment, control, amelioration, or reduction of risk of the aforementioned diseases, disorders and conditions in combination with other agents.

The present invention is further directed to administration of a β-catenin/BCL9 protein-protein interaction inhibitor for improving treatment outcomes in the context of disorders of uncontrolled cellular proliferation, including cancer. That is, in one aspect, the invention relates to a cotherapeutic method comprising the step of administering to a mammal an effective amount and dosage of at least one compound of the invention in connection with cancer therapy.

In a further aspect, administration improves treatment outcomes in the context of cancer therapy. Administration in connection with cancer therapy can be continuous or intermittent. Administration need not be simultaneous with therapy and can be before, during, and/or after therapy. For example, cancer therapy can be provided within 1, 2, 3, 4, 5, 6, 7 days before or after administration of the compound. As a further example, cancer therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In one aspect, the compound can be employed in combination with anti-cancer therapeutic agents. In a further aspect, the anti-cancer therapeutic agent is selected from 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab TiuxetanIdamycin®, Idarubicin, Ifex®, IFN-alphalfosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone®, Oxaliplatin, P, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In another aspect, the subject compounds can be administered in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab TiuxetanIdamycin®, Idarubicin, Ifex®, IFN-alphalfosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone®, Oxaliplatin, P, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In another aspect, the subject compound can be used in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel R Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab TiuxetanIdamycin®, Idarubicin, Ifex®, IFN-alphalfosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone®, Oxaliplatin, P, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In the treatment of conditions which require inhibition or negative modulation of β-catenin/BCL9 protein-protein interaction, an appropriate dosage level will generally be about 0.01 to 1000 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one aspect, the invention relates to methods for inhibiting or negatively modulating β-catenin/BCL9 protein-protein interaction in at least one cell, comprising the step of contacting the at least one cell with at least one compound of the invention, in an amount effective to negatively modulate β-catenin/BCL9 protein-protein interaction in the at least one cell. In a further aspect, the cell is mammalian, for example human. In a further aspect, the cell has been isolated from a subject prior to the contacting step. In a further aspect, contacting is via administration to a subject.

In various aspects, disclosed are methods for the treatment of a disorder of uncontrolled cellular proliferation associated with a β-catenin/BCL9 dysfunction in a mammal comprising the step of administering to the mammal an effective amount of at least one compound having a structure represented by a formula:

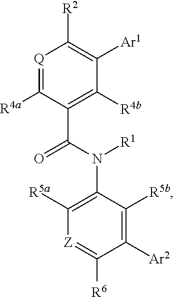

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

Also disclosed are methods for the treatment of a disorder of uncontrolled cellular proliferation associated with a β-catenin/BCL9 protein-protein interaction dysfunction in a mammal comprising the step of administering to the mammal an effective amount of at least one compound having a structure represented by a formula:

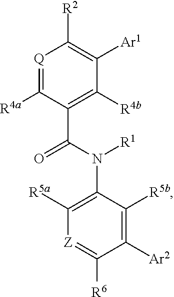

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

Also disclosed are methods for inhibiting β-catenin/BCL9 protein-protein interactions in a mammal comprising the step of administering to the mammal an effective amount of at least one compound having a structure represented by a formula:

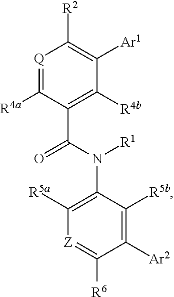

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

Also disclosed are methods for down-regulation of the Wnt pathway in a mammal comprising the step of administering to the mammal an effective amount of at least one compound having a structure represented by a formula:

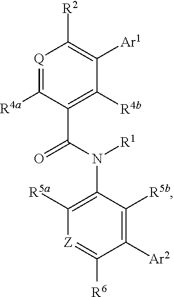

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

In a further aspect, the effective amount is a therapeutically effective amount. In a still further aspect, the effective amount is a prophylactically effective amount.

In a further aspect, the mammal is a human. In a still further aspect, the mammal has been diagnosed with a need for treatment of the disorder prior to the administering step. In yet a further aspect, the mammal has been diagnosed with a need for inhibiting protein-protein interactions of β-catenin and BCL9 activity prior to the administering step. In an even further aspect, the method further comprises the step of identifying a mammal in need of treatment of the disorder. In a still further aspect, the method further comprises the step of identifying a mammal in need for inhibiting protein-protein interactions of β-catenin and BCL9. In yet a further aspect, inhibiting protein-protein interactions of β-catenin and BCL9 is associated with treating a cancer.

In a further aspect, the mammal is human; and wherein the human has been identified to have a 1q21 chromosomal abnormality.

In a further aspect, the mammal is human; and wherein the step of identifying the human in need of treatment of the disorder comprises the steps of: (a) obtaining a sample from the human; wherein the sample comprises cells suspected of being associated with the disorder of uncontrolled cellular proliferation; (b) determining if the sample comprises cells with a 1q21 chromosomal abnormality; and (c) administering to the human the compound when the sample is positive for a 1q21 chromosomal abnormality.

Also disclosed are methods for inhibiting β-catenin/BCL9 protein-protein interactions in at least one cell comprising the step of contacting the cell with an effective amount of at least one compound having a structure represented by a formula:

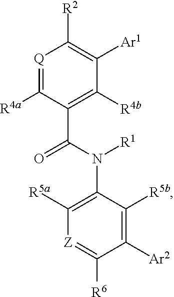

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

In a further aspect, the cell is mammalian. In a still further aspect, the cell is human. In yet a further aspect, the cell has been isolated from a mammal prior to the contacting step. In an even further aspect, contacting is via administration to a mammal. In a still further aspect, the mammal has been diagnosed with a need for inhibiting protein-protein interactions of β-catenin and BCL9 prior to the administering step. In yet a further aspect, the mammal has been diagnosed with a need for treatment of a disorder related to protein-protein interactions of β-catenin and BCL9 prior to the administering step. In an even further aspect, the disorder is a disorder of uncontrolled cellular proliferation. In a still further aspect, the disorder of uncontrolled cellular proliferation is a cancer.

### 2. Manufacture of a Medicament

In one aspect, the invention relates to a method for the manufacture of a medicament for inhibition of β-catenin/BCL9 protein-protein interaction in a mammal comprising combining a therapeutically effective amount of a disclosed compound or product of a disclosed method with a pharmaceutically acceptable carrier or diluent. In a further aspect, the invention relates to a method for the manufacture of a medicament for inhibition of the Wnt signaling pathway in a mammal comprising combining a therapeutically effective amount of a disclosed compound or product of a disclosed method with a pharmaceutically acceptable carrier or diluent.

### 3. Use of Compounds

In one aspect, the invention relates to the use of a compound having a structure represented by a formula:

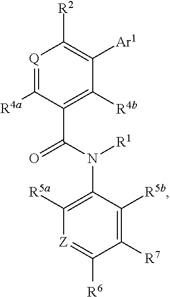

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; wherein each occurrence of R²⁰, when present, is independently selected from C1-C3 alkyl, C1-C3 monohaloalkyl, C1-C3 polyhaloalkyl, and cyclopropyl; wherein each occurrence of R^{21a} and R^{21b}, when present, is independently selected from hydrogen, C1-C3 alkyl, C1-C3 monohaloalkyl, C1-C3 polyhaloalkyl, and cyclopropyl; wherein R⁷ is selected from Ar², -A¹-A²-Ar², and

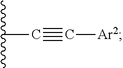

wherein each of A¹ and A², when present, is independently selected from O, NH, and CH₂, provided that each of A¹ and A² is simultaneously O; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; or a pharmaceutically acceptable salt thereof.

In one aspect, the invention relates to the use of a compound having a structure represented by a formula:

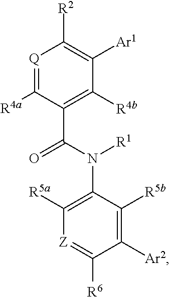

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

In a further aspect, the invention relates to the use of at least one disclosed compound for inhibiting β-catenin/BCL9 activity. In a still further aspect, inhibiting β-catenin/BCL9 activity is for treatment of a disorder of uncontrolled cellular proliferation.

In a further aspect, the invention relates to the use of at least one disclosed compound for administration to a subject; wherein the subject has a disorder of uncontrolled cellular proliferation.

In a further aspect, the compound of the use is a disclosed compound or a product of a disclosed method of making a compound.

In a still further aspect, the use is therapeutic treatment of a mammal. In a yet further aspect, the mammal is human.

In a further aspect, the use is inhibition of β-catenin/BCL9 protein-protein interactions. In a still further aspect, the use is inhibition of the Wnt signaling pathway. In a still further aspect, the need for inhibition of β-catenin/BCL9 protein-protein interactions is associated with treatment of a disorder of uncontrolled cellular proliferation. In a yet further aspect, inhibition of the Wnt signaling pathway treats a disorder of uncontrolled cellular proliferation.

In a further aspect, the disorder of uncontrolled cellular proliferation is a cancer. In an even further aspect, cancer is a leukemia. In a still further aspect, the cancer is a myeloma. In a yet further aspect, cancer is a solid tumor. In an even further aspect, the cancer is a lymphoma.

In a further aspect, the cancer is selected from the cancer is selected from cancers of the blood, brain, prostate, genitourinary tract, gastrointestinal tract, colon, rectum, breast, livery, kidney, lymphatic system, stomach, lung, pancreas, and skin. In an even further aspect, the cancer is selected from a cancer of the colon, rectum, breast, prostate, liver, skin and lung. In a still further aspect, the cancer is selected from a cancer of the breast, ovary, testes and prostate. In a yet further aspect, the cancer is a cancer of the breast. In various aspect, the cancer is a cancer of the liver. In a still further aspect, the cancer is a cancer of the prostate. In a yet further aspect, the cancer is a cancer of the colon or rectum.

In a further aspect, the disorder is characterized by fibrosis. In a yet further aspect, the fibrotic disorder is selected from pulmonary fibrosis, liver fibrosis, and polycystic kidney disease.

### 4. Kits

In one aspect, the invention relates to a kit comprising at least one compound having a structure represented by a formula:

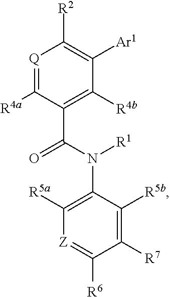

wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; wherein each occurrence of R²⁰, when present, is independently selected from C1-C3 alkyl, C1-C3 monohaloalkyl, C1-C3 polyhaloalkyl, and cyclopropyl; wherein each occurrence of R^{21a} and R^{21b}, when present, is independently selected from hydrogen, C1-C3 alkyl, C1-C3 monohaloalkyl, C1-C3 polyhaloalkyl, and cyclopropyl; wherein R⁷ is selected from Ar², -A¹-A²-Ar², and

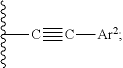

wherein each of A¹ and A², when present, is independently selected from O, NH, and CH₂, provided that each of A¹ and A² is simultaneously O; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole; or a pharmaceutically acceptable salt thereof, and one or more of:
(a) at least one agent known to increase BCL9 activity;
(b) at least one agent known to increase β-catenin activity;
(c) at least one agent known to decrease BCL9 activity;
(d) at least one agent known to decrease β-catenin activity;
(e) at least one agent known to treat a disease of uncontrolled cellular proliferation;
(f) instructions for treating a disorder associated uncontrolled cellular proliferation; or
(g) instructions for treating a disorder associated with a β-catenin/BCL9 dysfunction.

In one aspect, the invention relates to a kit comprising at least one compound having a structure represented by a formula:

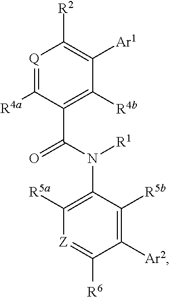

wherein wherein Q is selected from N and CR^{4c}; wherein Z is selected from N and CR^{5c}; wherein R¹ is selected from hydrogen and C1-C4 alkyl; wherein R² is selected from —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂-Cy¹, —NHCH₂-Cy²; —OCH₂-Cy¹, and —OCH₂-Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, —(C2-C8 alkyl)-OH, —(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂-Cy³, —NHCH₂-Cy⁴; —OCH₂-Cy³, and —OCH₂-Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof, and one or more of:
(h) at least one agent known to increase BCL9 activity;
(i) at least one agent known to increase β-catenin activity;
(j) at least one agent known to decrease BCL9 activity;
(k) at least one agent known to decrease β-catenin activity;
(l) at least one agent known to treat a disease of uncontrolled cellular proliferation;
(m) instructions for treating a disorder associated uncontrolled cellular proliferation; or
(n) instructions for treating a disorder associated with a β-catenin/BCL9 dysfunction.

In a further aspect, the compound of the kit is a disclosed compound or a product of a disclosed method of making a compound.

In a further aspect, the at least one compound and the at least one agent are co-formulated. In a still further aspect, the at least one compound or the at least one product and the at least one agent are co-packaged.

In a further aspect, the at least one agent is a hormone therapy agent. In a yet further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the at least one agent is a chemotherapeutic agent. In a yet further aspect, the chemotherapeutic agent is selected from one or more of the group consisting of an alkylating agent, an antimetabolite agent, an antineoplastic antibiotic agent, a mitotic inhibitor agent, a mTor inhibitor agent or other chemotherapeutic agent.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

In a further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In a further aspect, the mTor inhibitor agent is selected from one or more of the group consisting of everolimus, siroliumus, and temsirolimus, or a pharmaceutically acceptable salt, hydrate, solvate, or polymorph thereof.

In a further aspect, the disorder of uncontrolled cellular proliferation is associated with a β-catenin/BCL9 protein-protein interaction dysfunction. In a still further aspect, the disorder of uncontrolled cellular proliferation is a cancer. In an even further aspect, cancer is a leukemia. In a still further aspect, the cancer is a sarcoma. In a yet further aspect, cancer is a solid tumor. In an even further aspect, the cancer is a lymphoma. In a still further aspect, the cancer is selected from chronic lymphocytic leukemia, small lymphocytic lymphoma, B-cell non-Hodgkin lymphoma, and large B-cell lymphoma. In a yet further aspect, the cancer is selected from cancers of the blood, brain, genitourinary tract, gastrointestinal tract, colon, rectum, breast, livery, kidney, lymphatic system, stomach, lung, pancreas, and skin. In an even further aspect, the cancer is selected from a cancer of the lung and liver. In a still further aspect, the cancer is selected from a cancer of the breast, ovary, testes and prostate. In a yet further aspect, the cancer is a cancer of the breast. In various aspect, the cancer is a cancer of the ovary. In a still further aspect, the cancer is a cancer of the prostate. In a yet further aspect, the cancer is a cancer of the testes.

In a further aspect, the instructions further comprise providing the compound in connection with a surgical procedure. In a still further aspect, the instructions provide that surgery is performed prior to the administering of at least one compound. In yet a further aspect, the instructions provide that surgery is performed after the administering of at least one compound. In an even further aspect, the instructions provide that the administering of at least one compound is to effect presurgical debulking of a tumor. In a still further aspect, the instructions provide that the administering of at least one compound is to effect presurgical debulking of a tumor.

In a further aspect, the the instructions further comprise providing the compound in connection with radiotherapy. In a still further aspect, the instructions provide that radiotherapy is performed prior to the administering of at least one compound. In yet a further aspect, the instructions provide that radiotherapy is performed after the step of the administering of at least one compound. In an even further aspect, the instructions provide that radiotherapy is performed at about the same time as the step of the administering of at least one compound.

In a further aspect, the the instructions further comprise providing the compound in connection with at least one agent that is a chemotherapeutic agent.

### 5. Non-Medical Uses

Also provided are the uses of the disclosed compounds and products as pharmacological tools in the development and standardization of in vitro and in vivo test systems for the evaluation of the effects of inhibitors of β-catenin/BCL9 protein-protein interactions in laboratory animals such as cats, dogs, rabbits, monkeys, rats and mice, as part of the search for new therapeutic agents that inhibit β-catenin/BCL9 protein-protein interactions.

### E. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

As indicated, some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

### 1. Preparation of Compounds 1-3

The synthesis scheme (Synthesis Scheme 1) for the preparation of Compounds 1, 2, and 3 is shown below.

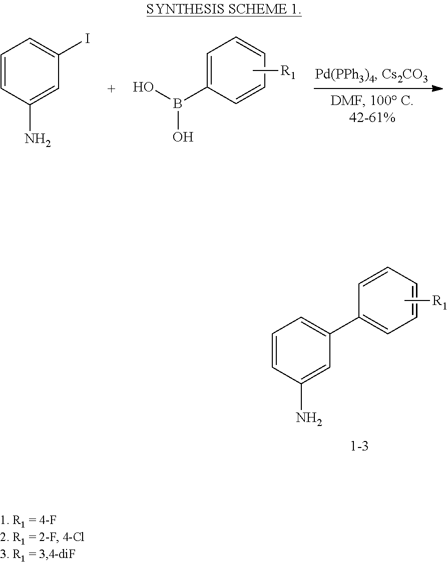

### a. Preparation of 4′-Fluoro-[1,1′-biphenyl]-3-amine (Compound 1)

To a solution of 3-iodoaniline (5.00 g, 22.83 mmol) in dry DMF (40 mL) under anhydrous conditions was added (4-fluorophenyl) boronic acid (3.83 g, 27.40 mmol), Pd(PPh₃)₄ (1.32 g, 1.14 mmol), and Cs₂CO₃ (22.31 g, 68.49 mmol). The mixture was heated to 80° C. under nitrogen and stirred for 19 h. The solvent was then removed under reduced pressure, and the residue was taken into EtOAc (100 mL). The solution was washed with water (2×50 mL), brine (50 mL), and dried over MgSO₄. After the filtration, the solvent was removed under reduced pressure. The residue was purified by column chromatography (silica gel, hexanes:EtOAc=3:1) to afford 1 (2.53 g, 59% yield) as an off-white solid. ¹H NMR (500 MHz, CDCl₃): δ ppm 7.53-7.50 (m, 2H), 7.23 (t, J=7.8 Hz, 1H), 7.11 (t, J=8.8 Hz, 1H), 6.94 (d, J=7.6 Hz, 1H), 6.86 (s, 1H), 6.69-6.67 (m, 1H), 3.74 (brs, 2H). ¹³C NMR (125 MHz, CDCl₃): δ ppm 163.51, 161.55, 146.89, 141.58, 137.63, 137.60, 129.88, 128.76, 128.70, 117.62, 115.67, 115.50, 114.18, 113.84. HRMS (ESI) Calcd. for C₁₂H₁₁FN (M+H)⁺ 188.0876, found 188.0877.

### b. Preparation of 4′-Chloro-2′-fluoro-[1,1′-biphenyl]-3-amine (Compound 2)

(0.45 g, 42% yield) as a yellow oil. ¹H NMR (500 MHz, CDCl₃): δ ppm 7.36 (t, J=8.4 Hz, 1H), 7.23 (t, J=7.8 Hz, 1H), 7.19-7.16 (m, 2H), 6.90 (d, J=7.7 Hz, 1H), 6.83 (s, 1H), 6.71 (d, J=8.0 Hz, 1H), 3.74 (s, 2H). ¹³C NMR (125 MHz, CDCl₃): δ ppm 160.70, 158.70, 146.75, 136.02, 133.90, 133.82, 131.61, 131.57, 129.71, 124.89, 124.86, 119.44, 119.42, 117.11, 116.89, 115.74, 115.72, 114.98. HRMS (ESI) Calcd. for C₁₂H₁₀ClFN (M+H)⁺ 222.0486, found 222.0484.

### c. Preparation of 3′,4′-Difluoro-[1,1′-biphenyl]-3-amine (Compound 3)

(0.57 g, 61% yield) as an off-white solid. ¹H NMR (500 MHz, CDCl₃): δ ppm 7.36 (ddd, J=2.2, 7.6, 11.6 Hz, 1H,) 7.28-7.17 (m, 3H), 6.92 (d, J=7.6 Hz, 1H), 6.82 (s, 1H) 6.70 (dd, J=2.0, 7.9 Hz, 1H,) 3.78 (s, 2H). ¹³C NMR (125 MHz, d⁶-DMSO/CDCl₃): δ ppm 151.66, 151.56, 151.11, 151.01, 149.69, 149.59, 149.14, 149.04, 147.18, 140.56, 138.82, 138.79, 138.77, 138.74, 130.14, 123.20, 123.17, 123.15, 123.124, 117.647, 117.550, 117.510, 116.215, 116.075, 114.802, 113.773. MS (ESI) m/z=206.4 [M+H]⁺. HRMS (ESI) Calcd. for C₁₂H₁₀F₂N (M+H)⁺ 206.0781, found 206.0777.

### 2. Preparation of Compound 4

The synthesis scheme (Synthesis Scheme 2) for the preparation of Compound 4 is shown below.

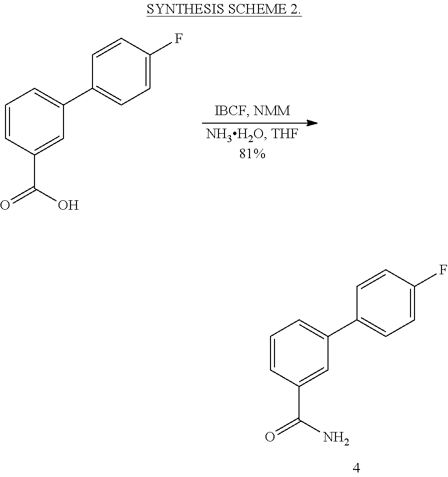 [SMILES: O=C(O)c1cccc(-c2ccc(F)cc2)c1 ; NC(=O)c1cccc(-c2ccc(F)cc2)c1]

### a. Preparation of 4′-Fluoro-[1,1′-biphenyl]-3-carboxamide (Compound 4)

To a solution of 3-(4-fluorophenyl) benzoic acid (0.2 g, 0.92 mmol) and N-methyl morpholine (0.14 g, 1.38 mmol) in THF (15 mL) was added isobutyl chloroformate (0.19 g, 1.38 mmol) at −15° C. The resulting mixture was stirred for 1 h at the same temperature. Then, 28% ammonium hydroxide aqueous solution (15 mL) was added slowly. The temperature was allowed to rise to room temperature gradually and stir for another 1 h. The mixture was diluted with EtOAc (100 mL), washed with brine (30 mL×3), dried over Na₂SO₄, filtered, and concentrated. The residue was purified by column chromatography (silica gel, hexanes:acetone=3:1 to 1:1) to afford 4 (0.16 g, 81% yield) as a white solid. ¹H NMR (300 MHz, d⁶-DMSO): δ ppm 8.10 (s, 1H), 8.08 (brs, 1H), 7.83 (d, J=7.5 Hz, 1H), 7.79-7.72 (m, 3H), 7.51 (t, J=7.8 Hz, 1H), 7.43 (brs 1H), 7.33-7.27 (m, 2H). ¹³C NMR (125 MHz, CD₃OD): δ ppm 171.02, 169.28, 163.91, 161.96, 140.56, 136.68, 136.63, 134.425, 130.09, 128.96, 128.80, 128.74, 126.30, 125.97, 115.58, 115.41. HRMS (ESI) Calcd. for C₁₃H₁₀FNO (M+Na)⁺ 238.0639, found 238.0647.

### 3. Preparation of Compound 5

The synthesis scheme (Synthesis Scheme 3) for the preparation of Compound 5 is shown below. The synthesis proceeds through the intermediates indicated (Compounds 30, 31, and 32). The yield for each synthetic step was as indicated.

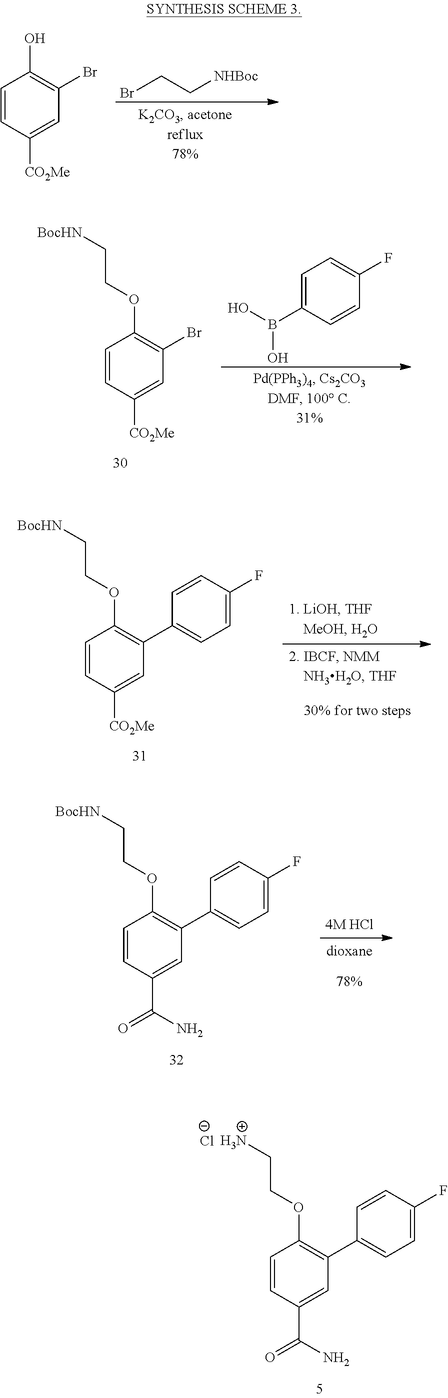

### a. Preparation of Methyl-3-bromo-4-(2-((tert-butoxycarbonyl) amino) ethoxy) benzoate (Compound 30)

To a solution of methyl 3-bromo-4-hydroxybenzoate (0.50 g, 2.16 mmol) in acetone (80 mL) was added tert-butyl (2-bromoethyl) carbamate (0.97 g, 4.33 mmol), K₂CO₃ (0.60 g, 4.33 mmol). The mixture was heated to gentle reflux and stirred overnight. Acetone was removed under vacuum, and the residue was dissolved into CH₂Cl₂ (150 mL) and washed with NaOH (1 M) (50 mL×2), brine (50 mL×2), dried over Na₂SO₄, filtered, and concentrated to give the crude product as pale yellow oil. To this residue was added hexane (50 mL) and stirred for 30 min. The resulting precipitate was filtered to give desired product 30 (0.63 g, 78% yield) as a white solid. ¹H NMR (500 MHz, CDCl₃): δ ppm 8.20 (d, J=2.0 Hz, 1H), 7.35 (dd, J=2.0, 8.5 Hz, 1H), 6.87 (d, J=8.5 Hz, 1H), 5.08 (brs, 1H), 4.12 (t, J=5.0 Hz, 2H), 3.87 (s, 3H), 3.62-3.58 (m, 2H), 1.43 (s, 9H). ¹³C NMR (125 MHz, CDCl₃): δ ppm 165.82, 158.71, 156.08, 135.03, 130.79, 124.31, 112.31, 112.06, 79.94, 68.91, 52.39, 40.06, 28.60. MS (ESI) m/z=374.3 [M+H]⁺.

### b. Preparation of Methyl-6-(2-((tert-butoxycarbonyl) amino) ethoxy)-4′-fluoro-[1,1′-biphenyl]-3-carboxylate (Compound 31)

To a solution of 30 (0.50 g, 1.34 mmol) in dry DMF (30 mL) was added (4-fluorophenyl) boronic acid (0.22 g, 1.60 mmol), Pd(PPh₃)₄ (0.15 g, 0.13 mmol), and Cs₂CO₃ (1.31 g, 4.02 mmol). The mixture was then heated to 100° C. under argon and stirred for 24 h. Then it was cooled to room temperature, and diluted with diethyl ether (150 mL), washed with water (50 mL×2), brine (50 mL×2), dried over Na₂SO₄, and concentrated under vacuum. The residue was then purified by column chromatography (silica gel, hexanes:acetone=15:1 to 10:1) to yield 31 (0.16 g, 31% yield) as a white solid. ¹H NMR (300 MHz, CDCl₃): δ ppm 8.02-7.93 (m, 2H), 7.50-7.45 (m, 2H), 7.11 (t, J=8.7 Hz, 2H), 6.97 (d, J=7.8 Hz, 1H), 4.74 (t, J=5.1 Hz, 1H), 4.09 (t, J=5.1 Hz, 2H), 3.89 (s 3H), 3.46 (q, J=5.1 Hz, 2H), 1.43 (s, 9H). ¹³C NMR (75 MHz, CDCl₃): δ ppm 166.89, 164.05, 160.78, 159.22, 155.97, 133.59, 132.54, 131.33, 131.22, 131.06, 130.11, 123.43, 115.41, 115.13, 112.25, 79.87, 68.04, 52.24, 40.08, 28.59. MS (ESI) m/z=390.2 [M+H]⁺.

### c. Preparation of tert-Butyl(2-((5-((aminooxy) carbonyl)-4′-fluoro-[1,1′-biphenyl]-2-yl) oxy) ethyl) carbamate (Compound 32)

To the solution of 31 (0.20 g, 0.51 mmol) in a solvent mixture (14 mL, THF:MeOH:H₂O=4:2:1) was added LiOH (0.098 g, 4.10 mmol). The mixture was stirred for 8 h at room temperature. Then, the pH value was adjusted to 4-5 with HCl (1 M), and diluted with water (50 mL), extracted with EtOAc (50 mL×3). The combined organic phase was dried over Na₂SO₄ and concentrated under vacuum. The resulting white solid (0.19 g, 0.51 mmol) was dissolved into THF (20 mL). To this solution was added N-methyl morpholine (0.077 g, 0.76 mmol) and isobutyl chloroformate (0.10 g, 0.76 mmol) at −15° C. The mixture was stirred for 1 h at the same temperature. Then, 28% ammonium hydroxide aqueous solution (20 mL) was added slowly. The temperature was allowed to rise to room temperature gradually and stir for another 1 h. The mixture was diluted with EtOAc (100 mL), washed with brine (30 mL×3), dried over Na₂SO₄, and concentrated under vacuum. The residue was then purified by column chromatography (silica gel, CH₂Cl₂:MeOH=30:1 to 20:1) to yield 32 (0.06 g, 30% two steps yield) as a white solid. ¹H NMR (300 MHz, d⁶-DMSO): δ ppm 7.92 (brs, 1H), 7.86-7.82 (m, 2H), 7.62-7.58 (m, 2H), 7.27-7.21 (m, 3H), 7.14 (d, J=9.0 Hz, 1H), 6.93 (t, J=5.1 Hz, 1H), 4.06 (t, J=5.1 Hz, 2H), 3.27 (q, J=5.1 Hz, 2H), 1.35 (s, 9H). ¹³C NMR (75 MHz, d⁶-DMSO): δ ppm 167.97, 163.00, 161.05, 158.06, 156.27, 134.41, 132.04, 131.98, 130.56, 129.31, 128.75, 127.48, 115.52, 115.35, 112.79, 78.44, 67.44, 39.95, 28.68. MS (ESI) m/z=391.3 [M+H]⁺.

### d. Preparation of 2-((5-((aminooxy) carbonyl)-4′-fluoro-[1,1′-biphenyl]-2-yl) oxy) ethanaminium chloride (Compound 5)

To a solution of 32 (0.060 g, 0.15 mmol) in CH₂Cl₂ (3 mL) was added 4 M HCl in dioxane (3 mL), and the mixture was then stirred at room temperature for 1 h. The solvent was then removed under reduced pressure to yield the crude product. It was dissolved with deionized water (10 mL), washed with EtOAc (5 ml×3), and lyophilized to give 5 (0.038 g, 78% yield) as a white solid. ¹H NMR (500 MHz, d⁶-DMSO): δ ppm 8.18 (brs, 3H), 7.97 (brs, 1H), 7.89-7.86 (m, 2H), 7.67-7.64 (m, 2H), 7.26-7.20 (m, 4H), 4.27 (t, J=5.1 Hz, 2H), 3.16 (t, J=5.1 Hz, 2H). ¹³C NMR (125 MHz, CD₃OD): δ ppm 163.51, 161.56, 157.81, 133.84, 133.82, 131.32, 131.25, 130.63, 130.26, 128.80, 114.96, 114.79, 112.83, 65.39, 38.95. HRMS (ESI) Calcd for C₁₅H₁₅FN₂O₂ (M+H)⁺ 275.1190, found 275.1197.

### 4. Preparation of Compound 6

The synthesis scheme (Synthesis Scheme 4) for the preparation of Compound 6 is shown below. The synthesis proceeds through the intermediates indicated (Compounds 33, 34, 35, 36, 37, 8, 38, and 39). The yield for each synthetic step was as indicated.

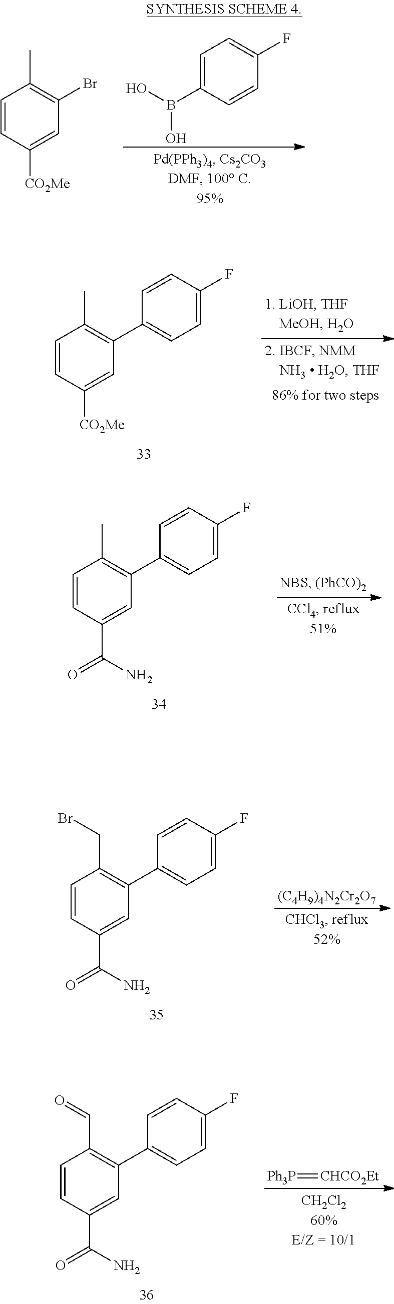

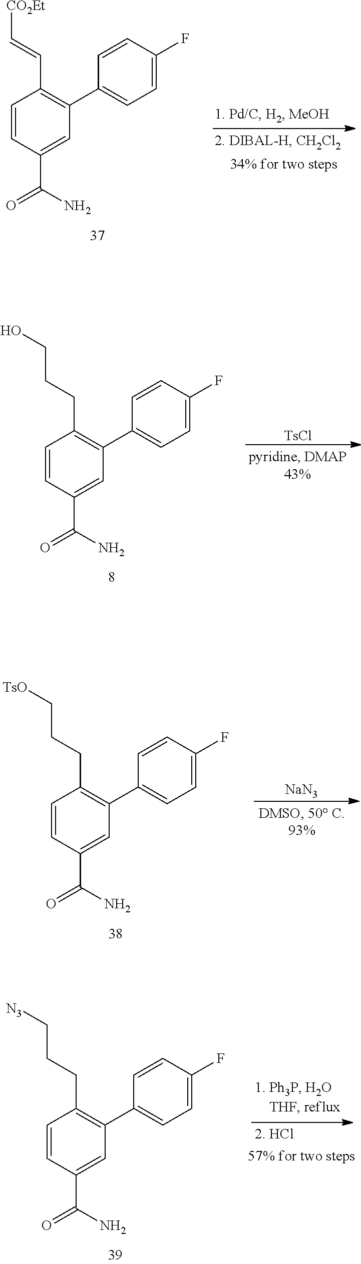

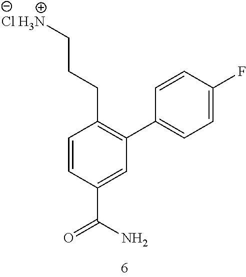

### a. Preparation of Methyl 4′-fluoro-6-methyl-[1,1′-biphenyl]-3-carboxylate (Compound 33)

To a solution of methyl 3-bromo-4-methylbenzoate (1.00 g, 4.37 mmol) in dry DMF (50 mL) was added (4-fluorophenyl) boronic acid (0.73 g, 5.24 mmol), Pd(PPh₃)₄ (0.51 g, 0.44 mmol), and Cs₂CO₃ (4.27 g, 13.11 mmol). The mixture was heated to 100° C. under argon and stirred for 24 h. Then, the reaction mixture was cooled to room temperature, diluted with diethyl ether (200 mL), washed with water (50 mL×2) and brine (50 mL×2), dried over Na₂SO₄, and concentrated under vacuum. The residue was purified by column chromatography (silica gel, hexanes:acetone=15:1 to 12:1) to yield 33 (1.02 g, 95% yield) as a pale yellow oil. ¹H NMR (500 MHz, CDCl₃): δ ppm 7.92 (d, J=8.0 Hz, 1H), 7.89 (s, 1H), 7.32 (d, J=8.0 Hz, 1H), 7.26 (dd, J=5.5, 8.5 Hz, 2H), 7.10 (t, J=8.5 Hz, 2H), 3.89 (s, 3H), 3.29 (s, 3H). ¹³C NMR (125 MHz, CDCl₃): δ ppm 167.20, 163.32, 161.36, 141.24, 141.23, 137.05, 137.02, 131.14, 130.95, 130.88, 130.73, 128.71, 128.12, 115.45, 115.28, 52.22, 20.89. MS (ESI) m/z=245.1 [M+H]⁺.

### b. Preparation of 4′-Fluoro-6-methyl-[1,1′-biphenyl]-3-carboxamide (Compound 34)

To the solution of 33 (1.00 g, 4.09 mmol) in a solvent mixture (28 mL, THF:MeOH:H₂O=4:2:1) was added LiOH (0.78 g, 32.75 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum. The resulting product (0.50 g, 2.17 mmol) and N-methyl morpholine (0.33 g, 3.26 mmol) in THF (20 mL) was added isobutyl chloroformate (0.45 g, 3.26 mmol) at −15° C. The resulting mixture was stirred for 1 h at the same temperature. Then, 28% ammonium hydroxide aqueous solution (20 mL) was added slowly. The temperature was allowed to rise to room temperature gradually and stir for another 1 h. The mixture was diluted with EtOAc (150 mL), washed with brine (50 mL×3), dried over Na₂SO₄, filtered, and concentrated. The residue was purified by column chromatography (silica gel, hexanes:acetone=3:1 to 1:1) to yield 34 (0.43 g, 86% yield for two steps) as a white solid. ¹H NMR (500 MHz, d⁶-acetone): δ ppm 7.85 (dd, J=2.0, 8.0 Hz, 1H), 7.78 (d, J=2.0 Hz, 1H), 7.52 (brs, 1H), 7.41 (dd, J=5.0, 8.5 Hz, 2H), 7.38 (d, J=8.0 Hz, 1H), 7.24 (t, J=9.0 Hz, 2H), 6.68 (brs, 1H), 2.29 (s, 3H). ¹³C NMR (125 MHz, d⁶-acetone): δ ppm 168.03, 163.25, 161.31, 140.87, 139.05, 137.71, 137.68, 132.46, 131.26, 131.20, 130.61, 129.00, 126.87, 115.26, 115.09, 19.71. MS (ESI) m/z=230.2 [M+H]⁺.

### c. Preparation of 6-(Bromomethyl)-4′-fluoro-[1,1′-biphenyl]-3-carboxamide (Compound 35)

To the solution of 34 (0.50 g, 2.18 mmol) and N-bromosuccinimide (0.47 g, 2.62 mmol) in CCl₄ (30 mL) was added (PhCO)₂ (0.16 g, 0.66 mmol). The mixture was heated to gentle reflux and stirred for 7 h. After cooling to room temperature, the reaction mixture was diluted with CH₂Cl₂ (100 mL), washed with brine (30 mL×3), dried over Na₂SO₄, filtered, and concentrated. The residue was purified by column chromatography (silica gel, hexanes:acetone=3:1 to 2:1) to yield 35 (0.41 g, 51% yield) as a white solid. ¹H NMR (500 MHz, d⁶-acetone): δ ppm 7.95 (dd, J=2.0, 8.0 Hz, 1H), 7.82 (d, J=2.0 Hz, 1H), 7.68 (d, J=8.0 Hz, 1H), 7.62 (brs, 1H), 7.52 (dd, J=5.5, 9.0 Hz, 2H), 7.28 (t, J=9.0 Hz, 2H), 6.80 (brs, 1H), 4.59 (s, 2H). ¹³C NMR (125 MHz, d⁶-acetone): δ ppm 167.52, 163.60, 161.65, 141.49, 138.86, 136.18, 136.15, 134.82, 131.36, 131.20, 131.13, 129.71, 127.48, 115.50, 115.33, 31.34. MS (ESI) m/z=308.0 [M+H]⁺.

### d. Preparation of 6-Formyl-[1,1′-biphenyl]-3-carboxamide (Compound 36)

To the solution of 35 (0.40 g, 1.30 mmol) in chloroform (30 mL) was added bis (tetrabutyl ammonium) dichromate (1.82 g, 2.60 mmol). The resulting mixture was heated under reflux for 3 h and then filtered through silica gel to remove all the inorganic products. The silica pad was washed with diethyl ether (30 mL×3). The combined organic filtrates were removed under reduced pressure. The residue was purified by column chromatography on silica gel (hexanes:acetone=2:1 to 1:1) to afford 36 (0.15 g, 52% yield) as a white solid. ¹H NMR (300 MHz, d⁶-DMSO): δ ppm 9.90 (s, 1H), 8.25 (brs, 1H), 8.03-7.95 (m, 3H), 7.67 (brs, 1H), 7.56 (dd, J=5.7, 8.4 Hz, 2H), 7.36 (t, J=8.4 Hz, 2H). ¹³C NMR (75 MHz, d⁶-DMSO): δ ppm 192.02, 167.47, 164.58, 161.33, 144.35, 139.26, 135.53, 134.07, 134.03, 132.89, 132.78, 130.65, 128.47, 127.66, 116.30, 116.01. MS (ESI) m/z=243.3 [M+H]⁺.

### e. Preparation of (E)-ethyl 3-(5-carbamoyl-4′-fluoro-[1,1′-biphenyl]-2-yl) acrylate (Compound 37)

To a solution of 36 (0.13 g, 0.53 mmol) in CH₂Cl₂ (15 mL) was added ethyl 2-(triphenylphosphoranylidene) acetate (0.48 g, 1.60 mmol). The resulting mixture was stirred overnight at room temperature for 15 h. The reaction mixture was then diluted with CH₂Cl₂ (50 mL), washed with brine (30 mL×3), dried over Na₂SO₄, filtered, and concentrated. The residue was purified by column chromatography (silica gel, hexanes:acetone=3:1 to 2:1) to yield 37 (0.10 g, 60% yield) as a white solid. ¹H NMR (500 MHz, d⁶-DMSO): δ ppm 8.11 (s, 1H), 7.99 (d, J=8.5 Hz, 1H), 7.89 (d, J=8.0 Hz, 1H), 7.85 (s, 1H), 7.49 (d, J=16.0 Hz, 1H), 7.48 (s, 1H), 7.39 (dd, J=5.5, 9.0 Hz, 2H), 7.33 (t, J=9.0 Hz, 2H), 6.66 (d, J=16.0 Hz, 1H), 4.11 (q, J=7.0 Hz, 2H), 1.18 (t, J=7.0 Hz, 3H). ¹³C NMR (125 MHz, d⁶-DMSO): δ ppm 167.61, 166.58, 163.57, 161.62, 142.13, 141.75, 136.04, 134.85, 132.44, 132.37, 132.18, 130.05, 129.46, 129.37, 127.66, 121.25, 116.16, 115.99, 60.87, 14.80. MS (ESI) m/z=314.1 [M+H]⁺.

### f. Preparation of 4′-Fluoro-6-(3-hydroxypropyl)-[1,1′-biphenyl]-3-carboxamide (Compound 8)

To a solution of 37 (0.10 g, 0.32 mmol) in MeOH (20 mL) was added 10% Pd on activated carbon (0.010 g, 10% by weight). The air was evacuated and exchanged with the H₂ gas three times, and the reaction was allowed to stir under H₂ for 8 h. The reaction mixture was then filtered through celite, and the solvent was removed under reduced pressure. The resulting crude product (0.10 g, 0.32 mmol) was dissolved into CH₂Cl₂ (20 mL) and cooled to −78° C. with a dry ice/acetone bath. To this solution, diisobutylaluminum hydride (1M) (0.48 mL, 0.48 mmol) was added slowly. The temperature was allowed to rise to room temperature and stir for 8 h. The reaction mixture was then cooled to 0° C., quenched with water (10 mL) and NaOH (1M) (10 mL), stirred for another 15 min, extracted with CH₂Cl₂ (30 mL×3), dried over Na₂SO₄, filtered, and concentrated. The residue was purified by column chromatography (silica gel, hexanes:acetone=2:1 to 1:1) to yield 8 (0.03 g, 34% two steps yield) as a white solid. ¹H NMR (500 MHz, d⁶-acetone): δ ppm 7.89 (dd, J=2.0, 8.5 Hz, 1H), 7.75 (d, J=2.0 Hz, 1H), 7.55 (brs, 1H), 7.43 (d, J=8.0 Hz, 1H), 7.39 (dd, J=5.5, 8.5 Hz, 2H), 7.22 (t, J=8.5 Hz, 2H), 6.71 (brs 1H), 3.54 (brs, 1H), 3.44 (t, J=6.5 Hz, 2H), 2.70 (t, J=6.5 Hz, 2H), 1.68 (m, 2H). ¹³C NMR (125 MHz, d⁶-acetone): δ ppm 170.89, 169.28, 163.39, 161.44, 144.10, 141.19, 137.34, 137.31, 131.32, 130.95, 130.89, 129.53, 129.28, 126.69, 114.98, 114.80, 61.15, 33.68, 29.35. HRMS (ESI) Calcd. for C₁₆H₁₆FNNaO₂ (M+Na)⁺ 296.1063, found 296.1061.

### g. Preparation of 3-(5-carbamoyl-4′-fluoro-[1,1′-biphenyl]-2-yl) propyl-4-methylbenzenesulfonate (Compound 38)

To the solution of 8 (0.030 g, 0.11 mmol) and 4-dimethylaminopyridine (0.0040 g, 0.033 mmol) in pyridine (5 mL) was added 4-methylbenzenesulfonyl chloride (0.041 g, 0.022 mmol). The mixture was stirred overnight at room temperature. After 15 h, the reaction mixture was diluted with EtOAc (80 mL), washed with HCl (1M) (30 mL×3) and brine (30 mL×2), dried over Na₂SO₄, filtered, and concentrated. The residue was purified by column chromatography (silica gel, hexanes:acetone=3:1-2:1) to yield 38 (0.02 g, 43% yield) as a white solid. ¹H NMR (500 MHz, d⁶-acetone): δ ppm 7.85 (dd, J=2.0, 8.0 Hz, 1H), 7.73-7.71 (m 3H), 7.50 (brs 1H), 7.46 (d, J=8.0 Hz, 2H), 7.36-7.32 (m, 3H), 7.21 (t, J=8.5 Hz, 2H), 6.63 (brs, 1H), 3.94 (t, J=6.0 Hz, 2H), 2.66 (t, J=6.0 Hz, 2H), 2.46 (s, 3H), 1.80 (m, 2H). ¹³C NMR (125 MHz, d⁶-acetone): δ ppm 168.06, 167.79, 163.30, 161.36, 145.15, 141.99, 140.93, 137.39, 137.36, 132.65, 131.22, 131.16, 130.17, 129.62, 129.42, 127.94, 127.09, 115.34, 115.17, 69.95, 29.82, 28.80, 20.84. MS (ESI) m/z=428.2 [M+H]⁺.

### h. Preparation of 6-(3-Azidopropyl)-4′-fluoro-[1,1′-biphenyl]-3-carboxamide (Compound 39)

To the solution of 38 (0.020 g, 0.047 mmol) in DMSO (5 mL) was added NaN₃ (0.024 g, 0.37 mmol). The mixture was heated to 50° C. and stirred for 7 h, and then cooled to room temperature, diluted with EtOAc (50 mL), washed with water (20 mL×3), dried over Na₂SO₄, filtered, and concentrated. The residue was purified by column chromatography (silica gel, hexanes:acetone=3:1 to 2:1) to yield 39 (0.013 g, 93% yield) as a white solid. ¹H NMR (500 MHz, d⁶-acetone): δ ppm 7.90 (dd, J=2.0, 8.0 Hz, 1H), 7.76 (d, J=2.0 Hz, 1H), 7.51 (brs, 1H), 7.45 (d, J=8.0 Hz, 1H), 7.41 (dd, J=5.5, 8.5 Hz, 2H), 7.24 (t, J=8.5 Hz, 2H), 6.62 (brs, 1H), 3.24 (t, J=6.5 Hz, 2H), 2.74 (t, J=6.5 Hz, 2H), 1.75 (m, 2H). ¹³C NMR (125 MHz, d⁶-acetone): δ ppm 168.05, 167.82, 163.33, 161.39, 142.53, 140.96, 137.55, 132.60, 131.28, 131.21, 129.69, 129.43, 127.13, 115.33, 115.16, 50.76, 30.06, 30.00. MS (ESI) m/z=299.1 [M+H]⁺.

### i. Preparation of 3-(5-Carbamoyl-4′-fluoro-[1,1′-biphenyl]-2-yl) propan-1-aminium chloride (Compound 6)

To the solution of 39 (0.013 g, 0.044 mmol) and triphenylphosphine (0.023 g, 0.088 mmol) in THF (5 mL), water (0.0020 g, 0.11 mmol) was added. The resulting mixture was heated to gentle reflux and stirred for 8 h. The solvent was removed under vacuum, and the residue was dissolved into 4 M HCl in dioxane (3 mL). The mixture was then stirred at room temperature for 15 min. The solvent was removed under reduced pressure to yield the crude product, which was further dissolved with deionized water (5 mL), washed with EtOAc (5 mL×3), and lyophilized to give 6 (8.0 mg, 57% yield) as a white solid. ¹H NMR (500 MHz, d⁶-DMSO): δ ppm 8.03 (brs, 3H), 8.00 (brs 1H), 7.83 (d, J=7.5 Hz, 1H), 7.69 (s, 1H), 7.41-7.36 (m, 3H), 7.32 (brs, 1H), 7.28 (t, J=8.5 Hz, 2H), 2.63-2.60 (m, 4H), 1.74-1.68 (m, 2H). ¹³C NMR (125 MHz, CD₃OD): δ ppm 168.12, 163.14, 161.20, 142.24, 140.75, 137.46, 137.43, 132.86, 131.76, 131.69, 129.83, 127.51, 115.99, 115.82, 39.00, 29.95, 28.69. HRMS (ESI) Calcd. for C₁₆H₁₇FN₂O (M+H)⁺ 273.1398, found 273.1404.

### 5. Preparation of Compound 7

The synthesis scheme (Synthesis Scheme 5) for the preparation of Compound 7 is shown below. The synthesis proceeds through the intermediate indicated (Compound 40). The yield for each synthetic step was as indicated.

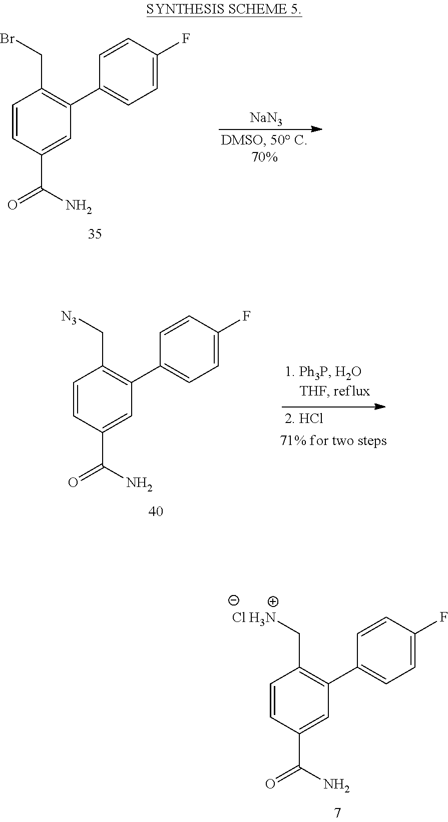

### a. Preparation of 6-(Azidomethyl)-4′-fluoro-[1,1′-biphenyl]-3-carboxamide (Compound 40)

To the solution of 35 (0.10 g, 0.32 mmol) in DMSO (10 mL) was added NaN₃ (0.17 g, 2.60 mmol), the mixture was heated to 50° C. and stirred for 7 h. Then, the reaction mixture was cooled to room temperature, diluted with EtOAc (50 mL), washed with water (20 mL×3), dried over Na₂SO₄, filtered, and concentrated. The residue was then purified by column chromatography (silica gel, hexanes:acetone=2:1 to 1:1) to yield 40 (0.060 g, 70% yield) as a white solid. ¹H NMR (500 MHz, d⁶-acetone): δ ppm 8.00 (dd, J=2.0, 8.0 Hz, 1H), 7.88 (d, J=2.0 Hz, 1H), 7.64-7.62 (m, 2H), 7.47 (dd, J=5.5, 8.0 Hz, 2H), 7.26 (t, J=8.0 Hz, 2H), 4.45 (s, 2H). ¹³C NMR (125 MHz, d⁶-acetone): δ ppm 167.65, 163.62, 161.67, 141.23, 136.58, 136.26, 136.24, 134.67, 131.37, 131.31, 130.00, 129.68, 127.26, 115.49, 115.32, 52.08. MS (ESI) m/z=271.1 [M+H]⁺.

### b. Preparation of (5-Carbamoyl-4′-fluoro-[1,1′-biphenyl]-2-yl)methanaminium chloride (Compound 7)

To the solution of 40 (0.040 g, 0.15 mmol) and Ph₃P (0.080 g, 0.30 mmol) in THF (10 mL), water (0.0070 g, 0.37 mmol) was added. The resulting mixture was heated to gentle reflux and stirred for 8 h. The solvent was then removed under vacuum, and the residue was dissolved into 4 M HCl in dioxane (3 mL). The mixture was stirred at room temperature for 15 min. The solvent was then removed under reduced pressure to yield the crude product. It was dissolved with deionized water (10 mL), washed with EtOAc (5 mL×3), and lyophilized to give 7 (0.03 g, 71% yield) as a white solid. ¹H NMR (500 MHz, CD₃OD): δ ppm 7.99 (dd, J=2.0, 8.0 Hz, 1H), 7.85 (d, J=2.0 Hz, 1H), 7.69 (d, J=8.0 Hz, 1H), 7.42 (dd, J=5.0, 8.0 Hz, 2H), 7.25 (t, J=8.0 Hz, 2H), 4.15 (s, 2H). ¹³C NMR (125 MHz, CD₃OD): δ ppm 170.66, 169.97, 163.91, 161.94, 141.69, 135.33, 135.30, 134.46, 131.21, 131.14, 129.84, 128.45, 127.39, 115.59, 115.42, 39.99. HRMS (ESI) Calcd for C₁₄H₁₃FN₂O (M+H)⁺ 245.1085, found 245.1079.

### 6. Preparation of Compounds 9-15

The synthesis scheme (Synthesis Scheme 6) for the preparation of Compounds 9-15 is shown below. The synthesis proceeds through the intermediates indicated (Compounds 1, 2, 3, 31, 41, 42a-c, 43a-d, and 44a-g). The yield for each synthetic step was as indicated.

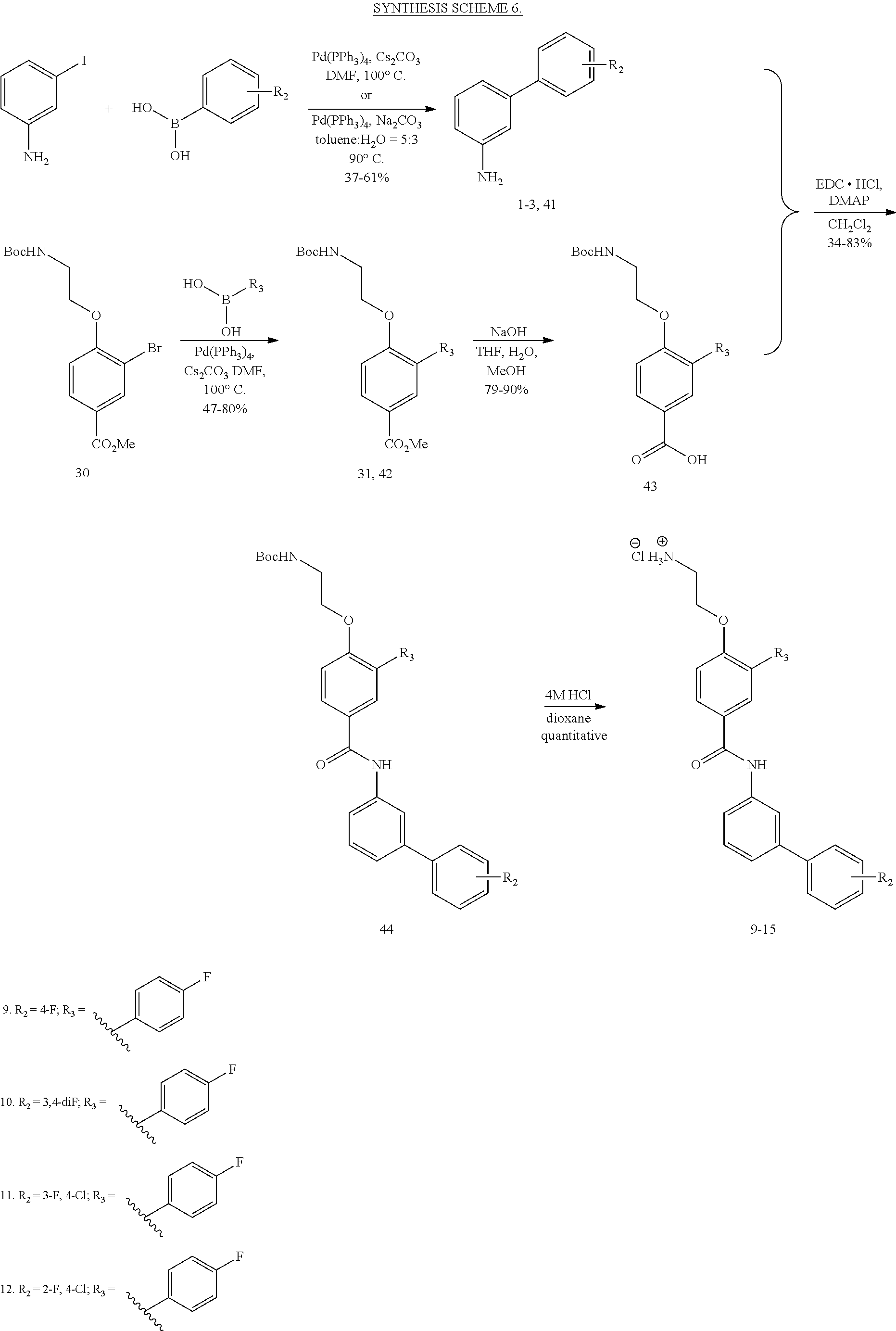

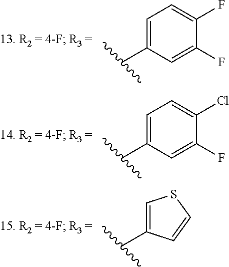

### a. Preparation of 4′-Chloro-3′-fluoro-[1,1′-biphenyl]-3-amine (Compound 41)

To a solution of 3-iodoaniline (0.96 g, 5.48 mmol) in a solvent mixture (48 mL, toluene:H₂O=5:3) was added (4-chloro-3-fluorophenyl) boronic acid (0.96 g, 5.48 mmol), Pd(PPh₃)₄ (0.26 g, 0.23 mmol), and Na₂CO₃ (1.45 g, 13.71 mmol). The mixture was then heated to 90° C. under argon and stirred for 16 h. The mixture was diluted into EtOAc (50 mL) and washed with water (2×50 mL), brine (50 mL), dried over MgSO₄. After the filtration, the solvent was removed under reduced pressure. The residue was then purified by column chromatography (silica gel, hexanes:EtOAc=3:1) to yield 41 (0.37 g, 37% yield) as a colorless oil. ¹H NMR (500 MHz, CDCl₃): δ ppm 7.59 (dd, J=2.2, 7.1 Hz, 1H), 7.40 (ddd, J=2.3, 4.5, 8.5 Hz, 1H), 7.23 (t, J=7.8 Hz, 1H), 7.18 (t, J=8.7 Hz, 1H), 6.91 (d, J=7.7 Hz, 1H), 6.81 (t, J=1.9 Hz, 1H), 6.70 (dd, J=2.2, 8.0 Hz, 1H), 3.77 (s, 2H). ¹³C NMR (125 MHz, CDCl₃): δ ppm 158.78, 156.80, 147.16, 140.40, 138.92, 138.89, 130.14, 129.38, 126.96, 126.91, 117.57, 116.98, 116.81, 114.77, 113.77.

### b. Preparation of Methyl 6-(2-((tert-butoxycarbonyl) amino) ethoxy)-3′,4′-difluoro-[1,1′-biphenyl]-3-carboxylate (Compound 42a)

42a (0.25 g, 47% yield) as an off-white solid. ¹H NMR (500 MHz, CDCl₃): δ ppm 8.01 (dd, J=2.2, 8.6 Hz, 1H), 7.96 (d, J=2.2 Hz, 1H), 7.36-7.33 (m, 1H), 7.32-7.16 (m, 2H), 6.98 (d, J=8.7 Hz, 1H), 4.75 (s, 1H), 4.10 (t, J=5.1 Hz, 2H), 3.89 (s, 3H), 3.48 (dd, J=5.2, 10.7 Hz, 2H), 1.42 (s, 9H). ¹³C NMR (125 MHz, CDCl₃): δ ppm 166.60, 158.97, 155.87, 151.02, 150.92, 150.87, 150.77, 149.05, 148.95, 148.89, 148.79, 134.41, 134.38, 134.37, 134.34, 132.33, 131.43, 131.37, 128.79, 125.67, 125.64, 125.62, 125.59, 123.37, 118.64, 118.54, 118.50, 117.08, 116.95, 112.11, 79.81, 67.94, 52.15, 28.43.

### c. Preparation of Methyl 6-(2-((tert-butoxycarbonyl) amino) ethoxy)-4′-chloro-3′-fluoro-[1,1′-biphenyl]-3-carboxylate (Compound 42b)

42b (0.31 g, 56% yield) as a white solid. ¹H NMR (500 MHz, d⁶-DMSO): δ ppm 8.01 (dd, J=2.2, 8.6 Hz, 1H), 7.96 (d, J=2.2 Hz, 1H), 7.57 (dd, J=1.9, 7.1 Hz, 1H), 7.37 (ddd, J=2.2, 4.6, 8.1 Hz, 1H), 7.18 (t, J=8.7 Hz, 1H), 6.98 (d, J=8.7 Hz, 1H), 4.75 (s, 1H), 4.10 (t, J=5.3 Hz, 2H), 3.89 (s, 1H), 3.48 (dd, J=5.0, 10.3 Hz, 2H), 1.42 (s, 9H). ¹³C NMR (125 MHz, d⁶-DMSO): δ ppm 166.60, 158.98, 158.52, 156.53, 134.57, 134.54, 132.27, 131.69, 131.42, 129.33, 129.28, 128.60, 123.37, 116.41, 116.25, 112.08, 112.04, 79.82, 67.94, 52.17, 28.46.

### d. Preparation of Methyl 4-(2-((tert-butoxycarbonyl) amino) ethoxy)-3-(thiophen-3-yl) benzoate (Compound 42c)

42c (0.36 g, 71% yield) as a white solid. ¹H NMR (500 MHz, CDCl₃): δ ppm 8.18 (d, J=2.2 Hz, 1H), 7.95 (dd, J=2.2, 8.7 Hz, 1H,), 7.61 (d, J=2.0 Hz, 1H), 7.44 (dd, J=0.9, 5.0 Hz, 1H), 7.37 (dd, J=3.0, 5.0 Hz, 1H), 6.97 (d, J=8.7 Hz, 1H), 4.85 (s, 1H), 4.14 (t, J=5.1 Hz, 2H), 3.90 (s, 3H), 3.56 (dd, J=5.1, 10.4 Hz, 2H), 1.44 (s, 9H). ¹³C NMR (125 MHz, CDCl₃): δ ppm 173.14, 166.83, 159.10, 137.39, 131.57, 130.42, 128.42, 125.41, 124.98, 123.80, 123.24, 112.07, 79.81, 68.05, 52.11, 40.13, 28.50.

### e. Preparation of 6-(2-((tert-Butoxycarbonyl) amino) ethoxy)-4′-fluoro-[1,1′-biphenyl]-3-carboxylic acid (Compound 43a)

To a solution of 31 (2.43 g, 6.33 mmol) in a solvent mixture (12 mL THF:H₂O:MeOH=4:1:1) was added 6 M NaOH (10 mL), and the reaction stirred at room temperature for 50 h. THF and MeOH were then removed under reduced pressure. The remaining aqueous solution was acidified with 6 M HCl to pH=4. The product was extracted with EtOAc (50 mL), and the organic layer was washed with water (25 mL), brine (25 mL), and dried over MgSO₄. After the filtration, the solvent was removed under reduced pressure to yield 43a (2.10 g, 90%) as a white solid. ¹H NMR (500 MHz, d⁶-DMSO): δ ppm 7.46 (d, J=8.5 Hz, 1H), 7.40 (s, 1H), 7.15-7.13 (m, 2H), 6.78-6.74 (m, 3H), 6.50 (t, J=5.5 Hz, 1H), 3.64 (t, J=5.5 Hz, 2H), 2.84 (q, J=5.4 Hz, 2H), 0.91 (s, 9H). ¹³C NMR (125 MHz, d⁶-DMSO): δ ppm 167.77, 163.05, 161.11, 159.24, 156.26, 134.12, 132.26, 131.94, 131.88, 131.28, 129.15, 124.283, 115.63, 115.46, 112.97, 78.45, 67.58, 67.52, 28.87.

### f. Preparation of 6-(2-((tert-Butoxycarbonyl) amino) ethoxy)-3′,4′-difluoro-[1,1′-biphenyl]-3-carboxylic acid (Compound 43b)

43b (0.55 g, 89%) as a white solid. ¹H NMR (500 MHz, d⁶-DMSO): δ ppm 7.92 (dd, J=2.1, 8.6 Hz, 1H), 7.85 (d, J=1.9 Hz, 1H), 7.61 (dd, J=8.3, 10.2 Hz, 1H), 7.43-7.37 (m, 1H), 7.36-7.35 (m, 1H), 7.20 (d, J=8.7 Hz, 1H), 6.95 (t, J=5.4 Hz, 1H), 4.10 (t, J=5.5 Hz, 2H), 3.28 (q, J=5.4 Hz, 2H), 1.33 (s, 9H). ¹³C NMR (125 MHz, d⁶-DMSO): δ ppm 167.05, 167.01, 158.50, 155.62, 150.01, 149.91, 149.81, 149.71, 148.06, 147.96, 147.85, 147.75, 134.61, 134.59, 134.55, 134.54, 131.75, 131.68, 131.08, 127.43, 126.15, 126.11, 126.08, 126.06, 123.66, 123.63, 118.50, 118.35, 117.13, 116.99, 112.32, 94.33, 77.75, 66.89, 28.15. MS (ESI) m/z=394.6 [M+H]⁺.

### g. Preparation of 6-(2-((tert-Butoxycarbonyl) amino) ethoxy)-4′-chloro-3′-fluoro-[1,1′-biphenyl]-3-carboxylic acid (Compound 43c)

43c (0.32 g, 85%) as a white solid. ¹H NMR (500 MHz, d⁶-DMSO): δ ppm 7.94 (d, J=8.4 Hz, 1H), 7.86 (s, 1H), 7.73 (d, J=6.3 Hz, 1H), 7.56-7.53 (m, 1H), 7.40 (t, J=8.9 Hz, 1H), 7.20 (d, J=8.2 Hz, 1H), 6.93 (t, J=5.3 Hz, 1H), 4.11 (t, J=5.1 Hz, 2H), 3.28 (dd, J=5.3, 10.8 Hz, 2H), 1.34 (s, 9H). ¹³C NMR (125 MHz, d⁶-DMSO): δ ppm 167.38, 158.37, 158.35, 157.44, 155.62, 134.97, 134.94, 131.70, 131.15, 130.00, 129.94, 127.20, 127.18, 127.15, 127.14, 127.13, 119.20, 119.06, 116.53, 116.36, 112.24, 77.76, 66.85, 28.17.

### h. Preparation of 4-(2-((tert-Butoxycarbonyl) amino) ethoxy)-3-(thiophen-3-yl) benzoic acid (Compound 43d)

(0.27 g, 79%) as a white solid. ¹H NMR (500 MHz, d⁶-DMSO): δ ppm 12.74 (brs, 1H), 8.10 (d, J=1.7 Hz, 1H), 7.94 (s, 1H), 7.85 (dd, J=2.0, 8.6 Hz, 1H), 7.56-7.54 (m, 2H), 7.18 (d, J=8.7 Hz, 1H), 7.09 (t, J=5.5 Hz, 1H), 4.13 (t, J=5.3 Hz, 2H), 3.39 (dd, J=5.2, 10.7 Hz, 2H), 1.38 (s, 9H). ¹³C NMR (125 MHz, d⁶-DMSO): δ ppm 166.98, 158.70, 155.68, 136.50, 130.29, 129.97, 127.97, 125.34, 124.45, 123.88, 123.12, 112.32, 77.84, 67.20, 28.26. MS (ESI) m/z=364.7 [M+H]⁺.

### i. Preparation of tert-Butyl (2-((4′-fluoro-5-((4′-fluoro-[1,1′-biphenyl]-3-yl) carbamoyl)-[1,1′-biphenyl]-2-yl) oxy) ethyl) carbamate (Compound 44a)

To a solution of 43a (0.25 g, 0.67 mmol) in CH₂Cl₂ (20 mL) was added 1 (0.14 g, 0.73 mmol), Et₃N (0.23 mL, 1.67 mmol), EDC□HCl (0.17 g, 0.88 mmol), and DMAP (0.09 g, 0.73 mmol). The mixture was then stirred at room temperature for 20 h. The mixture was diluted with CH₂Cl₂ (50 mL) and washed with water (50 mL), brine (50 ml), and dried over MgSO₄. After the filtration, the solvent was removed under reduced pressure. The residue was then purified by column chromatography (silica gel, hexanes:EtOAc=1:1) to yield 44a (0.12 g, 34% yield) as a white solid. ¹H NMR (500 MHz, d⁶-Acetone): δ ppm 9.65 (s, 1H), 8.16 (d, J=1.9 Hz, 1H), 8.06-8.04 (m, 2H), 7.87 (s, 1H), 7.69-7.65 (m, 4H), 7.43 (ddd, J=1.8, 6.0, 7.9 Hz, 1H), 7.36 (dd, J=1.4, 6.3 Hz, 1H), 7.30-7.16 (m, 5H), 6.08 (s, 1H), 4.19 (t, J=5.5 Hz, 2H), 3.48-3.45 (m, 2H), 1.41 (s, 9H). ¹³C NMR (125 MHz, d⁶-Acetone): δ ppm 165.46, 164.01, 163.58, 162.07, 161.64, 158.85, 140.95, 140.73, 137.84, 137.81, 134.57, 134.54, 132.04, 131.98, 130.44, 129.79, 129.41, 129.30, 129.23, 128.31, 128.28, 122.52, 122.50, 119.59, 119.50, 119.12, 119.03, 116.17, 115.99, 115.38, 115.21, 112.87, 78.61, 67.91, 40.21, 28.30.

### j. Preparation of tert-Butyl (2-((5-((3′,4′-difluoro-[1,1′-biphenyl]-3-yl) carbamoyl)-4′-fluoro-[1,1′-biphenyl]-2-yl) oxy) ethyl) carbamate (Compound 44b)

44b (0.30 g, 79% yield) as a white solid. ¹H NMR (500 MHz, d⁶-Acetone): δ ppm 9.65 (s, 1H), 8.17 (d, J=1.9 Hz, 1H), 8.05-8.03 (m, 2H), 7.89 (d, J=7.9 Hz, 1H), 7.68-7.66 (m, 2H), 7.59 (ddd, J=2.1, 7.7, 11.9 Hz, 1H), 7.56-7.38 (m, 4H), 7.25 (d, J=8.5 Hz, 1H), 7.20-7.17 (m, 2H), 6.08 (s, 1H), 4.20 (t, J=5.6 Hz, 2H), 3.47 (q, J=5.8 Hz, 2H), 1.41 (s, 9H). ¹³C NMR (125 MHz, d⁶-Acetone): δ ppm 165.76, 163.90, 161.95, 159.19, 152.20, 152.10, 151.62, 151.52, 150.25, 150.14, 149.66, 149.56, 141.13, 140.08, 134.86, 134.84, 132.35, 132.29, 130.75, 130.22, 129.72, 128.53, 124.25, 124.22, 124.20, 124.17, 122.84, 120.42, 119.43, 118.63, 118.49, 116.62, 116.47, 115.69, 115.52, 113.18, 78.92, 68.23, 40.52, 28.60.

### k. Preparation of tert-Butyl (2-((5-((4′-chloro-3′-fluoro-[1,1′-biphenyl]-3-yl) carbamoyl)-4′-fluoro-[1,1′-biphenyl]-2-yl) oxy) ethyl) carbamate (Compound 44c)

44c (0.64 g, 83% yield) as a white solid. ¹H NMR (500 MHz, d⁶-Acetone): δ ppm 9.64 (s, 1H), 8.15 (s, 1H), 8.13-8.10 (m, 2H), 7.96 (d, J=8.0 Hz, 1H), 7.83 (dd, J=2.3, 7.1 Hz, 1H,), 7.73-7.71 (m, 3H), 7.49-7.46 (m, 3H), 7.33 (d, J=8.6 Hz, 1H), 7.27-7.24 (m, 2H), 6.14 (s, 1H), 4.27 (t, J=5.7 Hz, 2H), 3.53 (q, J=5.8 Hz, 2H), 1.47 (s, 9H). ¹³C NMR (125 MHz, d⁶-Acetone): δ ppm 165.73, 163.91, 161.97, 159.34, 159.21, 157.37, 141.17, 139.90, 139.53, 139.50, 134.88, 134.86, 132.37, 132.30, 130.76, 130.26, 129.73, 129.71, 128.54, 128.13, 128.07, 122.84, 120.40, 119.40, 118.03, 117.86, 115.70, 115.53, 113.20, 78.90, 68.24, 40.53, 28.60.

### l. Preparation of tert-Butyl(2-((5-((4′-chloro-2′-fluoro-[1,1′-biphenyl]-3-yl)carbamoyl)-4′-fluoro-[1,1′-biphenyl]-2-yl) oxy) ethyl) carbamate (Compound 44d)

44d (0.30 g, 79% yield) as a white solid. ¹H NMR (500 MHz, d⁶-Acetone): δ ppm 9.66 (s, 1H), 8.07-8.03 (m, 3H), 7.92 (dd, J=2.0, 8.2 Hz, 1H), 7.67 (dd, J=5.5, 8.8 Hz, 2H), 7.55 (d, J=8.6 Hz, 1H), 7.46 (t, J=7.9 Hz, 1H), 7.36 (ddd, J=1.9, 5.0, 8.2 Hz, 2H), 7.29 (d, J=7.7 Hz, 1H), 7.25 (d, J=8.5 Hz, 1H), 7.20-7.17 (m, 2H), 6.07 (s, 1H), 4.20 (t, J=5.7 Hz, 2H), 3.46 (q, J=5.8 Hz, 2H), 1.40 (s, 9H). ¹³C NMR (125 MHz, d⁶-Acetone): δ ppm 165.74, 163.90, 161.96, 161.32, 159.18, 140.82, 135.88, 135.87, 134.88, 134.86, 134.47, 134.39, 132.77, 132.73, 132.36, 132.29, 130.76, 130.09, 129.81, 129.74, 128.56, 125.90, 125.87, 124.84, 124.82, 121.34, 121.32, 120.65, 117.60, 117.38, 115.69, 115.52, 113.18, 78.91, 78.90, 68.24, 40.53, 28.60.

### m. Preparation of tert-Butyl (2-((3′,4′-difluoro-5-((4′-fluoro-[1,1′-biphenyl]-3-yl) carbamoyl)-[1,1′-biphenyl]-2-yl) oxy) ethyl) carbamate (Compound 44e)

44e (0.26 g, 71% yield) as a white solid. ¹H NMR (500 MHz, d⁶-Acetone): δ ppm 9.64 (s, 1H), 8.14 (t, J=1.7 Hz, 1H), 8.05-8.03 (m, 2H), 7.85 (dd, J=2.0, 8.0 Hz, 1H), 7.68 (ddd, J=2.7, 6.0, 7.7 Hz, 2H), 7.62 (ddd, J=2.0, 7.9, 12.0 Hz, 1H,), 7.45-7.40 (m, 4H), 7.26-7.23 (m, 3H), 6.14 (s, 1H), 4.22 (t, J=5.5 Hz, 2H), 3.50 (q, J=5.7 Hz, 2H), 1.40 (s, 9H). ¹³C NMR (125 MHz, d⁶-Acetone): δ ppm 165.61, 164.33, 162.38, 159.06, 156.70, 156.69, 151.50, 151.41, 151.29, 151.18, 149.55, 149.45, 149.33, 149.22, 141.27, 140.99, 138.13, 138.11, 136.03, 136.00, 130.77, 130.22, 130.10, 129.61, 129.54, 128.88, 128.64, 127.11, 127.08, 127.06, 127.03, 122.86, 119.90, 119.80, 119.49, 119.43, 119.34, 117.77, 117.63, 116.48, 116.31, 113.20, 78.91, 68.27, 40.50, 28.57; MS (ESI) m/z=563.9 [M+H]⁺.

### n. Preparation of tert-Butyl (2-((4′-chloro-3′-fluoro-5-((4′-fluoro-[1,1′-biphenyl]-3-yl) carbamoyl)-[1,1′-biphenyl]-2-yl) oxy) ethyl) carbamate (Compound 44f)

44f (0.22 g, 62% yield) as a white solid. ¹H NMR (500 MHz, d⁶-Acetone): δ ppm 9.63 (s, 1H), 8.14 (s, 1H), 8.06-8.04 (m, 2H), 7.85 (d, J=7.6 Hz, 1H), 7.78 (d, J=6.0 Hz, 1H), 7.68-7.64 (m, 3H), 7.46-7.19 (m, 6H), 6.10 (s, 1H), 4.24-4.21 (m, 2H), 3.48 (dd, J=5.5, 11.3 Hz, 2H), 1.40 (s, 9H). ¹³C NMR (125 MHz, d⁶-Acetone): δ ppm 165.57, 164.35, 162.41, 159.09, 157.01, 141.30, 141.02, 138.17, 136.27, 132.39, 131.01, 130.95, 130.72, 130.31, 130.11, 129.63, 129.56, 128.72, 122.86, 119.88, 119.79, 119.42, 119.33, 117.19, 117.02, 116.50, 116.32, 113.24, 78.91, 68.28, 40.52, 28.59.

### o. Preparation of tert-Butyl (2-(4-((4′-fluoro-[1,1′-biphenyl]-3-yl) carbamoyl)-2-(thiophen-3-yl) phenoxy) ethyl) carbamate (Compound 44g)

44g (0.24 g, 67% yield) as a white solid. ¹H NMR (500 MHz, d⁶-DMSO): δ ppm 9.79 (s, 1H), 7.80 (s, 1H), 7.63 (s, 1H), 7.56 (s, 1H), 7.50 (d, J=8.7 Hz, 1H), 7.37 (d, J=8.0 Hz, 1H), 7.24-7.22 (m, 3H), 7.16-7.14 (m, 1H), 7.00 (t, J=7.9 Hz, 1H), 6.92 (d, J=7.7 Hz, 1H), 6.87 (t, J=8.5 Hz, 2H), 6.81 (d, J=8.7 Hz, 1H), 6.64 (t, J=5.0 Hz, 1H), 3.72 (t, J=5.2 Hz, 2H), 2.98 (d, J=5.3 Hz, 2H), 0.95 (s, 9H). ¹³C NMR (125 MHz, d⁶-DMSO): δ ppm 164.84, 162.83, 160.884, 157.77, 155.66, 139.84, 139.50, 136.62, 136.60, 129.17, 128.62, 128.56, 128.50, 128.34, 128.27, 126.96, 125.11, 124.41, 123.64, 121.73, 119.30, 118.62, 115.79, 115.62, 112.25, 77.80, 67.18, 39.41, 28.22.

### p. Preparation of 2-((4′-Fluoro-5-((4′-fluoro-[1,1′-biphenyl]-3-yl)carbamoyl)-[1,1′-biphenyl]-2-yl) oxy) ethanaminium chloride (Compound 9)

To a solution of 44a (0.06 g, 0.11 mmol) in MeOH (10 mL) under anhydrous conditions was added 4 M HCl in dioxane (10 mL, 0.04 mmol), and the mixture was stirred at room temperature for 1 h. The solvent was then removed under reduced pressure to yield 9 (0.53 g, quantitative yield) as an off-white solid. ¹H NMR (500 MHz, d⁶-DMSO): δ ppm 10.36 (s, 1H), 8.29 (s, 3H), 8.10-8.05 (m, 3H), 7.84 (d, J=8.1 Hz, 1H), 7.70-7.67 (m, 4H), 4.35 (t, J=4.6 Hz, 2H), 3.39-3.19 (m, 2H). ¹³C NMR (125 MHz, d⁶-DMSO): δ ppm 164.71, 162.87, 162.51, 160.92, 160.57, 157.24, 139.86, 139.51, 136.64, 136.61, 133.51, 133.49, 131.59, 131.53, 130.22, 129.22, 129.15, 128.66, 128.61, 128.54, 127.69, 121.81, 119.35, 118.67, 115.90, 115.85, 115.68, 115.09, 114.92, 112.80, 65.25, 38.01. HRMS (ES+) m/z [C₂₇H₂₃F₂N₂O₂⁺] calculated 445.1722, found=445.1728.

### q. Preparation of 2-((5-((3′,4′-Difluoro-[1,1′-biphenyl]-3-yl) carbamoyl)-4′-fluoro-[1,1′-biphenyl]-2-yl) oxy) ethanaminium chloride (Compound 10)

10 (0.42 g, 86% yield) as an off-white solid. ¹H NMR (500 MHz, d⁶-DMSO): δ ppm 10.35 (s, 1H), 8.26 (s, 3H), 8.09 (s, 1H), 8.05-8.03 (m, 2H), 7.85 (d, J=8.0 Hz, 1H), 7.73-7.70 (m, 3H), 7.47-7.44 (m, 4H), 7.29-7.26 (m, 3H), 4.33 (t, J=5.4 Hz, 2H), 3.18 (s, 2H). ¹³C NMR (125 MHz, d⁶-DMSO): δ ppm 165.40, 163.20, 161.25, 157.94, 151.45, 151.35, 150.81, 150.71, 149.50, 149.40, 148.85, 148.75, 140.59, 139.01, 138.54, 134.19, 132.28, 132.21, 130.89, 129.99, 129.84, 129.35, 128.31, 124.07, 122.61, 120.58, 119.44, 118.75, 118.62, 116.35, 116.21, 115.78, 115.61, 113.49, 65.93, 38.70. HRMS (ES+) m/z [C₂₇H₂₂F₃N₂O₂⁺] calculated 463.1628, found=463.1638.

### r. Preparation of 2-((5-((4′-Chloro-3′-fluoro-[1,1′-biphenyl]-3-yl) carbamoyl)-4′-fluoro-[1,1′-biphenyl]-2-yl) oxy) ethanaminium chloride (Compound 11)

11 (0.45 g, 84% yield) as an off-white solid. ¹H NMR (500 MHz, d⁶-DMSO): δ ppm 10.38 (s, 1H), 8.36 (s, 3H), 8.07-8.03 (m, 3H), 7.75-7.70 (m, 5H), 7.40-7.35 (m, 6H), 4.32 (t, J=4.8 Hz, 2H), 3.16 (t, 2H, J=4.8 Hz, 2H). ¹³C NMR (125 MHz, d⁶-DMSO): δ ppm 165.41, 165.32, 163.18, 161.23, 158.51, 157.93, 156.54, 140.63, 140.53, 138.77, 138.68, 138.65, 134.16, 134.14, 132.28, 132.21, 130.90, 129.99, 129.83, 129.32, 129.14, 128.27, 128.22, 127.92, 127.86, 122.55, 120.79, 120.65, 120.54, 120.44, 119.42, 119.31, 118.15, 117.98, 115.75, 115.58, 113.45, 65.86, 38.60. HRMS (ES+) m/z [C₂₇H₂₂ClF₂N₂O₂⁺] calculated 479.1332, found=479.1345.

### s. Preparation of 2-((5-((4′-Chloro-2′-fluoro-[1,1′-biphenyl]-3-yl) carbamoyl)-4′-fluoro-[1,1′-biphenyl]-2-yl) oxy) ethanaminium chloride (Compound 12)

12 (0.45 g, 90% yield) as an off-white solid. ¹H NMR (500 MHz, d⁶-DMSO): δ ppm 10.38 (s, 1H), 8.29 (s, 3H), 8.06-8.03 (m, 3H), 7.87 (d, J=8.0 Hz, 1H), 7.75-7.72 (m, 2H), 7.57-7.54 (m, 2H), 7.42-7.40 (m, 2H), 7.33-7.26 (m, 4H), 4.33 (t, J=5.4 Hz, 2H), 3.17 (t, J=5.3 Hz, 2H). ¹³C NMR (125 MHz, d⁶-DMSO): δ ppm 165.45, 163.19, 161.24, 160.56, 158.57, 157.94, 140.30, 134.97, 134.18, 134.16, 133.72, 133.64, 132.58, 132.54, 132.27, 132.21, 130.92, 129.86, 129.64, 129.33, 128.30, 128.02, 127.92, 125.91, 125.88, 124.54, 124.52, 121.31, 121.29, 120.78, 117.52, 117.31, 115.77, 115.60, 113.47, 65.92, 38.68. HRMS (ES+) m/z [C₂₇H₂₂ClF₂N₂O₂⁺] calculated 479.1332, found=479.1339.

### t. Preparation of 2-((3′,4′-Difluoro-5-((4′-fluoro-[1,1′-biphenyl]-3-yl) carbamoyl)-[1,1′-biphenyl]-2-yl) oxy) ethanaminium chloride (Compound 13)

13 (0.15 g, quantitative yield) as an off-white solid. ¹H NMR (500 MHz, d⁶-DMSO): δ ppm 10.39 (s, 1H), 8.33 (s, 3H), 8.12-7.85 (m, 3H), 7.85-7.81 (m, 2H), 7.70-7.67 (m, 2H), 7.66-7.29 (m, 7H), 4.34 (t, J=5.3 Hz, 2H), 3.19 (s, 2H). ¹³C NMR (125 MHz, d⁶-DMSO): δ ppm 165.30, 163.55, 161.60, 157.82, 150.81, 150.71, 150.55, 150.45, 148.87, 148.77, 148.59, 148.49, 140.53, 140.18, 137.31, 137.29, 135.34, 135.31, 135.29, 135.26, 130.97, 130.35, 129.89, 129.28, 129.22, 128.35, 128.13, 127.25, 127.23, 127.20, 127.18, 122.50, 120.06, 119.40, 119.38, 119.26, 117.89, 117.76, 116.53, 116.36, 113.41, 65.93, 38.66. HRMS (ES+) m/z [C₂₇H₂₂F₃N₂O₂⁺] calculated 463.1628, found=463.1637.

### u. Preparation of 2-((4′-Chloro-3′-fluoro-5-((4′-fluoro-[1,1′-biphenyl]-3-yl) carbamoyl)-[1,1′-biphenyl]-2-yl) oxy) ethanaminium chloride (Compound 14)

14 (0.21 g, quantitative yield) as an off-white solid. ¹H NMR (500 MHz, d⁶-DMSO): δ ppm 10.35 (s, 1H), 8.26 (s, 3H), 8.06-8.01 (m, 3H), 7.89 (dd, J=2.2, 7.3 Hz, 1H), 7.82 (d, J=8.2 Hz, 1H), 7.70-7.67 (m, 3H), 7.57-7.28 (m, 6H), 4.33 (t, J=5.4 Hz, 2H), 3.18 (s, 2H). ¹³C NMR (125 MHz, d⁶-DMSO): δ ppm 165.27, 163.55, 161.60, 158.22, 157.84, 156.26, 140.50, 140.19, 137.31, 137.28, 135.62, 135.58, 132.00, 131.12, 131.06, 130.91, 130.40, 129.91, 129.29, 129.22, 128.36, 127.99, 122.53, 120.06, 119.98, 119.84, 119.37, 117.34, 117.17, 116.54, 116.37, 113.45, 65.92, 38.68. HRMS (ES+) m/z [C₂₇H₂₂ClF₂N₂O₂⁺] calculated 479.1332, found=479.1343.

### v. Preparation of 2-(4-((4′-Fluoro-[1,1′-biphenyl]-3-yl) carbamoyl)-2-(thiophen-3-yl) phenoxy) ethanaminium chloride (Compound 15)

15 (0.15 g, quantitative yield) as an off-white solid. ¹H NMR (500 MHz, d⁶-DMSO): δ ppm 10.37 (s, 1H), 8.34 (s, 3H), 8.24 (d, J=2.3 Hz, 1H), 8.05-8.01 (m, 3H), 7.83 (d, J=8.0 Hz, 1H), 7.66-7.60 (m, 4H), 7.38-7.30 (m, 5H), 4.37 (t, J=5.3 Hz, 2H), 3.28 (t, J=5.1 Hz, 2H). ¹³C NMR (125 MHz, d⁶-DMSO): δ ppm 165.49, 163.54, 161.60, 157.86, 140.56, 140.17, 137.43, 137.33, 137.30, 129.90, 129.80, 129.35, 129.29, 129.23, 128.28, 126.05, 125.58, 124.87, 122.48, 120.08, 119.39, 116.54, 116.37, 113.42, 66.09, 38.82. HRMS (ES+) m/z [C₂₅H₂₂FN₂O₂S⁺] calculated 433.1381, found=433.1393.

### 7. Preparation of Compounds 16 and 17

The synthesis scheme (Synthesis Scheme 7) for the preparation of Compounds 16 and 17 is shown below. The synthesis proceeds through the intermediates indicated (Compounds 45, 46, 47a, and 47b). The yield for each synthetic step was as indicated.

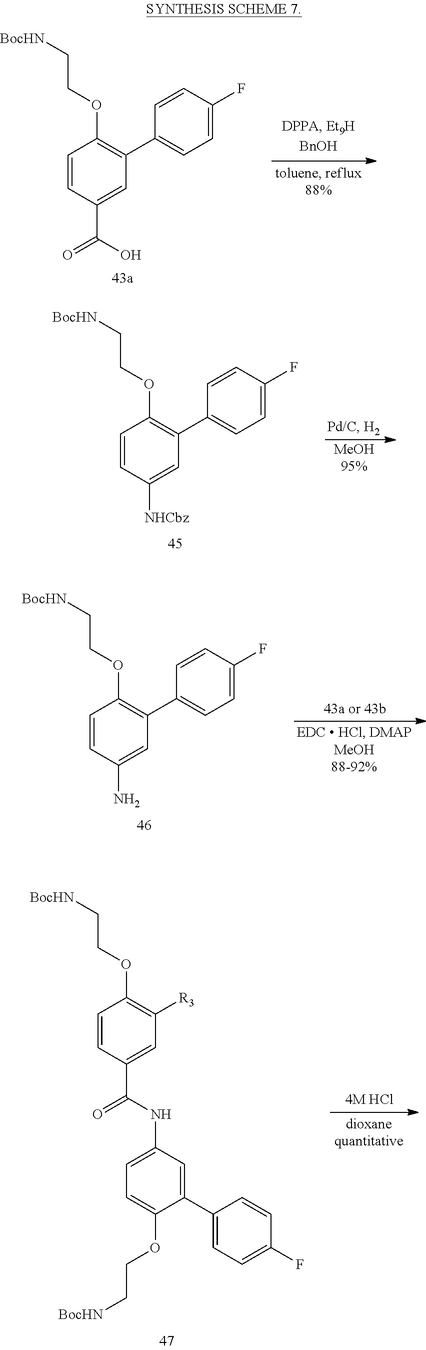

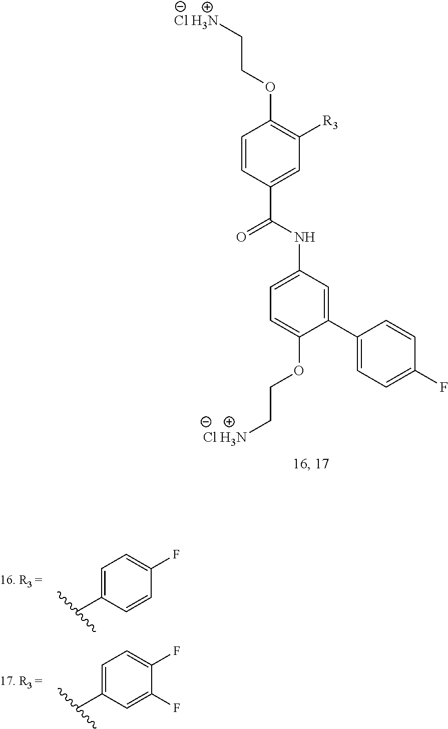

### a. Preparation of tert-Butyl (2-((5-(((benzyloxy) carbonyl) amino)-4′-fluoro-[1,1′-biphenyl]-2-yl) oxy) ethyl) carbamate (Compound 45)

To a solution of 43a (0.50 g, 1.33 mmol) in dry toluene (30 mL) under anhydrous conditions was added DPPA (0.29 mL, 1.33 mmol), Et₃N (0.19 ml, 2.59 mmol), benzyl alcohol (4.00 mL, 0.019 mmol), and activated molecular sieves (1 g). The mixture was stirred at room temperature for 10 min and then heated to 80° C. under nitrogen for 22 h. Upon completion, the molecule sieves were filtered, and the solution diluted with EtOAc (50 mL). The organic layer was washed with water (2×50 mL), brine (50 mL), and dried over MgSO₄. After the filtration, the solvent was removed under reduced pressure. The residue was purified by column chromatography (silica gel, hexanes:EtOAc=3:1) to yield 45 (0.56 g, 88% yield) as a white solid. ¹H NMR (500 MHz, CDCl₃): δ ppm 7.45-7.39 (m, 2H), 7.37-7.31 (m, 6H), 7.08 (t, J=8.5 Hz, 2H), 6.97 (s, 1H), 6.88 (d, J=8.7 Hz, 1H), 5.18 (s, 2H), 4.68 (s, 1H), 3.91 (t, J=4.8 Hz, 2H), 3.37 (d, J=4.9 Hz, 2H), 1.43 (s, 9H). ¹³C NMR (125 MHz, CDCl₃): δ ppm 163.25, 161.29, 156.00, 153.91, 151.81, 136.30, 134.00, 132.15, 131.23, 131.17, 128.78, 128.51, 128.46, 121.96, 119.69, 115.23, 115.06, 114.72, 79.65, 68.90, 67.16, 40.20, 28.55. MS (ESI) m/z=481.8 [M+H]⁺.

### b. Preparation of tert-Butyl (2-((5-amino-4′-fluoro-[1,1′-biphenyl]-2-yl) oxy) ethyl) carbamate (Compound 46)

To a solution of 45 (0.64 g, 1.33 mmol) in MeOH (20 mL) was added 10% Pd on activated carbon (0.06 g, 10% by weight). The air was evacuated and exchanged with the H₂ gas three times, and the reaction was allowed to stir under H₂ for 1.5 h. The mixture was filtered through celite and, the solvent was removed under reduced pressure to yield 46 (0.42 g, 92% yield) as an off-white solid. ¹H NMR (500 MHz, CDCl₃): δ ppm 7.40 (dd, J=5.6, 8.3 Hz, 2H), 7.04 (t, J=8.6 Hz, 2H), 6.87 (s, 2H), 6.80 (d, J=8.6 Hz, 1H), 6.22 (s, 2H), 4.69 (s, 1H), 3.84 (t, J=4.3 Hz, 2H), 3.32 (d, J=4.9 Hz, 2H), 1.41 (s, 9H). ¹³C NMR (125 MHz, CDCl₃): δ ppm 173.08, 163.11, 161.14, 155.89, 150.58, 135.82, 135.80, 133.84, 133.82, 131.76, 131.04, 130.98, 120.04, 117.90, 115.80, 115.13, 114.96, 79.47, 69.24, 40.07, 28.43. MS (ESI) m/z=347.6 [M+H]⁺.

### c. Preparation of tert-Butyl (2-((5-((6-(2-((tert-butoxycarbonyl) amino) ethoxy)-4′-fluoro-[1,1′-biphenyl]-3-yl) carbamoyl)-4′-fluoro-[1,1′-biphenyl]-2-yl) oxy) ethyl) carbamate (Compound 47a)

To a solution of 46 (0.37 g, 1.07 mmol) in CH₂Cl₂ (20 mL) was added 43a (0.44 g, 1.18 mmol), Et₃N (0.37 mL, 2.69 mmol), EDC.HCl (0.27 g, 1.42 mmol), and DMAP (0.14 g, 1.18 mmol). The mixture was stirred at room temperature for 3 h. The mixture was diluted with CH₂Cl₂ (50 mL), washed with water (2×50 ml) and brine (50 mL), and dried over MgSO₄. After the filtration, the solvent was removed under reduced pressure. The residue was purified by column chromatography (silica gel, hexanes:EtOAc=1:1) to yield 47a (0.54 g, 86% yield) as an off-white solid. ¹H NMR (500 MHz, CDCl₃): δ ppm 8.26 (s, 1H), 7.84 (d, J=9.0 Hz, 2H), 7.56 (s, 2H), 7.56-7.43 (m, 4H), 7.10-7.04 (m, 4H), 6.95 (d, J=9.0 Hz, 1H), 6.90 (d, J=9.0 Hz, 1H), 4.74 (brs, 1H), 4.69 (brs, 1H), 4.03 (t, 2H, J=5.2 Hz), 3.93 (t, 2H, J=5.1 Hz), 3.42-3.37 (m, 4H), 1.42 (s, 18H). ¹³C NMR (125 MHz, CDCl₃): δ ppm 165.23, 163.32, 163.17, 161.35, 161.21, 158.17, 155.94, 152.29, 133.90, 133.44, 132.30, 131.19, 131.12, 130.93, 130.32, 130.02, 128.28, 127.86, 123.48, 121.25, 115.28, 115.15, 115.11, 114.98, 114.34, 112.54, 79.83, 79.60, 68.70, 67.98, 40.12, 39.94, 28.48.

### d. Preparation of tert-Butyl (2-((5-((6-(2-((tert-butoxycarbonyl) amino) ethoxy)-4′-fluoro-[1,1′-biphenyl]-3-yl) carbamoyl)-3′,4′-difluoro-[1,1′-biphenyl]-2-yl) oxy) ethyl) carbamate (Compound 47b)

47b (0.22 g, 41% yield) as an off-white solid. ¹H NMR (500 MHz, CDCl₃): δ ppm 8.32 (s, 1H), 7.85-7.83 (m, 2H), 7.56 (d, J=7.3 Hz, 2H), 7.43 (dd, J=5.7, 8.0 Hz, 2H), 7.31-7.29 (m, 1H), 7.19-7.16 (m, 2H), 7.05 (t, J=8.7 Hz, 2H), 6.93 (d, J=9.0 Hz, 1H), 6.89 (d, J=9.0 Hz, 1H), 4.05 (t, J=5.3 Hz, 2H), 3.92 (t, J=5.2 Hz, 2H), 3.44-3.37 (m, 4H), 1.42 (s, 18H). ¹³C NMR (125 MHz, CDCl₃): δ ppm 165.09, 163.17, 161.21, 158.02, 155.96, 152.32, 134.34, 133.84, 132.23, 131.15, 131.08, 130.91, 130.02, 129.08, 128.68, 127.86, 125.67, 125.64, 125.62, 125.59, 123.52, 121.30, 118.63, 118.48, 117.11, 116.97, 115.14, 114.97, 114.30, 112.42, 79.91, 79.63, 68.66, 67.95, 40.11, 39.89, 28.48, 28.45. MS (ESI) m/z=744.3 [M+Na]⁺.

### e. Preparation of 2-((5-((6-(2-Ammonioethoxy)-4′-fluoro-[1,1′-biphenyl]-3-yl) carbamoyl)-4′-fluoro-[1,1′-biphenyl]-2-yl) oxy) ethanaminium chloride (Compound 16)

To a solution of 47a (0.465 g, 0.66 mmol) in MeOH (10 mL) under anhydrous conditions was added 4 M HCl in dioxane (10 mL, 0.04 mmol). The mixture was stirred at room temperature for 1 h. The solvent was then removed under reduced pressure to yield 16 (0.38 g, quantitative yield) as an off-white solid. ¹H NMR (500 MHz, d⁶-DMSO): δ ppm 10.36 (s, 1H), 8.34 (brs, 6H), 8.13-8.10 (m, 2H), 7.89 (dd, J=2.6, 12.1 Hz, 2H), 7.82 (dd, J=5.6, 8.5 Hz, 2H), 7.73-7.70 (m, 2H), 7.41-7.27 (m, 6H), 4.43 (t, J=5.4 Hz, 2H), 4.29 (t, J=5.4 Hz, 2H), 3.29 (t, J=5.3 Hz, 2H), 3.23 (t, J=5.3 Hz, 2H). ¹³C NMR (125 MHz, d⁶-DMSO): δ ppm 165.07, 163.17, 163.04, 161.23, 161.10, 157.81, 151.45, 134.69, 134.67, 134.18, 134.16, 134.14, 132.25, 132.19, 131.91, 131.84, 130.77, 129.85, 129.83, 129.71, 129.30, 128.36, 123.73, 121.60, 115.81, 115.76, 115.59, 114.77, 113.47, 66.21, 65.89, 38.92, 38.70. HRMS (ES+) m/z [C₂₉H₂₉F₂N₃O₃²⁺] calculated 505.2166, found=505.2128.

### f. Preparation of 2-((5-(6-(2-Ammonioethoxy)-3′,4′-difluoro-[1,1′-biphenyl]-3-ylcarboxamido)-4′-fluoro-[1,1′-biphenyl]-2-yl) oxy) ethanaminium chloride (Compound 17)

17 (0.18 g, quantitative yield) as an off-white solid. ¹H NMR (500 MHz, d⁶-DMSO): δ ppm 10.31 (s, 1H), 8.32 (brs, 6H), 8.03 (d, J=9.8 Hz, 2H), 7.84-7.80 (m, 3H), 7.65-7.62 (m, 2H), 7.54-7.50 (m, 2H), 7.32-7.17 (m, 4H), 4.33 (t, J=5.2 Hz, 2H), 4.18 (t, J=5.4 Hz, 2H), 3.18 (t, J=5.2 Hz, 2H), 3.11 (t, J=5.4 Hz, 2H). ¹³C NMR (125 MHz, d⁶-DMSO): δ ppm 164.95, 163.03, 161.09, 157.72, 151.45, 150.79, 150.69, 150.42, 148.85, 148.75, 148.56, 148.46, 135.35, 135.32, 135.29, 135.26, 134.71, 134.68, 134.15, 131.92, 131.85, 130.85, 130.23, 130.19, 129.81, 128.36, 128.09, 127.25, 127.22, 127.20, 127.17, 123.74, 121.60, 119.38, 119.24, 117.87, 117.74, 117.71, 115.75, 115.58, 114.74, 113.38, 66.18, 65.88, 38.86, 38.63. HRMS (ES+) m/z [C₂₉H₂₈F₃N₃O₃²⁺] calculated 523.2072, found=523.2039.

### 8. Preparation of Compound 18

The synthesis scheme (Synthesis Scheme 8) for the preparation of Compound 18 is shown below. The synthesis proceeds through the intermediates indicated (Compounds 48, 49, 50, 51, 52, 53, and 54). The yield for each synthetic step was as indicated.

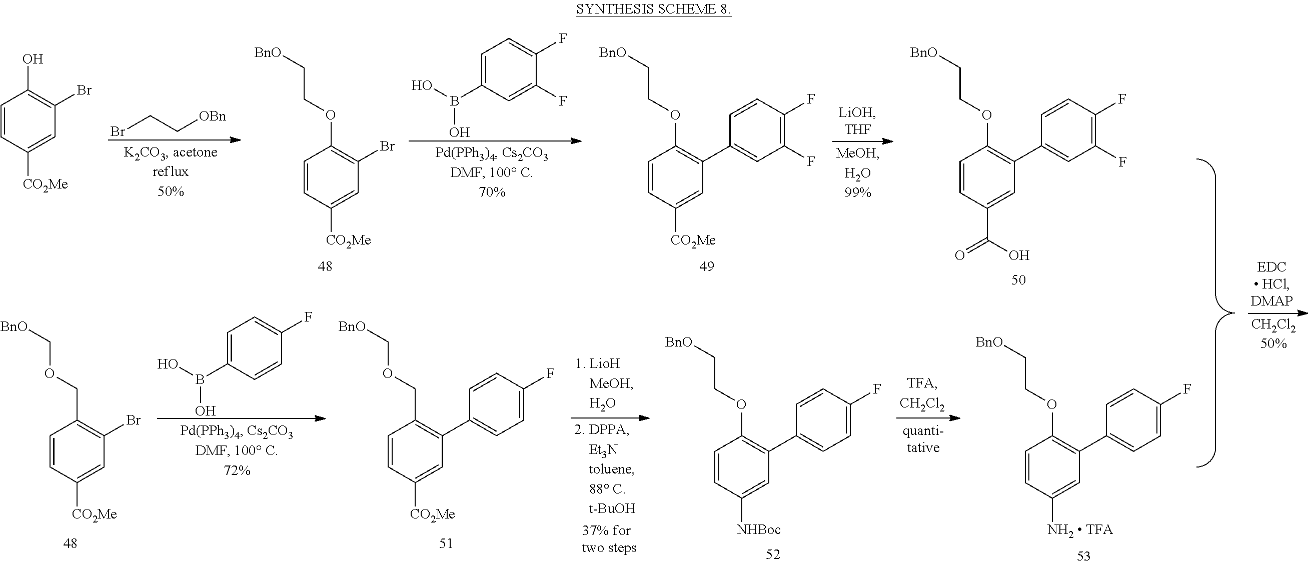

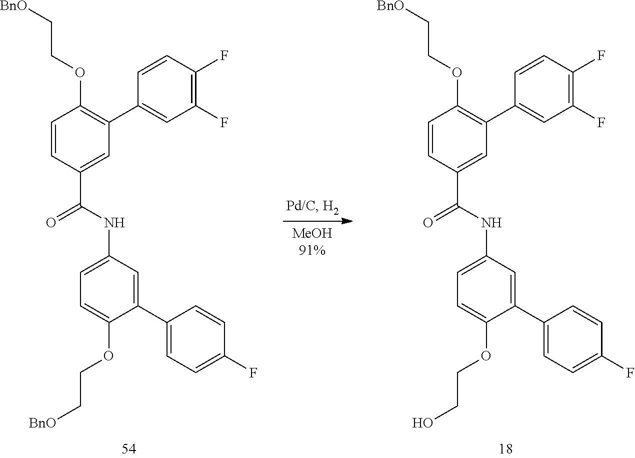 [SMILES: O=C(Nc1ccc(OCCOCc2ccccc2)c(-c2ccc(F)cc2)c1)c1ccc(OCCOCc2ccccc2)c(-c2ccc(F)c(F)c2)c1 ; O=C(Nc1ccc(OCCO)c(-c2ccc(F)cc2)c1)c1ccc(OCCOCc2ccccc2)c(-c2ccc(F)c(F)c2)c1]

### a. Preparation of Methyl 4-(2-(benzyloxy) ethoxy)-3-bromobenzoate (Compound 48)

To a solution of methyl 3-bromo-4-hydroxybenzoate (1.00 g, 4.33 mmol) in acetone (50 mL) was added ((2-bromoethoxy) methyl) benzene (1.40 g, 6.49 mmol), and K₂CO₃ (0.90 g, 6.49 mmol). The mixture was heated to gentle reflux and stirred for 15 h. Acetone was removed under vacuum. The residue was dissolved into dichloromethane (150 mL), washed with NaOH (1 M) (50 mL×2) and brine (50 mL×2), dried over Na₂SO₄, filtered, and concentrated to give the crude product as a pale yellow oil. To this residue was added hexane (50 mL) and stirred for 30 min. The resulting precipitate was filtered to give desired product 48 (0.80 g, 50% yield) as a white solid. ¹H NMR (500 MHz, CDCl₃) δ ppm 8.24 (d, J=2.5 Hz, 1H), 7.95 (dd, J=2.0, 9.0 Hz, 1H), 7.38-7.28 (m, 5H), 6.91 (d, J=8.5 Hz, 1H), 4.69 (s, 2H), 4.27 (t, J=4.5 Hz, 2H), 3.91 (t, J=4.5 Hz, 2H), 3.89 (s, 3H). ¹³C NMR (125 MHz, CDCl₃): δ ppm 165.91, 159.12, 138.22, 135.09, 130.71, 128.68, 127.97, 127.93, 124.11, 112.31, 112.11, 73.79, 69.31, 68.26, 52.36. MS (ESI) m/z=365.0 [M+H]⁺.

### b. Preparation of Methyl 6-(2-(benzyloxy) ethoxy)-3′,4′-difluoro-[1,1′-biphenyl]-3-carboxylate (Compound 49)

To a solution of 48 (0.50 g, 1.40 mmol) in dry DMF (30 mL) was added (3,4-difluorophenyl) boronic acid (0.27 g, 1.68 mmol), Pd(PPh₃)₄ (0.16 g, 0.14 mmol), and Cs₂CO₃ (1.40 g, 4.20 mmol). The mixture was then heated to 100° C. under argon and stirred for 24 h. It was then cooled to room temperature, and diluted with diethyl ether (150 mL), washed with water (50 mL×2), brine (50 mL×2), dried over Na₂SO₄, and concentrated under vacuum. The residue was purified by column chromatography (silica gel, hexanes:acetone=15:1 to 10:1) to yield 49 (0.39 g, 70% yield) as a pale yellow oil. ¹H NMR (500 MHz, CDCl₃) δ ppm 8.03-8.00 (m, 2H), 7.51-7.47 (m, 1H), 7.36-7.28 (m, 6H), 7.18-7.13 (m, 1H), 6.99 (d, J=8.5 Hz, 1H), 4.56 (s, 2H), 4.24 (t, J=4.5 Hz, 2H), 3.91 (s, 3H), 3.81 (t, J=4.5 Hz, 2H). ¹³C NMR (125 MHz, CDCl₃): δ ppm 166.83, 159.45, 151.05, 150.96, 150.95, 150.86, 149.09, 148.99, 148.88, 138.05, 134.56, 134.52, 134.50, 134.47, 132.41, 131.37, 128.68, 128.01, 127.83, 125.92, 125.89, 125.87, 125.85, 123.22, 119.02, 118.88, 117.03, 116.89, 111.98, 73.74, 68.49, 68.47, 52.24. MS (ESI) m/z=399.2 [M+H]⁺.

### c. Preparation of 6-(2-(benzyloxy) ethoxy)-3′,4′-difluoro-[1,1′-biphenyl]-3-carboxylic acid (Compound 50)

To the solution of 49 (0.35 g, 0.88 mmol) in a solvent mixture (28 mL, THF:MeOH:H₂O=4:2:1) was added LiOH (0.16 g, 7.00 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum. 50 (0.30 g, 88% yield) as an off-white solid. It was used directly in next step without further purification.

### d. Preparation of Methyl 6-(2-(benzyloxy) ethoxy)-4′-fluoro-[1,1′-biphenyl]-3-carboxylate (Compound 51)

To a solution of 48 (0.50 g, 1.40 mmol) in dry DMF (30 mL) was added (4-fluorophenyl) boronic acid (0.23 g, 1.68 mmol), Pd(PPh₃)₄ (0.16 g, 0.14 mmol), and Cs₂CO₃ (1.40 g, 4.20 mmol). The mixture was then heated to 100° C. under argon and stirred for 24 h. It was then cooled to room temperature, diluted with diethyl ether (150 mL), washed with water (50 mL×2), brine (50 mL×2), dried over Na₂SO₄, and concentrated under vacuum. The residue was purified by column chromatography (silica gel, hexanes:acetone=15:1 to 10:1) to yield 51 (0.38 g, 72% yield) as a pale yellow oil. ¹H NMR (500 MHz, CDCl₃): δ ppm 8.01-7.99 (m, 2H), 7.57-7.54 (m, 2H), 7.35-7.27 (m 5H), 7.06 (t, J=9.0 Hz, 2H), 6.99 (d, J=9.0 Hz, 1H), 4.54 (s, 2H), 4.23 (t, J=4.5 Hz, 2H), 3.90 (s, 3H), 3.80 (t, J=4.5 Hz, 2H). ¹³C NMR (125 MHz, CDCl₃): δ ppm 166.99, 163.38, 161.42, 159.58, 138.18, 133.63, 133.60, 132.55, 131.51, 131.45, 130.94, 128.66, 127.96, 127.73, 123.15, 115.16, 114.99, 111.93, 73.67, 68.55, 68.45, 52.18. MS (ESI) m/z=381.1 [M+H]⁺.

### e. Preparation of tert-Butyl (6-(2-(benzyloxy) ethoxy)-4′-fluoro-[1,1′-biphenyl]-3-yl) carbamate (Compound 52)

To the solution of 51 (0.35 g, 0.92 mmol) in a solvent mixture (14 mL, THF:MeOH:H₂O=4:2:1) was added LiOH (0.18 g, 7.36 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum. The resulting crude product (0.35 g, 0.95 mmol) was dissolved into a solvent mixture (25 mL, toluene:t-BuOH=4:1). To this solution was added Et₃N (0.18 g, 1.90 mmol), 4 Å molecular sieve (1 g), and diphenyl phosphoryl azide (0.26 g, 0.95 mmol). The resulting mixture was heated to 88° C. and stirred for 18 h, cooled to rt, filtered, diluted with EtOAc (100 mL), washed with brine (50 mL×3), dried over Na₂SO₄, and concentrated under vacuum. The residue was then purified by column chromatography (silica gel, hexanes:acetone=12:1 to 10:1) to yield 52 (0.15 g, 37% two steps yield) as a pale yellow oil. ¹H NMR (500 MHz, CDCl₃): δ ppm 7.56-7.53 (m, 2H), 7.37-7.26 (m, 7H), 7.04 (t, J=9.0 Hz, 2H), 6.91 (d, J=9.0 Hz, 1H), 6.60 (brs, 1H), 4.54 (s, 2H), 4.10 (t, J=5.0 Hz, 2H), 3.75 (t, J=5.0 Hz, 2H), 1.54 (s, 9H). ¹³C NMR (125 MHz, CDCl₃): δ ppm 163.24, 161.29, 153.41, 151.94, 138.38, 134.29, 134.27, 132.34, 131.49, 131.43, 130.79, 128.64, 127.89, 127.78, 122.07, 119.50, 115.05, 114.88, 114.04, 80.55, 73.55, 68.98, 68.87, 28.64. MS (ESI) m/z=438.1 [M+H]⁺.

### f. Preparation of 6-(2-(benzyloxy) ethoxy)-4′-fluoro-[1,1′-biphenyl]-3-aminium 2,2,2-trifluoroacetate (Compound 53)

To a solution of 52 (0.15 g, 0.34 mmol) in CH₂Cl₂ (3 mL) was added trifluoroacetic acid (3 mL). The mixture was stirred for 1 h at room temperature. The pH value was then adjusted to 9-10 with aqueous Na₂CO₃ solution, extracted with CH₂Cl₂ (20 mL×3). The organic phase was combined, dried over Na₂SO₄, and concentrated under vacuum. Compound 53 (0.12 g, >99% yield) was obtained as a yellow oil. It was used directly in next step without further purification.

### g. Preparation of 6-(2-(benzyloxy) ethoxy)-N-(6-(2-(benzyloxy) ethoxy)-4′-fluoro-[1,1′-biphenyl]-3-yl)-3′,4′-difluoro-[1,1′-biphenyl]-3-carboxamide (Compound 54)

Compound 53 (0.12 g, 0.36 mmol) was dissolved into CH₂Cl₂ (20 mL). To this solution was added 50 (0.14 g, 0.36 mmol), dimethylaminopyridine (0.065 g, 0.53 mmol), and EDC.HCl (0.10 g, 0.54 mmol) at 0° C. The mixture was stirred at room temperature overnight, and then diluted with CH₂Cl₂ (80 mL), washed with brine (50 mL×3), dried over Na₂SO₄, and concentrated under vacuum. The residue was purified by column chromatography (silica gel, hexanes:acetone=5:1 to 3:1) to yield 54 (0.12 g, 50% two steps yield) as a white solid. ¹H NMR (500 MHz, CDCl₃): δ ppm 7.86-7.79 (m, 3H), 7.61-7.45 (m, 5H), 7.38-7.27 (m, 10H), 7.20-7.11 (m, 1H), 7.03 (t, J=8.7 Hz, 2H), 7.02 (d, J=8.7 Hz, 1H), 6.97 (d, J=8.7 Hz, 1H), 4.57 (s, 2H), 4.54 (s, 2H), 4.24 (t, J=7.5 Hz, 2H), 4.14 (t, J=7.5 Hz, 2H), 7.83-3.75 (m, 4H). ¹³C NMR (125 MHz, CDCl₃): δ ppm 165.45, 163.22, 161.26, 158.49, 152.83, 151.01, 150.93, 150.92, 150.83, 149.04, 148.95, 148.85, 138.86, 138.09, 134.56, 134.53, 134.52, 134.48, 134.08, 134.05, 131.92, 131.46, 131.40, 130.49, 130.00, 128.23, 128.78, 128.70, 128.65, 128.04, 127.91, 127.86, 127.78, 127.60, 125.93, 125.89, 125.88, 125.86, 123.84, 121.51, 118.98, 118.84, 117.04, 116.90, 115.05, 114.88, 113.54, 112.24, 73.72, 73.57, 68.82, 68.78, 68.53, 68.48. MS (ESI) m/z=704.5 [M+H]⁺.

### h. Preparation of 3′,4′-Difluoro-N-(4′-fluoro-6-(2-hydroxyethoxy)-[1,1′-biphenyl]-3-yl)-6-(2-hydroxyethoxy)-[1,1′-biphenyl]-3-carboxamide (Compound 18)

To a solution of 54 (0.12 g, 0.17 mmol) in MeOH (10 mL) was added 10% Pd on activated carbon (0.012 g, 10% by weight). The air was evacuated and exchanged with H₂ gas three times. The reaction was allowed to stir under H₂ gas for 2 h. The mixture was filtered through celite, and the solvent was removed under reduced pressure to give 18 (0.08 g, 91% yield). ¹H NMR (500 MHz, d⁶-DMSO) δ ppm 10.10 (s, 1H), 8.00 (s, 1H), 7.99 (d, J=8.5 Hz, 1H), 7.79-7.73 (m, 3H), 7.63-7.45 (m, 4H), 7.26-7.21 (m, 3H), 7.10 (d, J=9.0 Hz, 1H), 4.89 (t, J=5.0 Hz, 1H), 4.80 (t, J=5.0 Hz, 1H), 4.15 (t, J=5.0 Hz, 2H), 4.01 (t, J=5.0 Hz, 2H), 3.71 (t, J=5.0 Hz, 2H), 3.66 (t, J=5.0 Hz, 2H). ¹³C NMR (125 MHz, d⁶-DMSO): δ ppm 165.05, 162.90, 160.96, 158.54, 152.26, 150.65, 150.55, 150.38, 150.28, 148.73, 148.61, 148.42, 148.33, 135.59, 135.56, 135.54, 135.51, 135.03, 135.01, 133.38, 131.84, 131.78, 130.60, 130.20, 129.18, 127.74, 127.58, 127.06, 127.03, 127.01, 126.98, 123.67, 119.26, 119.12, 117.76, 117.63, 115.55, 115.39, 113.92, 113.25, 70.96, 60.16, 59.95. HRMS (ESI) Calcd. for C₂₉H₂₄F₃NO₅ (M+Na)⁺ 546.1499, found 546.1519.

### 9. Preparation of Compound 19

The synthesis scheme (Synthesis Scheme 9) for the preparation of Compound 19 is shown below. The synthesis proceeds through the intermediates indicated (Compounds 55, 56, 57, and 58). The yield for each synthetic step was as indicated.

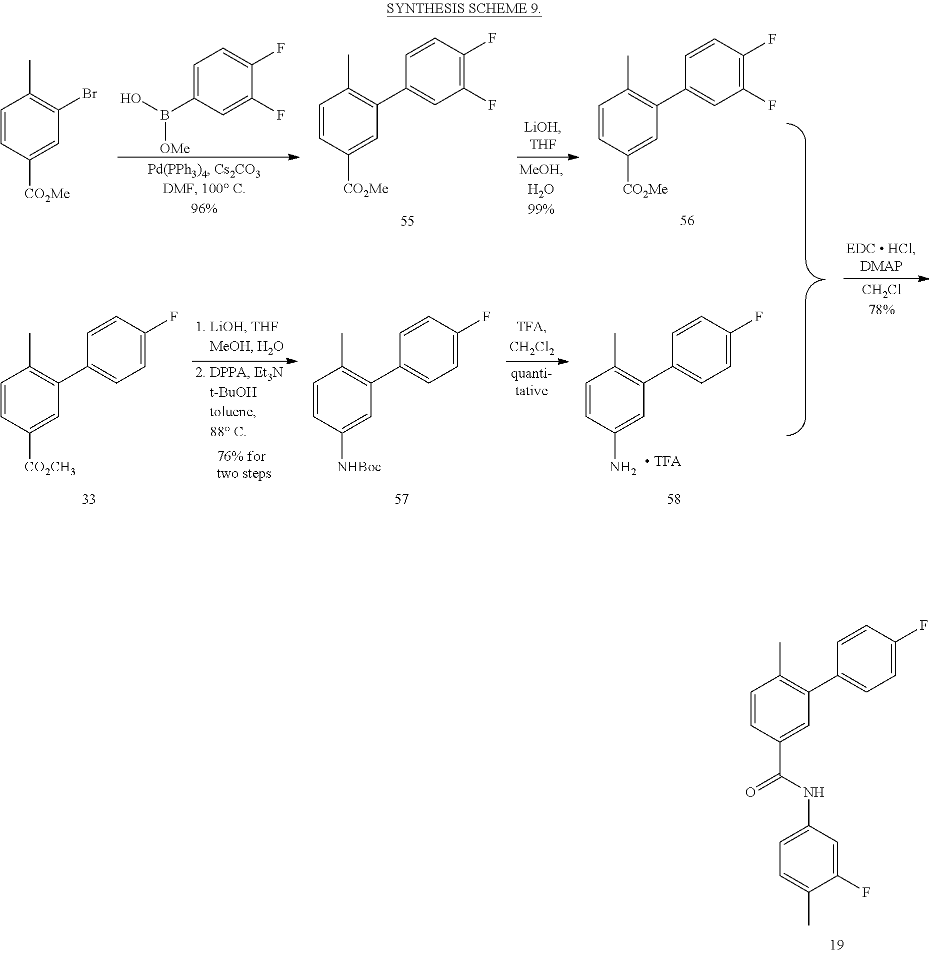

### a. Preparation of Methyl 3′,4′-difluoro-6-methyl-[1,1′-biphenyl]-3-carboxylate (Compound 55)

To a solution of methyl 3-bromo-4-methylbenzoate (1.00 g, 4.37 mmol) in dry DMF (50 mL) was added (3,4-difluorophenyl) boronic acid (0.83 g, 5.24 mmol), Pd(PPh₃)₄ (0.51 g, 0.44 mmol), and Cs₂CO₃ (4.27 g, 13.11 mmol). The mixture was heated to 100° C. under argon and stirred for 24 h. The reaction mixture was then cooled to room temperature, diluted with diethyl ether (200 mL), washed with water (50 mL×2) and brine (50 mL×2), dried over Na₂SO₄, and concentrated under vacuum. The residue was purified by column chromatography (silica gel, hexanes:acetone=15:1 to 12:1) to yield 55 (1.10 g, 96% yield) as a pale yellow oil. ¹H NMR (500 MHz, CDCl₃) δ ppm 7.92 (d, J=8.0 Hz, 1H), 7.86 (s, 1H), 7.32 (d, J=8.0 Hz, 1H), 7.22-7.09 (m, 2H), 7.03-7.00 (m, 1H), 3.89 (s, 3H), 2.27 (s, 3H). ¹³C NMR (125 MHz, CDCl₃): δ ppm 167.01, 151.19, 151.09, 150.96, 150.86, 149.21, 149.11, 148.98, 148.88, 141.07, 140.15, 140.14, 137.99, 137.96, 137.95, 137.92, 130.96, 130.86, 129.09, 128.23, 125.54, 125.51, 125.49, 125.46, 118.43, 118.29, 117.39, 117.26, 52.26, 20.78. MS (ESI) m/z=263.2 [M+H]⁺.

### b. Preparation of 3′,4′-Difluoro-6-methyl-[1,1′-biphenyl]-3-carboxylic acid (Compound 56)

To the solution of 55 (1.00 g, 3.81 mmol) in a solvent mixture (28 mL, THF:MeOH:H₂O=4:2:1) was added LiOH (0.71 g, 30.48 mmol). The mixture was stirred for 8 h at room temperature. The pH value was then adjusted to 4-5 with HCl (1 M), diluted with water (50 mL), and extracted with EtOAc (50 mL×3). The combined organic phase was dried over Na₂SO₄ and concentrated under vacuum. Compound 50 (0.94 g, >99% yield) was obtained as an off-white solid. It was used directly in next step without further purification.

### c. Preparation of tert-Butyl (4′-fluoro-6-methyl-[1,1′-biphenyl]-3-yl) carbamate (Compound 57)

To the solution of 33 (1.00 g, 4.09 mmol) in a solvent mixture (28 mL, THF:MeOH:H₂O=4:2:1) was added LiOH (0.78 g, 32.75 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum. The resulting crude product (0.95 g, 4.13 mmol) was dissolved into a solvent mixture (50 mL, toluene:t-BuOH=4:1). To this solution was added triethylamine (0.84 g, 8.26 mmol), 4 Å molecular sieve (1 g), and diphenyl phosphoryl azide (1.14 g, 4.13 mmol). The resulting mixture was heated to 88° C. and stirred for 18 h. Then, the reaction mixture was cooled to room temperature, filtered, diluted with EtOAc (150 mL), washed with brine (50 mL×3), dried over Na₂SO₄, and concentrated under vacuum. The residue was purified by column chromatography (silica gel, hexanes:acetone=12:1 to 10:1) to yield 57 (0.93 g, 76% yield) as an off-white solid. ¹H NMR (500 MHz, CDCl₃): δ ppm 7.26-7.22 (m, 4H), 7.16 (d, J=8.0 Hz, 1H), 7.07 (t, J=8.5 Hz, 2H), 6.61 (brs, 1H), 2.18 (s, 3H), 1.52 (s, 9H). ¹³C NMR (125 MHz, CDCl₃): δ ppm 163.13, 161.18, 153.16, 141.60, 137.86, 137.83, 136.38, 131.01, 130.91, 130.84, 130.19, 120.36, 118.02, 115.22, 115.05, 80.63, 28.58, 19.91. MS (ESI) m/z=302.2 [M+H]⁺.

### d. Preparation of 4′-Fluoro-6-methyl-[1,1′-biphenyl]-3-aminium 2,2,2-trifluoroacetate (Compound 58)

To a solution of 57 (0.90 g, 3.00 mmol) in CH₂Cl₂ (5 mL) was added trifluoroacetic acid (5 mL). The mixture was stirred for 1 h at room temperature. Then, the pH value was adjusted to 9-10 with Na₂CO₃ (aq) and extracted with CH₂Cl₂ (50 mL×3). The organic phase was combined, dried over Na₂SO₄, and concentrated under vacuum to yield 58 (0.50 g, 83% yield) as a pale yellow oil. It was used directly in next step without further purification.

### e. Preparation of 3′,4′-Difluoro-N-(4′-fluoro-6-methyl-[1,1′-biphenyl]-3-yl)-6-methyl-[1,1′-biphenyl]-3-carboxamide (Compound 19)

Compound 58 (0.50 g, 2.48 mmol) was dissolved into CH₂Cl₂ (30 mL). To this solution was added 56 (0.62 g, 2.48 mmol), dimethylaminopyridine (0.30 g, 2.48 mmol), and EDC.HCl (0.71 g, 3.72 mmol) at 0° C. The mixture was stirred at room temperature overnight. Then, the reaction mixture was diluted with CH₂Cl₂ (150 mL), washed with brine (50 mL×3), dried over Na₂SO₄, and concentrated under vacuum. The residue was purified by column chromatography (silica gel, hexanes:acetone=8:1 to 6:1) to yield 19 (1.00 g, 78%) as a white solid. ¹H NMR (500 MHz, d⁶-DMSO) δ ppm 10.18 (s, 1H), 7.88 (d, J=8.0 Hz, 1H), 7.85 (s, 1H), 7.71 (d, J=8.0 Hz, 1H), 7.64 (s, 1H), 7.57-7.50 (m, 2H), 7.45 (d, J=8.0 Hz, 1H), 7.37 (dd, J=5.5, 8.5 Hz, 2H), 7.28-7.24 (m, 4H), 2.29 (s, 3H), 2.17 (s, 3H). ¹³C NMR (125 MHz, d⁶-DMSO): δ ppm 165.42, 165.33, 162.97, 161.04, 150.83, 150.73, 150.56, 150.46, 148.87, 148.77, 148.60, 148.50, 140.95, 139.73, 139.63, 138.72, 138.68, 138.66, 138.64, 138.22, 138.19, 137.73, 137.63, 133.16, 133.11, 131.52, 131.45, 131.32, 131.19, 130.62, 129.26, 127.89, 126.89, 126.86, 126.84, 126.81, 122.19, 122.09, 120.04, 119.94, 119.06, 118.93, 118.14, 118.00, 115.83, 115.66, 20.80, 20.23. HRMS (ESI) Calcd for C₂₇H₂₀F₃NO (M+Na)⁺ 454.1389, found 454.1406.

### 10. Preparation of Compounds 20-23 and 26-28

The synthesis scheme (Synthesis Scheme 10) for the preparation of Compounds 20-23 and 26-28 is shown below. The synthesis proceeds through the intermediates indicated (Compounds 59a-c, 60a-f, 61a-f, 62a-d, 63a-d, and 64a-g). The yield for each synthetic step was as indicated.

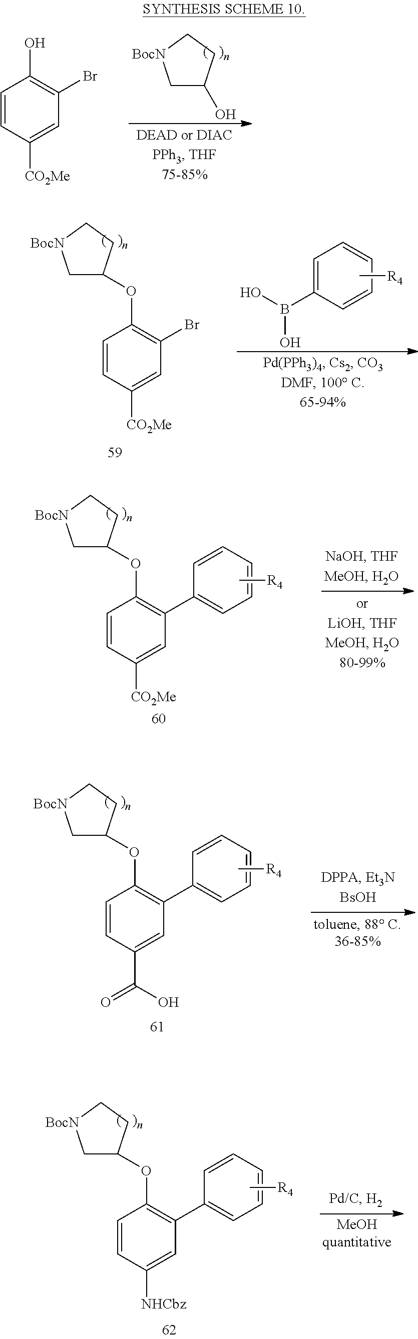

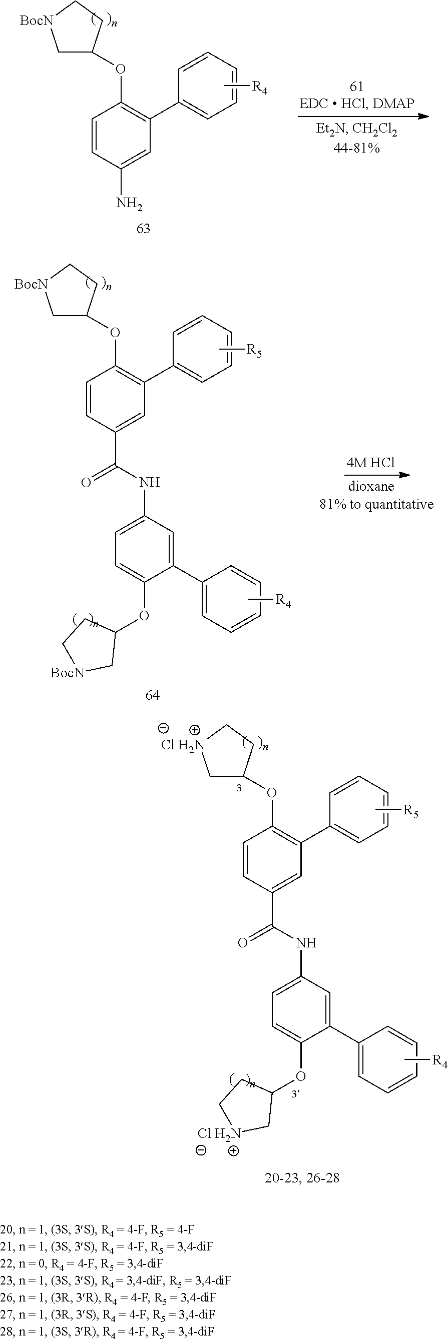

### a. Preparation of tert-Butyl (S)-3-(2-bromo-4-(methoxycarbonyl) phenoxy) pyrrolidine-1-carboxylate (Compound 59a)

To a solution of methyl 3-bromo-4-hydroxybenzoate (7.40 g, 32.04 mmol) in dry THF (100 mL) under anhydrous conditions was added (R)-tert-butyl 3-hydroxypyrrolidine-1-carboxylate (5.00 g, 26.70 mmol), DEAD (6.31 mL, 40.01 mmol), triphenyl phosphine (10.51 g, 40.01 mmol), and stirred for 1 h at room temperature under argon. Upon completion, the reaction was diluted with CH₂C₂ (100 mL). The organic layer was washed with water (2×50 ml), brine (50 mL), and dried over MgSO₄. After the filtration, the solvent removed under reduced pressure. The residue was then purified by column chromatography (silica gel, hexanes:EtOAc=3:1) to yield 59a (7.21 g, 68% yield) as a white solid. ¹H NMR (500 MHz, CDCl₃): δ ppm 8.22 (s, 1H), 7.93 (d, J=8.0 Hz, 1H), 6.85 (d, J=8.0 Hz, 1H), 5.00-4.98 (m, 1H), 3.88 (s, 3H), 3.63-3.56 (m, 4H), 2.23-2.13 (m, 2H), 1.45 (s, 9H). ¹³C NMR (125 MHz, CDCl₃): δ ppm 186.39, 165.67, 157.47, 143.72, 135.31, 130.41, 124.36, 113.33, 79.76, 79.74, 78.32, 52.29, 51.57, 51.22, 44.25, 43.84, 31.85, 31.02, 28.60. MS (ESI) m/z=400.6 [M+H]⁺.

### b. Preparation of tert-Butyl 3-(2-bromo-4-(methoxycarbonyl) phenoxy) azetidine-1-carboxylate (Compound 59b)

To a solution of methyl 3-bromo-4-hydroxybenzoate (0.50 g, 2.16 mmol), tert-butyl 3-hydroxyazetidine-1-carboxylate (0.40 g, 2.16 mmol) and triphenylphosphine (0.68 g, 2.59 mmol) in dry THF (30 mL) was added diisopropyl azodicarboxylate (0.52 g, 2.59 mmol) at 0° C. Then, the temperature was allowed to rise to room temperature and stir for another 1 h. Upon completion, the reaction was diluted with CH₂Cl₂ (150 mL), washed with NaOH (1M) (50 mL×2), brine (50 mL×2), dried over Na₂SO₄, and concentrated to give the crude product. The crude product was then purified by column chromatography (silica gel, hexanes:acetone=10:1 to 8:1) to yield 59b (0.71 g, 85% yield) as a white solid. ¹H NMR (500 MHz, CDCl₃) δ ppm 8.23 (s, 1H), 7.92 (dd, J=2.0, 8.5 Hz, 1H), 6.53 (d, J=8.5 Hz, 1H), 4.97-4.93 (m, 1H), 4.33 (dd, J=6.5, 10.0 Hz, 2H), 4.06 (dd, J=5.0, 10.0 Hz, 2H), 3.88 (s, 3H), 1.44 (s, 9H). ¹³C NMR (125 MHz, CDCl₃): δ ppm 165.84, 156.96, 156.16, 135.51, 130.71, 124.90, 112.09, 112.04, 80.30, 67.25, 52.45, 28.55. MS (ESI) m/z=385.9 [M+H]⁺.

### c. Preparation of tert-Butyl (R)-3-(2-bromo-4-(methoxycarbonyl) phenoxy) pyrrolidine-1-carboxylate (Compound 59c)

59c (1.04 g, 54% yield) as a white solid. The NMR data is the same as 59a.

### d. Preparation of tert-Butyl (S)-3-((4′-fluoro-5-(methoxycarbonyl)-[1,1′-biphenyl]-2-yl) oxy) pyrrolidine-1-carboxylate (Compound 60a)

To a solution of 59a (1.00 g, 2.50 mmol) in dry DMF (25 mL) under anhydrous conditions was added (4-fluorophenyl) boronic acid (0.42 g, 3.00 mmol), Pd(PPh₃)₄ (0.14 g, 0.13 mmol), and Cs₂CO₃ (1.22 g, 3.75 mmol). The mixture was heated to 80° C. under argon and stirred for 20 h. The solvent was then removed under reduced pressure, and the residue was taken into EtOAc (100 mL). The solution was washed with water (50 mL) and brine (50 mL), and dried over MgSO₄. After the filtration, the solvent was removed under reduced pressure. The residue was purified by column chromatography (silica gel, hexanes:EtOAc=1:3) to yield 60a (0.71 g, 69% yield) as a white solid. ¹H NMR (500 MHz, CDCl₃): δ ppm 8.00 (d, J=6.8 Hz, 2H), 7.43 (dd, J=5.6, 8.2 Hz, 2H), 7.08 (t, J=8.5 Hz, 2H), 6.94 (d, J=9.0 Hz, 1H), 4.97-4.95 (m, 1H), 3.90 (s, 3H), 3.66-3.29 (m, 4H), 2.11-2.08 (m, 2H), 1.44 (s, 9H). ¹³C NMR (125 MHz, CDCl₃): δ ppm 166.76, 163.29, 161.33, 157.67, 154.61, 132.81, 131.17, 131.11, 130.69, 123.46, 115.15, 115.02, 113.14, 112.85, 79.77, 52.16, 51.60, 51.24, 51.23, 51.21, 44.23, 44.21, 44.20, 43.92, 43.89, 31.70, 30.98, 30.97, 28.61. MS (ESI) m/z=416.7 [M+H]⁺.

### e. Preparation of tert-Butyl (S)-3-((3′,4′-difluoro-5-(methoxycarbonyl)-[1,1′-biphenyl]-2-yl) oxy) pyrrolidine-1-carboxylate (Compound 60b)

60b (0.38 g, 70% yield) as a white solid. ¹H NMR (500 MHz, CDCl₃): δ ppm 7.99 (d, J=9.0 Hz, 2H,), 7.29 (d, J=8.1 Hz, 1H), 7.16 (d, J=3.5 Hz, 2H), 6.94 (d, J=8.4 Hz, 1H), 4.98-4.96 (m, 1H), 3.89 (s, 3H), 3.66-3.31 (m, 4H), 2.11-2.09 (m, 2H), 1.44 (s, 9H). ¹³C NMR (125 MHz, CDCl₃): δ ppm 166.57, 157.44, 154.66, 150.84, 148.87, 132.64, 131.12, 129.65, 125.62, 125.59, 125.57, 125.54, 123.47, 118.63, 118.58, 118.56, 118.48, 118.45, 118.42, 117.00, 116.97, 116.89, 116.83, 113.01, 112.81, 79.86, 76.47, 52.18, 51.58, 51.12, 44.22, 43.83, 31.69, 30.88, 30.87, 28.53. MS (ESI) m/z=434.6 [M+H]⁺.

### f. Preparation of tert-Butyl 3-((4′-fluoro-5-(methoxycarbonyl)-[1,1′-biphenyl]-2-yl) oxy) azetidine-1-carboxylate (Compound 60c)

60c (0.54 g, 65% yield) as a white solid. ¹H NMR (300 MHz, CDCl₃): δ ppm 8.01 (d, J=2.1 Hz, 1H), 7.97 (dd, J=2.1, 8.7 Hz, 1H), 7.53-7.48 (m, 2H), 7.11 (t, J=8.7 Hz, 2H), 6.53 (d, J=8.7 Hz, 1H), 4.98-4.91 (m, 1H), 4.33 (dd, J=6.3, 9.9 Hz, 2H), 4.06 (dd, J=3.9, 9.9 Hz, 2H), 3.89 (s, 3H), 1.43 (s, 9H). ¹³C NMR (75 MHz, CDCl₃): δ ppm 166.72, 164.13, 160.86, 157.13, 156.18, 133.15, 133.11, 132.95, 131.40, 131.29, 130.93, 130.12, 123.96, 115.44, 115.16, 111.72, 80.24, 66.55, 52.29, 28.55. MS (ESI) m/z=402.2 [M+H]⁺.

### g. Preparation of tert-Butyl 3-((3′,4′-difluoro-5-(methoxycarbonyl)-[1,1′-biphenyl]-2-yl) oxy) azetidine-1-carboxylate (Compound 60d)

60d (0.61 g, 94% yield) as a white solid. ¹H NMR (300 MHz, CDCl₃): δ ppm 8.00 (s, 1H), 7.98 (d, J=8.1 Hz, 1H), 7.40-7.16 (m, 3H), 6.60 (d, J=8.4 Hz, 1H), 4.97-4.94 (m, 1H), 4.32 (dd, J=6.9, 9.6 Hz, 2H), 3.96 (dd, J=3.6, 9.6 Hz, 2H), 3.89 (s, 3H), 1.43 (s, 9H). ¹³C NMR (75 MHz, CDCl₃): δ ppm 166.55, 157.07, 156.17, 151.82, 151.78, 151.66, 151.62, 148.55, 148.48, 148.38, 148.33, 134.12, 134.06, 134.03, 133.97, 132.83, 131.38, 129.01, 125.89, 125.84, 125.81, 125.77, 124.04, 118.85, 118.61, 117.29, 117.06, 111.79, 80.31, 66.66, 52.33, 28.53. MS (ESI) m/z=420.2 [M+H]⁺.

### h. Preparation of tert-Butyl (R)-3-((4′-fluoro-5-(methoxycarbonyl)-[1,1′-biphenyl]-2-yl) oxy) pyrrolidine-1-carboxylate (Compound 60e)

60e (1.20 g, 57% yield) as a white solid. The NMR data is the same as 60a.

### i. Preparation of tert-Butyl (R)-3-((3′,4′-difluoro-5-(methoxycarbonyl)-[1,1′-biphenyl]-2-yl) oxy) pyrrolidine-1-carboxylate (Compound 60f)

60f (0.20 g, 47% yield) as a white solid. The NMR data is the same as 60b.

### j. Preparation of (S)-6-((1-(tert-butoxycarbonyl) pyrrolidin-3-yl) oxy)-4′-fluoro-[1,1′-biphenyl]-3-carboxylic acid (Compound 61a)

To a solution of 60a (1.13 g, 2.72 mmol) in a solvent mixture (10 mL, THF:H₂O:MeOH=4:1:1) was added 6 M NaOH (15 mL), and the reaction stirred at room temperature for 5 h. THF and MeOH were then removed under reduced pressure. The remaining aqueous solution was acidified with 6 M HCl to pH=4 and extracted with EtOAc (50 mL). The organic layer was washed with water (50 mL), brine (50 mL), and dried over MgSO₄. After the filtration, the solvent was removed under reduced pressure to yield 61a (0.94 g, 86%) as a white solid. ¹H NMR (500 MHz, CDCl₃): δ 8.07-8.04 (m, 2H), 7.46-7.42 (m, 2H), 7.09 (t, J=8.3 Hz, 2H), 6.97 (d, J=9.5 Hz, 1H), 5.00-4.97 (m, 1H), 3.69-3.30 (m, 4H), 2.14-2.12 (m, 2H), 1.45 (s, 9H). ¹³C NMR (125 MHz, CDCl₃): δ 171.39, 171.24, 163.33, 161.35, 158.29, 158.22, 154.83, 154.62, 133.41, 133.32, 131.38, 131.17, 131.14, 130.93, 130.87, 122.65, 115.23, 115.09, 114.92, 113.09, 112.75, 79.98, 77.26, 76.48, 51.63, 51.19, 44.26, 43.91, 31.67, 30.92, 28.59.

### k. Preparation of (S)-6-((1-(tert-butoxycarbonyl) pyrrolidin-3-yl) oxy)-3′,4′-difluoro-[1,1′-biphenyl]-3-carboxylic acid (Compound 61b)

61b (0.51 g, 80%) as a white solid. ¹H NMR (500 MHz, CDCl₃): δ ppm 8.10-8.02 (m, 2H), 7.31-7.26 (m, 1H), 7.19-7.16 (m, 2H), 6.97 (d, J=8.7 Hz, 1H), 5.03-5.00 (m, 1H), 3.71-3.33 (m, 4H), 2.16-2.12 (m, 2H), 1.46 (s, 9H). ¹³C NMR (125 MHz, d⁶-DMSO/CDCl₃): δ ppm 171.12, 170.88, 158.18, 158.03, 154.96, 154.62, 150.92, 150.90, 148.96, 148.94, 148.93, 148.85, 148.83, 134.23, 133.19, 131.80, 129.74, 129.68, 125.60, 122.73, 118.65, 118.51, 117.00, 116.86, 112.98, 112.81, 80.18, 76.50, 51.63, 51.13, 44.29, 43.87, 31.69, 30.89, 28.55.

### l. Preparation of 6-((1-(tert-butoxycarbonyl) azetidin-3-yl) oxy)-4′-fluoro-[1,1′-biphenyl]-3-carboxylic acid (Compound 61c)

To the solution of 60c (0.30 g, 0.75 mmol) in a solvent mixture (14 mL, THF:MeOH:H₂O=4:2:1) was added LiOH (0.14 g, 6.00 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum to afford 61c (0.29 g, >99% yield) as a white solid. It was used directly in next step without further purification.

### m. Preparation of 6-((1-(tert-butoxycarbonyl)azetidin-3-yl)oxy)-3′,4′-difluoro-[1,1′-biphenyl]-3-carboxylic acid (Compound 61d)

61d (0.45 g, 92% yield) as a white solid. It was used directly in next step without further purification.

### n. Preparation of (R)-6-((1-(tert-butoxycarbonyl) pyrrolidin-3-yl) oxy)-4′-fluoro-[1,1′-biphenyl]-3-carboxylic acid (Compound 61e)

61e (0.28 g, 97% yield) as an off-white solid. The NMR data is the same as 61a.

### o. Preparation of (R)-6-((1-(tert-butoxycarbonyl) pyrrolidin-3-yl) oxy)-3′,4′-difluoro-[1,1′-biphenyl]-3-carboxylic acid (Compound 61f)

61f (0.18 g, 95% yield) as an off-white solid. The NMR data is the same as 61b.

### p. Preparation of tert-Butyl (S)-3-((5-(((benzyloxy) carbonyl) amino)-4′-fluoro-[1,1′-biphenyl]-2-yl) oxy) pyrrolidine-1-carboxylate (Compound 62a)

To a solution of 61a (0.94 g, 2.35 mmol) in dry toluene (30 mL) under anhydrous conditions was added DPPA (5.07 mL, 23.52 mmol), Et₃N (3.28 mL, 23.52 mmol), benzyl alcohol (5.00 mL, 0.048 mmol), and activated molecular sieves (2 g). The mixture was stirred at room temperature for 10 min and then heated to 80° C. under nitrogen for 24 h. Upon completion, the molecule sieves were filtered and the solution diluted with EtOAc (50 mL). The organic layer was washed with water (50 mL), brine (50 mL), and dried over MgSO₄. After the filtration, the solvent was removed under reduced pressure. The residue was purified by column chromatography (silica gel, hexanes:EtOAc=3:1) to yield 62a (0.72 g, 65% yield) as a white solid. ¹H NMR (500 MHz, CDCl₃): δ ppm 7.43-7.26 (m, 9H), 7.04 (t, J=8.0 Hz, 2H), 6.88 (d, J=8.2 Hz, 1H), 6.73 (brs, 1H), 5.19 (s, 2H), 4.72-4.70 (m, 1H), 3.58-3.17 (m, 4H), 2.02-1.93 (m, 2H), 1.43 (s, 9H). ¹³C NMR (125 MHz, CDCl₃): δ ppm 162.93, 160.97, 154.52, 153.93, 149.80, 136.21, 133.81, 132.55, 132.47, 131.89, 131.62, 130.92, 128.45, 128.13, 122.01, 121.81, 119.36, 116.08, 115.59, 114.86, 114.74, 114.69, 114.57, 79.46, 79.35, 77.69, 66.73, 51.37, 50.97, 44.03, 43.75, 31.28, 30.60, 28.40. MS (ESI) m/z=507.7 [M+H]⁺.

### q. Preparation of tert-Butyl (S)-3-((5-(((benzyloxy) carbonyl) amino)-3′,4′-difluoro-[1,1′-biphenyl]-2-yl) oxy) pyrrolidine-1-carboxylate (Compound 62b)

62b (0.27 g, 85% yield) as a white solid. ¹H NMR (500 MHz, CDCl₃): δ ppm 7.37-7.26 (m, 9H), 7.13-7.10 (m, 2H), 6.83 (s, 1H), 5.17 (s, 2H), 4.71-4.69 (m, 1H), 3.56-3.19 (m, 4H), 2.02-1.93 (m, 2H), 1.44 (s, 9H). ¹³C NMR (125 MHz, CDCl₃): δ ppm 154.62, 154.48, 153.91, 150.60, 149.86, 149.67, 148.63, 136.19, 134.82, 132.43, 130.67, 130.47, 128.57, 128.29, 128.24, 127.44, 126.93, 125.48, 121.84, 119.88, 119.87, 119.86, 118.50, 118.38, 118.36, 118.25, 116.78, 116.71, 116.64, 116.57, 115.71, 115.51, 79.59, 79.58, 77.84, 77.07, 66.92, 51.43, 50.95, 44.14, 43.77, 31.45, 30.71, 28.45.

### r. Preparation of tert-Butyl-3-((5-(((benzyloxy) carbonyl) amino)-4′-fluoro-[1,1′-biphenyl]-2-yl) oxy) azetidine-1-carboxylate (Compound 62c)

62c (0.14 g, 38%) as a white solid. ¹H NMR (500 MHz, d⁶-acetone): δ ppm 8.68 (brs, 1H), 7.62-7.58 (m, 3H), 7.51 (d, J=9.0 Hz, 1H), 7.43-7.31 (m, 5H), 7.19 (t, J=9.0 Hz, 2H), 6.77 (d, J=9.0 Hz, 1H), 5.17 (s, 2H), 5.00-4.95 (m, 1H), 4.34-4.24 (m, 2H), 3.87-3.80 (m, 2H), 1.41 (s, 9H). ¹³C NMR (125 MHz, d⁶-acetone): δ ppm 163.22, 161.28, 156.00, 153.85, 149.45, 137.25, 134.59, 134.56, 133.70, 131.52, 131.46, 130.13, 128.62, 128.27, 128.17, 12.1.57, 119.03, 115.01, 114.84, 113.48, 78.94, 66.81, 66.23, 27.84. MS (ESI) m/z=493.1 [M+H]⁺.

### s. Preparation of tert-Butyl (R)-3-((5-(((benzyloxy) carbonyl) amino)-4′-fluoro-[1,1′-biphenyl]-2-yl) oxy) pyrrolidine-1-carboxylate (Compound 62d)

62d (0.80 g, 58% yield) as an amorphous solid. The NMR data is the same as 62a.

### t. Preparation of tert-Butyl (S)-3-((5-amino-4′-fluoro-[1,1′-biphenyl]-2-yl) oxy) pyrrolidine-1-carboxylate (Compound 63a)

To a solution of 62a (0.68 g, 1.42 mmol) in MeOH (20 ml) was added 10% Pd on activated carbon (0.07 g, 10% by weight). The air was evacuated and exchanged with the H₂ gas three times. The reaction mixture was allowed to stir under H₂ for 2 h and then filter through celite. The solvent was removed under reduced pressure to yield 63a (0.47 g, 89% yield) as an off-white solid. ¹H NMR (500 MHz, CDCl₃): δ ppm 7.41 (dd, J=5.8 Hz, J=8.0 Hz, 2H), 7.03 (d, J=6.0 Hz, 2H), 6.78 (t, J=8.2 Hz, 1H), 6.65-6.59 (m, 2H), 4.53-4.50 (m, 1H), 3.57 (brs, 2H), 3.44-3.09 (m, 4H), 1.97-1.83 (m, 2H), 1.43 (s, 9H). ¹³C NMR (125 MHz, CDCl₃): δ ppm 162.93, 160.97, 154.52, 153.93, 149.80, 136.21, 133.81, 132.55, 132.47, 131.89, 131.62, 130.92, 128.45, 128.13, 122.01, 121.81, 119.36, 116.08, 115.59, 114.86, 114.74, 114.69, 114.57, 79.46, 79.35, 77.69, 66.73, 51.37, 50.97, 44.03, 43.75, 31.28, 30.60, 28.40. MS (ESI) m/z=373.7 [M+H]⁺.

### u. Preparation of tert-Butyl (S)-3-((5-amino-3′,4′-difluoro-[1,1′-biphenyl]-2-yl) oxy) pyrrolidine-1-carboxylate (Compound 63b)

63b (0.19 g, quantitative yield) as an off-white solid. ¹H NMR (500 MHz, CDCl₃): δ ppm 7.29-7.26 (m, 1H), 7.15-7.13 (m, 2H), 6.79-6.76 (m, 1H), 6.64-6.60 (m, 2H), 4.59-4.56 (m, 1H), 3.58 (brs, 2H), 3.55-3.14 (m, 4H), 1.89-1.87 (m, 2H), 1.43 (s, 9H). ¹³C NMR (125 MHz, CDCl₃): δ ppm 154.58, 154.46, 150.78, 150.51, 148.81, 148.43, 146.62, 146.43, 141.53, 135.41, 135.36, 131.67, 125.46, 125.43, 125.41, 125.38, 118.50, 118.35, 118.20, 118.00, 117.89, 117.57, 117.46, 116.80, 116.72, 116.67, 116.58, 116.42, 115.70, 115.58, 79.37, 78.68, 77.90, 51.41, 50.90, 44.16, 43.79, 31.52, 30.80, 28.51. MS (ESI) m/z=391.7 [M+H]⁺.

### v. Preparation of tert-Butyl 3-((5-amino-3′,4′-difluoro-[1,1′-biphenyl]-2-yl) oxy) azetidine-1-carboxylate (Compound 63c)

63c (0.09 g, 96% yield) as a pale yellow oil. It was used directly in next step without further purification.

### w. Preparation of tert-Butyl (R)-3-((5-amino-4′-fluoro-[1,1′-biphenyl]-2-yl) oxy) pyrrolidine-1-carboxylate (Compound 63d)

63d (0.18 g, >99% yield) as an off-white solid. It was used directly in next step without further purification. The NMR data is the same as 63a.

### x. Preparation of tert-Butyl (S)-3-((5-((6-(((S)-1-(tert-butoxycarbonyl) pyrrolidin-3-yl) oxy)-4′-fluoro-[1,1′-biphenyl]-3-yl) carbamoyl)-4′-fluoro-[1,1′-biphenyl]-2-yl) oxy) pyrrolidine-1-carboxylate (Compound 64a)

To a solution of 63a (0.23 g, 0.69 mmol) in CH₂Cl₂ (20 ml) was added 61a (0.28 g, 0.69 mmol), Et₃N (0.22 mL, 1.56 mmol), EDC.HCl (0.18 g, 0.93 mmol), and DMAP (0.09 g, 0.75 mmol). The mixture was stirred at room temperature for 68 h. The mixture was diluted with CH₂Cl₂ (50 mL) and washed with water (50 mL), brine (50 mL), and dried over MgSO₄. After the filtration, the solvent was removed under reduced pressure. The residue was purified by column chromatography (silica gel, hexanes:EtOAc=1:3) to yield 64a (0.21 g, 44% yield) as a white solid. ¹H NMR (500 MHz, CDCl₃): δ ppm 8.66-8.56 (m, 1H), 7.86-7.79 (m, 2H), 7.58-7.53 (m, 2H), 7.38-7.34 (m, 4H), 6.99-6.82 (m, 6H), 4.88-4.85 (m, 1H), 4.72-4.70 (m, 1H), 3.37-3.16 (m, 8H), 2.04-1.90 (m, 4H), 1.41 (s, 18H). ¹³C NMR (125 MHz, CDCl₃): δ ppm 165.39, 165.35, 165.33, 165.26, 163.20, 163.14, 163.05, 163.01, 161.23, 161.17, 161.10, 161.05, 156.59, 156.39, 154.70, 154.63, 154.52, 154.46, 150.52, 150.46, 133.85, 133.36, 133.33, 133.28, 133.26, 132.81, 132.71, 132.70, 132.58, 131.71, 131.54, 131.10, 131.03, 130.82, 130.81, 130.25, 128.36, 128.21, 128.02, 123.78, 123.64, 123.61, 123.58, 123.47, 121.15, 121.01, 115.65, 115.27, 115.11, 114.96, 114.85, 114.82, 114.69, 113.37, 113.26, 79.78, 79.57, 79.51, 77.72, 77.23, 77.08, 76.39, 51.54, 51.07, 50.97, 44.23, 44.19, 43.84, 31.60, 31.51, 30.81, 28.54, 28.52.

### y. Preparation of tert-Butyl (S)-3-((5-(6-(((S)-1-(tert-butoxycarbonyl) pyrrolidin-3-yl) oxy)-3′,4′-difluoro-[1,1′-biphenyl]-3-ylcarboxamido)-4′-fluoro-[1,1′-biphenyl]-2-yl) oxy) pyrrolidine-1-carboxylate (Compound 64b)

64b (0.26 g, 63% yield) as a white solid. ¹H NMR (500 MHz, d⁶-Acetone): δ ppm 9.54 (s, 1H), 8.06-8.04 (m, 2H), 7.84-7.81 (m, 2H), 7.53 (dd, J=5.5, 8.5 Hz, 2H), 7.52-7.47 (m, 1H), 7.38-7.35 (m, 2H), 7.29 (d, J=8.0 Hz, 1H), 7.17-7.13 (m, 3H), 5.24-5.20 (m, 1H), 5.02-4.97 (m, 1H), 3.65-3.19 (m, 8H), 2.23-2.06 (m, 4H), 1.43 (s, 9H), 1.40 (s, 9H); ¹³C NMR (125 MHz, CDCl₃): δ ppm 165.13, 163.00, 161.04, 156.48, 156.21, 154.66, 154.53, 150.62, 150.38, 148.73, 134.27, 133.79, 132.77, 132.70, 132.58, 132.43, 131.64, 131.52, 131.02, 130.19, 129.52, 128.85, 128.72, 127.99, 127.83, 125.55, 123.61, 123.47, 121.16, 121.02, 118.51, 118.376, 116.83, 116.70, 115.55, 115.25, 114.97, 114.83, 114.67, 113.16, 79.94, 79.84, 79.57, 77.73, 77.36, 77.05, 76.39, 51.55, 51.07, 50.91, 44.20, 43.84, 31.62, 31.53, 30.80, 28.53, 28.46.

### z. Preparation of tert-Butyl-3-((5-(6-((1-(tert-butoxycarbonyl) azetidin-3-yl) oxy)-3′,4′-difluoro-[1,1′-biphenyl]-3-ylcarboxamido)-4′-fluoro-[1,1′-biphenyl]-2-yl) oxy) azetidine-1-carboxylate (Compound 64c)

64c (0.11 g, 58% two steps yield) as a white solid. ¹H NMR (500 MHz, CDCl₃): δ ppm 8.79 (brs 1H), 7.87-7.86 (m, 2H), 7.64 (dd, J=2.5, 8.5 Hz, 1H), 7.49 (d, J=2.5 Hz, 1H), 7.45-7.42 (m, 2H), 7.34-7.09 (m, 3H), 7.07 (t, J=8.5 Hz, 2H), 6.53 (d, J=9.0 Hz, 1H), 6.49 (d, J=8.5 Hz, 1H), 4.87-4.84 (m, 1H), 4.79-4.75 (m, 1H), 4.26-4.17 (m, 4H), 3.58-3.54 (m, 4H), 1.41 (s, 9H), 1.40 (s, 9H). ¹³C NMR (125 MHz, CDCl₃): δ ppm 165.11, 163.30, 161.34, 156.29, 156.19, 156.02, 151.11, 151.04, 151.02, 150.94, 150.29, 149.15, 149.05, 148.96, 134.13, 134.10, 134.08, 134.05, 133.65, 133.62, 132.74, 132.71, 129.23, 128.94, 128.92, 128.53, 125.88, 125.86, 125.84, 125.81, 124.12, 121.33, 118.73, 118.59, 117.18, 117.04, 115.20, 115.03, 112.85, 112.83, 112.03, 80.46, 80.18, 66.59, 66.50, 28.53, 28.52. MS (ESI) m/z=746.3 [M+H]⁺.

### aa. Preparation of tert-Butyl (S)-3-((5-((6-(((S)-1-(tert-butoxycarbonyl) pyrrolidin-3-yl) oxy)-3′,4′-difluoro-[1,1′-biphenyl]-3-yl) carbamoyl)-3′,4′-difluoro-[1,1′-biphenyl]-2-yl) oxy) pyrrolidine-1-carboxylate (Compound 64d)

64d (0.33 g, 81% yield) as a white solid. ¹H NMR (500 MHz, CDCl₃): δ ppm 8.91-8.61 (dd, 1H), 7.90-7.78 (m, 2H), 7.67-7.61 (m, 2H), 7.26-7.09 (m, 6H), 6.94-6.83 (m, 2H), 4.93-4.90 (m, 1H), 4.77-4.75 (s, 1H), 3.63-3.25 (m, 8H), 2.08-2.02 (m, 4H), 1.42 (s, 9H), 1.41 (s, 9H). ¹³C NMR (125 MHz, CDCl₃): δ ppm 173.11, 165.11, 156.26, 154.81, 154.73, 154.52, 154.45, 150.74, 150.65, 150.60, 150.48, 150.20, 148.76, 134.78, 134.72, 134.27, 132.77, 132.50, 132.46, 130.26, 130.18, 129.58, 128.86, 128.76, 127.90, 127.88, 127.75, 125.54, 123.39, 123.27, 121.59, 121.55, 121.41, 118.51, 118.39, 116.83, 116.70, 116.56, 115.08, 113.16, 80.00, 79.88, 79.70, 77.79, 76.42, 51.56, 51.02, 44.26, 43.81, 31.66, 31.62, 30.81, 28.50.

### bb. Preparation of tert-Butyl (R)-3-((5-(6-(((R)-1-(tert-butoxycarbonyl) pyrrolidin-3-yl) oxy)-3′,4′-difluoro-[1,1′-biphenyl]-3-ylcarboxamido)-4′-fluoro-[1,1′-biphenyl]-2-yl) oxy) pyrrolidine-1-carboxylate (Compound 64e)

64e (0.13 g, 88% yield) as an amorphous solid. The NMR data is the same as 64b.

### cc. Preparation of tert-Butyl (S)-3-((5-(6-(((R)-1-(tert-butoxycarbonyl) pyrrolidin-3-yl) oxy)-3′,4′-difluoro-[1,1′-biphenyl]-3-carboxamido)-4′-fluoro-[1,1′-biphenyl]-2-yl) oxy) pyrrolidine-1-carboxylate (Compound 64f)

64f (0.14 g, 95% yield) as an amorphous solid. ¹H NMR (500 MHz, d⁶-Acetone): δ ppm 9.54 (s 1H), 8.07-8.04 (m, 2H), 7.84-7.82 (m, 2H), 7.54 (dd, J=5.5, 8.5 Hz, 2H), 7.52-7.48 (m, 1H), 7.39-7.36 (m, 2H), 7.30 (d, J=8.0 Hz, 1H), 7.18-7.13 (m, 3H), 5.25-5.20 (m, 1H), 5.03-4.98 (m, 1H), 3.65-3.19 (m, 8H), 2.24-2.09 (m, 4H), 1.43 (s, 9H), 1.40 (s, 9H).

### dd. Preparation of tert-Butyl (R)-3-((5-(6-(((S)-1-(tert-butoxycarbonyl) pyrrolidin-3-yl) oxy)-3′,4′-difluoro-[1,1′-biphenyl]-3-carboxamido)-4′-fluoro-[1,1′-biphenyl]-2-yl) oxy) pyrrolidine-1-carboxylate (Compound 64g)

64g (0.05 g, 81% yield) as an amorphous solid. The NMR data is the same as 64f.

### ee. Preparation of (S)-3-((4′-Fluoro-5-((4′-fluoro-6-((S)-pyrrolidin-1-ium-3-yloxy)-[1,1′-biphenyl]-3-yl) carbamoyl)-[1,1′-biphenyl]-2-yl) oxy) pyrrolidin-1-ium chloride (Compound 20)

To a solution of 64a (0.21 g, 0.27 mmol) in MeOH (10 mL) under anhydrous conditions was added 4 M HCl in dioxane (10 mL, 0.04 mmol). The mixture was then stirred at room temperature for 1 h. The solvent was removed under reduced pressure to yield 20 (0.08 g, 48% yield) as an off-white solid. ¹H NMR (500 MHz, d⁶-DMSO): δ ppm 10.29 (s, 1H), 9.70 (brs, 4H), 8.02-8.00 (m, 2H), 7.80-7.78 (m, 2H), 7.70-7.67 (m, 2H), 7.60-7.58 (m, 2H), 7.30-7.23 (m, 5H), 7.15 (d, J=9.5 Hz, 1H), 5.25-5.22 (m, 1H), 5.02-5.00 (m, 1H), 3.55-3.08 (m, 8H), 2.22-2.04 (m, 4H). ¹³C NMR (125 MHz, d⁶-DMSO): δ ppm 165.04, 163.16, 163.01, 161.21, 161.07, 156.37, 149.89, 134.69, 134.66, 134.23, 134.14, 134.11, 132.32, 132.26, 131.97, 131.91, 130.95, 130.35, 129.77, 129.65, 128.37, 123.84, 121.48, 115.73, 115.70, 115.56, 115.53, 115.36, 113.93, 77.01, 76.79, 50.13, 50.11, 44.35, 44.23, 31.656, 31.54. HRMS (ES+) m/z [C₃₃H₃₃F₂N₃O₃²⁺] calculated 557.2479, found=557.2467.

### ff. Preparation of (S)-3-((3′,4′-difluoro-5-((4′-fluoro-6-((S)-pyrrolidin-1-ium-3-yloxy)-[1,1′-biphenyl]-3-yl) carbamoyl)-[1,1′-biphenyl]-2-yl) oxy) pyrrolidin-1-ium chloride (Compound 21)

21 (0.08 g, quantitative yield) as an off-white solid. ¹H NMR (500 MHz, d⁶-DMSO): δ ppm 10.22 (s, 1H), 9.37 (brs, 4H), 8.02-8.00 (m, 2H), 7.79-7.73 (m, 3H), 7.58 (dd, J=5.5, 9.0 Hz, 2H), 7.50-7.47 (m, 2H), 7.28 (d, J=9.0 Hz, 1H), 7.24 (t, J=9.0 Hz, 2H), 7.14 (d, J=9.0 Hz, 1H), 5.26-5.22 (m, 1H), 5.03-4.99 (m, 1H), 3.54 (dd, J=5.0, 13.0 Hz, 1H), 3.45 (dd, J=5.0, 13.0 Hz, 1H), 3.37-3.26 (m, 4H), 3.15-3.03 (m, 2H), 2.25-1.99 (m, 4H). ¹³C NMR (125 MHz, CD₃OD): δ ppm 166.42, 163.38, 161.43, 156.22, 150.99, 150.89, 150.87, 150.77, 150.30, 149.03, 148.93, 148.90, 148.80, 134.89, 134.86, 134.84, 134.80, 134.27, 134.25, 133.46, 131.56, 131.54, 131.30, 131.24, 130.64, 129.47, 129.30, 128.21, 128.19, 126.24, 126.21, 126.19, 126.16, 124.28, 121.88, 118.51, 118.37, 117.02, 116.88, 115.65, 114.93, 114.76, 113.44, 113.42, 77.37, 76.73, 50.64, 50.56, 44.45, 44.27, 30.99, 30.80; HRMS (ES+) m/z [C₃₃H₃₂F₃N₃O₃²⁺] calculated 575.2385, found=575.2341. [α]²⁵_{D}=+13.2 (c=0.2 in MeOH).

### gg. Preparation of 3-((5-(6-(azetidin-1-ium-3-yloxy)-3′,4′-difluoro-[1,1′-biphenyl]-3-ylcarboxamido)-4′-fluoro-[1,1′-biphenyl]-2-yl)oxy)azetidin-1-ium chloride (Compound 22)

22 (0.060 g, 81% yield) as a white solid. ¹H NMR (500 MHz, CD₃OD): δ ppm 8.00-7.98 (m, 2H), 7.69-7.67 (m, 2H), 7.60-7.57 (m, 3H), 7.43-7.30 (m, 2H), 7.15 (t, J=8.5 Hz, 2H), 6.93 (d, J=9.0 Hz, 1H), 6.82 (d, J=8.5 Hz, 1H), 5.30-5.28 (m, 1H), 5.15-5.13 (m, 1H), 4.61 (dd, J=6.5, 11.5 Hz, 2H), 4.51 (dd, J=6.5, 11.5 Hz, 2H), 4.14 (dd, J=4.0, 12.0 Hz, 2H), 4.07 (dd, J=4.5, 12.0 Hz, 2H). ¹³C NMR (125 MHz, CD₃OD): δ ppm 166.34, 163.44, 161.49, 155.53, 151.00, 150.97, 150.90, 150.87, 149.78, 149.04, 149.00, 148.94, 148.90, 134.58, 134.55, 134.53, 134.50, 133.97, 133.94, 133.52, 131.33, 131.26, 130.64, 129.41, 128.74, 126.25, 126.23, 126.20, 126.18, 121.85, 118.60, 118.45, 117.03, 116.89, 114.84, 114.66, 113.51, 112.57, 68.41, 68.35, 53.45, 53.20. HRMS (ESI) Calcd for C₃₁H₂₆F₃N₃O₃ (M+H)⁺ 546.1999, found 546.2012.

### hh. Preparation of (S)-3-((5-((3′,4′-difluoro-6-((S)-pyrrolidin-1-ium-3-yloxy)-[1,1′-biphenyl]-3-yl) carbamoyl)-3′,4′-difluoro-[1,1′-biphenyl]-2-yl) oxy) pyrrolidin-1-ium chloride (Compound 23)

23 (0.04 g, quantitative yield) as an off-white solid. ¹H NMR (500 MHz, d⁶-DMSO): δ ppm 10.34 (s, 1H), 9.72 (brs, 4H), 8.04-8.03 (m, 2H), 7.81-7.76 (m, 3H), 7.61-7.58 (m, 1H), 7.50-7.24 (m, 5H), 7.15 (d, J=9.2 Hz, 1H), 5.25-5.23 (m, 1H), 5.06-5.03 (m, 1H), 3.56-3.08 (m, 8H), 2.22-2.04 (m, 4H). ¹³C NMR (125 MHz, d⁶-DMSO): δ ppm 164.95, 156.31, 150.75, 150.71, 150.65, 150.61, 150.50, 150.40, 150.36, 150.26, 149.83, 148.80, 148.77, 148.70, 148.66, 148.54, 148.44, 148.40, 148.30, 135.86, 135.83, 135.81, 135.78, 135.28, 135.25, 135.23, 135.20, 134.12, 131.02, 130.16, 129.03, 128.53, 128.29, 128.28, 127.36, 127.34, 127.32, 127.29, 126.94, 126.91, 126.88, 126.86, 123.89, 122.01, 119.36, 119.22, 118.98, 118.84, 117.91, 117.83, 117.78, 117.69, 115.03, 115.01, 113.86, 76.92, 76.87, 50.07, 50.05, 44.32, 44.24, 31.66, 31.59. HRMS (ES+) m/z [C₃₃H₃₁F₄N₃O₃²⁺] calculated 593.2291, found=593.2261.

### ii. Preparation of (R)-3-((3′,4′-difluoro-5-((4′-fluoro-6-(((R)-pyrrolidin-1-ium-3-yl) oxy)-[1,1′-biphenyl]-3-yl) carbamoyl)-[1,1′-biphenyl]-2-yl) oxy) pyrrolidin-1-ium chloride (Compound 26)

26 (0.086 g, 78% yield) as a white solid. The NMR data is the same as 21; [α]²⁵_{D}=−13.2 (c=0.2 in MeOH).

### jj. Preparation of (R)-3-((3′,4′-difluoro-5-((4′-fluoro-6-(((S)-pyrrolidin-1-ium-3-yl) oxy)-[1,1′-biphenyl]-3-yl) carbamoyl)-[1,1′-biphenyl]-2-yl) oxy) pyrrolidin-1-ium chloride (Compound 27)

27 (0.076 g, 63% yield) as a white solid. ¹H NMR (500 MHz, d⁶-DMSO): δ ppm 10.27 (s, 1H), 9.56 (brs, 4H), 8.02-8.01 (m, 2H), 7.80-7.74 (m, 3H), 7.58 (dd, J=5.5, 9.0 Hz, 2H), 7.50-7.45 (m, 2H), 7.28 (d, J=9.5 Hz, 1H), 7.24 (t, J=9.0 Hz, 2H), 7.14 (d, J=9.5 Hz, 1H), 5.26-5.23 (m, 1H), 5.03-4.99 (m, 1H), 3.54 (dd, J=5.0, 13.0 Hz, 1H), 3.45 (dd, J=5.0, 13.0 Hz, 1H), 3.35-3.25 (m, 4H), 3.14-3.03 (m, 2H), 2.21-2.01 (m, 4H). ¹³C NMR (125 MHz, CD₃OD): δ ppm 166.44, 163.39, 161.44, 156.23, 151.00, 150.89, 150.78, 150.30, 149.04, 148.94, 148.92, 148.82, 134.90, 134.87, 134.85, 134.82, 134.28, 134.26, 133.47, 131.59, 131.58, 131.29, 131.23, 130.64, 129.51, 129.50, 129.29, 128.24, 126.22, 126.19, 126.17, 126.14, 124.27, 121.88, 118.51, 118.37, 117.02, 116.88, 115.66, 114.92, 114.75, 113.44, 77.38, 76.73, 50.65, 50.57, 44.45, 44.26, 30.98, 30.78; [α]²⁵_{D}=+2.8 (c=0.6 in MeOH).

### kk. Preparation of (S)-3-((3′,4′-difluoro-5-((4′-fluoro-6-(((R)-pyrrolidin-1-ium-3-yl) oxy)-[1,1′-biphenyl]-3-yl) carbamoyl)-[1,1′-biphenyl]-2-yl) oxy) pyrrolidin-1-ium chloride (Compound 28)

28 (0.013 g, 76% yield) as a white solid. The NMR data is the same as 27; [α]²⁵_{D}=−2.8 (c=0.6 in MeOH).

### 11. Preparation of Compounds 24 and 25

The synthesis scheme (Synthesis Scheme 11) for the preparation of Compounds 24 and 25 is shown below. The synthesis proceeds through the intermediates indicated (Compounds 59a, 65, 66, 67a, and 67b). The yield for each synthetic step was as indicated.

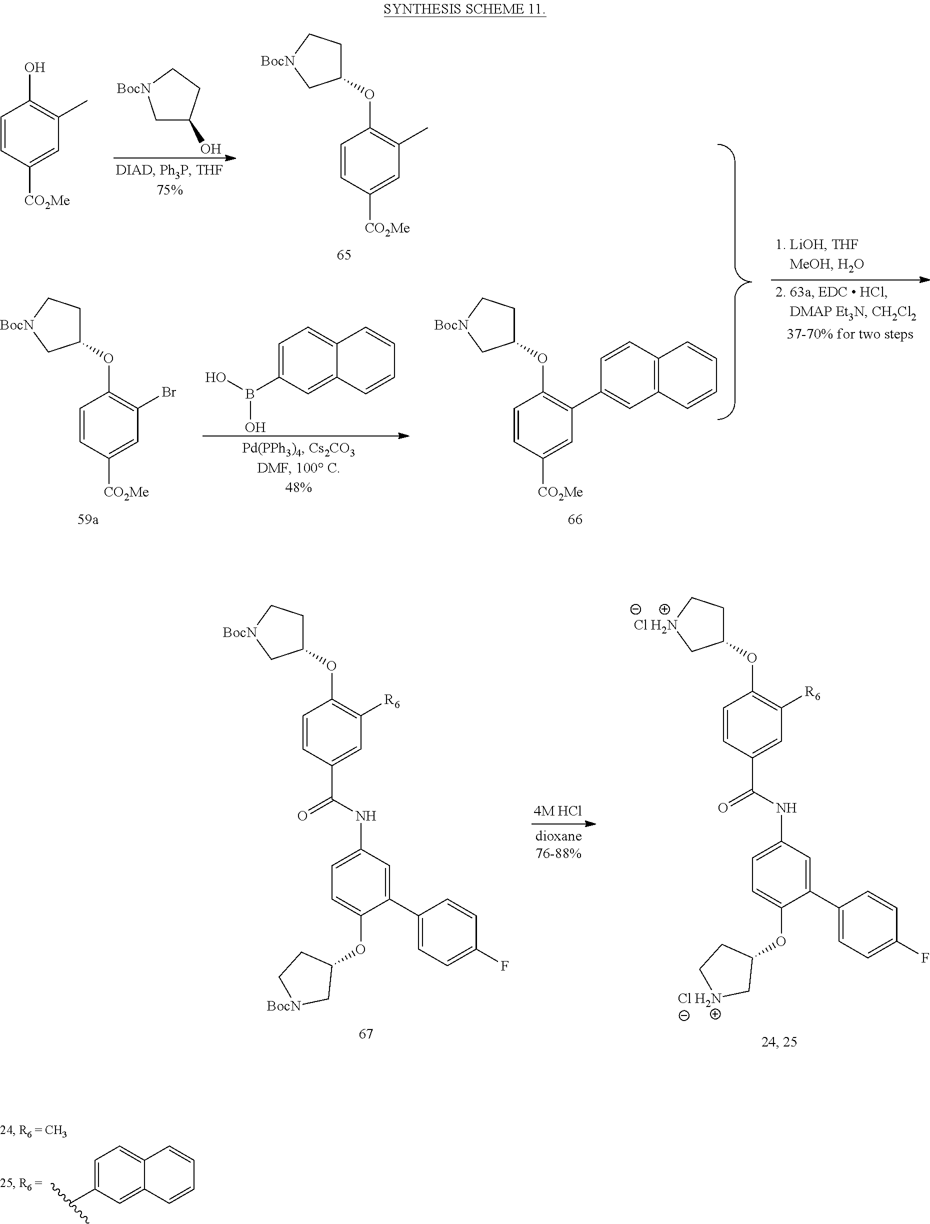

### a. Preparation of tert-Butyl (S)-3-(4-(methoxycarbonyl)-2-methylphenoxy) pyrrolidine-1-carboxylate (Compound 65)

To a solution of methyl 4-hydroxy-3-methylbenzoate (0.30 g, 1.80 mmol), (R)-tert-butyl-3-hydroxypyrrolidine-1-carboxylate (0.34 g, 1.80 mmol), and triphenylphosphine (0.58 g, 2.20 mmol) in dry THF (15 mL) was added diisopropyl azodicarboxylate (0.44 g, 2.20 mmol) at 0° C. Then, the temperature was allowed to rise to room temperature and stir for another 1 h. Upon completion, the reaction was diluted with CH₂Cl₂ (150 mL), washed with NaOH (1M) (50 mL×2) and brine (50 mL×2), dried over Na₂SO₄, and concentrated to give the crude product. The residue was purified by column chromatography (silica gel, hexanes:acetone=8:1 to 6:1) to yield 65 (0.45 g, 75% yield) as a white solid. ¹H NMR (500 MHz, CDCl₃): δ ppm 7.71-7.68 (m, 2H), 6.64 (d, J=8.5 Hz, 1H), 4.84-4.80 (m, 1H), 3.72 (s, 3H), 3.51-3.36 (m, 4H), 2.05 (s, 3H), 2.04-2.00 (m, 2H), 1.32 (s, 9H). ¹³C NMR (125 MHz, CDCl₃): δ ppm 166.87, 159.15, 154.61, 154.53, 132.44, 129.18, 127.59, 127.54, 122.47, 111.21, 79.53, 76.73, 75.91, 51.82, 51.76, 51.51, 44.27, 43.91, 31.72, 31.00, 28.55. MS (ESI) m/z=336.2 [M+H]⁺.

### b. Preparation of tert-Butyl (S)-3-(4-(methoxycarbonyl)-2-(naphthalen-2-yl) phenoxy) pyrrolidine-1-carboxylate (Compound 66)

To a solution of 59a (0.80 g, 2.00 mmol) in dry DMF (50 mL) was added naphthalen-2-ylboronic acid (0.41 g, 2.40 mmol), Pd(PPh₃)₄ (0.23 g, 0.20 mmol), and Cs₂CO₃ (1.95 g, 6.00 mmol). The mixture was heated to 100° C. under argon and stirred for 24 h. Then, the reaction mixture was cooled to room temperature, diluted with diethyl ether (150 mL), washed with water (50 mL×2) and brine (50 mL×2), dried over Na₂SO₄, and concentrated under vacuum. The residue was purified by column chromatography (silica gel, hexanes:acetone=15:1 to 10:1) to yield 66. ¹H NMR (500 MHz, d⁶-acetone): δ ppm 8.10 (s, 1H), 8.05-7.96 (m, 3H), 7.94 (d, J=8.5 Hz, 2H), 7.66 (d, J=8.5 Hz, 1H), 7.54-7.52 (m, 2H), 7.30 (d, J=8.5 Hz, 1H), 5.28-5.22 (m, 1H), 3.88 (s, 3H), 3.65-3.35 (m, 4H), 2.22-2.10 (m, 2H), 1.41 (s, 4.5H), 1.38 (s, 4.5H). ¹³C NMR (125 MHz, d⁶-acetone): δ ppm 166.17, 158.18, 154.11, 135.40, 135.29, 133.70, 132.89, 132.61, 131.49, 130.75, 128.46, 128.36, 127.72, 127.61, 127.49, 126.34, 123.52, 113.62, 113.43, 78.66, 77.40, 76.60, 51.62, 51.54, 51.25, 44.31, 44.07, 31.45, 30.56, 27.96; MS (ESI) m/z=448.2 [M+H]⁺.

### c. Preparation of tert-Butyl (S)-3-((5-(4-(((S)-1-(tert-butoxycarbonyl) pyrrolidin-3-yl) oxy)-3-methylbenzamido)-4′-fluoro-[1,1′-biphenyl]-2-yl) oxy) pyrrolidine-1-carboxylate (Compound 67a)

To the solution of 65 (0.40 g, 1.19 mmol) in a solvent mixture (28 mL, THF:MeOH:H₂O=4:2:1) was added LiOH (0.23 g, 9.54 mmol). The mixture was stirred for 8 h at room temperature. Then, the pH value was adjusted to 4-5 with HCl (1 M), diluted with water (50 mL), and extracted with EtOAc (50 mL×3). The combined organic phase was dried over Na₂SO₄ and concentrated under vacuum. The resulting crude product (0.40 g, 1.24 mmol) was dissolved into CH₂Cl₂ (20 mL). To this solution was added 63a (0.46 g, 1.24 mmol), dimethylaminopyridine (0.15 g, 1.24 mmol), and EDC.HCl (0.36 g, 1.78 mmol) at 0° C. The mixture was stirred at room temperature overnight, diluted with CH₂Cl₂ (150 mL), washed with brine (50 mL×3), dried over Na₂SO₄, and concentrated under vacuum. The residue was purified by column chromatography (silica gel, hexanes:acetone=5:1 to 3:1) to yield 67a (0.30 g, 37% two steps yield) as a white solid. ¹H NMR (500 MHz, d⁶-acetone): δ ppm 8.45 (s, 1H), 7.87-7.82 (m, 4H), 7.54-7.51 (m, 2H), 7.14 (t, J=8.5 Hz, 2H), 7.10 (d, J=8.5 Hz, 1H), 7.02 (d, J=8.5 Hz, 1H), 5.11-5.14 (m, 1H), 4.98-4.96 (m, 1H), 3.26-3.20 (m, 8H), 2.20-2.14 (m, 7H), 1.52-1.40 (m 18H). ¹³C NMR (125 MHz, d⁶-acetone): δ ppm 165.14, 163.09, 161.15, 158.24, 154.28, 154.15, 154.06, 150.22, 150.17, 134.85, 134.02, 131.43, 131.37, 131.17, 131.06, 130.36, 127.62, 127.27, 127.12, 123.13, 123.08, 121.99, 120.87, 115.85, 115.58, 115.37, 114.94, 114.90, 114.77, 114.73, 112.03, 111.98, 78.69, 78.56, 77.88, 77.08, 76.26, 51.81, 51.58, 51.49, 51.22, 44.33, 44.24, 44.00, 31.62, 31.39, 30.79, 30.63, 28.05. MS (ESI) m/z=676.2 [M+H]⁺.

### d. Preparation of (S)-tert-Butyl 3-((5-(4-(((S)-1-(tert-butoxycarbonyl) pyrrolidin-3-yl) oxy)-3-(naphthalen-2-yl) benzamido)-4′-fluoro-[1,1′-biphenyl]-2-yl) oxy) pyrrolidine-1-carboxylate (Compound 67b)

67b (0.49 g, 70% two steps yield) as a white solid. ¹H NMR (500 MHz, d⁶-acetone): δ ppm 9.58 (s, 1H), 8.15 (d, J=5.0 Hz, 1H), 8.07 (d, J=8.5 Hz, 1H), 8.04 (s, 1H), 7.98-7.93 (m, 3H), 7.87-7.85 (m, 2H), 7.69 (d, J=8.5 Hz, 1H), 7.55-7.53 (m, 4H), 7.31 (d, J=8.5 Hz, 1H), 7.17-7.12 (m, 3H), 5.25-5.23 (m, 1H), 5.00-4.98 (m, 1H), 3.66-3.20 (m, 8H), 2.21-2.09 (m, 4H), 1.43-1.38 (m, 18H). ¹³C NMR (125 MHz, d⁶-acetone): δ ppm 164.82, 163.11, 161.16, 156.95, 154.11, 150.29, 135.70, 135.61, 134.85, 133.97, 133.69, 132.83, 131.43, 131.37, 131.19, 131.09, 130.64, 128.94, 128.41, 128.34, 127.93, 127.76, 127.53, 127.42, 126.29, 123.09, 120.85, 115.57, 115.35, 114.95, 114.77, 113.80, 113.62, 78.66, 78.48, 77.85, 77.32, 77.05, 76.53, 51.53, 51.54, 51.22, 51.18, 44.33, 44.21, 44.09, 43.96, 31.47, 31.37, 30.61, 30.59, 27.99, 27.98. MS (ESI) m/z=788.5 [M+H]⁺.

### e. Preparation of (S)-3-((4′-fluoro-5-(3-methyl-4-((S)-pyrrolidin-1-ium-3-yloxy) benzamido)-[1,1′-biphenyl]-2-yl) oxy) pyrrolidin-1-ium chloride (Compound 24)

To a solution of 67a (0.10 g, 0.15 mmol) in CH₂Cl₂ (3 mL) was added 4 M HCl in dioxane (3 mL). The mixture was stirred at room temperature for 1 h. The solvent was then removed under reduced pressure to yield the crude product, which was dissolved with deionized water (10 mL), washed with EtOAc (5 mL×3), and lyophilized to give 24 (0.072 g, 88% yield) as a white solid. ¹H NMR (500 MHz, CD₃OD): δ ppm 7.84-7.82 (m, 2H), 7.88-7.66 (m, 2H), 7.56-7.53 (m, 2H), 7.15 (t, J=8.5 Hz, 2H), 7.13 (d, J=8.5 Hz, 1H), 7.06 (d, J=8.5 Hz, 1H), 5.34-5.30 (m, 1H), 5.06-5.02 (m, 1H), 3.66-3.13 (m, 8H), 2.37-2.12 (m, 7H). ¹³C NMR (125 MHz, CD₃OD): δ ppm 167.08, 163.38, 161.43, 157.66, 150.24, 134.32, 134.29, 133.57, 131.58, 131.31, 131.24, 130.43, 127.61, 127.43, 127.10, 124.29, 121.89, 115.71, 114.92, 114.75, 111.53, 77.39, 75.82, 50.83, 50.63, 44.28, 44.26, 30.84, 30.79. HRMS (ESI) Calcd for C₂₈H₃₀FN₃O₃(M+H)⁺ 476.2344, found 476.2357.

### f. Preparation of (S)-3-((4′-fluoro-5-(3-(naphthalen-2-yl)-4-((S)-pyrrolidin-1-ium-3-yloxy) benzamido)-[1,1′-biphenyl]-2-yl) oxy) pyrrolidin-1-ium chloride (Compound 25)

25 (0.060 g, 76% yield) as a white solid. ¹H NMR (500 MHz, CD₃OD): δ ppm 8.04 (d, J=2.5 Hz, 1H), 8.00 (s, 1H), 7.99 (d, J=8.5 Hz, 1H), 7.91-7.85 (m, 3H), 7.69-7.67 (m, 3H), 7.53-7.47 (m, 4H), 7.23 (d, J=9.0 Hz, 1H), 7.12 (t, J=9.0 Hz, 2H), 7.09 (d, J=9.5 Hz, 1H), 5.70-5.40 (m, 1H), 5.20-4.90 (m, 1H), 3.62-3.11 (m, 8H), 2.30-2.10 (m, 4H). ¹³C NMR (125 MHz, CD₃OD): δ ppm 166.68, 163.35, 161.40, 156.58, 150.27, 135.25, 134.27, 134.24, 133.67, 133.48, 132.89, 131.81, 131.51, 131.31, 131.25, 130.98, 128.84, 128.30, 128.24, 128.06, 127.63, 127.45, 127.40, 126.13, 124.31, 121.92, 115.61, 114.93, 114.75, 113.73, 77.35, 76.79, 50.62, 50.57, 44.41, 44.26, 31.04, 30.81. HRMS (ESI) Calcd for C₃₇H₃₄FN₃O₃(M+H)⁺ 588.2657, found 588.2670.

### 12. Preparation of Compound 29

The synthesis scheme (Synthesis Scheme 12) for the preparation of Compound 29 is shown below. The synthesis proceeds through the intermediates indicated (Compounds 59a, 68, 69, 70, 71, 72, 73, and 74). The yield for each synthetic step was as indicated.

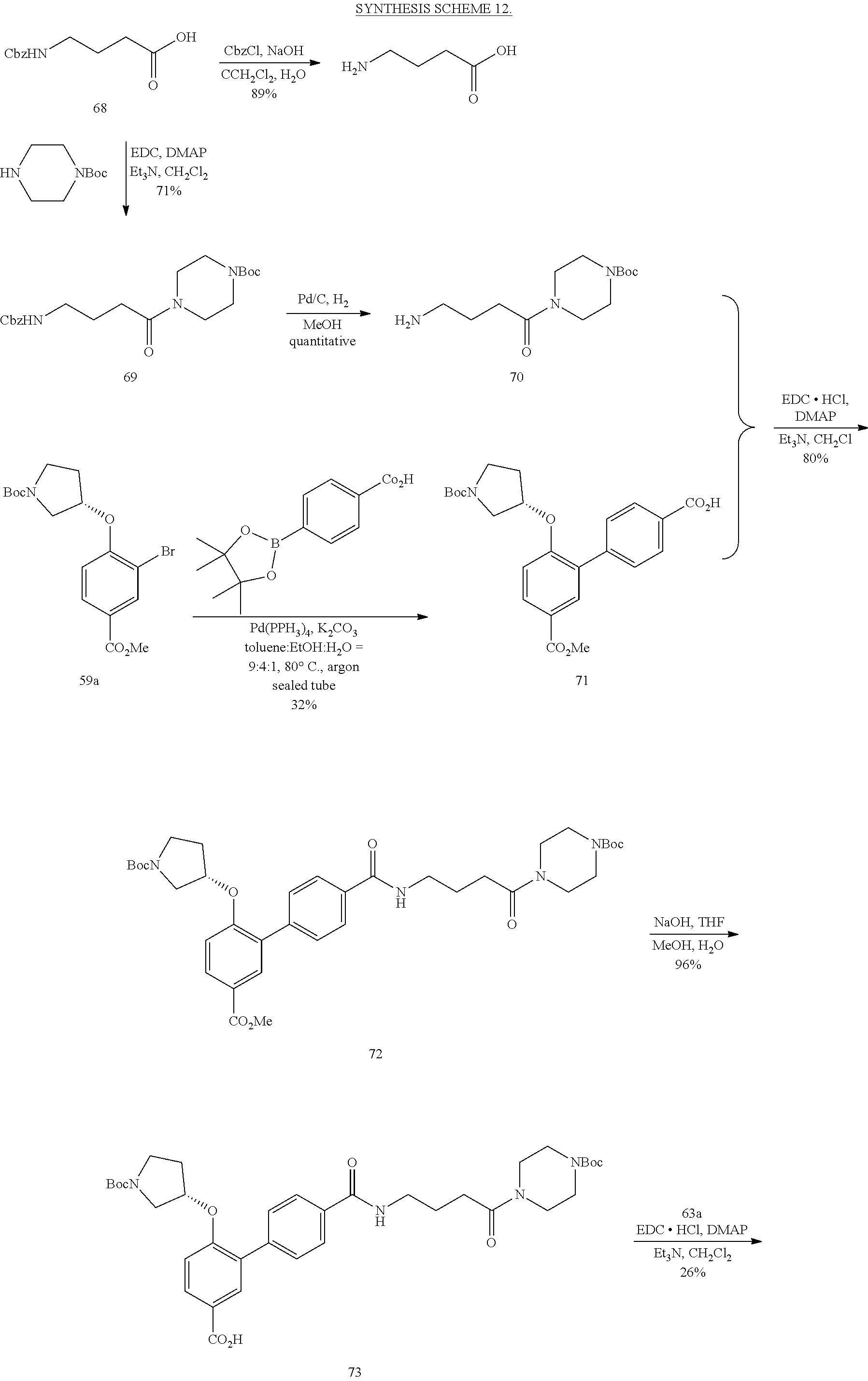

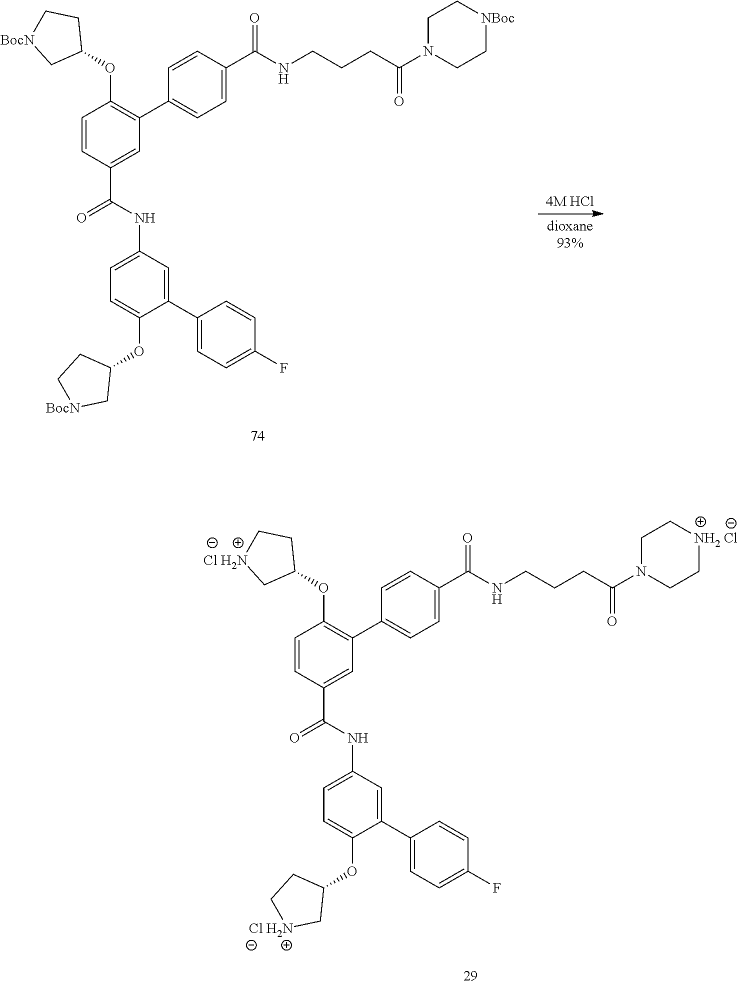

### a. Preparation of 4-(((Benzyloxy) carbonyl) amino) butanoic acid (Compound 68)

To a solution of 4-aminobutanoic acid (2.00 g, 19.40 mmol) in CH₂Cl₂ (20 mL) was added 3 M NaOH aqueous solution (30 mL). The mixture was then cooled to −5° C., and CbzCl (4.153 mL, 29.09 mmol) in CH₂Cl₂ was slowly added. The solution was allowed to warm to room temperature and stir for 16 h. Upon completion, the reaction layers were separated and the aqueous was washed with CH₂Cl₂ (20 mL). The aqueous was then acidified with 6 M HCl to a pH of 3. The aqueous was extracted with CH₂Cl₂ (50 mL), and the organic layer was dried over MgSO₄. After the filtration, the solvent was removed under reduced pressure to yield 68 (4.10 g, 89%) as a white solid. ¹H NMR (500 MHz, CDCl₃): δ ppm 11.28 (brs, 1H), 7.32-7.37 (m, 5H), 4.99-5.14 (m, 3H), 3.23-3.27 (m, 2H), 2.39 (t, 2H, J=7.1 Hz), 1.82-1.86 (m, 2H). ¹³C NMR (500 MHz, CDCl₃): δ ppm 178.75, 173.23, 156.83, 136.64, 128.76, 128.38, 67.03, 40.47, 31.37, 25.16. MS (ESI) m/z=238.4 [M+H]⁺

### b. Preparation of tert-Butyl 4-(4-(((benzyloxy) carbonyl) amino) butanoyl) piperazine-1-carboxylate (Compound 69)

To a solution of 68 (2.00 g, 8.43 mmol) in CH₂Cl₂ (30 mL) was added tert-butyl piperazine-1-carboxylate (1.73 g, 9.27 mmol), Et₃N (2.92 mL, 21.08 mmol), EDC□HCl (2.42 g, 12.64 mmol), and DMAP (1.03 g, 8.43 mmol). The mixture was then stirred at room temperature for 4 h. The mixture was diluted with CH₂Cl₂ (50 mL) and washed with water (50 mL), brine (50 mL), and dried over MgSO₄. After the filtration, the solvent was removed under reduced pressure. The residue was then purified by column chromatography (silica gel, hexanes:EtOAc=1:3) to yield 69 (2.44 g, 71% yield) as a white solid. ¹H NMR (500 MHz, CDCl₃): δ ppm 7.32-7.35 (m, 5H), 5.08 (s, 2H), 3.39-3.56 (m, 8H), 3.26 (q, J=7.0 Hz, 2H), 2.37 (t, 2H, J=6.9 Hz), 1.85-1.87 (m, 2H), 1.47 (s, 9H). ¹³C NMR (500 MHz, CDCl₃): δ ppm 171.26, 156.77, 154.73, 136.88, 128.70, 128.29, 80.51, 66.75, 45.46, 41.62, 40.93, 30.69, 28.59, 25.33. MS (ESI) m/z=406.7 [M+H]⁺.

### c. Preparation of tert-Butyl 4-(4-aminobutanoyl) piperazine-1-carboxylate (Compound 70)

To a solution of 69 (2.44 g, 6.02 mmol) in MeOH (30 mL) was added 10% Pd on activated carbon (0.244 g, 10% by weight). The air was evacuated and exchanged with H₂ gas three times, and the reaction was allowed to stir under H₂ gas for 16 h. The mixture was filtered through celite, and the solvent was removed under reduced pressure to afford 70 (1.69 g, quantitative yield) as an off-white solid. ¹H NMR (500 MHz, CDCl₃): δ ppm 3.57-3.59 (m, 2H), 3.39-3.44 (m, 6H), 2.79 (t, J=6.8 Hz, 2H), 2.37-2.44 (m, 4H), 1.80-1.82 (m, 2H), 1.47 (s, 9H). ¹³C NMR (500 MHz, CDCl₃): δ ppm 171.45, 154.73, 80.46, 45.51, 41.58, 41.29, 30.73, 28.56, 27.78. MS (ESI) m/z=272.7 [M+H]⁺.

### d. Preparation of (S)-tert-Butyl 4-(4-(2′-((1-(tert-butoxycarbonyl) pyrrolidin-3-yl) oxy)-5′-(methoxycarbonyl)-[1,1′-biphenyl]-4-ylcarboxamido) butanoyl) piperazine-1-carboxylate (Compound 71)

To a solution of 59a (0.50 g, 1.25 mmol) in toluene:EtOH:H₂O (9:4:1, 25 mL) was added 4-(4,4,5,5-tetramethyl-1,3,2-dioxaborolan-2-yl) benzoic acid (0.59 g, 1.38 mmol), Pd(PPh₃)₄ (0.07 g, 0.06 mmol), and K₂CO₃ (0.52 g, 3.75 mmol). The mixture was then heated to 80° C. in a sealed tube under argon and stirred for 16 h. The solvent was then removed under reduced pressure, and the residue was taken into EtOAc (50 mL). The solution was washed with water (2×50 mL), brine (50 mL), and dried over MgSO₄. After the filtration, the solvent was removed under reduced pressure. The residue was then purified by column chromatography (silica gel, CH₂Cl₂:MeOH=9:1) to give a crude mixture (0.551 g, 32%). The crude product (0.17 g, 0.38 mmol) was taken into CH₂Cl₂ (30 mL), 70 (0.115 g, 0.42 mmol), Et₃N (0.053 mL, 0.38 mmol), EDC.HCl (0.11 g, 0.58 mmol), and DMAP (0.046 g, 0.38 mmol). The reaction was then stirred at room temperature for 16 h. The mixture was diluted with CH₂Cl₂ (50 mL) and washed with water (50 mL), brine (50 mL), and dried over MgSO₄. After the filtration, the solvent was removed under reduced pressure. The residue was purified by column chromatography (silica gel, CH₂Cl₂:MeOH=9:1) to yield 72 (0.21 g, 80% yield) as a white solid. ¹H NMR (500 MHz, CDCl₃): δ ppm 7.93 (t, J=9.5 Hz, 2H), 7.78 (d, J=8.0 Hz, 2H), 7.37-7.45 (m, 3H), 6.89 (d, J=7.8 Hz, 1H), 4.88-4.91 (m, 1H), 3.81 (s, 3H), 3.23-3.53 (m, 14H), 2.43 (t, J=6.5 Hz, 2H,) 1.94-2.08 (m, 2H), 1.36-1.39 (m, 18H). ¹³C NMR (500 MHz, CDCl₃): δ ppm 173.00, 171.61, 167.12, 167.06, 167.04, 166.44, 157.52, 154.40, 140.37, 140.17, 133.33, 133.12, 132.57, 130.89, 130.66, 130.50, 129.36, 128.34, 126.89, 126.68, 126.63, 123.28, 123.19, 112.92, 112.87, 112.81, 80.18, 79.52, 76.25, 53.44, 51.94, 51.32, 50.93, 45.26, 44.10, 43.68, 41.44, 40.15, 40.10, 31.57, 31.298, 31.25, 30.73, 28.39, 28.27, 24.09, 24.00.

### e. Preparation of (S)-4′-((4-(4-(tert-butoxycarbonyl) piperazin-1-yl)-4-oxobutyl) carbamoyl)-6-((1-(tert-butoxycarbonyl) pyrrolidin-3-yl) oxy)-[1,1′-biphenyl]-3-carboxylic acid (Compound 73)

To a solution of 72 (0.10 g, 0.14 mmol) in a solvent mixture (10 mL, THF:H₂O:MeOH=4:1:1) was added 6 M NaOH (20 mL) and the reaction was stirred at 50° C. for 72 h. THF and MeOH were then removed under reduced pressure. The remaining aqueous solution was then acidified with 6 M HCl to pH=4. The product was then extracted with EtOAc (50 mL) and the organic layer was washed with water (50 mL), brine (50 mL), and dried over MgSO₄. After the filtration, the solvent was removed under reduced pressure to yield 73 (0.09 g, 96%) as a white solid. ¹H NMR (500 MHz, CDCl₃): δ ppm 8.01-8.07 (m, 2H), 7.84 (d, J=7.8 Hz, 2H), 7.47-7.49 (m, 3H), 6.92-6.95 (m, 1H), 4.93-4.95 (m, 1H), 3.29-3.62 (m, 14H), 2.50-2.53 (m, 2H), 2.02-2.07 (m, 4H), 1.40-1.43 (m, 18H). ¹³C NMR (500 MHz, CDCl₃): δ ppm 172.32, 171.42, 169.61, 169.47, 167.66, 158.13, 154.73, 140.70, 140.49, 133.38, 131.73, 131.02, 130.73, 130.71, 129.64, 128.67, 127.05, 127.01, 126.96, 123.38, 123.37, 123.24, 113.20, 112.99, 80.64, 80.08, 76.50, 60.62, 51.64, 51.19, 45.67, 44.43, 43.98, 41.86, 40.29, 31.87, 31.42, 31.40, 31.01, 29.89, 28.68, 28.56, 24.439, 24.39, 24.36, 21.26, 14.40.

### f. Preparation of tert-Butyl 4-(4-(2′-(((S)-1-(tert-butoxycarbonyl) pyrrolidin-3-yl) oxy)-5′-((6-(((S)-1-(tert-butoxycarbonyl) pyrrolidin-3-yl) oxy)-4′-fluoro-[1,1′-biphenyl]-3-yl) carbamoyl)-[1,1′-biphenyl]-4-ylcarboxamido) butanoyl) piperazine-1-carboxylate (Compound 74)

To a solution of 73 (0.85 g, 0.13 mmol) in CH₂Cl₂ (30 mL) was added amine 63a (0.05 g, 0.14 mmol), Et₃N (0.04 mL, 0.28 mmol), EDC□HCl (0.36 g, 0.19 mmol), and DMAP (0.015 g, 0.13 mmol). The mixture was then stirred at room temperature for 16 h. The mixture was diluted with CH₂Cl₂ (50 mL) and washed with water (50 mL), brine (50 mL), and dried over MgSO₄. After the filtration, the solvent was removed under reduced pressure. The residue was purified by column chromatography (silica gel, CH₂Cl₂:MeOH=9:1) to yield 74 (2.44 g, 71% yield) as a white solid. ¹H NMR (500 MHz, CDCl₃): δ ppm 8.71 (brs, 1H), 7.88-7.92 (m, 2H), 7.75 (d, J=8.3 Hz, 2H), 7.62-7.64 (m, 2H), 7.42-7.46 (m, 4H), 6.90-7.03 (m, 4H), 4.90-3.93 (m, 1H), 4.73-4.76 (m, 1H), 3.27-3.60 (m, 18H), 2.42-2.46 (m, 2H), 1.85-2.05 (m, 6H), 1.38-1.42 (m, 27H). ¹³C NMR (500 MHz, CDCl₃): δ ppm 172.00, 171.98, 167.36, 165.29, 163.24, 163.20, 161.28, 161.25, 156.77, 156.74, 154.78, 150.76, 134.09, 134.06, 132.03, 131.23, 129.70, 126.93, 126.90, 126.87, 124.04, 123.91, 121.40, 121.39, 116.04, 116.02, 115.18, 115.06, 113.84, 113.61, 113.58, 113.56, 80.64, 79.92, 51.65, 51.61, 51.24, 51.16, 51.14, 45.51, 44.40, 44.31, 43.99, 41.73, 40.45, 40.42, 40.40, 31.68, 31.01, 29.91, 27.30, 24.07, 24.06, 24.04, 24.03, 23.99, 22.91, 14.34.

### g. Preparation of 4-(4-(5′-((4′-fluoro-6-((S)-pyrrolidin-1-ium-3-yloxy)-[1,1′-biphenyl]-3-yl) carbamoyl)-2′-((S)-pyrrolidin-1-ium-3-yloxy)-[1,1′-biphenyl]-4-ylcarboxamido) butanoyl) piperazin-1-ium (Compound 75)

To a solution of 74 (0.03 g, 0.03 mmol) in CH₂Cl₂ (1 mL) under anhydrous conditions was added 4 M HCl in dioxane (1 mL, 0.004 mmol) and the mixture was then stirred at room temperature for 1 h. The solvent was removed under reduced pressure to yield 29 (0.03 g, 93% yield) as a white solid. ¹H NMR (500 MHz, d⁶-DMSO): δ ppm 10.30 (s, 1H), 9.51-9.71 (m, 6H), 8.62 (t, J=5.2 Hz, 1H), 8.03-8.05 (m, 2H), 7.93 (d, J=8.2 Hz, 2H), 7.79-7.80 (m, 2H), 7.72 (d, J=8.1 Hz, 2H), 7.58 (dd, J=5.8, 8.2 Hz, 2H), 7.25-7.32 (m, 3H), 7.14 (d, J=9.5 Hz, 1H), 5.24-5.27 (m, 1H), 5.00-5.03 (m, 1H), 3.01-3.69 (m, 18H), 2.41 (t, J=7.0 Hz, 2H), 2.09-2.18 (m, 4H), 1.77 (t, J=6.5 Hz, 2H). ¹³C NMR (500 MHz, d⁶-DMSO): δ ppm 170.65, 165.97, 164.33, 162.31, 160.37, 155.76, 149.20, 139.79, 133.98, 133.96, 133.50, 133.26, 131.28, 131.21, 130.33, 129.64, 129.37, 129.33, 129.28, 127.70, 127.04, 123.16, 120.80, 115.04, 114.87, 114.64, 113.25, 76.29, 76.10, 49.46, 49.40, 43.71, 43.56, 42.58, 42.46, 41.72, 38.84, 37.77, 31.01, 30.85, 29.46, 24.57. HRMS (ES+) m/z [C₄₂H₅₀FN₆O₅³⁺] calculated 737.3810, found=737.3773.

### 13. Preparation of Compound 80

The synthesis scheme (Synthesis Scheme 13) for the preparation of Compound 80 is shown below. The synthesis proceeds through the intermediates indicated (Compounds 76, 77a, 77b, 78a, 78b, and 79). The yield for each synthetic step was as indicated.

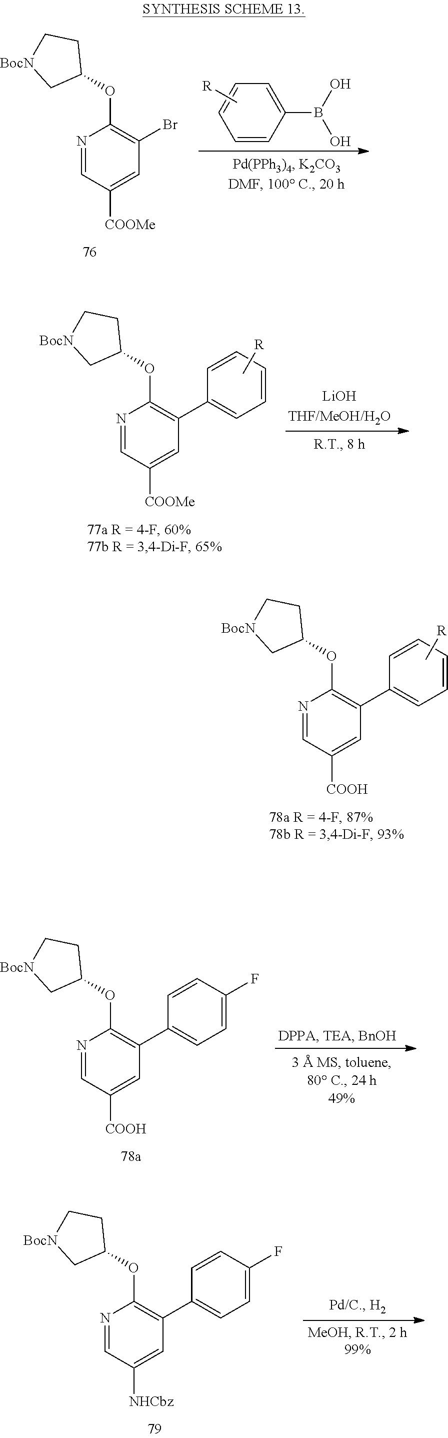

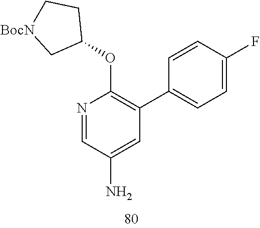 [SMILES: CC(C)(C)OC(=O)N1CC[C@H](Oc2ncc(N)cc2-c2ccc(F)cc2)C1]

### a. Preparation of (S)-tert-butyl-3-((5-(((benzyloxy)carbonyl)amino)-3-(4-fluorophenyl)pyridin-2-yl)oxy)pyrrolidine-1-carboxylate (Compound 79)

To a solution of 78a (0.42 g, 1.01 mmol) in dry toluene (20 mL) under anhydrous conditions was added DPPA (0.28 g, 1.01 mmol), Et₃N (0.20 g, 2.02 mmol), benzyl alcohol (5.00 mL), and activated molecular sieves (2 g). The mixture was stirred at room temperature for 10 min and then heated to 80° C. under nitrogen for 24 h. Upon completion the molecule sieves were filtered and the solution diluted with EtOAc (50 mL). The organic layer was washed with water (50 mL), brine (50 mL), dried over MgSO₄, solids filtered, and the solvent removed under reduced pressure. The residue was purified by column chromatography (hexanes:acetone=10:1-8:1) to yield 79 (0.25 g, 49%) as an amorphous solid. ¹H NMR (500 MHz, d⁶-Acetone): δ ppm 8.83 (brs, 1H), 8.32 (s, 1H), 8.03 (s, 1H), 7.63-7.60 (m, 2H), 7.45-7.30 (m, 5H), 7.22-7.17 (m, 2H), 5.62-5.68 (m, 1H), 6.20 (s, 2H), 3.66-3.36 (m, 4H), 2.21-2.10 (m, 2H), 1.43-1.40 (m, 9H).

### b. Preparation of tert-butyl (S)-3-((5-amino-3-(4-fluorophenyl)pyridin-2-yl)oxy)pyrrolidine-1-carboxylate (Compound 80)

To a solution of 79 (0.20 g, 0.39 mmol) in MeOH (15 ml) was added 10% Pd on activated carbon (0.02 g, 10% by weight). The air was evacuated and exchanged with the H₂ gas three times. The reaction mixture was allowed to stir under H₂ for 2 h and then filtered through celite. The solvent was removed under reduced pressure to yield 80 (0.15 g, >99%) as yellow oil. It was used directly in next step without further purification.

### 14. Preparation of Compounds 84a-c

The synthesis scheme (Synthesis Scheme 14) for the preparation of Compounds 84a-c is shown below. The synthesis proceeds through the intermediates indicated (Compounds 79a, 80, 81, 82, and 83a-c). The yield for each synthetic step was as indicated.

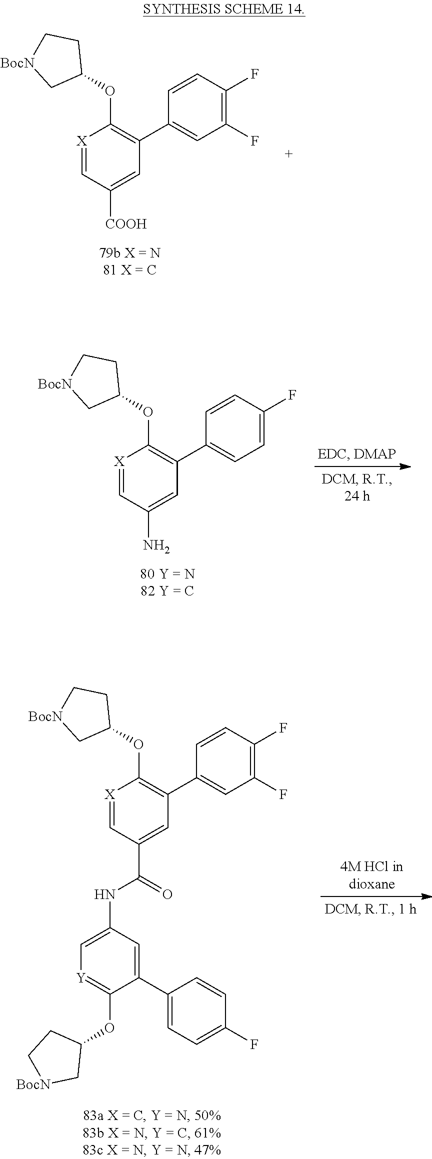

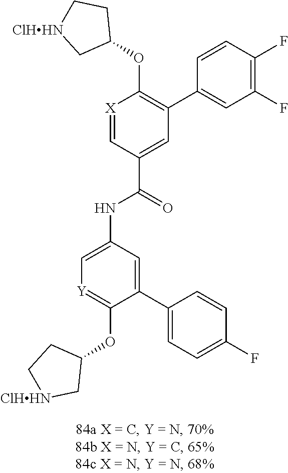

### a. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-3-(4-fluorophenyl)pyridin-2-yl)oxy)pyrrolidine-1-carboxylate (Compound 83a)

To a solution of 80 (0.08 g, 0.21 mmol) in CH₂Cl₂ (20 ml) was added 81 (0.09 g, 0.21 mmol), EDC.HCl (0.06 g, 0.31 mmol), and DMAP (0.026 g, 0.21 mmol). The mixture was then stirred at room temperature for 24 h. The mixture was diluted with CH₂Cl₂ (50 mL) and washed with water (50 mL), brine (50 mL), dried over MgSO₄, solids filtered, and solvent removed under reduced pressure. The residue was then purified by column chromatography (hexanes:acetone=3:1-2:1) to yield 83a (0.08 g, 50%) as a white solid. ¹H NMR (500 MHz, d⁶-Acetone): δ ppm 9.69 (s, 1H), 8.57 (s, 1H), 8.22 (s, 1H), 8.06-8.04 (m, 2H), 7.62-7.61 (m, 2H), 7.51-7.45 (m, 1H), 7.37-7.33 (m, 2H), 7.29 (d, J=8.5 Hz, 1H), 7.20-7.17 (m, 2H), 5.63-5.60 (m, 1H); 5.24-5.19 (m, 1H), 3.67-3.28 (m, 8H), 2.25-2.08 (m, 4H), 1.43-1.40 (m, 18H). ¹³C NMR (125 MHz, d⁶-Acetone): δ ppm 164.90, 163.45, 161.49, 156.88, 156.78, 155.77, 154.27, 154.20, 154.15, 153.99, 150.64, 150.55, 148.68, 148.58, 137.83, 135.27, 135.25, 135.22, 135.21, 132.81, 132.05, 131.95, 131.23, 131.17, 130.27, 129.58, 129.17, 129.08, 122.77, 126.28, 123.14, 118.67, 118.54, 117.15, 117.02, 115.27, 115.23, 115.10, 115.07, 113.91, 113.81, 78.72, 78.68, 78.54, 77.79, 77.78, 77.76, 76.85, 75.35, 74.52, 52.15, 51.83, 51.63, 51.18, 44.45, 44.25, 44.12, 43.93, 31.53, 31.39, 30.75, 30.53, 28.00, 27.94.

### b. Preparation of (S)-tert-butyl-3-((5-((6-(((S)-1-(tert-butoxycarbonyl)pyrrolidin-3-yl)oxy)-4′-fluoro-[1,1′-biphenyl]-3-yl)carbamoyl)-3-(3,4-difluorophenyl)pyridin-2-yl)oxy)pyrrolidine-1-carboxylate (83b)

The synthesis of 83b follows the same procedure as 83a to yield a white solid (61%). ¹H NMR (500 MHz, d⁶-Acetone): δ ppm 9.65 (s, 1H), 8.81 (s, 1H), 8.22 (s, 1H), 8.35-8.32 (m, 1H), 7.82-7.78 (m, 2H), 7.64-7.37 (m, 4H), 7.15-7.10 (m, 3H), 5.76-5.70 (m, 1H), 5.08-4.96 (m, 1H), 3.71-3.21 (m, 8H), 2.25-2.04 (m, 4H), 1.43-1.41 (m, 18H).

### c. Preparation of (S)-tert-butyl-3-((5-(6-(((S)-1-(tert-butoxycarbonyl)pyrrolidin-3-yl)oxy)-5-(3,4-difluorophenyl)nicotinamido)-3-(4-fluorophenyl)pyridin-2-yl)oxy)pyrrolidine-1-carboxylate (83c)

The synthesis of 83c follows the same procedure as 83a to yield a white solid (47%). ¹H NMR (500 MHz, d⁶-Acetone): δ ppm 9.76 (s, 1H), 8.83 (d, J=2.5 Hz, 1H), 8.56 (s, 1H), 8.35 (d, J=8.0 Hz, 1H), 8.21 (s, 1H), 7.64-7.59 (m, 3H), 7.52-7.46 (m, 1H), 7.43-7.38 (m, 1H), 7.21-7.18 (m, 2H), 5.76-5.71 (m, 1H), 5.65-5.60 (m, 1H), 3.70-3.30 (m, 8H), 2.22-2.14 (m, 4H), 1.43-1.41 (m, 18H).

### d. Preparation of 3′,4′-Difluoro-N-(5-(4-fluorophenyl)-6-((S)-pyrrolidin-3-yloxy)pyridin-3-yl)-6-((S)-pyrrolidin-3-yloxy)-[1,1′-biphenyl]-3-carboxamide dihydrochloride (84a)

To a solution of 83a (0.08 g, 0.10 mmol) in DCM (2 mL) under anhydrous conditions was added 4M HCl in dioxane (2 mL) and the mixture was then stirred at room temperature for 1 h. The solvent was removed under reduced pressure to yield 84a (0.45 g, 70%) as a white solid. ¹H NMR (500 MHz, CD₃OD): δ ppm 8.51 (s, 1H), 8.09 (s, 1H), 8.05 (dd, J=2.0 Hz, 8.5 Hz, 1H), 7.99 (s2, 1H), 7.66-7.60 (m, 2H), 7.56-7.49 (m, 1H), 7.42-7.32 (m, 2H), 7.27 (d, J=9.0 Hz, 1H), 7.22-7.14 (m, 2H), 5.77-5.73 (m, 1H), 5.35-5.30 (m, 1H), 3.67-3.34 (m, 8H), 2.39-2.28 (m, 4H). ¹³C NMR (125 MHz, CD₃OD): δ ppm 166.61, 156.43, 155.58, 138.44, 133.21, 133.15, 132.22, 131.20, 131.10, 131.04, 130.70, 129.41, 129.39, 127.85, 126.19, 123.71, 118.51, 118.37, 117.05, 116.91, 115.11, 114.93, 113.55, 76.80, 74.40, 51.11, 50.61, 44.58, 44.46, 30.96, 30.93.

### e. Preparation of 5-(3,4-difluorophenyl)-N-(4′-fluoro-6-((S)-pyrrolidin-3-yloxy)-[1,1′-biphenyl]-3-yl)-6-((S)-pyrrolidin-3-yloxy)nicomide dihydrochloride (84b)

The synthesis of 84b follows the same procedure as 84a to yield a white solid (65%). ¹H NMR (500 MHz, CD₃OD): δ ppm 8.76 (s, 1H), 8.32 (s, 1H), 7.74-7.68 (m, 2H), 7.66-7.59 (m, 1H), 7.58-7.52 (m, 2H), 7.48 (brs, 1H), 7.41-7.32 (m, 1H), 7.22-7.12 (m, 3H), 5.84 (s, 1H), 5.05 (s, 1H), 3.72 (dd, J=5.0 Hz, 13.5 Hz, 1H), 3.59 (d, J=13.5 Hz, 1H), 3.54-3.47 (m, 3H), 3.44-3.35 (m, 2H), 3.21-3.12 (m, 1H), 2.47-2.40 (m, 1H), 2.39-2.30 (m, 1H), 2.23-2.15 (m, 1H). ¹³C NMR (125 MHz, CD₃OD): δ ppm 164.94, 163.44, 161.49, 160.78, 150.51, 146.54, 138.57, 134.23, 133.23, 132.81, 131.71, 131.28, 131.22, 126.07, 126.05, 126.02, 125.47, 124.28, 122.58, 121.89, 118.33, 118.19, 117.33, 117.20, 115.74, 114.95, 114.78, 77.43, 75.28, 50.96, 50.67, 44.57, 44.27, 30.90, 30.78.

### f. Preparation of 5-(3,4-difluorophenyl)-N-(5-(4-fluorophenyl)-6-((S)-pyrrolidin-3-yloxy)pyridin-3-yl)-6-((S)-pyrrolidin-3-yloxy)nicotinamide dihydrochloride (84c)

The synthesis of 84c follows the same procedure as 84a to yield a white solid (68%). ¹H NMR (500 MHz, CD₃OD): δ ppm 8.80 (s, 1H), 8.51 (s, 1H), 8.34 (s, 1H), 8.11 (s, 1H), 7.65-7.61 (m, 3H), 7.48 (brs, 1H), 7.39-7.34 (m, 1H), 7.18 (t, J=8.5 Hz, 2H), 5.86 (brs, 1H), 5.75 (brs, 1H), 3.74-3.63 (m, 2H), 3.61-3.53 (m, 2H), 3.52-3.45 (m, 2H), 3.42-3.32 (m, 2H), 2.47-2.27 (m, 4H). ¹³C NMR (125 MHz, CD₃OD): δ ppm 165.06, 160.91, 155.68, 146.67, 138.62, 138.40, 133.07, 132.17, 132.15, 131.11, 131.05, 130.72, 126.07, 125.08, 123.78, 122.64, 118.36, 118.21, 117.36, 117.22, 115.14, 114.96, 75.32, 74.44, 51.14, 51.01, 44.61, 39.11, 30.94, 30.90.

### 15. Preparation of Compounds 91a-j

The synthesis scheme (Synthesis Scheme 15) for the preparation of Compounds 91a-j is shown below. The synthesis proceeds through the intermediates indicated (Compounds 82, 85, 86, 87, 88, 89a-j, and 90a-j). The +yield for each synthetic step was as indicated.

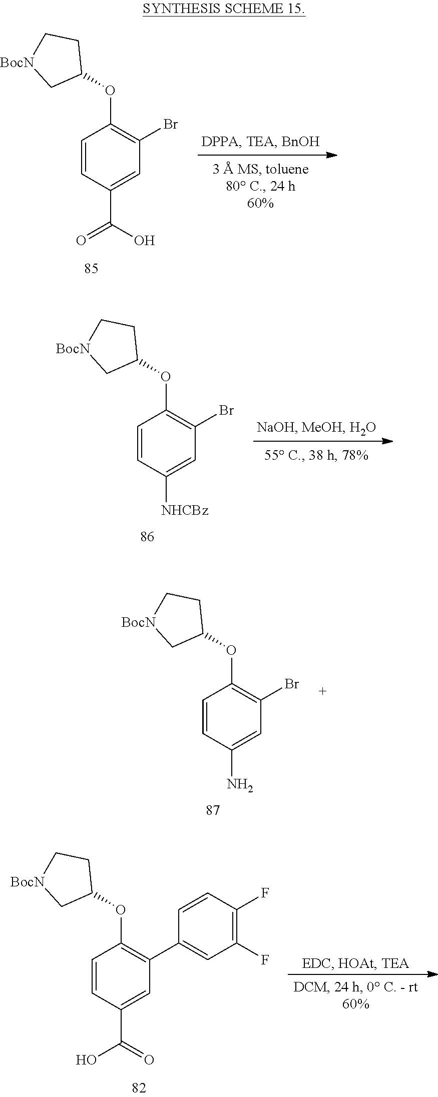

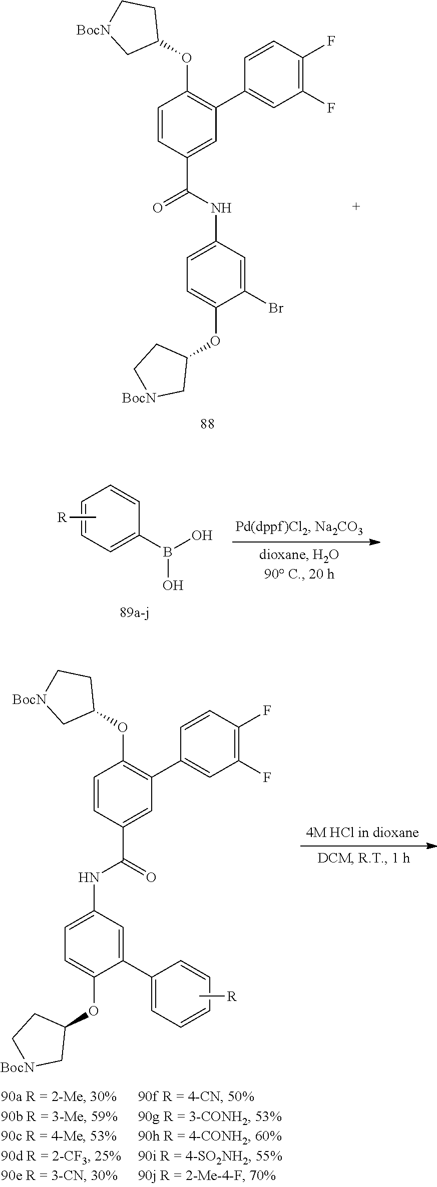

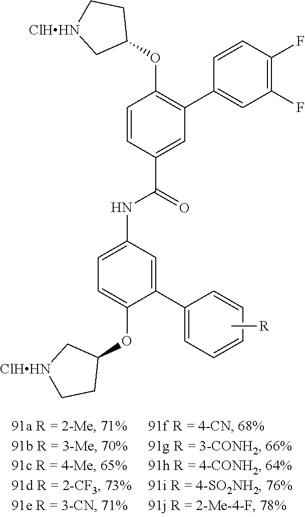

### a. Preparation of (S)-tert-butyl-3-(4-(((benzyloxy)carbonyl)amino)-2-bromophenoxy)pyrrolidine-1-carboxylate (Compound 86)

The synthesis of 86 uses the same procedure as 79 to yield an amorphous solid. The obtained product has to remain on the high vacuum pump for at least 48h to remove the benzyl alcohol. ¹H NMR (500 MHz, CDCl₃): δ ppm 7.63 (s, 1H), 7.41-7.26 (m, 5H), 6.81 (d, J=8.5 Hz, 2H), 5.18 (s, 2H), 4.82 (s, 1H), 3.64-3.50 (m, 4H), 2.24-2.15 (br.m, 1H), 2.08-1.99 (m, 1H), 1.46 (s, 9H). ¹³C NMR (500 MHz, CDCl₃): δ ppm 154.89, 154.76, 153.73, 150.12, 136.21, 133.18, 128.81, 128.50, 124.58, 119.25, 116.68, 114.37, 79.19, 78.31, 67.28, 51.69, 44.04, 31.92, 28.74.

### b. Preparation of (S)-tert-butyl-3-(4-amino-2-bromophenoxy)pyrrolidine-1-carboxylate (Compound 87)

The compound 86 (1.69 g, 3.44 mmol) was dissolved in MeOH and an aqueous solution of NaOH (3.6 g, 89.44 mmol) in 15 mL of water added to the stirring mixture. The reaction was heated to 55° C. for 38h. Upon completion, the reaction mixture was neutralized with HCl and the organic solvent was removed under reduced pressure. To the remaining aqueous layer was added saturated NaHCO₃ and washed with EtAcO three times. After removal of the organic solvent, a dark brown solid was obtained (0.96 g, 78%). ¹H NMR (500 MHz, CDCl₃): δ ppm 6.89 (s, 1H), 6.74 (d, J=14.0 Hz, 1H), 6.59-6.52 (m, 1H), 4.73 (s, 1H), 3.69-3.44 (m, 4H), 2.25-2.13 (m, 1H), 2.08-1.89 (m, 1H), 1.45 (s, 9H). ¹³C NMR (500 MHz, CDCl₃): δ ppm 154.90, 146.45, 142.83, 128.63, 127.13, 120.05, 119.38, 115.21, 80.03, 79.16, 51.67, 44.08, 31.05, 28.75.

### c. Preparation of (S)-tert-butyl-3-((5-((3-bromo-4-(((S)-1-(tert-butoxycarbonyl)pyrrolidin-3-yl)oxy)phenyl)carbamoyl)-3′,4′-difluoro-[1,1′-biphenyl]-2-yl)oxy)pyrrolidine-1-carboxylate (Compound 88)

Compound 82 (0.17 g, 0.41 mmol) was dissolved in anhydrous DCM at 0° C. EDC.HCl (0.16 g, 0.82 mmol) and HOAt (62.4 mg, 0.41 mmol) were added. The reaction mixture was allowed to stir at 0° C. for 30 min before 87 (0.15 g, 0.41 mmol) and Et₃N (0.20 ml, 1.31 mmol) were added. The reaction was stirred overnight while warming up to room temperature. The reaction was quenched after 24 h with 1M HCl. The aqueous layer was washed two times with EtAcO and the solvent of the combined organic layer was removed under reduced pressure. Flash column chromatography (hexane:EtAcO=1:1) purified the product 88. A white solid was isolated (186.62 mg, 60%). ¹H NMR (500 MHz, CDCl₃): δ ppm 8.98-8.80 (m, 1H), 7.87-7.78 (m, 3H), 7.71-7.47 (m, 1H), 7.19 (t, J=9.0 Hz, 1H), 7.12-7.04 (m, 2H), 7.00-6.72 (m, 2H), 4.91-4.80 (m, 2H), 3.69-3.40 (m, 7H), 3.37-3.23 (m, 1H), 2.23-1.99 (m, 4H), 1.42 (s, 18H). ¹³C NMR (500 MHz, CDCl₃): δ ppm 165.19, 156.63, 156.38, 154.81, 150.77, 150.49, 148.81, 134.26, 133.49, 130.26, 129.63, 128.82, 127.73, 125.98, 125.61, 120.90, 118.55, 116.94, 116.07, 113.87, 113.22, 80.00, 79.69, 51.58, 51.01, 44.29, 43.80, 31.66, 30.94, 28.60, 28.51.

### d. Preparation of (S)-tert-butyl-3-((5-((6-(((S)-1-(tert-butoxycarbonyl)pyrrolidin-3-yl)oxy)-2′-methyl-[1,1′-biphenyl]-3-yl)carbamoyl)-3,4′-difluoro-[1,1′-biphenyl]-2-yl)oxy)pyrrolidine-1-carboxylate (Compound 90a)

To a solution of 88 (0.15 g, 0.20 mmol) in solvent mixture (dioxane: H₂O=5:2, 35 mL) under anhydrous conditions was added (2-methylphenyl) boronic acid (89a) (0.04 g, 0.30 mmol), Pd(dppf)Cl₂ (0.015 g, 0.020 mmol), and Na₂CO₃ (0.063 g, 0.60 mmol). The mixture was heated to 90° C. under argon and stirred for 20 h. The solvent was then removed under reduced pressure, and the residue was taken into EtOAc (100 mL). The solution was washed with brine (50 mL) and dried over MgSO₄. After removal of the inorganic solid, the solvent was removed under reduced pressure. The residue was purified by column chromatography (hexanes:EtOAc=3:1-2:1) to yield 90a (0.05 g, 30%) as a white solid. ¹H NMR (500 MHz, d⁶-Acetone): δ ppm 9.51 (s, 1H), 8.05-8.03 (m, 2H), 7.87-7.84 (m, 1H), 7.62 (s, 1H), 7.53-7.50 (m, 1H), 7.39-7.36 (m, 2H), 7.29 (d, J=7.5 Hz, 1H), 7.24 (s, 1H), 7.21-7.19 (m, 2H), 7.13-7.09 (m, 2H), 5.24-5.20 (m, 1H), 4.94-4.89 (m, 1H), 3.65-3.04 (m, 8H), 2.26-1.90 (m, 7H), 1.45 (s, 9H), 1.39 (s, 9H).

### e. Preparation of (S)-tert-butyl-3-((5-((6-(((S)-1-(tert-butoxycarbonyl)pyrrolidin-3-yl)oxy)-3′-methyl-[1,1′-biphenyl]-3-yl)carbamoyl)-3′,4′-difluoro-[1,1′-biphenyl]-2-yl)oxy)pyrrolidine-1-carboxylate (Compound 90b)

Compound 90b was prepared using the same procedure as 90a (59%). ¹H NMR (500 MHz, d⁶-Acetone): δ ppm 9.60 (s, 1H), 8.05-8.03 (m, 2H), 7.85-7.82 (m, 1H), 7.79 (s, 1H), 7.53-7.45 (m, 1H), 7.35-7.23 (m, 6H), 7.13-7.07 (m, 2H), 5.19-5.16 (m, 1H), 4.99-4.94 (m, 1H), 3.63-3.26 (m, 8H), 2.36 (s, 3H), 2.20-2.04 (m, 4H), 1.43 (s, 9H), 1.40 (s, 9H).

### f. Preparation of (S)-tert-butyl-3-((5-((6-(((S)-1-(tert-butoxycarbonyl)pyrrolidin-3-yl)oxy)-4′-methyl-[1,1′-biphenyl]-3-yl)carbamoyl)-3′,4′-difluoro-[1,1′-biphenyl]-2-yl)oxy)pyrrolidine-1-carboxylate (Compound 90c)

Compound 90c was prepared through the same procedure with 90a as a white solid (53%). ¹H NMR (500 MHz, d⁶-Acetone): δ ppm 9.54 (s, 1H), 8.05-8.03 (m, 2H), 7.82-7.77 (m, 2H), 7.53-7.46 (m, 1H), 7.40 (d, J=8.0 Hz, 2H), 7.38-7.32 (m, 2H), 7.27 (d, J=8.5 Hz, 1H), 7.20 (d, J=7.5 Hz, 2H), 7.09 (d, J=9.0 Hz, 1H), 5.21-5.17 (m, 1H), 4.98-4.93 (m, 1H), 3.63-3.23 (m, 8H), 2.34 (s, 3H), 2.20-2.14 (m, 4H), 1.43 (s, 9H), 1.40 (s, 9H).

### g. Preparation of (S)-tert-butyl-3-((5-((6-(((S)-1-(tert-butoxycarbonyl)pyrrolidin-3-yl)oxy)-2′-(trifluoromethyl)-[1,1′-biphenyl]-3-yl)carbamoyl)-3′,4′-difluoro-[1,1′-biphenyl]-2-yl)oxy)pyrrolidine-1-carboxylate (Compound 90d)

Compound 90d was prepared through the same procedure with 90a as a white solid (27%). ¹H NMR (500 MHz, d⁶-Acetone): δ ppm 9.52 (s, 1H), 8.04-7.98 (m, 2H), 7.86 (s, 1H), 7.76 (s, 1H), 7.65-7.48 (m, 4H), 7.35-7.33 (m, 2H), 7.28-7.22 (m, 1H), 7.09-7.04 (m, 1H), 5.22-5.18 (m, 1H), 5.02-4.95 (m, 1H), 3.59-2.91 (m, 8H), 2.17-2.02 (m, 4H), 1.40 (s, 9H), 1.38 (s, 9H).

### h. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-3′-cyano-[1,1′-biphenyl]-2-yl)oxy)pyrrolidine-1-carboxylate (Compound 90e)

Compound 90e was prepared through the same procedure with 90a as a white solid (30%). ¹H NMR (500 MHz, d⁶-Acetone): δ ppm 9.64 (s, 1H), 8.05-8.03 (m, 2H), 7.87-7.79 (m, 4H), 7.71 (d, J=7.5 Hz, 1H), 7.59 (t, J=7.5 Hz, 1H), 7.51-7.44 (m, 1H), 7.35-7.31 (m, 2H), 7.26 (d, J=8.5 Hz, 1H), 7.15 (d, J=9.0 Hz, 1H), 5.22-5.17 (m, 1H), 5.05-5.01 (m, 1H), 3.61-3.27 (m, 8H), 2.19-2.13 (m, 4H), 1.42 (s, 9H), 1.40 (s, 9H).

### i. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-4′-cyano-[1,1′-biphenyl]-2-yl)oxy)pyrrolidine-1-carboxylate (Compound 90f)

Compound 90f was prepared through the same procedure with 90a as a white solid. (50%). ¹H NMR (500 MHz, d⁶-Acetone): δ ppm 9.60 (s, 1H), 8.07-8.04 (m, 2H), 7.89-7.87 (m, 2H), 7.80 (d, J=9.0 Hz, 2H), 7.72 (d, J=8.5 Hz, 1H), 7.55-7.51 (m, 1H), 7.30 (d, J=8.5 Hz, 1H), 7.19 (d, J=8.0 Hz, 1H), 5.25-5.21 (m, 1H), 5.08-5.04 (m, 1H), 3.63-3.20 (m, 8H), 2.20-2.08 (m, 4H), 1.43 (s, 9H), 1.40 (s, 9H).

### j. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-3′-carbamoyl-[1,1′-biphenyl]-2-yl)oxy)pyrrolidine-1-carboxylate (Compound 90g)

Compound 90g was prepared through the same procedure with 90a as a white solid. (53%). ¹H NMR (300 MHz, d⁶-Acetone): δ ppm 9.59 (s, 1H), 8.10-8.05 (m, 3H), 7.92-7.87 (m, 2H), 7.79 (s, 1H), 7.68-7.65 (m, 2H), 7.55-7.43 (m, 2H), 7.37-7.32 (m, 2H), 7.25 (d, J=9.0 Hz, 1H), 7.11 (d, J=9.0 Hz, 1H), 6.84 (brs, 1H), 5.23-5.17 (m, 1H), 5.00-4.95 (m, 1H), 3.61-3.25 (m, 8H), 2.22-2.09 (m, 4H), 1.42-1.39 (m, 18H).

### k. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-4′-carbamoyl-[1,1′-biphenyl]-2-yl)oxy)pyrrolidine-1-carboxylate (Compound 90h)

Compound 90h was prepared through the same procedure with 90a as a white solid (60%). ¹H NMR (300 MHz, d⁶-Acetone): δ ppm 9.69 (s, 1H), 8.07-7.84 (m, 6H), 7.61-7.45 (m, 4H), 7.37-7.25 (m, 3H), 7.14 (d, J=9.0 Hz, 1H), 6.82 (brs, 1H), 5.25-5.18 (m, 1H), 5.05-4.97 (m, 1H), 3.65-3.23 (m, 8H), 2.17-2.04 (m, 4H), 1.42-1.40 (m, 18H).

### l. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-4′-sulfamoyl-[1,1′-biphenyl]-2-yl)oxy)pyrrolidine-1-carboxylate (Compound 90i)

Compound 90i was prepared through the same procedure with 90a as a white solid (55%). ¹H NMR (500 MHz, d⁶-Acetone): δ ppm 9.62 (s, 1H), 8.05-8.03 (m, 2H), 7.92 (d, J=7.0 Hz, 2H), 7.88-7.84 (m, 2H), 7.66 (d, J=8.5 Hz, 2H), 7.54-7.46 (m, 1H), 7.37-7.32 (m, 2H), 7.28 (d, J=8.5 Hz, 1H), 7.16 (t, J=8.5 Hz, 1H), 6.65 (s, 1H), 6.63 (s, 1H), 5.24-5.19 (m, 1H), 5.07-5.02 (m, 1H), 3.64-3.14 (m, 8H), 2.23-2.10 (m, 4H), 1.43-1.40 (m, 18H).

### m. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-4′-fluoro-2′-methyl-[1,1′-biphenyl]-2-yl)oxy)pyrrolidine-1-carboxylate (Compound 90j)

Compound 90j was prepared through the same procedure with 90a as a white solid (70%). ¹H NMR (500 MHz, d⁶-Acetone): δ ppm 9.56 (s, 1H), 8.04-8.02 (m, 2H), 7.86-7.84 (m, 1H), 7.61 (s, 1H), 7.52-7.45 (m, 1H), 7.35-7.30 (m, 2H), 7.25 (d, J=9.0 Hz, 1H), 7.12-7.07 (m, 2H), 7.00 (d, J=10.0 Hz, 1H), 6.95-6.92 (m, 1H), 5.22-5.17 (m, 1H), 4.94-4.88 (m, 1H), 3.63-3.03 (m, 8H), 2.23-1.90 (m, 7H), 1.42-1.40 (m, 18H).

### n. Preparation of 3′,4′-Difluoro-N-(2′-methyl-6-((S)-pyrrolidin-3-yloxy)-[1,1′-biphenyl]-3-yl)-6-((S)-pyrrolidin-3-yloxy)-[1,1′-biphenyl]-3-carboxamide dihydrochloride (Compound 91a)

To a solution of 90a (0.05 g, 0.065 mmol) in DCM (2 mL) under anhydrous conditions was added 4 M HCl in dioxane (2 mL) and the mixture was then stirred at room temperature for 1 h. The solvent was then removed under reduced pressure to yield 91a (0.030 g, 71%) as a white solid. ¹H NMR (500 MHz, CD₃OD): δ ppm 8.01 (dd, J=2.0 Hz, 8.5 Hz, 1H), 7.96 (s, 1H), 7.73 (d, J=8.0 Hz, 1H), 7.54-7.47 (m, 2H), 7.39-7.08 (m, 8H), 5.31 (s, 1H), 4.94 (s, 1H), 3.64 (dd, J=4.5 Hz, 13 Hz, 1H), 3.55-3.43 (m, 3H), 3.28-3.20 (m, 1H), 3.08 (brs, 1H), 2.86 (brs, 1H), 2.39-2.23 (m, 2H), 2.17 (s, 3H), 2.09-1.95 (m, 3H). ¹³C NMR (125 MHz, CD₃OD): δ ppm 166.50, 156.24, 156.22, 150.79, 130.63, 129.75, 129.74, 129.27, 128.44, 127.62, 126.16, 126.13, 125.49, 121.84, 118.51, 118.37, 117.03, 116.89, 113.51, 76.77, 50.62, 44.45, 30.94, 19.06.

### o. Preparation of 3′,4′-Difluoro-N-(3′-methyl-6-((S)-pyrrolidin-3-yloxy)-[1,1′-biphenyl]-3-yl)-6-((S)-pyrrolidin-3-yloxy)-[1,1′-biphenyl]-3-carboxamide dihydrochloride (Compound 91b)

Compound 91b was prepared through the same procedure with 91a as a white solid (70%). ¹H NMR (500 MHz, CD₃OD): δ ppm 8.02 (d, J=8.0 Hz, 1H), 7.97 (s, 1H), 7.71-7.64 (m, 2H), 7.53-7.49 (m, 1H), 7.41-7.23 (m, 6H), 7.16 (d, J=6.5 Hz, 1H), 7.13 (d, J=8.5 Hz, 1H), 5.31 (s, 1H), 4.97 (s, 1H), 3.65 (dd, J=4.5 Hz, 13 Hz, 1H), 3.56-3.41 (m, 4H), 3.39-3.35 (m, 1H), 3.28-3.22 (m, 1H), 3.20-3.11 (m, 1H), 2.39 (s, 3H), 2.36-2.24 (m, 2H), 2.21-2.04 (m, 2H). ¹³C NMR (125 MHz, CD₃OD): δ ppm 163.08, 152.84, 147.10, 134.77, 134.50, 130.25, 129.85, 127.23, 126.56, 126.49, 126.22, 125.87, 125.00, 124.60, 124.52, 123.09, 122.78, 121.00, 118.29, 115.10, 114.97, 113.62, 113.48, 113.08, 110.14, 74.37, 73.39, 47.29, 47.21, 41.06, 40.82, 27.56, 27.33, 16.93.

### p. Preparation of 3′,4′-Difluoro-N-(4′-methyl-6-((S)-pyrrolidin-3-yloxy)-[1,1′-biphenyl]-3-yl)-6-((S)-pyrrolidin-3-yloxy)-[1,1′-biphenyl]-3-carboxamide dihydrochloride (Compound 91c)

Compound 91c was prepared through the same procedure with 91a to yield a white solid (65%). ¹H NMR (500 MHz, CD₃OD): δ ppm 8.02 (d, J=8.5 Hz, 1H), 7.97 (s, 1H), 7.68-7.66 (m, 2H), 7.51 (t, J=9.5 Hz, 1H), 7.42 (d, J=7.5 Hz, 2H), 7.39-7.30 (m, 2H), 7.27-7.23 (m, 3H), 7.13 (d, J=9.0 Hz, 1H), 5.31 (s, 1H), 4.98 (s, 1H), 3.64 (dd, J=4.5 Hz, 13 Hz, 1H), 3.54-3.43 (m, 4H), 3.38-3.33 (m, 1H), 3.28-3.22 (m, 1H), 3.16-3.11 (m, 1H), 2.37 (s, 3H), 2.35-2.25 (m, 2H), 2.20-2.06 (m, 2H). ¹³C NMR (125 MHz, CD₃OD): δ ppm 156.23, 150.47, 141.93, 137.17, 135.25, 133.64, 133.09, 130.62, 129.63, 129.26, 129.22, 128.76, 128.44, 126.16, 124.32, 121.57, 118.50, 118.36, 117.02, 116.88, 116.42, 116.39, 113.53, 77.71, 76.78, 50.73, 50.63, 44.45, 44.21, 30.94, 30.67, 20.01.

### q. Preparation of 3′,4′-Difluoro-6-((S)-pyrrolidin-3-yloxy)-N-(6-((S)-pyrrolidin-3-yloxy)-2′-(trifluoromethyl)-[1,1′-biphenyl]-3-yl)-[1,1′-biphenyl]-3-carboxamide dihydrochloride (Compound 91d)

Compound 91d was prepared through the same procedure with 91a as a white solid (73%). ¹H NMR (500 MHz, CD₃OD): δ ppm 8.01 (dd, J=2.0 Hz, 9.0 Hz, 1H), 7.96 (s, 1H), 7.87-7.73 (m, 2H), 7.72-7.62 (m, 1H), 7.59-7.47 (m, 2H), 7.42-7.29 (m, 2H), 7.25 (d, J=9.0 Hz, 1H), 7.11 (t, J=9.0 Hz, 1H), 5.30 (s, 1H), 5.15-5.04 (m, 1H), 3.64 (dd, J=4.5 Hz, 13 Hz, 1H), 3.59-3.41 (m, 3H), 3.40-3.31 (m, 1H), 3.27-3.18 (m, 2H), 3.10-2.98 (m, 1H), 2.38-2.22 (m, 2H), 2.20-2.03 (m, 2H). ¹³C NMR (125 MHz, CD₃OD): δ ppm 166.54, 156.21, 132.47, 132.43, 132.26, 132.20, 131.73, 131.67, 131.65, 131.55, 130.63, 130.20, 129.85, 129.62, 129.26, 128.46, 127.98, 127.90, 126.15, 124.51, 122.42, 122.42, 118.51, 117.02, 116.88, 113.78, 113.56, 113.50, 113.16, 76.77, 76.65, 76.25, 50.76, 50.63, 44.45, 44.30, 44.23, 30.95, 30.94, 30.81.

### r. Preparation of N-(3′-cyano-6-((S)-pyrrolidin-3-yloxy)-[1,1′-biphenyl]-3-yl)-3′,4′-difluoro-6-((S)-pyrrolidin-3-yloxy)-[1,1′-biphenyl]-3-carboxamide dihydrochloride (Compound 91e)

Compound 91e was prepared through the same procedure with 91a as a white solid (71%). ¹H NMR (500 MHz, CD₃OD): δ ppm 8.03 (dd, J=2.0 Hz, 8.5 Hz, 1H), 7.97 (s, 1H), 7.90 (s, 1H), 7.86 (d, J=7.5 Hz, 1H), 7.80-7.68 (m, 3H), 7.62 (t, J=9.0 Hz, 1H), 7.58-7.47 (m, 1H), 7.43-7.30 (m, 2H), 7.26 (d, J=8.5 Hz, 1H), 7.17 (d, J=9 Hz, 1H), 5.32 (s, 1H), 5.12 (s, 1H), 3.65 (dd, J=4.5 Hz, 13 Hz, 1H), 3.61-3.51 (m, 2H), 3.51-3.38 (m, 3H), 3.28-3.15 (m, 2H), 2.24-2.14 (m, 4H). ¹³C NMR (125 MHz, CD₃OD): δ ppm 166.48, 156.28, 150.93, 150.26, 148.98, 139.57, 134.91, 134.16, 133.49, 132.85, 130.82, 130.63, 129.98, 129.62, 129.32, 128.28, 126.15, 124.26, 122.74, 118.52, 118.37, 117.04, 116.90, 115.00, 113.52, 112.23, 77.14, 76.78, 50.63, 44.46, 44.33, 30.96, 30.87.

### s. Preparation of N-(4′-cyano-6-((S)-pyrrolidin-3-yloxy)-[1,1′-biphenyl]-3-yl)-3′,4′-difluoro-6-((S)-pyrrolidin-3-yloxy)-[1,1′-biphenyl]-3-carboxamide dihydrochloride (Compound 91f)

Compound 91f was prepared through the same procedure with 91a as a white solid (68%). ¹H NMR (500 MHz, CD₃OD): δ ppm 8.02 (dd, J=2 Hz, 8.5 Hz, 1H), 7.96 (s, 1H), 7.84-7.68 (m, 5H), 7.58-7.47 (m, 1H), 7.43-7.29 (m, 2H), 7.26 (d, J=9.0 Hz, 1H), 7.17 (d, J=9.5 Hz, 1H), 5.31 (s, 1H), 5.13 (s, 1H), 3.65 (dd, J=4.5 Hz, 11.5 Hz, 1H), 3.60-3.37 (m, 4H), 3.29-3.15 (m, 1H), 2.49-2.14 (m, 3H). ¹³C NMR (125 MHz, CD₃OD): δ ppm 166.45, 156.28, 150.23, 143.13, 134.91, 133.50, 131.96, 130.63, 130.38, 130.36, 129.60, 129.30, 128.27, 126.19, 126.16, 126.14, 124.19, 122.92, 118.59, 118.51, 117.37, 116.89, 115.05, 113.53, 110.74, 77.16, 76.78, 50.65, 50.62, 44.46, 44.35, 30.96, 30.81.

### t. Preparation of N-(3′-carbamoyl-6-((S)-pyrrolidin-3-yloxy)-[1,1′-biphenyl]-3-yl)-3′,4′-difluoro-6-((S)-pyrrolidin-3-yloxy)-[1,1′-biphenyl]-3-carboxamide dihydrochloride (Compound 91g)

Compound 91g was prepared through the same procedure with 91a as a white solid (66%). ¹H NMR (500 MHz, CD₃OD): δ ppm 8.08 (s, 1H), 8.02 (dd, J=2 Hz, 8.5 Hz, 1H), 7.96 (s, 1H), 7.84 (d, J=7.5 Hz, 1H), 7.80-7.69 (m, 3H), 7.65-7.46 (m, 2H), 7.43-7.29 (m, 2H), 7.25 (d, J=9.0 Hz, 1H), 7.15 (d, J=9.0 Hz, 1H), 5.31 (s, 1H), 5.10 (s, 1H), 3.65 (dd, J=4.5 Hz, 13 Hz, 1H), 3.60-3.45 (m, 4H), 3.44-3.36 (m, 1H), 3.29-3.16 (m, 1H), 2.44-2.13 (m, 3H). ¹³C NMR (125 MHz, CD₃OD): δ ppm 171.02, 166.46, 156.25, 150.35, 138.50, 134.89, 134.86, 133.63, 133.48, 132.67, 131.51, 130.63, 129.58, 129.29, 128.96, 128.41, 128.31, 126.18, 126.15, 124.28, 122.26, 118.51, 118.36, 117.02, 116.88, 115.36, 113.51, 77.27, 76.78, 50.76, 50.62, 44.46, 44.28, 30.96, 30.80.

### u. Preparation of N-(4′-carbamoyl-6-((S)-pyrrolidin-3-yloxy)-[1,1′-biphenyl]-3-yl)-3′,4′-difluoro-6-((S)-pyrrolidin-3-yloxy)-[1,1′-biphenyl]-3-carboxamide dihydrochloride (Compound 91h)

Compound 91h was prepared through the same procedure with 91a as a white solid (64%). ¹H NMR (500 MHz, CD₃OD): δ ppm 8.02 (dd, J=2.0 Hz, 8.5 Hz, 1H), 7.96 (s, 1H), 7.93 (d, J=8.0 Hz, 2H), 7.81-7.69 (m, 2H), 7.63 (d, J=8.0 Hz, 2H), 7.56-7.45 (m, 1H), 7.43-7.28 (m, 1H), 7.25 (d, J=9.0 Hz, 1H), 7.15 (d, J=9.5 Hz, 1H), 5.31 (s, 1H), 5.07 (s, 1H), 3.65 (dd, J=4.5 Hz, 13 Hz, 1H), 3.55-3.44 (m, 4H), 3.42-3.35 (m, 1H), 3.29-3.23 (m, 1H), 3.21-3.12 (m, 1H), 2.42-2.25 (m, 2H), 2.22-2.14 (m, 2H). ¹³C NMR (125 MHz, CD₃OD): δ ppm 170.81, 166.45, 156.26, 150.37, 141.83, 134.91, 134.88, 134.86, 133.53, 132.52, 131.53, 130.63, 129.57, 129.50, 129.30, 128.28, 127.47, 126.20, 126.15, 124.26, 122.40, 118.51, 118.37, 117.02, 116.88, 115.58, 113.50, 77.41, 76.77, 50.68, 50.61, 44.46, 44.30, 30.98, 30.79.

### v. Preparation of 3′,4′-Difluoro-6-((S)-pyrrolidin-3-yloxy)-N-(6-((S)-pyrrolidin-3-yloxy)-4′-sulfamoyl-[1,1′-biphenyl]-3-yl)-[1,1′-biphenyl]-3-carboxamide dihydrochloride (Compound 91i)

Compound 91i was prepared through the same procedure with 91a as a white solid (76%). ¹H NMR (500 MHz, CD₃OD): δ ppm 8.02 (dd, J=2.0 Hz, 8.5 Hz, 1H), 7.97-7.94 (m, 3H), 7.73-7.70 (m, 4H), 7.58-7.47 (m, 1H), 7.42-7.29 (m, 2H), 7.26 (d, J=9.0 Hz, 1H), 7.16 (d, J=9.0 Hz), 5.31 (s, 1H), 5.11 (s, 1H), 3.65 (dd, J=5.0 Hz, 13 Hz, 1H), 3.59-3.45 (m, 4H), 3.43-3.36 (m, 1H), 3.28-3.16 (m, 2H), 2.42-2.16 (m, 4H). ¹³C NMR (125 MHz, CD₃OD): δ ppm 166.47, 156.28, 150.34, 142.58, 142.14, 133.47, 130/80, 130.66, 130.63, 129.98, 129.93, 129.31, 128.28, 126.20, 126.19, 125.89, 124.34, 122.65, 118.52, 118.37, 117.03, 116.89, 115.21, 113.53, 77.22, 76.78, 50.68, 50.64, 44.47, 44.34, 30.97, 30.82.

### w. Preparation of 3′,4′-Difluoro-N-(4′-fluoro-2′-methyl-6-((S)-pyrrolidin-3-yloxy)-[1,1′-biphenyl]-3-yl)-6-((S)-pyrrolidin-3-yloxy)-[1,1′-biphenyl]-3-carboxamide dihydrochloride (Compound 91j)

Compound 91j was prepared through the same procedure with 91a as a white solid (78%). ¹H NMR (500 MHz, CD₃OD): 3 ppm 8.00 (d, J=6 Hz, 1H), 7.95 (s, 1H), 7.72 (d, J=6 Hz, 1H), 7.49 (s, 2H), 7.39-7.29 (m, 2H), 7.25 (d, J=7.0 Hz, 1H), 7.20-7.09 (m, 2H), 7.01 (d, J=10 Hz, 1H), 6.95 (t, J=7.5 Hz, 1H), 5.30 (s, 1H), 4.97 (s, 1H), 3.64 (dd, J=2.5 Hz, 8 Hz, 1H), 3.57-3.41 (m, 3H), 3.41-3.39 (m, 1H), 3.26-3.20 (m, 1H), 2.39-2.24 (m, 1H), 2.16 (s, 3H), 2.11-2.00 (m, 1H). ¹³C NMR (125 MHz, CD₃OD): δ ppm 166.50, 166.43, 156.24, 150.82, 134.92, 130.65, 129.59, 129.30, 128.37, 126.14, 122.03, 118.54, 118.39, 117.06, 116.93, 116.19, 116.02, 113.60, 112.21, 112.04, 76.84, 50.77, 44.57, 31.06, 19.24

### 16. Preparation of Compounds 91k and 91l

The synthesis scheme (Synthesis Scheme 16) for the preparation of Compounds 91k and 91l is shown below. The synthesis proceeds through the intermediates indicated (Compounds 90e, 90f, 90k, and 90l). The yield for each synthetic step was as indicated.

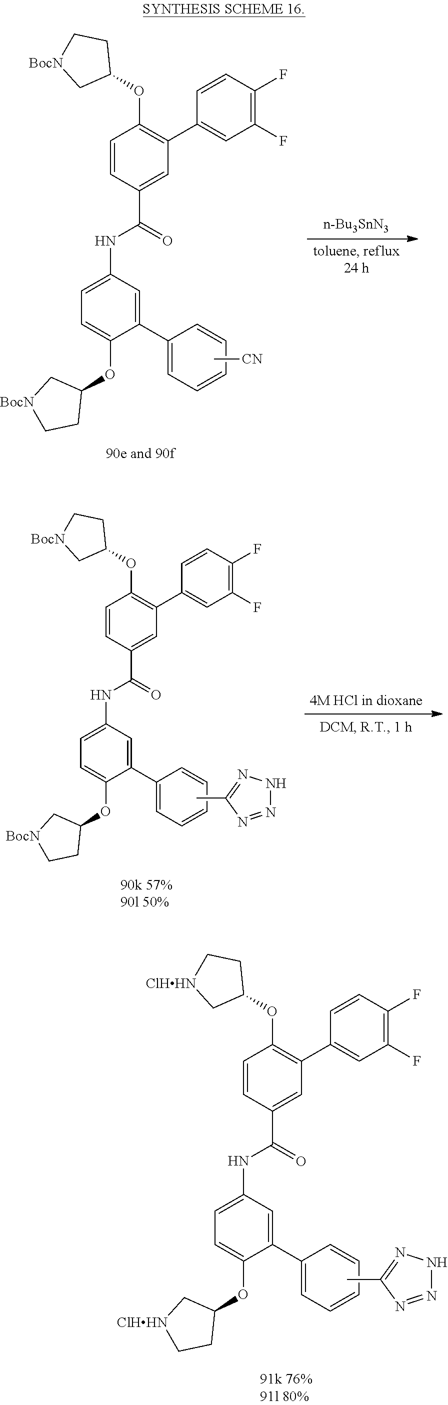

### a. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-3′-(2h-tetrazol-5-yl)-[1,1′-biphenyl]-2-yl)oxy)pyrrolidine-1-carboxylate (Compound 90k)

To a stirred solution of 90e (0.05 g, 0.064 mmol) in anhydrous toluene (5 mL) was added n-Bu₃SnN₃ (0.064 g, 0.19 mmol. The resulting solution was refluxed for 24 h. Upon completion, the reaction was diluted with EtOAc (50 mL), washed with brine (20 mL×3), dried over Na₂SO₄. The solid was filtered and solvent removed under reduced pressure. The residue was purified by column chromatography (DCM:MeOH=15:1-10:1) to yield 90k (0.03 g, 57% yield) as a yellow solid. ¹H NMR (300 MHz, d⁶-Acetone): δ ppm 9.75 (s, 1H), 8.31-8.06 (m, 3H), 7.98 (s, 1H), 7.94-7.90 (m, 1H), 7.86 (s, 1H), 7.71 (d, J=7.8 Hz, 1H), 7.59 (t, J=7.5 Hz, 1H), 7.53-7.337.28 (d, J=7.8 Hz, 1H), 7.15 (d, J=9.0 Hz, 1H), 5.24-5.19 (m, 1H), 5.05-4.98 (m, 1H), 3.66-3.24 (m, 8H), 2.30-2.10 (m, 4H), 1.43-1.35 (m, 18H).

### b. Preparation of (S)-tert-butyl-3-((5-(6-(((S)-1-(tert-butoxycarbonyl)pyrrolidin-3-yl)oxy)-3′,4′-difluoro-[1,1′-biphenyl]-3-ylcarboxamido)-4′-(2h-tetrazol-5-yl)-[1,1′-biphenyl]-2-yl)oxy)pyrrolidine-1-carboxylate (Compound 90l)

The procedure for 90k was used to prepare 901. A white solid was isolated (50%). ¹H NMR (500 MHz, d⁶-Acetone): δ ppm 9.67 (s, 1H), 8.14 (d, J=8.0 Hz, 2H), 8.06-8.04 (m, 2H), 7.88-7.83 (m, 2H), 7.68 (d, J=8.0 Hz, 2H), 7.52-7.44 (m, 1H), 7.35-7.30 (m, 2H), 7.26 (s, 1H), 7.14 (d, J=8.0 Hz, 1H), 5.22-5.18 (m, 1H), 5.04-5.00 (m, 1H), 3.63-3.23 (m, 8H), 2.21-2.08 (m, 4H), 1.45-1.37 (m, 18H).

### c. Preparation of 3′,4′-Difluoro-6-((S)-pyrrolidin-3-yloxy)-N-(6-((S)-pyrrolidin-3-yloxy)-3′-(2h-tetrazol-5-yl)-[1,1′-biphenyl]-3-yl)-[1,1′-biphenyl]-3-carboxamide dihydrochloride (Compound 91k)

Compound 91k was prepared through the same procedure with 91a as a white solid (76%). ¹H NMR (500 MHz, CD₃OD): δ ppm 8.30 (s, 1H), 8.03 (dd, J=2.5 Hz, 9.0 Hz, 1H), 7.99-7.97 (m, 2H), 7.80-7.72 (m, 3H), 7.65 (t, J=8.0 Hz, 1H), 7.57-7.47 (m, 1H), 7.41-7.30 (m, 2H), 7.26 (d, J=9.0 Hz, 1H), 7.19 (d, J=9.0 Hz, 1H), 5.31 (s, 1H), 5.14 (s, 1H), 3.66-3.62 (m, 1H), 3.56-3.52 (m, 3H), 3.52-3.37 (m, 3H), 3.29-3.23 (m, 1H), 2.39-2.17 (m, 4H). ¹³C NMR (125 MHz, CD₃OD): δ ppm

### d. Preparation of 3′,4′-Difluoro-6-((S)-pyrrolidin-3-yloxy)-N-(6-((S)-pyrrolidin-3-yloxy)-4′-(2h-tetrazol-5-yl)-[1,1′-biphenyl]-3-yl)-[1,1′-biphenyl]-3-carboxamide dihydrochloride (Compound 91l)

Compound 911 was prepared through the same procedure with 91a as a white solid. (80%). ¹H NMR (500 MHz, CD₃OD): δ ppm 8.09 (dd, J=2.0 Hz, 8.5 Hz, 2H), 8.03 (dd, J=2.0 Hz, 8.5 Hz, 1H), 8.00-7.97 (m, 1H), 7.76 (dd, J=3.0 Hz, 9.0 Hz, 1H), 7.74-7.66 (m, 3H), 7.57-7.46 (m, 1H), 7.39-7.29 (m, 2H), 7.25 (d, J=8.5 Hz, 1H), 7.17 (d, J=8.5 Hz, 1H), 5.31 (s, 1H), 5.08 (s, 1H), 3.65-3.62 (m, 1H), 3.56-3.43 (m, 4H), 3.41-3.33 (m, 1H), 3.29-3.22 (m, 1H), 3.20-3.11 (m, 1H), 2.40-2.25 (m, 2H), 2.25-2.11 (m, 2H). ¹³C NMR (125 MHz, CD₃OD): δ ppm 156.00, 150.20, 139.59, 133.38, 131.73, 130.38, 129.37, 129.05, 128.15, 126.40, 126.18, 125.82, 123.94, 121.99, 118.27, 118.12, 116.78, 116.64, 115.59, 113.26, 77.37, 76.53, 50.54, 50.41, 44.22, 44.04, 30.70, 30.48.

### 17. Preparation of Compound 91m

The synthesis scheme (Synthesis Scheme 17) for the preparation of Compound 91m is shown below. The synthesis proceeds through the intermediates indicated (Compounds 88, 92, and 90m). The yield for each synthetic step was as indicated.

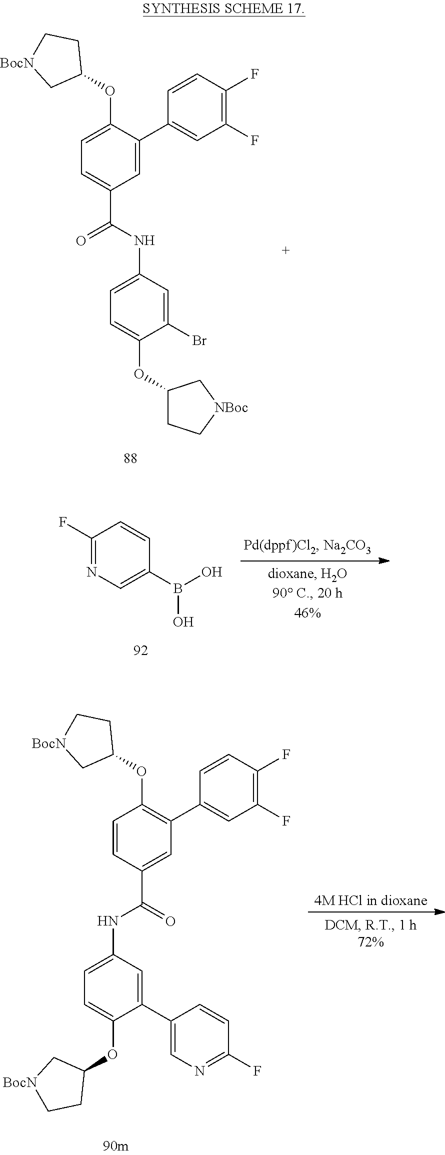 [SMILES: CC(C)(C)OC(=O)N1CC[C@H](Oc2ccc(NC(=O)c3ccc(O[C@H]4CCN(C(=O)OC(C)(C)C)C4)c(-c4ccc(F)c(F)c4)c3)cc2Br)C1 ; OB(O)c1ccc(F)nc1 ; CC(C)(C)OC(=O)N1CC[C@H](Oc2ccc(NC(=O)c3ccc(O[C@H]4CCN(C(=O)OC(C)(C)C)C4)c(-c4ccc(F)c(F)c4)c3)cc2-c2ccc(F)nc2)C1]

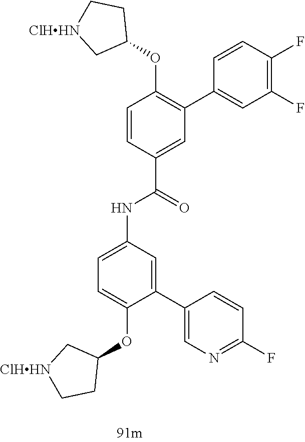

### a. Preparation of (S)-tert-butyl-3-(4-(6-(((S)-1-(tert-butoxycarbonyl)pyrrolidin-3-yl)oxy)-3′,4′-difluoro-[1,1′-biphenyl]-3-ylcarboxamido)-2-(6-fluoropyridin-3-yl)phenoxy)pyrrolidine-1-carboxylate (Compound 90m)

Compound 90m was prepared through the same procedure with 90a as a white solid (46%). ¹H NMR (500 MHz, d⁶-Acetone): δ ppm 9.65 (s, 1H), 8.29 (d, J=10.5, 1H), 8.04-8.02 (m, 3H), 7.87-7.83 (m, 2H), 7.54-7.52 (m, 1H), 7.35-7.30 (m, 2H), 7.25 (d, J=7.5 Hz, 1H), 7.15 (d, J=8.5 Hz, 1 h), 7.08 (dd, J=3.0, 8.5 Hz, 1H), 5.22-5.17 (m, 1H), 5.06-5.02 (m, 1H), 3.64-3.20 (m, 8H), 2.21-2.09 (m, 4H), 1.42-1.40 (m, 18H).

### b. Preparation of 3′,4′-Difluoro-N-(3-(6-fluoropyridin-3-yl)-4-((S)-pyrrolidin-3-yloxy)phenyl)-6-((S)-pyrrolidin-3-yloxy)-[1,1′-biphenyl]-3-carboxamide dihydrochloride (Compound 91m)

Compound 91m was prepared through the same procedure with 91a as a white solid (72%). ¹H NMR (500 MHz, CD₃OD): δ ppm 8.34 (s, 1H), 8.14 (td, J=2.0 Hz, 8.5 Hz, 1H), 8.02 (dd, J=2.0 Hz, 9.0 Hz, 1H), 7.97 (s, 1H), 7.79-7.71 (m, 2H), 7.57-7.48 (m, 1H), 7.39-7.30 (m, 2H), 7.26 (d, J=9.0 Hz, 1H), 7.21-7.12 (m, 2H), 5.32 (s, 1H), 5.17 (s, 1H), 3.65 (dd, J=5.0 Hz, 13 Hz, 1H), 3.58 (dd, J=4.5 Hz, 13 Hz, 1H), 3.53-3.41 (m, 4H), 3.29-3.20 (m, 2H), 2.42-2.19 (m, 4H). ¹³C NMR (125 MHz, CD₃OD): δ ppm 166.47, 156.29, 150.36, 147.41, 147.39, 147.28, 143.09, 143.03, 133.40, 130.63, 129.61, 129.30, 128.27, 127.12, 126.19, 126.16, 126.14, 124.20, 122.82, 118.51, 118.37, 117.04, 116.90, 114.51, 113.54, 109.11, 108.82, 76.89, 76.79, 50.66, 50.63, 44.47, 44.37, 30.97, 30.87.

### 18. Preparation of Compound 95

The synthesis scheme (Synthesis Scheme 18) for the preparation of Compound 95 is shown below. The synthesis proceeds through the intermediates indicated (Compounds 93 and 94). The yield for each synthetic step was as indicated.

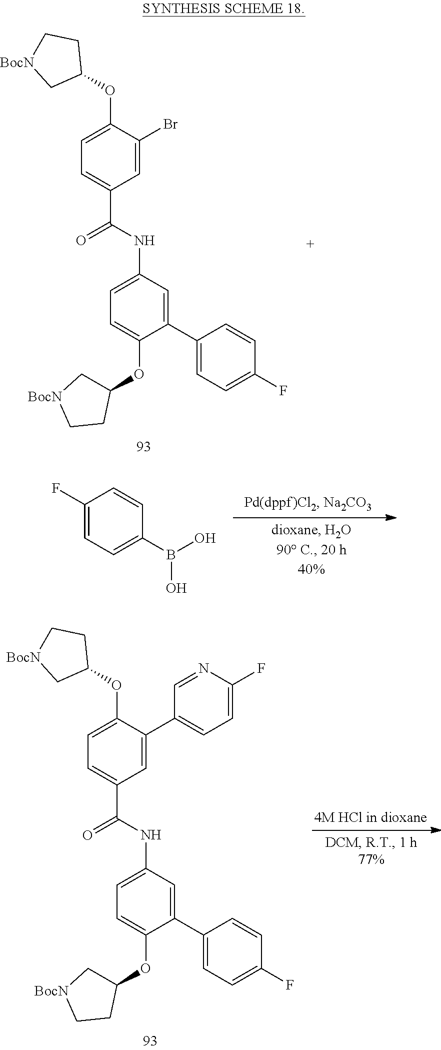 [SMILES: CC(C)(C)OC(=O)N1CC[C@H](Oc2ccc(C(=O)Nc3ccc(O[C@H]4CCN(C(=O)OC(C)(C)C)C4)c(-c4ccc(F)cc4)c3)cc2Br)C1 ; OB(O)c1ccc(F)cc1 ; CC(C)(C)OC(=O)N1CC[C@H](Oc2ccc(NC(=O)c3ccc(O[C@H]4CCN(C(=O)OC(C)(C)C)C4)c(-c4ccc(F)nc4)c3)cc2-c2ccc(F)cc2)C1]

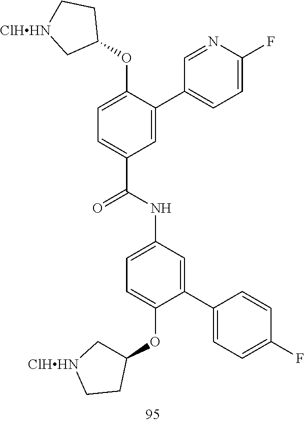

### a. Preparation of (S)-tert-butyl-3-((5-(4-(((S)-1-(tert-butoxycarbonyl)pyrrolidin-3-yl)oxy)-3-(6-fluoropyridin-3-yl)benzamido)-4′-fluoro-[1,1′-biphenyl]-2-yl)oxy)pyrrolidine-1-carboxylate (Compound 94)

Compound 94 was prepared through the same procedure with 90a to yield a white solid (40%). ¹H NMR (500 MHz, d⁶-Acetone): δ ppm 9.62 (s, 1H), 8.35 (d, J=14.5 Hz, 1H), 8.08-8.06 (m, 2H), 7.85-7.81 (m, 2H), 7.52-7.50 (m, 2H), 7.27 (d, J=8.0 Hz, 1H), 7.15-7.09 (m, 4H), 5.22-5.17 (m, 1H), 5.02-4.95 (m, 1H), 3.65-3.20 (m, 8H), 2.21-2.10 (m, 4H), 1.42 (s, 9H), 1.40 (s, 9H).

### b. Preparation of N-(4′-fluoro-6-((S)-pyrrolidin-3-yloxy)-[1,1′-biphenyl]-3-yl)-3-(6-fluoropyridin-3-yl)-4-((S)-pyrrolidin-3-yloxy)benzamide dihydrochloride (Compound 95)

Compound 95 was prepared through the same procedure with 91a as a white solid (77%). ¹H NMR (500 MHz, CD₃OD): δ ppm 8.38 (s, 1H), 8.19 (t, J=8.0 Hz, 1H), 8.06 (dd, J=2.0 Hz, 8.5 Hz, 1H), 8.00 (s, 1H), 7.69 (d, J=8.0 Hz, 2H), 7.55-7.53 (m, 2H), 7.28 (d, J=8.5 Hz, 1H), 7.16-7.12 (m, 4H), 5.35 (s, 1H), 5.04 (s, 1H), 3.66 (dd, J=4.0 Hz, 13 Hz, 1H), 3.56-3.44 (m, 4H), 3.41-3.36 (m, 1H), 3.27-3.24 (m, 1H), 3.21-3.11 (m, 1H), 2.46-2.25 (m, 2H), 2.22-2.09 (m, 2H). ¹³C NMR (125 MHz, CD₃OD): δ ppm 166.33, 156.40, 150.36, 147.54, 147.43, 143.27, 143.25, 143.25, 143.19, 134.29, 134.27, 133.49, 131.77, 131.74, 131.67, 131.29, 131.22, 130.58, 129.83, 128.41, 126.69, 124.32, 121.92, 115.78, 114.97, 114.93, 114.76, 113.23, 109.18, 108.88, 77.48, 77.45, 76.74, 50.72, 50.65, 44.48, 44.31, 30.98, 30.80.

### 19. Compounds 96-102

The structures of synthesized compounds 96-103 are as shown below.

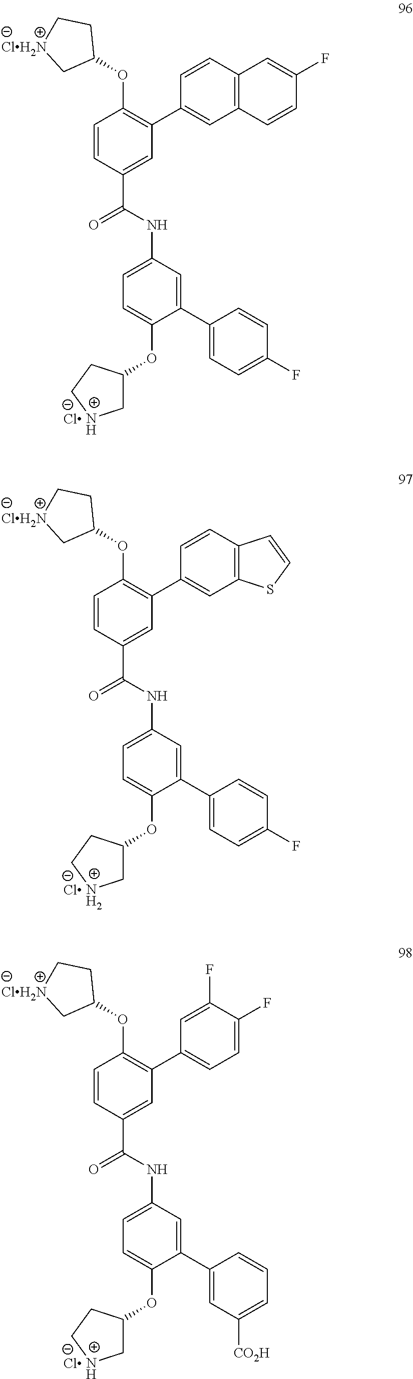

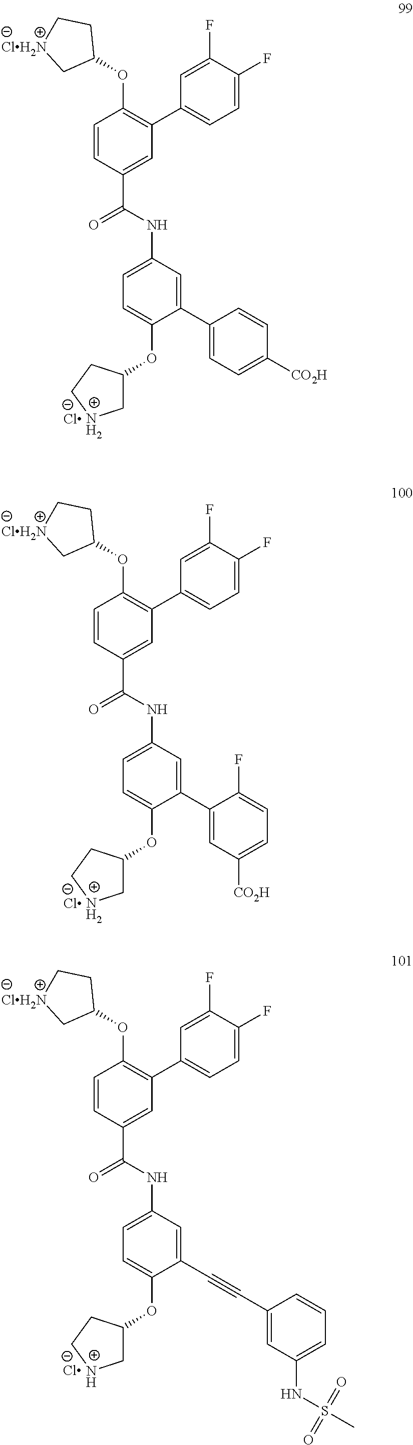

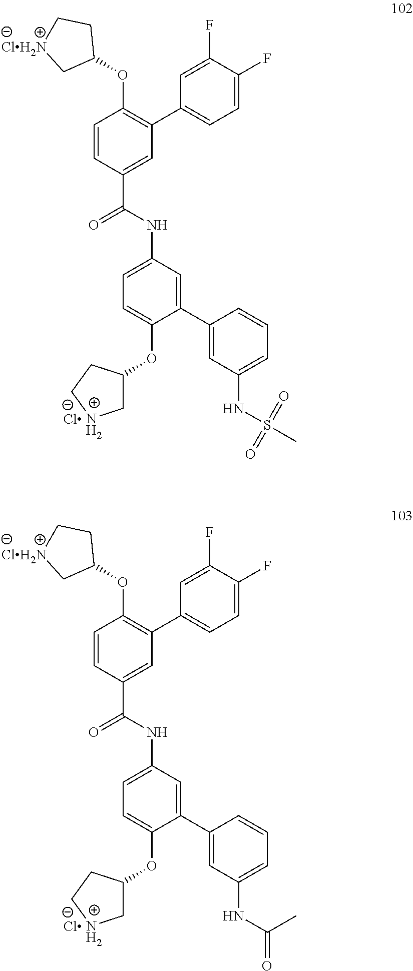

### 20. HPLC Conditions

The purity of final compounds 1-29 was determined by HPLC analysis. The instrument was a Waters Acquity H-class with a Waters TQD detector. The column was an Acquity UPLC BEH C18 1.7 um 2.1×50 mm pn 188002350. Two sets of mobile phase were used for each compound. Condition A: a gradient starting with 0.1% formic acid in water and ending with 0.1% formic acid in acetonitrile. Condition B: a gradient starting with 0.1% formic acid in water and ending with methanol. The purity for all compounds was ≥95%.

### 21. Protein Expression and Purification

β-Catenin or its mutants (residues 138-686) was cloned into a pET-28b vector carrying a C-terminal 6× histidine (Novagen), and transformed into E. coli BL21 DE3 (Novagen). Cells were cultured in LB medium with 30 μg/mL kanamycin until the OD₆₀₀ was approximately 0.8, and then protein expression was induced with 400 μM of IPTG at 20° C. overnight. Cells were lysed by sonication. The proteins were purified by Ni-NTA affinity chromatography (30210, Qiagen) and dialyzed against a buffer containing 20 mM of Tris (pH 8.5), 100 mM NaCl, 10% glycerol, and 3 mM DTT. The purity of β-catenin was greater than 95% as determined by SDS-PAGE gel analysis. Native non-denaturing gel electrophoresis was performed to confirm the homogeneity of the purified proteins. Thermal-shift assay was performed on an iCycler iQ Real Time Detection System (Bio-Rad, Hercules, Calif.) to monitor protein stability and detect protein aggregation. Protein unfolding was evaluated through measuring the fluorescence changes of fluorescent dye Sypro Orange when interacting with wild-type or mutant β-catenin proteins. A temperature increment of 1°/min was applied. All proteins were stable and no aggregation was observed under storage or assay conditions. Proteins were aliquoted and stored at −80 OC.

### 22. Site-Directed Mutagenesis Experiments

Mutants β-catenin D145A, E155A, L156S, L159S, D145A/E155A, L156S/L159S, and L156S/L178S were generated using the overlapping PCR method. Template for the mutagenesis reactions was wide type full-length β-catenin in pET-28b. KOD hot start DNA polymerase (Novagen) was used through this experiment. Mutants were confirmed by direct sequencing (Core facility, University of Utah). Following the confirmation of the sequence, mutant β-catenin were cloned into a pET-28b vector and transformed into E. coli BL21 DE3. The primers for site-directed mutagenesis are shown in Table 1.

TABLE 1

| Mutant | Primer 1 | Primer 2 |
|---|---|---|
| D145A | ggcagaccatcatcgcgttctt- | aattataagaacgcgatgatgg- |
| | ataattattg | tctgccaag |
| E155A | ctggtcagatgagcaagagcac- | catctgtgctcttgctcatctg- |
| | agatg | accag |
| L156S | gtgcaatccctgaatcgacaaa- | catttagcagttttgtcgattc- |
| | actgctaaatg | agggattgcac |
| L159S | ctgaactgacaaaatcgctaaa- | gtcctcgtcatttagcgatttt- |
| | tgacgaggac | gtcagttcag |
| L156S/L159S | ctgaatcgacaaaatcgctaaa- | gtcctcgtcatttagcgatttt- |
| | tgacgaggac | gtcgattcag |
| L178S | gttatggtccatcagtcttcta- | gaagcttcctttttagaagact- |
| | aaaaggaagcttc | gatggaccataac |

The full-length mutant β-catenin DNA was used as the template to make mutant β-catenin DNA fragments (residue 138-686). The primers to produce β-catenin fragment (residue 138-686) are forward, 5′-GGGGGGTCATGATCAACTTGATTAACTATCAAG-3′; and reverse, 5′-AAAACCCTCGAGCTCTGTTCTGAAGAGAG-3′.

### 23. BCL9 Peptide Synthesis and Purification

Human BCL9 (residues 350-375), N-terminally biotinylated human BCL9 (residues 350-375), N-terminally fluorescein-labeled human BCL9 (residues 350-375), and N-terminally biotinylated human E-cadherin (residues 824-877) were synthesized by InnoPep Inc. (San Diego, Calif., www.innopep.com). All synthesized peptides were purified by HPLC with purity >95%. The structures were validated by LC/MS. The sequences are shown in Table 2 (Ahx, 6-aminohexanoic acid).

TABLE 2

| Peptides | Sequences |
|---|---|
| BCL9 26-mer | H-³⁵⁰GLSQEQLEHRERSLQTLRDIQRMLFP³⁷⁵-NH₂ |
| Biotinylated BCL9 | Biotin-Ahx-³⁵⁰GLSQEQLEHRERSLQTLRDIQRMLFP³⁷⁵- |
| 26-mer | NH₂ |
| FITC-labeled BCL9 | FITC-Ahx-³⁵⁰GLSQEQLEHRERSLQTLRDIQRMLFP³⁷⁵- |
| 26-mer | NH₂ |
| Biotinylated E- | Biotin- |
| cadherin 54-mer | ⁸²⁴APPYDSLLVFDYEGSGSEAASLSSLNSSESDKDQDYD |
| | YLNEWGNRFKKLADMYG⁸⁷⁷-NH₂ |

### 24. AlphaScreen Assays

All experiments were performed in white opaque 384-well plates from PerkinElmer (Waltham, Mass.) with an assay buffer of 25 mM HEPES (pH=7.4), 100 mM NaCl, 0.1% BSA, and 0.01% Triton X-100. All sample signals were read on a Synergy 2 plate reader (Biotek, Winooski, Vt.) with a sensitivity setting of 200. The excitation wavelength was set at 680 nm and emission at 570 nm. All of the final reaction volumes were set to 25 μL. In the cross-titration experiments of wild-type β-catenin/wild-type BCL9 and mutant β-catenin/wild-type BCL9 interactions, N-terminally biotinlyated BCL9 (from 0 to 60 nM) and C-terminally His₆-tagged β-catenin (2.5, 5, 10, 20, 40, and 80 nM) were titrated in 20 μL assay buffer. After 2 h incubation at 4° C. on an orbital shaker, 2.5 μL of nickel chelate acceptor beads (10 μg/mL) and 2.5 μL of streptavidin-coated donor beads (10 μg/mL) were added. The mixture was then covered black and incubated at 4° C. for 1 h before detection. All addition and incubation was made under subdued lighting conditions due to the photosensitivity of the beads. The data were analyzed by nonlinear least-square analyses using GraphPad Prism 5.0. Each experiment was repeated three times, and the results were expressed as mean±standard deviation. The competitive binding experiments were performed to determine the apparent K_{d} values. The rule of the competitive binding experiments for associating the IC₅₀ value with the K_{d} value are: (1) the expected K_{d} value should be 10 times higher than the concentration of either tested protein; (2) the concentrations of both tested proteins should be lower than the binding capacities of their respective beads; and (3) the concentration of the target protein (His₆-tagged β-catenin) should be 10 times lower than that of the ligand protein (biotinylated BCL9). In the competitive binding experiments to determine the K_{d} value for β-catenin/BCL9 interactions, 5 nM of N-terminally biotinylated BCL9, 0.5 nM of C-terminally His₆-tagged β-catenin, and different concentrations of unlabeled BCL9 peptide (0-50 μM) were incubated at 4° C. in 20 μL assay buffer for 2 h. The donor and acceptor beads were added to a final concentration of 10 μg/mL in 25 μL assay buffer. The mixture was covered black and incubated for 1 h at 4° C. before detection. The IC₅₀ values, which were also the apparent K_{d} values from the AlphaScreen assay, were determined by nonlinear least-square analyses using GraphPad Prism 5.0. Each experiment was repeated three times, and the results were expressed as mean±standard deviation.

### 25. Fluorescence Polarization (FP) Assays

The FP experiments were performed in 96-well Microfluor 2 black plates (Waltham, Mass.), and the sample signals were read by a Synergy 2 plate reader (Biotek, Winooski, Vt.). The polarization was measured at room temperature with an excitation wavelength at 485 nm and an emission wavelength at 535 nm. All of the FP experiments were performed in an assay buffer of 137 mM NaCl, 2.7 mM KCl, 10 mM Na₂HPO₄, 2 mM KH₂PO₄, 100 μg/mL of bovine γ-globulin, and 0.01% Triton X-100. The final reaction volume was set to 100 μL. In the FP saturation binding experiments, the concentration of human BCL9 fluorescent tracer was fixed at 5 nM. The concentrations of β-catenin were ranged from 0 to 10 μM in the assay buffer giving a final volume of 100 μL. After the addition, each assay plate was covered black and gently mixed on an orbital shaker for 3 h before the polarization signals were recorded. The data were analyzed by nonlinear least-square analyses using GraphPad Prism 5.0 to derive the apparent K_{d} value. Each experiment was repeated three times, and the results were expressed as mean±standard deviation.

### 26. Protein Structure for Computer Modeling

The crystallographic coordinates for human β-catenin (PDB id, 2GL7, 2.60 Å resolution, R_{cryst}=0.223) were obtained from the Research Collaboratory for Structural Bioinformatics (RCSB) protein data bank. The preparation of the crystal structure and molecular modeling were achieved with the commercially available Schrodinger (http://www.schrodinger.com/), Accelrys Discovery Studio 3.0 (http://accelrys.com/), and SYBYL X 2.0 (http://www.tripos.com/) software packages. The missing side chains of β-catenin were added in SYBYL X2.0. The protonation states of the residues were set to pH 7.0 when adding hydrogen. The AMBER 7 force field 99 and the AMBER FF99 charges within SYBYL X2.0 were used to optimize the orientation of hydrogen atoms and the missing side chains of the protein. After the protein complex was optimized. Chains B (Tcf4), C (BCL9), D (the second monomer of β-catenin), E (the second monomer of Tcf4), F (the second monomer of BCL9), and solvent molecules were removed, leaving only one monomer of β-catenin for further calculation. The residues in the BCL9 L366/1369/L373 binding site of β-catenin include D144-A146, L148, A149, A152, 1153, E155-L160, D162-A171, M174, V175, Q177, L178, K180, K181, A183, S184, A187, 1188, M194, and I198.

### 27. SiteMap Calculations

This calculation was performed with SiteMap 2.6 in Schrodinger. The evaluation on a single binding site region was selected, and all residues of the BCL9 L366/I369/L373 binding site were included. At least 15 site points per reported site were required to initiate the SiteMap calculation. The more restrictive definition for hydrophobicity was used. The grid spacing was set to 0.35 Å. The site maps were cropped at 4 Å from nearest site point, and the OPLS-2005 force field was used to map the hydrophobic, H-bond donor and H-bond acceptor regions. The druggability assessment score (Dscore) was determined by the equation:

Dscore=0.094n^{1/2}+0.60e−0.324p

n is the number of site points, e is the enclosure score, and p is the hydrophilic score.

A large validation study indicated that the average of SiteMap druggability Dscore for undruggable, difficult, and druggable pockets were 0.827, 0.995, and 1.091, respectively (Halgren (2009) J. Chem. Inf. Model. 49: 377-389).

### 28. Fragment Design and Linking

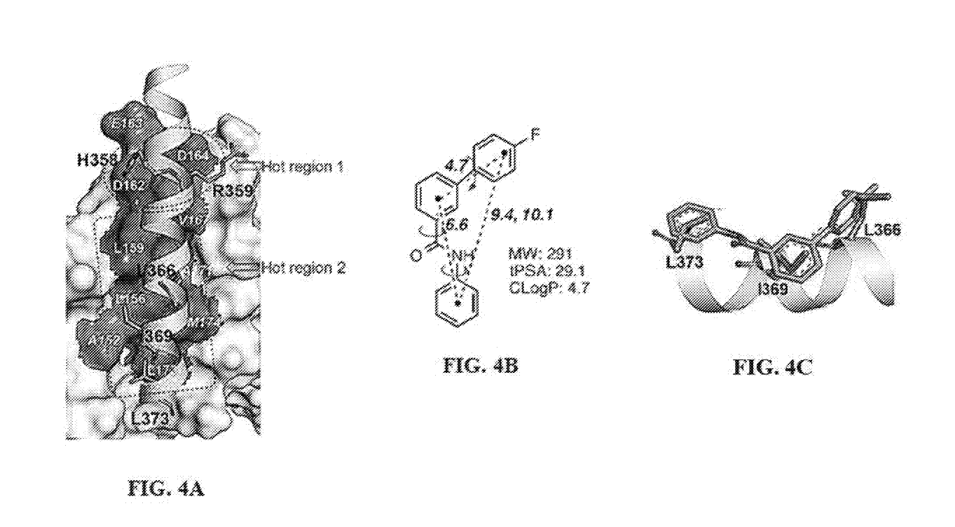

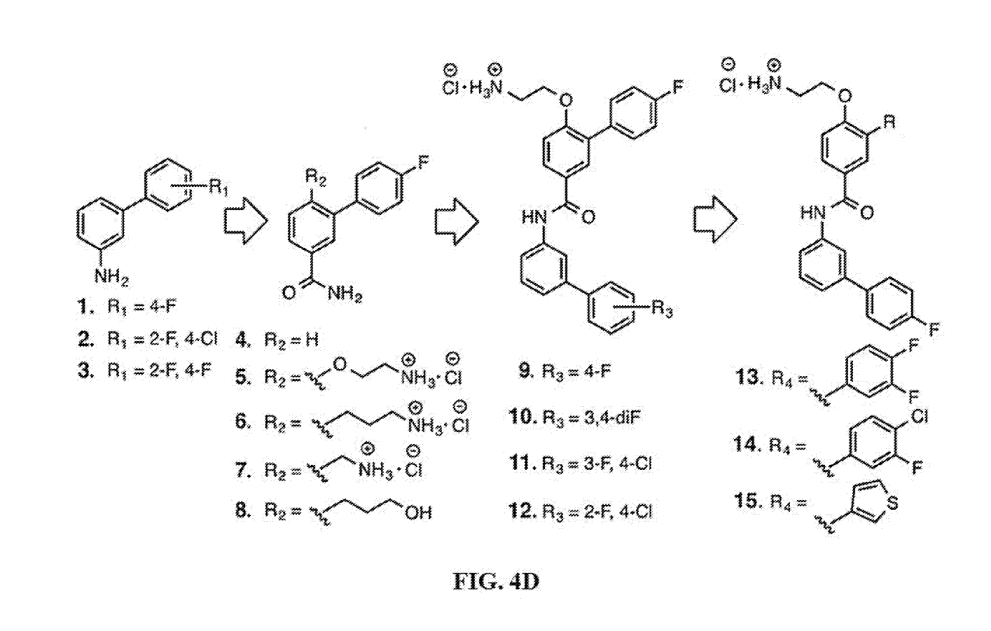

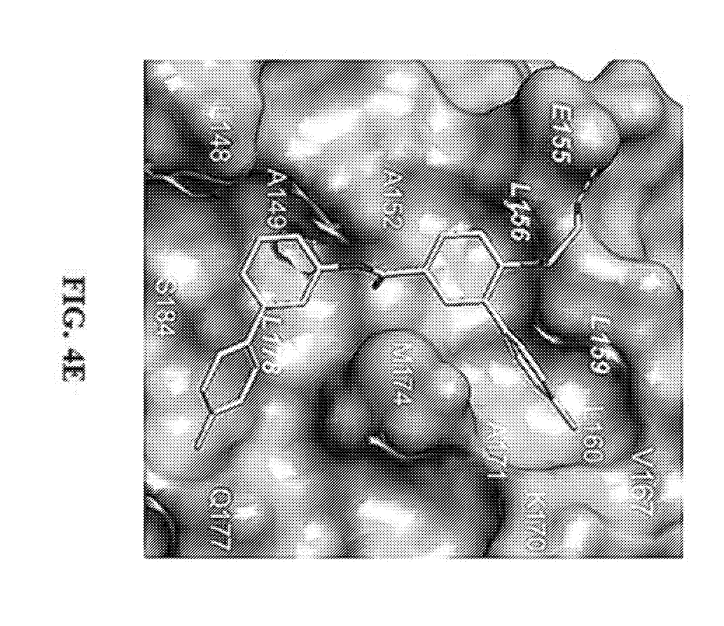

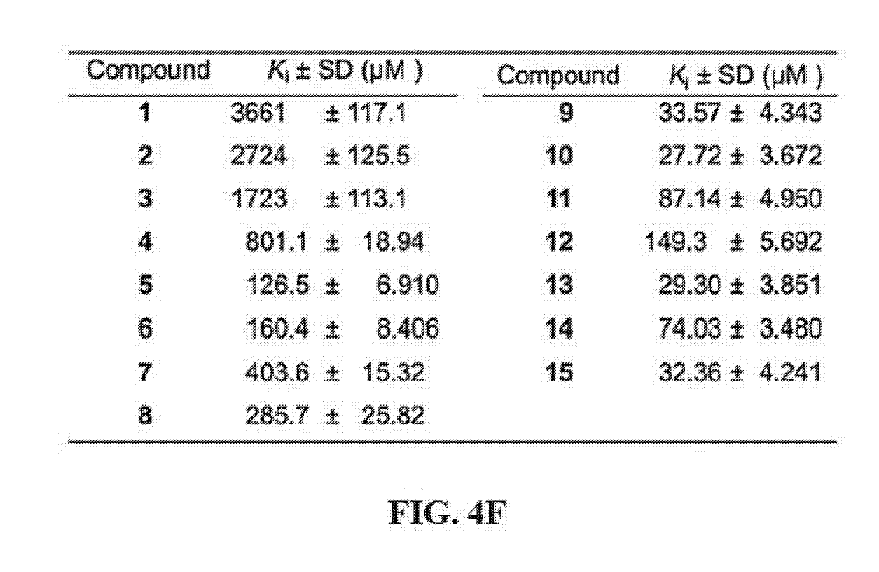

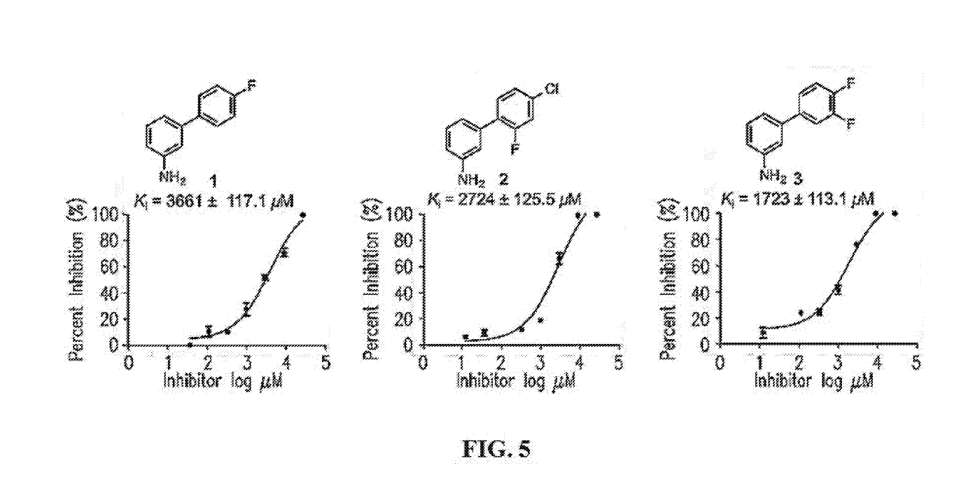

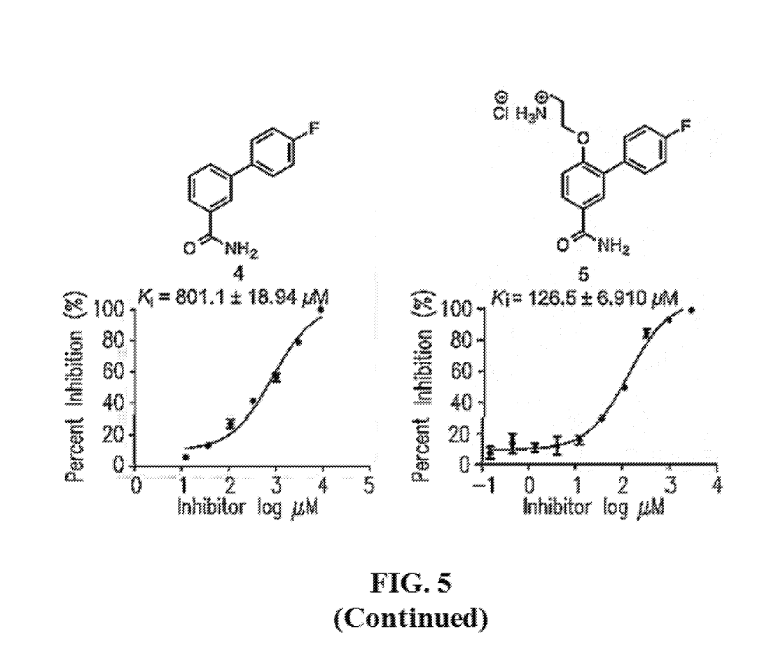

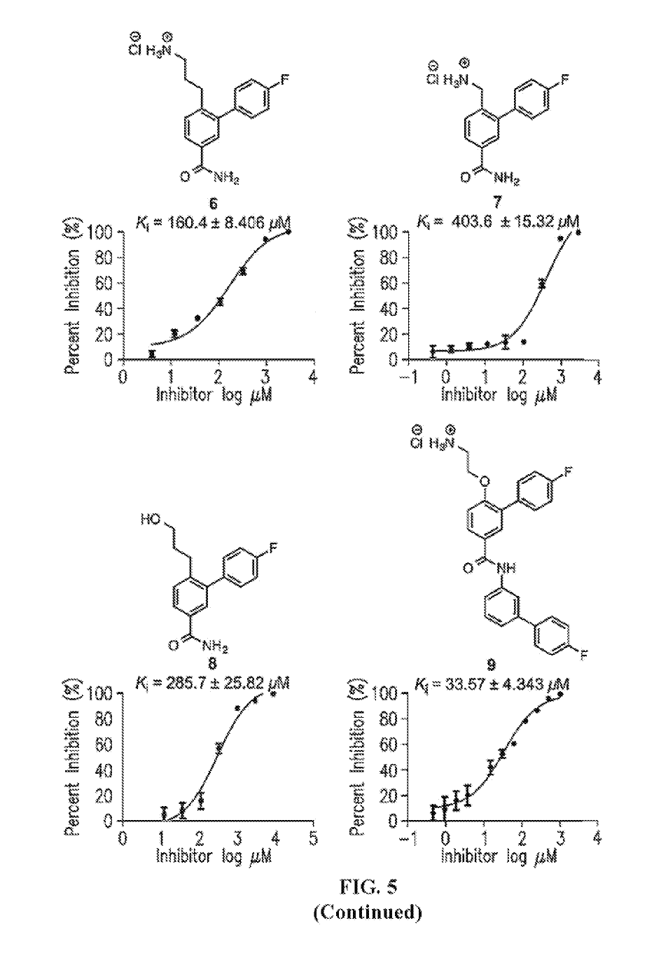

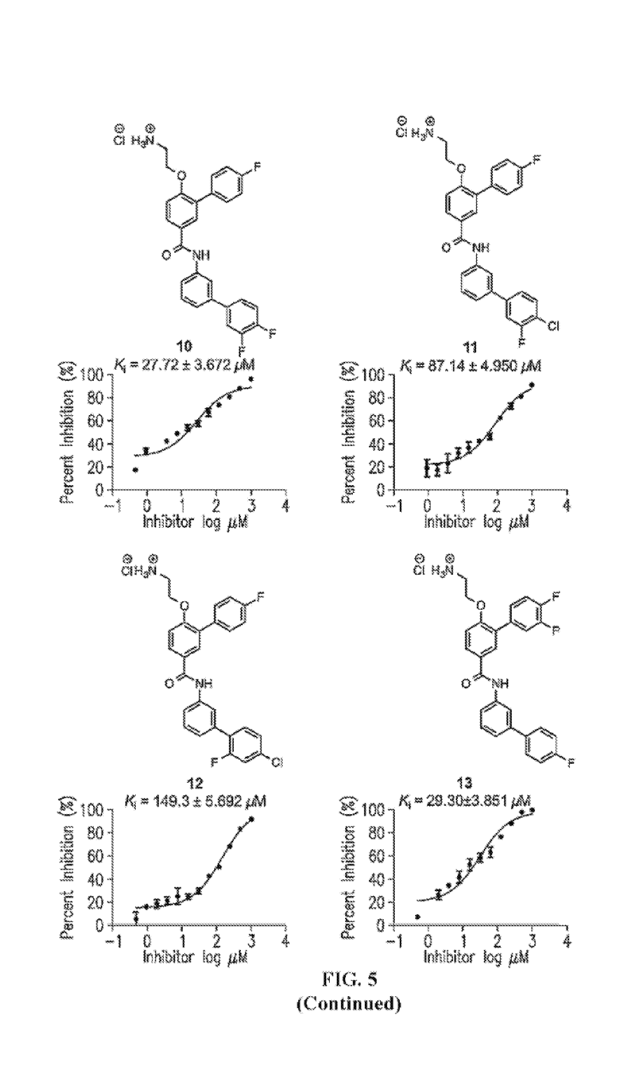

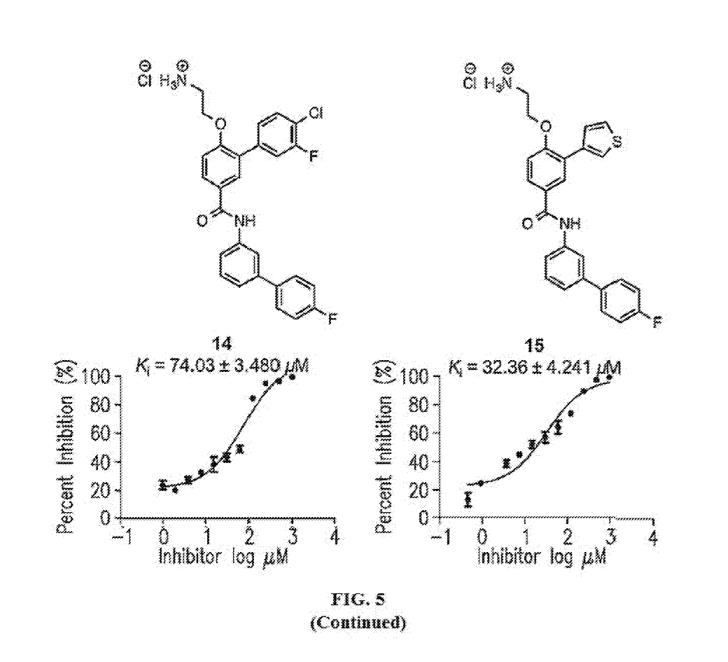

The fragments in Supplementary FIGS. 4 and 5 in ACS Chemical Biology 8 (3), 524-529 (2013) were built in the commercially available SYBYL X2.0 software package. The partial atomic charges were calculated using the Gasteiger-Marsili method. In the AutoDock 4.2 calculations, only the polar hydrogen atoms were kept on the protein structure, and the Kollman united atom charges were assigned. The grid maps were calculated using AutoGrid with the grid spacing of 0.375 Å. For the fragment docking any atoms within 6 Å from the proposal critical binding elements (hydrophobic: the side chains of L148, A149, A152, L156, L159, L160, V167, K170, A171, M174, and L178 of β-catenin; H-bond and charge-charge interactions: the side chain NH₃ of K181, the side chain OH of S184, the backbone NH of A18, the side chain carboxylic oxygens of D145, E155, D162, and S184, and the backbone carbonyl of L148) were used to define the grid box, which resulted in two pockets: one pocket included the side chains of A152, L156, L159, L160, V167, K170, A171, and M174 of β-catenin, and the side chain carboxylic oxygens of E155 and D162 of β-catenin; and the second pocket included the side chains of L148, A149, A152, M174, and L178 of β-catenin, the side chain NH₃ of K181, the side chain OH of S184, the backbone NH of A183 of β-catenin, the side chain carboxylic oxygens of β-catenin D145, and S184, and the backbone carbonyl oxygen of L148. Docking was performed using the Lamarckian genetic algorithm, and the pseudo-Solis and Wets method was applied for the local search. Each docking experiment was performed 50 times, yielding 50 docked conformations. The other settings were the standard default parameters. The results of the docking experiments were evaluated by the auxiliary clustering analysis and the visual inspection to match the proposed binding elements. The binding poses of the fragments that matched the proposed binding elements were stored in a SYBYL molecular database. The distance between each fragment of two pockets was measured, and the linkers in Supplementary FIG. 5 in ACS Chem. Biol. 8 (3), 524-529 (2013) were merged to generate the ligand structure.

### 29. Ligand Docking Using AutoDock 4.2

The three-dimensional (3D) structures of the ligands were built, and the partial atomic charges were calculated using the Gasteiger-Marsili method. The rotatable bonds in the ligands were defined using AutoTors, which also united the nonpolar hydrogens and partial atomic charges to the bonded carbon atoms. The grid maps were calculated using AutoGrid. The AutoDock area was defined to include all the residues of the BCL9 L366/I369/L373 binding site, and the grid spacing was set to 0.375 Å. Docking was performed using the Lamarckian genetic algorithm, and the pseudo-Solis and Wets method was applied for the local search. Each docking experiment was performed 100 times, yielding 100 docked conformations. The other settings were the default parameters. All of the ligands followed the same docking protocol. The results of the docking experiments were evaluated by the auxiliary clustering analysis and the visual inspection to match the proposed critical binding elements.

### 30. Ligand Docking Using Glide 5.8

The 3D coordinates of all ligands were generated by Schrodinger LigPrep with Epik to expand the protonation and tautomeric states at pH=7.0. The energy minimization was then applied to all ligands with the OPLS_2005 force field and the GB/SA water solvation condition. The partial charges of the ligands were calculated by the OPLS_2005 force field. The grid box was defined to include all the residues of the BCL9 L366/I369/L373 binding site. The default parameters were used in receptor grid generation. The standard precision mode (SP) was used in ligand docking. The ligand scaling factor was set to 0.5 for the atoms with the partial charges lower than 0.15. The number of poses per ligand for the initial phase of docking was increased to 10,000. The 1,000 best poses per ligand were kept for energy minimization with a maximum number of the minimization steps of 5,000. A maximum of 100,000 ligand poses per docking run and 50 poses per ligand were collected. Up to 100 poses per ligand were kept for the post-docking minimization. The default settings were used for the remaining parameters.

### 31. AlphaScreen Competitive Inhibition Assays

The purity of the wild-type and mutant β-catenin proteins was all greater than 95% as determined by the SDS-PAGE gel analysis. Both the wild-type and mutant β-catenin proteins are stable under the testing conditions. Native non-denaturing gel electrophoresis was performed to confirm the homogeneity of the purified proteins. Experiments were performed in white opaque 384-well plates from PerkinElmer, and the samples were read on a Synergy 2 plate reader (Biotek) with a sensitivity setting of 200 using AlphaScreen protocol with excitation at 680 nm and emission at 570 nm. All dilutions were made in 1×assay buffer containing 25 mM HEPES (pH=7.0), 100 mM NaCl, 0.01% Triton X-100, and 0.1% BSA. In the AlphaScreen competitive inhibition assay, 40 nM of C-terminally His₆-tagged wild-type or mutant β-catenin (residues 138-686) proteins was incubated with 5 nM of N-terminal biotinylated human BCL9 26-mer (residues 350-375) for 30 min at 4° C., and then different concentrations of the tested inhibitors in the assay buffer were added to make a final volume of 20 μL. The mixture was incubated at 4° C. for 2 h. The donor and acceptor beads were added to a final concentration of 10 μg/mL in 25 μL of assay buffer. The mixture was incubated at 4° C. for 1 h. The IC₅₀ value was determined by nonlinear least-square analysis of GraphPad Prism 5.0. For each inhibitor competition assay, the negative control (equivalent to 0% inhibition) refers to 5.0 nM of N-terminally biotinylated human BCL9 26-mer, 40 nM of C-terminally His₆-tagged human β-catenin, and 10 μg/mL of the donor and acceptor beads in a final volume of 25 μL assay buffer, but no tested compounds present. The positive control (equivalent to 100% inhibition) refers to 5.0 nM of biotinylated human BCL9 26-mer and 10 μg/mL of the donor and acceptor beads in a final volume of 25 μL assay buffer. The Kᵢ values were derived from the IC₅₀ values by a reported method (Nikolovska-Coleska et al. (2004) Anal. Biochem. 32: 261-273). Experiments were performed in triplicate and carried out in the presence of 1% DMSO.

### 32. Isothermal Titration Calorimetry Experiments

ITC measurements were performed at 28° C. using a VP-ITC (Microcal, GE Healthcare Life Sciences). Compound 21 and wild-type and mutant β-catenin proteins were concentrated to 100 μM and 8-12 μM, respectively, in buffer A (20 mM Tris, pH 8.8, 100 mM NaCl, 1 mM TCEP, and 8% glycerol). The homogeneity of the purified proteins were examined by native gel electrophoresis. Each titration experiment was initiated by a 2 μL injection, and followed by 30-35 times injections with an 8 μL volume each. Blank titrations, which were carried out by injecting the compound into the buffer, were subtracted from each data set. The association constant K_{A}, enthalpy change (ΔH), and stoichiometry N were obtained from fitting the data using the Origin software package. The dissociation constant K_{d}, the free energy change ΔG, and the entropy change ΔS were obtained from the basic thermodynamic equations, K_{d}=K_{A}⁻¹, ΔG=−RTlnK_{A}, and ΔG=ΔH−TΔS.

### 33. Cell Transfection and Luciferase Assay

FuGENE6 (E269 Å, Promega) 96 well plate format was used for the transfection of HEK293, SW480, and MDA-MB-231 cells according to the manufacturer's instruction. HEK293 cells were co-transfected with 45 ng of TOPFlash or FOPFlash reporter gene, 135 ng pcDNA3.1-β-catenin, and 20 ng of pCMV-RL normalization reporter gene. SW480 and MDA-MB-231 cells were co-transfected with 60 ng of TOPFlash or FOPFlash reporter gene and 40 ng of pCMV-RL normalization reporter. Cells were cultured in DMEM and 10% FBS at 37° C. for 24 h, and different concentrations of inhibitors or DMSO were then added. After 24 h, the luciferase reporter activity was measured using the Dual-Glo system (E2940, Promega). Normalized luciferase activity in response to the treatment with 20, 21, and camosic acid was compared with that obtained from cells treated with DMSO. Experiments were performed in triplicate.

### 34. Quantitative Real Time PCR Analysis

SW480 and MDA-MB-231 cells at 1×10⁶/mL were treated with different concentrations of 21 for 24 h. Total RNAs were extracted with TRIzol (Ser. No. 15/596,026, Life Technologies), and the cDNA was synthesized with the superscript III first-strand kit (18080-051, Invitrogen). Quantitative PCR (qPCR) was performed using the iQ™ SYBR green supermix kit (170-8880, BIO-RAD) on an iQ⁵ multicolor real-time PCR reaction system (BIO-RAD). The threshold cycle (C_{T}) values were normalized to that of internal reference GAPDH. The primer pairs for human GAPDH were forward: 5′-GAAGGTGAAGGTCGGAGTC-3′, and reverse: 5′-GAAGATGGTGATGGGATTTC-3′, for human AXIN2 forward: 5′-AGTGTGAGGTCCACGGAAAC-3′ and reverse: 5′-CTTCACACTGCGATGCATTT-3′, for human LGR5 forward: 5′-TGCTGGCTGGTGTGGATGCG-3′ and reverse: 5′-GCCAGCAGGGCACAGAGCAA-3′, for human LEF1 forward: 5′-GACGAGATGATCCCCTTCAA-3′ and reverse: 5′-AGGGCTCCT GAGAGGTTTGT-3′, and for human cyclin D1 forward: 5′-ACAAACAGATCATCCGCAAACAC-3′, and revers: 5′-TGTTGGGGCTCCTCAGGTTC-3′. Experiments were performed in triplicate.

### 35. Western Blotting

SW480 cells at 1×10⁶ cells/mL were treated with different concentrations of 21 for 24 h. Cells were lysed in buffer containing 50 mM Tris (pH 7.4), 150 mM NaCl, 1% Nonidet P-40, 0.5% sodium deoxycholate, 0.1% SDS, and protease inhibitors. After centrifugation at 12,000 rpm for 20 min at 4° C., the supernatant was loaded onto an 8% SDS polyacrylamide gel for electrophoretic analysis. Separated proteins were transferred onto nitrocellulose membranes for immunoblot analysis. The antibodies against total β-catenin (610153, BD Biosciences, most of which is phosphorylated β-catenin and represents the E-cadherin bound pool), the active form of β-catenin (ABC, 05-665, EMD Millipore, dephosphorylated at positions S37 and T41 of β-catenin), cyclin D1 (sc-853, Santa Cruz Biotechnology, Inc.), c-myc (D84C12, Cell Signaling), and β-tubulin (sc-55529, Santa Cruz Biotechnology, Inc) were incubated with the membranes overnight at 4° C. respectively. IRDye 680LT goat anti-mouse IgG (827-11080, LiCOR) or IRDye 800CW goat anti-rabbit IgG (827-08365, LiCOR) was used as the secondary antibodies. The images were detected by the Odyssey Infrared Imaging System (LiCOR). Experiments were performed in duplicate.

### 36. Co-Immunoprecipitation Assay

HCT116 cells at 1×10⁶/mL were treated with different concentrations of 21 for 24 h. Cells were lysed in buffer containing 50 mM Tris, pH 7.4, 150 mM NaCl, 1% Nonidet P-40, 2 mM EDTA, and protease inhibitors. The lysates were preadsorbed to A/G plus agarose (sc-2003, Santa Cruz Biotechnology, Inc.) at 4° C. for 1 h. Preadsorbed lysates were incubated with a specific primary antibody against β-catenin (610153, BD Biosciences) overnight at 4° C. A/G plus agarose was then added to the lysate mixture and incubated for 3 h. The beads were washed 5 times with the lysis buffer at 4° C. The bound protein was eluted by boiling in the SDS sample buffer and loaded onto 8% SDS polyacrylamide gel for electrophoretic analysis. Separated proteins were transferred onto nitrocellulose membranes for immunoblot analysis. The antibodies against BCL9 (ab37305, Abcam) and E-cadherin (610404, BD Biosciences) were incubated with the membranes, respectively. IRDye 680LT goat anti-mouse IgG (827-11080, LiCOR) was used as the secondary antibody. The images were detected by the Odyssey Infrared Imaging System (LiCOR). Experiments were performed in duplicate.

### 37. MTs Cell Viability Assay

Colorectal cancer cell lines, SW480, HT29, and HCT116, triple-negative breast cancer cell lines, MDA-MB-231 and MDA-MB-436, lung adenocarcinoma cell line A549, and normal mammary epithelial cell line MCF10A were seeded in 96-well plates at 4×10³ cells/well, maintained overnight at 37° C., and incubated in the presence of 20, 21, and camosic acid at various concentrations. Cell viability was monitored after 72 h using a freshly prepared mixture of 1 part phenazine methosulfate (PMS, Sigma) solution (0.92 mg/mL) and 19 parts 3-(4,5-dimethylthiazol-2-yl)-5-(3-carboxymethoxyphenyl)-2-(4-sulfophenyl)-2H-tetrazolium (MTs, Promega) solution (2 mg/mL). Cells were incubated in 10 μL of this solution at 37° C. for 3 h, and A₄₉₀ was measured. The effect of each compound is expressed as the concentration required to reduce A₄₉₀ by 50% (IC₅₀) relative to vehicle-treated cells. Experiments were performed in triplicate.

### 38. Anchorage-Independent Growth Assay

3 mL of 0.5% agar in DMEM supplemented with 10% FBS was layered onto the 6 cm tissue culture plates. HCT116 cells (5×10³) that were treated with different concentrations of 21 were added to 0.35% agar in DMEM supplemented with 10% FBS, and the mixture was then added to the top of the 0.5% agar-precoated tissue culture plates. Cells were incubated at 37° C. in 5% CO₂ for 18 d, and the number of colonies was scored by crystal violet staining. Relative colony number was calculated as [(colony number) treatment/(colony number)_{control}]×100%. Each assay was performed in duplicate.

### 39. Activity of Substituted N-([1,1′-biphenyl]-3-yl)-[1,1′-biphenyl]-3-carboxamide Analogs in a MTs Cell Viability Assay and AlphaScreen Competitive Inhibition Assay

Substituted N-([1,1′-biphenyl]-3-yl)-[1,1′-biphenyl]-3-carboxamide analogs were synthesized as described above. Activity (IC₅₀) was determined in the MTs cell viability assay as described below and binding affinity (Kᵢ) were determined in the AlphaScreen competitive inhibition assay as described below and are shown in Table 3 below.

TABLE 3

| Compound | | |
|---|---|---|
| No. | Kᵢ (μM) | IC₅₀ (μM) |
| 9 | 33.6 ± 4.34 | 34.9 ± 4.53 |
| 10 | 27.7 ± 3.67 | 28.9 ± 3.48 |
| 11 | 87.1 ± 4.95 | 90.7 ± 5.17 |
| 12 | 149 ± 5.69 | 155 ± 5.94 |
| 13 | 29.3 ± 3.85 | 30.5 ± 4.02 |
| 14 | 74.0 ± 3.48 | 77.0 ± 3.64 |
| 15 | 32.4 ± 4.24 | 33.7 ± 4.43 |
| 16 | 11.6 ± 1.34 | 12.3 ± 1.44 |
| 17 | 6.12 ± 1.53 | 6.60 ± 2.28 |
| 18 | 36.8 ± 2.72 | 39.3 ± 2.92 |
| 20 | 6.65 ± 0.782 | 7.05 ± 0.848 |
| 21 | 2.11 ± 0.414 | 2.47 ± 0.484 |
| 22 | 17.0 ± 1.99 | 18.2 ± 2.14 |
| 23 | 8.71 ± 0.893 | 9.23 ± 0.965 |
| 24 | 201 ± 9.29 | 220 ± 9.99 |
| 25 | 8.45 ± 2.65 | 9.03 ± 2.84 |
| 26 | 4.08 ± 0.355 | 4.37 ± 0.399 |
| 27 | 5.60 ± 0.514 | 6.37 ± 0.568 |
| 28 | 3.41 ± 0.354 | 3.66 ± 0.397 |
| 29 | 2.10 ± 0.432 | 2.20 ± 0.450 |
| 30 | 156 ± 3.96 | 165 ± 4.21 |
| 31 | 180 ± 4.84 | 192 ± 5.15 |
| 84a | 78.6 ± 10.4 | 83.8 ± 11.1 |
| 84b | 84.2 ± 14.8 | 89.8 ± 15.8 |
| 84c | 68.6 ± 14.9 | 73.1 ± 15.9 |
| 91a | 36.0 ± 7.21 | 38.4 ± 7.71 |
| 91b | 40.8 ± 5.94 | 43.5 ± 6.35 |
| 91c | 41.4 ± 9.16 | 44.2 ± 9.79 |
| 91d | 54.8 ± 6.24 | 58.4 ± 6.67 |
| 91e | 25.9 ± 5.58 | 27.6 ± 5.97 |
| 91f | 29.5 ± 5.77 | 31.4 ± 6.17 |
| 91g | 162 ± 8.69 | 173 ± 9.29 |
| 91h | 51.7 ± 6.15 | 55.1 ± 658 |
| 91i | 11.1 ± 1.16 | 11.9 ± 1.26 |
| 91j | 27.3 ± 4.83 | 29.1 ± 5.17 |
| 91k | 3.91 ± 0.734 | 4.19 ± 0.803 |
| 91l | 19.9 ± 4.87 | 21.2 ± 5.21 |
| 91m | 124 ± 10.4 | 132 ± 11.1 |
| 95 | 244 ± 13.3 | 260 ± 14.2 |
| 96 | 7.17 ± 1.30 | 7.66 ± 1.40 |
| 97 | 9.50 ± 1.12 | 10.2 ± 1.21 |
| 98 | 180 ± 4.84 | 191 ± 5.15 |
| 99 | 156 ± 3.96 | 165 ± 4.21 |
| 100 | n.d. | n.d. |
| 101 | 56.2 ± 7.64 | 60.0 ± 8.17 |
| 102 | 126 ± 10.3 | 135 ± 11.1 |
| 103 | 96.4 ± 9.78 | 103 ± 10.5 |

* “n.d.” indicates that the value was not determined for the indicated compound.

### 40. Prophetic Western Blot Assay

The following example of an in vitro effect of the disclosed compounds is prophetic. HCT116 cells bear a deletion of codon S45 in β-catenin that makes the protein refractory to phosphorylation and degradation. It results in Wnt targets such as Cylin D1 and c-myc are thus overexpressed. To test the inhibitory effect of compounds on the expression of endogenous Wnt/β-catenin target genes in HCT116 cells, a Western blot assay for Cyclin D1 can be performed. Briefly, HCT116 cells are seeded at 10⁶ cells/plate, cultured overnight at 37° C., treated with different concentrations of compounds for 24h and washed by PBS. Cells are lysed in buffer A (150 mM NaCl, 1% NP-40, 50 mM TrisHCl, pH 8.0, 5 mM EDTA, 1 mM PMSF, 5 μM pepstatin, 10 μM Bestatin, and 5 μM E64) for 30 min. Protein concentration was determined by BCA assay. 25 μg protein is resolved by 12% SDS-PAGE. The separated proteins are transferred to a PDVF membrane. After transfer, the membrane is saturated by incubation at 4° C. for 1 h with blocking buffer (LI-COR Biosciences) and then incubated with primary antibody cyclin D1Ab (sc753, Santa Cruz) or β-tubulin Ab (sc55529, Santa Cruz) overnight at 4° C. After washing with PBST, the membrane is incubated with secondary antibody IRDye 800CW goat anti-rabbit IgG (LI-COR Biosciences, cat. #827-08365) for cyclin D1 or IRDye 680LT goat anti-mouse IgG (LI-COR Biosciences, cat. #827-11080) for β-tubulin for 60 min at room temperature. The membrane is washed 5 times with PBST and blots were imaged using an Odyssey Infrared Imaging System (LI-COR Biosciences).

For example, compounds having a structure represented by a formula:

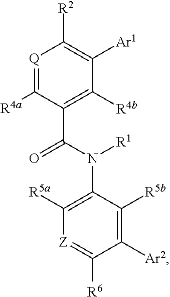

wherein wherein Q is selected from N and CR^{4c}; wherein Z is selected from N and CR^{5c}; wherein R¹ is selected from hydrogen and C1-C4 alkyl; wherein R² is selected from -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂—Cy¹, —NHCH₂—Cy²; —OCH₂—Cy¹, and —OCH₂—Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂—Cy³, —NHCH₂—Cy⁴; —OCH₂—Cy³, and —OCH₂—Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

### 41. Prophetic In Vivo Activity in a Tumor Xenograft Model

The following example of the in vivo effect of the disclosed compounds is prophetic. Generally agents which inhibit the Wnt pathway, including β-catenin/BCL9 protein-protein interaction inhibitors, are expected to display efficacy in preclinical models of cancer. In vivo effects of the compounds described in the preceding examples are expected to be shown in various animal models of cancer known to the skilled person, such as tumor xenograft models. These models are typically conducted in rodent, most often in mouse, but may be conducted in other animal species as is convenient to the study goals. Compounds, products, and compositions disclosed herein are expected to show in vivo effects in various animal models of cancer known to the skilled person, such as mouse tumor xenograft models.

In vivo effects of compounds can be assessed with in a mouse tumor xenograft study, one possible study protocol is described herein. Briefly, cells (2 to 5×10⁶ in 100 μl culture media) were implanted subcutaneously in the right hind flank athymic nu/nu nude mice (5 to 6 weeks old, 18-22 g). For test compounds of the present invention, a typical cell-line used for the tumor xenograft study can be HCT116 cells (a colon cancer cell line; ATCC CCL-247, ATCC, Manassas, Va.). Other suitable cell-lines for these studies are breast or prostate cancer cell lines available from ATCC. The cells are cultured prior to harvesting for this protocol as described herein.

Following implantation, the tumors are allowed to grow to 100 mm³ before the animals are randomized into treatment groups (e.g., vehicle, positive control and various dose levels of the test compound; the number of animals per group is typically 8-12. Day 1 of study corresponds to the day that the animals receive their first dose. The efficacy of a test compound can be determined in studies of various length dependent upon the goals of the study. Typical study periods are for 14, 21 and 28-days. The dosing frequency (e.g., whether animals are dosed with test compound daily, every other day, every third day or other frequencies) is determined for each study depending upon the toxicity and potency of the test compound. A typical study design would involve dosing daily (M-F) with the test compound with recovery on the weekend. Throughout the study, tumor volumes and body weights are measured twice a week. At the end of the study the animals are euthanized and the tumors harvested and frozen for further analysis.

For example, compounds having a structure represented by a formula:

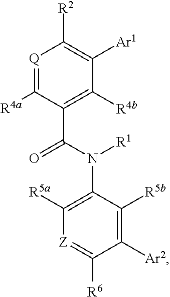

wherein wherein Q is selected from N and CR^{4c}; wherein Z is selected from N and CR^{5c}; wherein R¹ is selected from hydrogen and C1-C4 alkyl; wherein R² is selected from -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂—Cy¹, —NHCH₂—Cy²; —OCH₂—Cy¹, and —OCH₂—Cy²; wherein Cy¹, when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl; wherein each of R^{4a}, R^{4b}, and R^{4c}, when present, is independently selected from hydrogen, C1-C4 alkyl, C1-C4 monohaloalkyl, and C1-C4 polyhaloalkyl; wherein each of R^{5a}, R^{5b}, and R^{5c}, when present, is independently selected from hydrogen, C1-C4 alkyl, C1-C4 monohaloalkyl, and C1-C4 polyhaloalkyl; wherein R⁶ is selected from hydrogen, -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂—Cy³, —NHCH₂—Cy⁴; —OCH₂—Cy³, and —OCH₂—Cy⁴; wherein Cy³, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl; wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; and wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, and —CO₂H; or a pharmaceutically acceptable salt thereof.

### 42. Prophetic Pharmaceutical Composition Examples

“Active ingredient” as used herein throughout these examples relates to one or more disclosed compounds, or a product of a disclosed method of making, or a pharmaceutically acceptable salt, solvate, polymorph, hydrate and the stereochemically isomeric form thereof. The following examples of the formulation of the compounds of the present invention in tablets, suspension, injectables and ointments are prophetic.

Typical examples of recipes for the formulation of the invention are as given below. Various other dosage forms can be applied herein such as a filled gelatin capsule, liquid emulsion/suspension, ointments, suppositories or chewable tablet form employing the disclosed compounds in desired dosage amounts in accordance with the present invention. Various conventional techniques for preparing suitable dosage forms can be used to prepare the prophetic pharmaceutical compositions, such as those disclosed herein and in standard reference texts, for example the British and US Pharmacopoeias, Remington's Pharmaceutical Sciences (Mack Publishing Co.) and Martindale The Extra Pharmacopoeia (London The Pharmaceutical Press). The disclosure of this reference is hereby incorporated herein by reference.

### a. Pharmaceutical Composition for Oral Administration

A tablet can be prepared as follows:

| Component | Amount | |
|---|---|---|
| Active ingredient | 10 to 500 | mg |
| Lactose | 100 | mg |
| Crystalline cellulose | 60 | mg |
| Magnesium stearate | 5 | mg |
| Starch (e.g. potato starch) | Amount necessary to yield | |
| | total weight indicated below | |
| Total (per capsule) | 1000 | mg |

Alternatively, about 100 mg of a disclosed compound, 50 mg of lactose (monohydrate), 50 mg of maize starch (native), 10 mg of polyvinylpyrrolidone (PVP 25) (e.g. from BASF, Ludwigshafen, Germany) and 2 mg of magnesium stearate are used per tablet. The mixture of active component, lactose and starch is granulated with a 5% solution (m/m) of the PVP in water. After drying, the granules are mixed with magnesium stearate for 5 min. This mixture is moulded using a customary tablet press (e.g., tablet format: diameter 8 mm, curvature radius 12 mm). The moulding force applied is typically about 15 kN.

Alternatively, a disclosed compound can be administered in a suspension formulated for oral use. For example, about 100-5000 mg of the desired disclosed compound, 1000 mg of ethanol (96%), 400 mg of xanthan gum, and 99 g of water are combined with stirring. A single dose of about 10-500 mg of the desired disclosed compound according can be provided by 10 ml of oral suspension.

In these Examples, active ingredient can be replaced with the same amount of any of the compounds according to the present invention, in particular by the same amount of any of the exemplified compounds. In some circumstances it may be desirable to use a capsule, e.g., a filled gelatin capsule, instead of a tablet form. The choice of tablet or capsule will depend, in part, upon physicochemical characteristics of the particular disclosed compound used.

Examples of alternative useful carriers for making oral preparations are lactose, sucrose, starch, talc, magnesium stearate, crystalline cellulose, methyl cellulose, hydroxypropyl cellulose, hydroxypropylmethyl cellulose, carboxymethyl cellulose, glycerin, sodium alginate, gum arabic, etc. These alternative carriers can be substituted for those given above as required for desired dissolution, absorption, and manufacturing characteristics.

The amount of a disclosed compound per tablet for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

### b. Pharmaceutical Composition for Injectable Use

A parenteral composition can be prepared as follows:

| Component | Amount | |
|---|---|---|
| Active ingredient | 10 to 500 | mg |
| Sodium carbonate | 560 | mg* |
| Sodium hydroxide | 80 | mg* |

| | |
|---|---|
| Distilled, sterile water | Quantity sufficient to prepare |
| | total volumen indicated below. |
| Total (per capsule) | 10 ml per ampule |

*Amount adjusted as required to maintain physiological pH in the context of the amount of active ingredient, and form of active ingredient, e.g., a particular salt form of the active ingredient.

Alternatively, a pharmaceutical composition for intravenous injection can be used, with composition comprising about 100-5000 mg of a disclosed compound, 15 g polyethylenglycol 400 and 250 g water in saline with optionally up to about 15% Cremophor EL, and optionally up to 15% ethyl alcohol, and optionally up to 2 equivalents of a pharmaceutically suitable acid such as citric acid or hydrochloric acid are used. The preparation of such an injectable composition can be accomplished as follows: The disclosed compound and the polyethylenglycol 400 are dissolved in the water with stirring. The solution is sterile filtered (pore size 0.22 μm) and filled into heat sterilized infusion bottles under aseptic conditions. The infusion bottles are sealed with rubber seals.

In a further example, a pharmaceutical composition for intravenous injection can be used, with composition comprising about 10-500 mg of a disclosed compound, standard saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid. Preparation can be accomplished as follows: a desired disclosed compound is dissolved in the saline solution with stirring. Optionally Cremophor EL, ethyl alcohol or acid are added. The solution is sterile filtered (pore size 0.22 μm) and filled into heat sterilized infusion bottles under aseptic conditions. The infusion bottles are sealed with rubber seals.

In this Example, active ingredient can be replaced with the same amount of any of the compounds according to the present invention, in particular by the same amount of any of the exemplified compounds.

The amount of a disclosed compound per ampule for use in a pharmaceutical composition for human use is determined from both toxicological and pharmacokinetic data obtained in suitable animal models, e.g., rat and at least one non-rodent species, and adjusted based upon human clinical trial data. For example, it could be appropriate that a disclosed compound is present at a level of about 10 to 1000 mg per tablet dosage unit.

Carriers suitable for parenteral preparations are, for example, water, physiological saline solution, etc. which can be used with tris(hydroxymethyl)aminomethane, sodium carbonate, sodium hydroxide or the like serving as a solubilizer or pH adjusting agent. The parenteral preparations contain preferably 50 to 1000 mg of a disclosed compound per dosage unit.

### F. EXAMPLES

### 1. Hot Regions 1 and 2 of β-Catenin/BCL9 Protein-Protein Interactions (PPIs)

Hot region 1: D162, E163, and D164 of human β-catenin form an acidic knob (Hoffmans and Basler (2004) Development 131 (17): 4393-4400) and interact with H358 and R359 of human BCL9. The D162A mutation of β-catenin reduced it binding to BCL9 (Kawamoto et al. (2009) Biochemistry 48 (40): 9534-9541). β-Catenin D164A abrogated the interaction with BCL9 or B9L in vitro (Hoffmans and Basler (2007) Mech. Dev. 124 (1): 59-67) and BCL9-dependent Wnt transcription in vivo (Valenta et al. (2011) Genes Dev. 25 (24): 2631-2643). Mutation of either H358 or R359 of BCL9 to alanine significantly reduced its binding with β-catenin (Sampietro et al. (2006) Mol. Cell 24 (2): 293-300). The ³⁵⁸HRE^{360/358}AKQ³⁶⁰ mutation of BCL9 completely disrupted its binding with β-catenin (de la Roche et al. (2008) BMC Cancer 8: 199). Hot region 2: BCL9 L366, 1369, and L373 interact with a hydrophobic pocket on β-catenin that is lined with L159, V167, L160, A171, M174, L178, A149, A152, and L156. BCL9 L366K,¹⁰ L373A,¹⁰ or L366A/I369A⁹ prevented its binding with β-catenin in the pulldown experiment. A similar result was also observed in a cell-based study. BCL9 L366K or B9L L411K could not co-immunoprecipitate with β-catenin (Jochim and Arora (2009) Mol. Biosyst. 5 (9): 924-926). BCL9 L366A, 1369A, or L373A exhibited no observable inhibition of wild-type β-catenin/wild-type BCL9 PPIs in the fluorescence polarization (FP) competitive inhibition assay (Kawamoto et al. (2009) Biochemistry 48 (40): 9534-9541). The pulldown experiment showed that β-catenin L159A or L178A bound with BCL9, however, a double mutant, β-catenin L156A/L159A, abolished its interaction with BCL9 (Yu et al. (2013) ACS Chem. Biol. 8 (3): 524-529). Without wishing to be bound by theory, the site-directed mutagenesis experiments in this study indicate that β-catenin L156S or L159S reduced the binding affinity with β-catenin, while β-catenin D145A, E155A, and D145A/E155A had no affect on β-catenin/BCL9 PPIs. β-Catenin double mutants, L156S/L159S and L156S/L178S, abolished their binding with BCL9.

### 2. Fragment Hopping to Design 4′-fluoro-N-phenyl-[1,1′-biphenyl]-3-carboxamide for Mimicking the Hydrophobic Side Chains of Hot Spots i, i+3, and i+7 of an α-Helix

The protocol of fragment hopping has been described in the previous papers (Ji et al. (2008) J. Am. Chem. Soc. 130 (12): 3900-3914; Ji et al. (2009) J. Med. Chem. 52 (3): 779-797; Yu et al. (2013) ACS Chem. Biol. 8 (3): 524-529). The key of fragment hopping for designing protein-protein interaction inhibitors is the extraction of the critical binding elements based on the binding mode between the projecting hot spots and the concave hot spot pocket. The basic fragment library and the bioisostere library are then interrogated to identify new fragments that can match the proposed critical binding elements. The bioisosteric replacement technique can be used to produce new fragments with the chemotypes that do not exist in hot spots. Fragment docking, ligand docking, inhibitor structure-activity relationship analysis, and structure validation are then conducted to provide insight about the binding mode of new inhibitors.

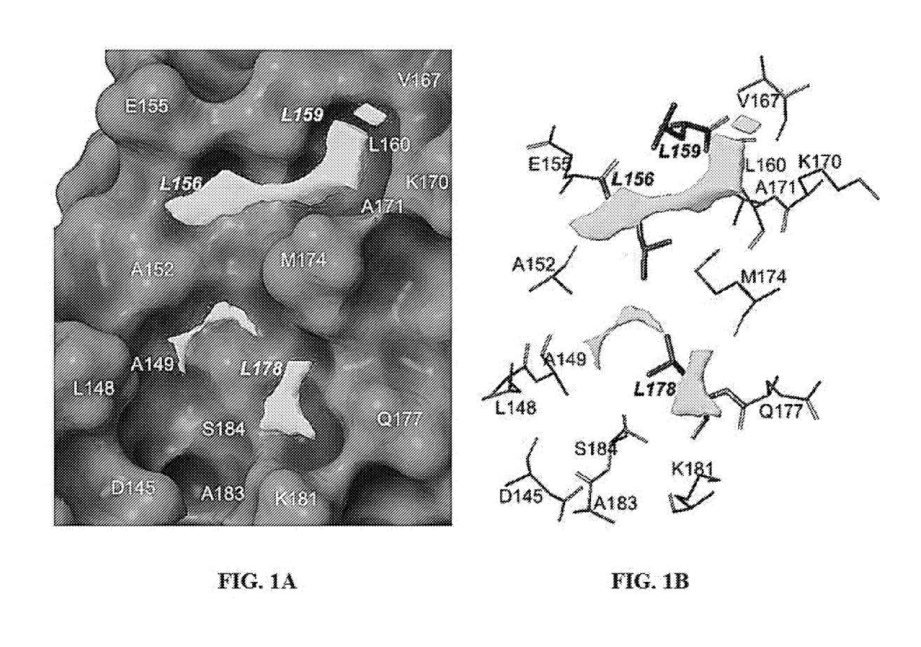

Residues L366, I369, and L371 of BCL9 have been identified as the projecting hot spots as detailed herein above, and the BCL9 L366/I369/L373 binding site of β-catenin was identified as a druggable hot spot pocket (main text). SiteMap identified that the molecular interaction fields (MIFs) for hydrophobic interaction were mainly from the pocket lined with the side chains of A152, L156, L159, L160, V167, K170, A171, and M174 of β-catenin, as shown in FIG. 1A and FIG. 1B below. SiteMap identified additional hydrophobic MIFs generated from the side chains of L148, A149, A152, M174, and L178 of β-catenin. The hydrophobic side chains of these residues were extracted as the critical binding elements for hydrophobic interactions in inhibitor design.

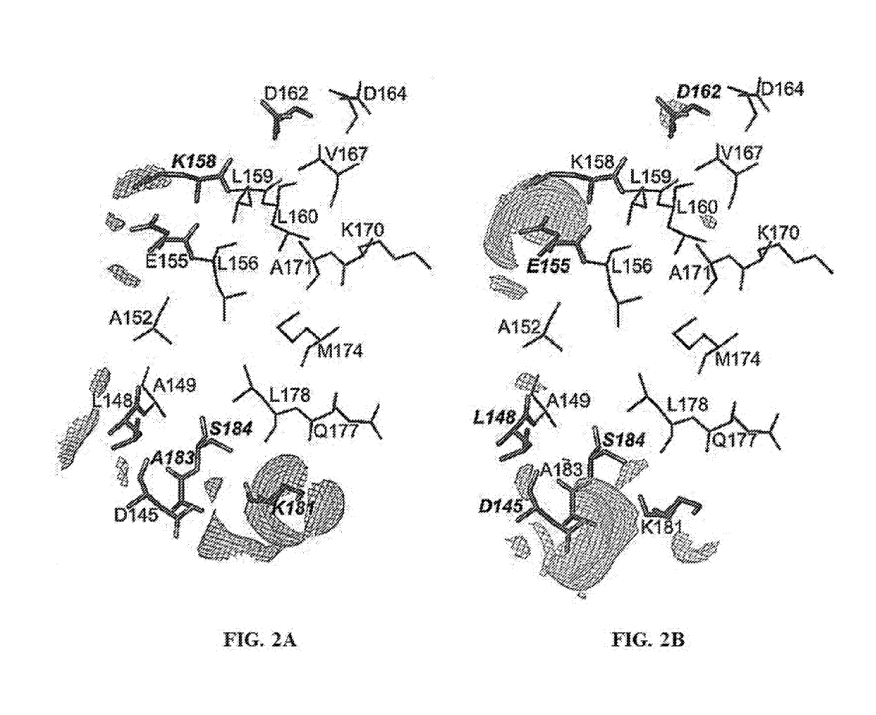

As shown in FIG. 2A and FIG. 2B, the SiteMap MIFs for H-bond donors were mainly from the side chain NH₃ of β-catenin K181, the side chain OH of β-catenin S184, and the backbone NH of β-catenin A183. The SiteMap MIFs of H-bond acceptors were determined by the side chain carboxylic oxygens of β-catenin D145, E155, D162, and S184, and the backbone carbonyl of L148. These atoms were extracted as the critical binding elements for H-bond and charge-charge interactions.

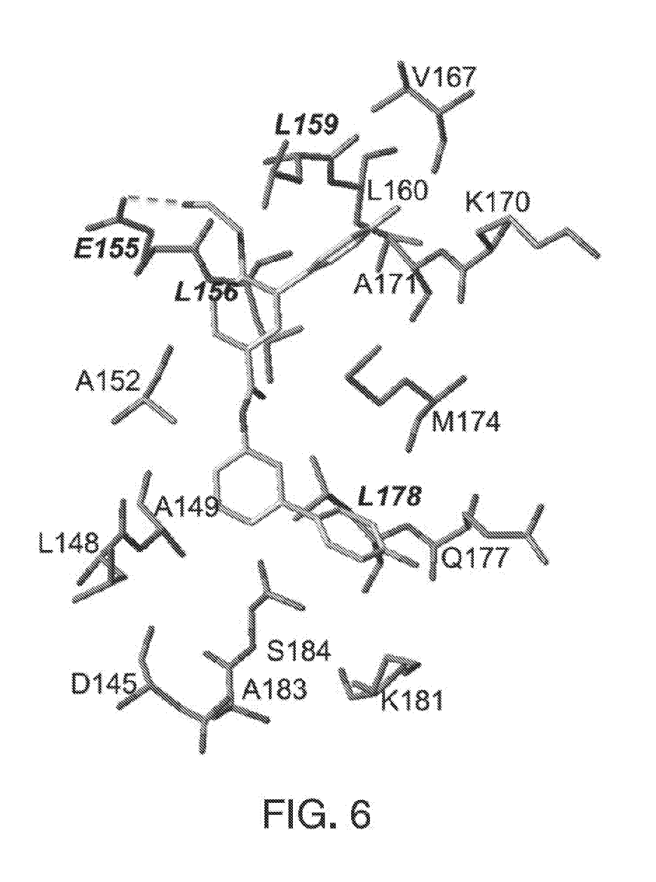

The results of the above analysis provide the base for fragment design. The side chains of three hot spots of BCL9, L366, 1369, and L371 were identified as the key binding features to mimic. The basic fragment library and the bioisostere library collected as Supplementary FIGS. 4 and 5 in ACS Chem. Biol. 8 (3), 524-529 (2013) were interrogated to identify new fragments that can match the proposed critical binding elements. The binding modes of new fragments were generated by fragment docking and prioritized by visual inspection to match the proposed critical binding elements. The assistant criterion was the docking scores. The side chain library in Supplementary FIG. 6 in ACS Chem. Biol. 8 (3), 524-529 (2013) was used to generate the linkers. The distance between each fragment of the different fragment pockets was measured, and the synthetically accessible linkers with an appropriate length was chosen with the assistance of Scifinder for synthetic accessibility. The generated ligand scaffolds were then docked in the binding site and visually inspected. If the newly generated ligand scaffold did not match the proposed critical binding elements, the ligand scaffold was rejected, and new structures were constructed by repeating the above steps. If the binding mode of the new ligand scaffold matched the proposed critical binding elements, they would be kept for further evaluation.

### 3. Scope of Application of 4′-fluoro-N-phenyl-[1,1′-biphenyl]-3-carboxamide to Mimic the Hydrophobic Side Chains of Residues i, i+3, and i+7 of an α-Helix

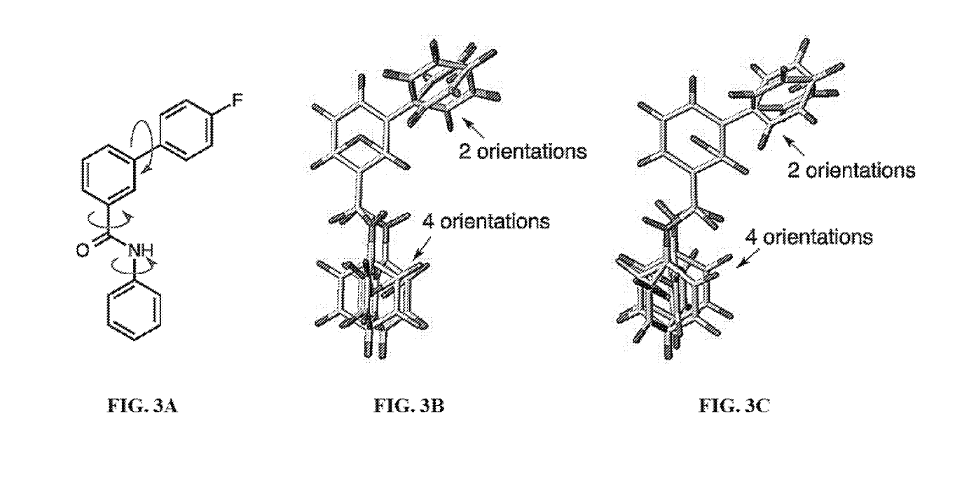

A conformational analysis of 4′-fluoro-N-phenyl-[1,1′-biphenyl]-3-carboxamide indicated that this compound had at least 16 low energy conformations, as indicated in FIG. 3A, FIG. 3B, and FIG. 3C. These 16 conformations were used as the base for database mining to superimpose with known protein structures in the RCSB protein data bank (PDB).

The protein-protein complexes in the RCSB PDB were retrieved to evaluate how well 4′-fluoro-N-phenyl-[1,1′-biphenyl]-3-carboxamide matches the hot spot side chains of α-helix-mediated PPIs. The helical PPI database, HippDB (Jochim and Arora (2009) Mol. Biosyst. 5 (9): 924-926; Jochim and Arora (2010) ACS Chem. Biol. 5 (10): 919-923; Bullock et al. (2011) J. Am. Chem. Soc. 133 (36): 14220-14223; Bergey et al. (2013) Bioinformatics 29 (21): 2806-2807), was interrogated to retrieve the protein-protein complexes that are predicted to have hydrophobic projecting hot spots at positions i, i+3, and i+7. The hydrophobic hot spots at each position could be leucine (L), isoleucine (I), methionine (M), valine (V), phenylalanine (F), and tryptophan (W).

Out of 27,746 protein-protein interaction entries in HippDB (on Jan. 15, 2014), 733 structures have the hydrophobic projecting hot spots at positions i, i+3, and i+7 of an α-helix and the concave hot spot pocket(s) at the protein-protein interfaces where the projecting hot spots were located. Among the 733 structures, the side chains of the projecting hot spots of 631 structures can be mimicked by 4′-fluoro-N-phenyl-[1,1′-biphenyl]-3-carboxamide. In other words, 86.1% of the protein-protein complexes can be mimicked by 4′-fluoro-N-phenyl-[1,1′-biphenyl]-3-carboxamide.

### 4. Rational Design of Small Molecules to Mimic the Side Chains of Residues i, i+3, and i+7 of an α-Helix

Crystallographic and biochemical analyses revealed two hot regions on the β-catenin/BCL9 interface (FIG. 4A) (de la Roche et al. (2008) BMC Cancer 8: 199; Sampietro et al. (2006) Mol. Cell 24: 293-300; Hoffmans and Basler (2004) Development 131: 4393-4400; Hoffmans and Basler (2007) Mech. Dev. 124: 59-67; Valenta et al. (2011) Genes Dev. 25: 2631-2643; Kawamoto et al. (2009) Biochemistry 48: 9534-9541). (1) D162 and D164 of β-catenin form salt bridges with H358 and R359 of BCL9; and (2) BCL9 L366, 1369, and L373 interact with a hydrophobic pocket on β-catenin that is lined with L159, V167, L160, A171, M174, L178, A149, A152, and L156. The ANCHOR (Meireles et al. (2010) Nucleic Acids Res. 38: W407-W411) and PocketQuery (Koes and Camacho (2012) Nucleic AcidRes. 40: W387-W392) analyses indicated that BCL9 L366, 1369, and L373 were anchor residues and the mimicry of BCL9 L366, 1369, and L373 could be a good starting point for inhibitor design (Tables 4 and 5). The SiteMap Dscore (Halgren (2009) J. Am. Chem. Soc. 130: 3900-3914) for the BCL9 L366/I369/L373 binding pocket of β-catenin was 1.076, indicating this is a druggable surface pocket for PPI targets.

TABLE 4*

| BCL9 | | SASA** | | |
|---|---|---|---|---|
| | Residue | ΔSASA | ΔSASA | Energy^{‡} |
| Residue | ID | (Å²)^{†} | (%) | (kcal/mol) |
| SER | 352 | 0 | 0 | −0.5 |
| GLN | 353 | 0 | 0 | 0.1 |
| GLU | 354 | 0 | 0 | 0.3 |
| GLN | 355 | 4 | 2.9 | 0.8 |
| LEU | 356 | 0 | 0 | 0.1 |
| GLU | 357 | 0 | 0 | 0.4 |
| HIS | 358 | 47.3 | 32.1 | 1.4 |
| ARG | 359 | 71.8 | 35.7 | −5.8 |
| GLU | 360 | 0 | 0 | 0.2 |
| ARG | 361 | 0 | 0 | −0.6 |
| SER | 362 | 33.2 | 42.5 | −1.2 |
| LEU | 363 | 49.8 | 35.3 | −1.2 |
| GLN | 364 | 0 | 0 | −0.1 |
| THR | 365 | 36.9 | 36.3 | −0.5 |
| LEU | 366 | 100.4 | 71.2 | −3.7 |
| ARG | 367 | 0.1 | 0.1 | 0.1 |
| ASP | 368 | 0 | 0 | 0.2 |
| ILE | 369 | 64.4 | 46.7 | −2.4 |
| GLN | 370 | 25.1 | 17.8 | −0.3 |
| ARG | 371 | 0 | 0 | 0 |
| MET | 372 | 12.9 | 8.2 | −0.4 |
| LEU | 373 | 86 | 60.9 | −2.6 |
| PHE | 374 | 86.2 | 52.5 | −1 |

*ANCHOR calculation result of the β-catenin/BCL9 interface. The residues that were regarded as the anchors are highlighted in bold and underlilne above. See Meireles, L. M. C., Dömling, A. S. & Camacho, C. J. Nucleic Acids Res. 38, W407-W411 (2010) for ANCHOR calculation method.
**SASA: solvent accessible surface area.
^{†}ΔSASA: the change in solvent accessible surface area.
^{‡}Energy: the associated binding free energy for each residue estimated by FastContact (Camacho, C. J. & Zhang, C. Bioinformatics 21 (10), 2534-2536 (2005); and Camacho, C. J., Ma, H. & Champ, P. C. Proteins 63 (4), 868-877 (2006)).

TABLE 5*

| | | | | Distance | |
|---|---|---|---|---|---|
| | | | | between | |
| PPIs | PDB | Chain | anchor residues | anchors | Av ΔG^{FC}** |
| BCL-x_{L}/Bak | 1BXL | B | V574, L578, I581 | 11.489 | −3.96333 |
| | | B | L578, I581, I585 | 10.3289 | −3.64333 |
| BCL-x_{L}/Bad | 1G5J | B | L312, M315, F319 | 9.6935 | −3.19 |
| | | B | Y308, L312, M315 | 10.8042 | −3.43333 |
| BCL2/Bax | 2XA0 | C | L59, L63, I66 | 10.4206 | −4.47 |
| | | C | L63, I66, L70 | 10.8244 | −3.88667 |
| MDM2/p53 | 1YCR | B | F19, W23, L26 | 10.2463 | −5.30333 |
| | 1T4F | P | F19, W23, L26 | 10.743 | −4.74667 |
| | 4HFZ | B | F19, W23, L26 | 10.2802 | −5.20667 |
| XDM2/p53 | 1YCQ | B | F19, W23, L26 | 10.1873 | −4.99667 |
| MDM4/p53 | 3DAB | B | F19, W23, L26 | 10.0466 | −4.93667 |
| XIAP | 1G73 | A | A1, P3, I4 | 10.0531 | −5.92 |
| BIR3/Smac | | | | | |
| XIAP | 1NW9 | B | A316, P318, F319 | 10.2591 | −2.8 |
| BIR3/caspase 9 | | | | | |
| cIAP | 3D9U | B | A1, P3, I4 | 9.8962 | −3.08333 |
| BIR3/Smac | | | | | |
| ZipA/FtsZ | 1F47 | A | I8, F11, L12 | 6.0083 | −2.32333 |
| IL2/IL-2R | 1Z92 | B | R36, L42, Y43 | 10.5964 | −5.26667 |
| HIV-1 | 2B4J | C | I365, D366 | 4.378 | −4.735 |
| intergrase/p75 | | C | I365, D366, N367 | 7.379 | −2.56667 |
| β-catenin/BCL9 | 2GL7 | C | L366, I369, L373 | 10.5061 | −2.1 |

*PocketQuery results of the β-catenin/BCL9 interface and the other protein-protein
interfaces that have known small-molecule inhibitors. The scores indicated that the
druggability of the BCL9 L366/I369/L373 binding pocket in β-catenin is similar to
that of the anti-apoptotic protein Bcl-xL/pro-apoptotic protein BAK interaction or the
interleukin-2 (IL2)/interleukin-2α receptor (IL-2αR) interaction. For PocketQuery method
see Koes, D. R. & Camacho, C. J. Nucleic Acids Res. 40, W387-W392 (2012).
**Av. ΔG^{FC}: an average of the changes in free energy (kcal/mol) for the anchor residues
upon complexation. It is calculated by FastContact (Jochim, A. L. & Arora, P. S.
ACS Chem. Biol. 5 (10), 919-923 (2010); and Bullock, B. N., Jochim, A. L. & Arora, P. S.
J. Am. Chem. Soc. 133 (36), 14220-14223 (2011)). A more negative value indicates a
stronger interaction.

| | Av | Av | Av | |
|---|---|---|---|---|
| PPIs | ΔΔGR^{‡} | ΔSASA^{†} | ΔSASA %^{††} | Score*** |
| BCL-x_{L}/Bak | −0.891167 | 97.0967 | 74.9333 | 0.767826 |
| | 0.0871 | 89.6067 | 64.4 | 0.715969 |
| BCL-x_{L}/Bad | 0.841667 | 107.583 | 69.6667 | 0.769361 |
| | 0.0189333 | 101.937 | 64.3 | 0.70176 |
| BCL2/Bax | 0.800033 | 106.547 | 76.0667 | 0.869186 |
| | 1.02093 | 100.977 | 72.1333 | 0.837731 |
| MDM2/p53 | 3.47917 | 113.723 | 65.5333 | 0.954928 |
| | 3.74377 | 128.023 | 76.5 | 0.970194 |
| | 2.61017 | 116.667 | 68.1333 | 0.947492 |
| XDM2/p53 | 2.89833 | 117.6 | 68.2 | 0.953374 |
| MDM4/p53 | 3.21237 | 115.1 | 66.5667 | 0.945673 |
| XIAP | 0.579667 | 88.9267 | 85.8333 | 0.903125 |
| BIR3/Smac | | | | |
| XIAP | 0.992667 | 87.34 | 80.6667 | 0.821808 |
| BIR3/caspase 9 | | | | |
| cIAP BIR3/Smac | −0.0189 | 84.9333 | 83.2333 | 0.803616 |
| ZipA/FtsZ | 1.68173 | 85.9633 | 58.4333 | 0.71793 |
| IL2/IL-2R | 0.460967 | 81.8633 | 49.1333 | 0.793064 |
| HIV-1 | 0.73375 | 116.575 | 97.4 | 0.857889 |
| intergrase/p75 | 0.541933 | 87.0767 | 73.7333 | 0.723532 |
| β-catenin/BCL9 | 2.00527 | 85.1467 | 60.7 | 0.763969 |

‡Av. ΔΔG^{R}: an average of the changes in free energy of an alanine mutation
for the anchor residues. It is calculated by Rosetta (Kortemme, T., Kim, D. E. & Baker, D.
Sci. STKE 2004 (219), p12 (2004).). A more positive value indicates the mutation destabilizes
the complex and thus the original residue has a stronger interaction.
†Av. ΔSASA: an average of the changes in solvent accessible surface area of the anchor residues.
††Av. ΔSASA %: an average of the relative ΔSASA.
***Score: a ‘druggability’ indicator. It is ranged from 0 to 1. A higher score indicates the
mimicry of the anchor residues provides a better starting point.

A small-molecule scaffold, 4′-fluoro-N-phenyl-[1,1′-biphenyl]-3-carboxamide (FIG. 4B), was designed as described herein above and used to mimic the hydrophobic side chains of residues i, i+3, and i+7 of an α-helix. Three rotatable single bonds of this scaffold can produce at least 16 low-energy conformations. The extensive PDB data mining indicated that this scaffold can mimic 86.1% of PPIs that have such hot spot patterns and hot spot pockets. An overlay of this scaffold with BCL9 L366, I369, and L373 is shown in FIG. 4C. To verify this design, compounds 1-15 in FIG. 4D were synthesized. The synthetic routes for compounds 1-15 are shown in Synthesis Schemes 1-6. Compounds 1-4 were designed to mimic L366 and 1369. Without wishing to be bound by theory, the AlphaScreen assay showed that these compounds inhibited β-catenin/BCL9 PPIs at a low mM range (FIG. 4F and FIG. 5). A 4-aminoethoxy or 4-aminopropyl side chain was introduced to 4 with an attempt to form charge-charge and H-bond interactions with E155. The AlphaScreen assay showed that the resulting compounds, 5 and 6, were 5-fold more potent than 4. The one-carbon-shorter derivative, 7, and the hydroxyl derivative, 8, were less potent than 6 probably due to the weakened electrostatic interactions. Compounds 9-12 were designed to explore the pocket adjacent to β-catenin L178. The para and meta positions of the terminal benzene ring can tolerate a fluorine substitution. Compounds 13-15 were designed to optimize the interactions with residues L159, L160, V167, and A171. The results showed that 4-fluoro and 3,4-difluoro derivatives, 9 and 13, exhibited higher potency than 14. On the other hand, the 3-thionyl derivative, 15, had a similar potency as 9. An AutoDock (Friesner et al. (2006) J. Med. Chem. 49: 6177-6196) result of 9 is shown in FIG. 4E and FIG. 6.

### 5. Structure-Activity Relationship-Based Hit Optimization

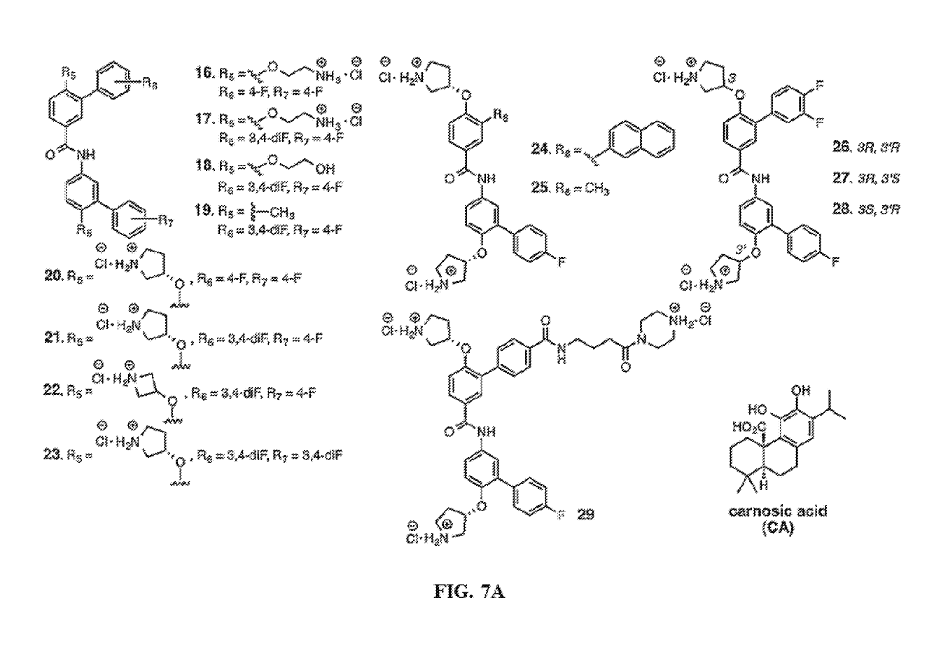

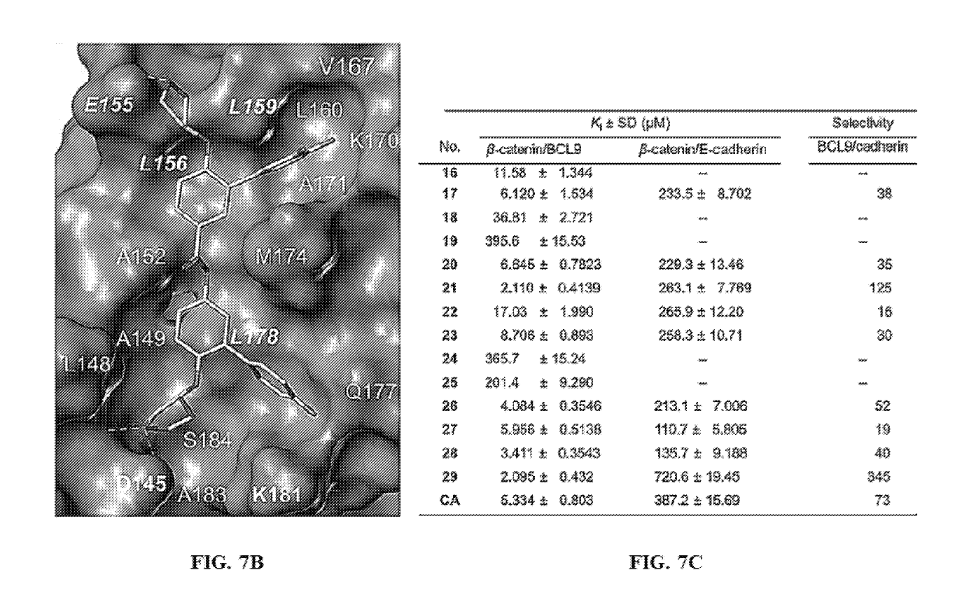

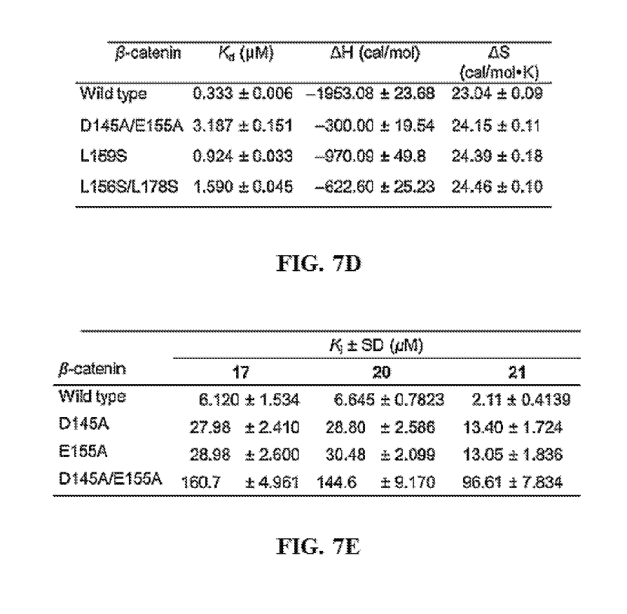

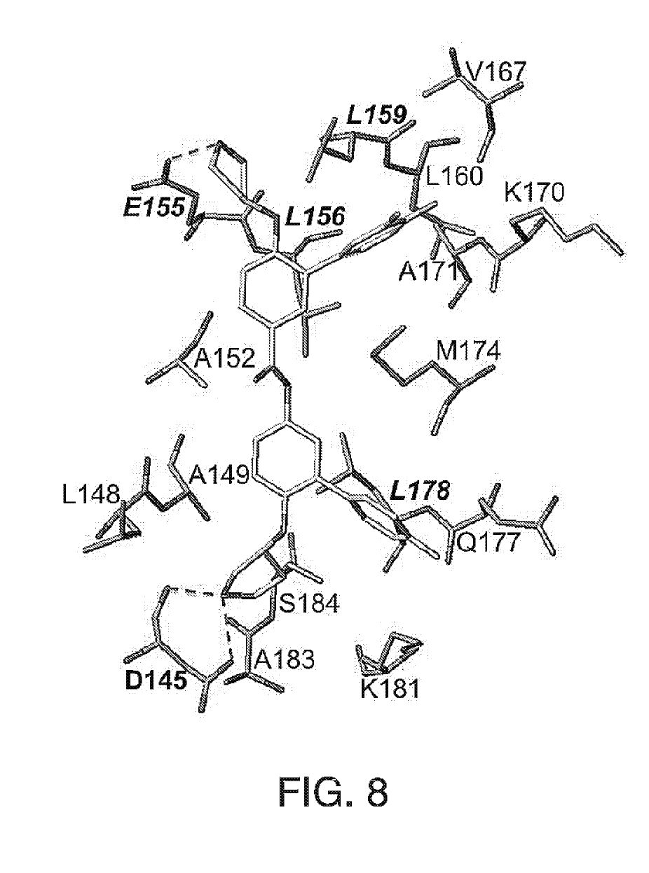

Compounds 16-28 in FIG. 7A were designed to generate more potent β-catenin/BCL9 inhibitors. The synthetic routes for 16-28 are shown in Synthesis Schemes 7-11. A second aminoethoxy group was introduced to 9 and 13 to produce additional charge-charge and H-bond interactions with β-catenin D145. As shown in FIG. 7C, the resulting compounds, 16 and 17, were more potent than 9 and 13. Compounds 18 and 19 were designed to explore the role of β-catenin D145 and E155 in inhibitor binding. Compound 18 is predicted to have H-bonds but no charge-charge interactions with D145 and E155, while 19 loses both H-bond and charge-charge interactions. The AlphaScreen assay showed that 18 and 19 were 6- and 65-fold less potent than 17, respectively. The (S)-pyrrolidin-3-yloxy group of 20 and 21 was designed to replace the aminoethoxy group of 16 and 17 with the attempt to increase cellular permeability. These two compounds were 2- to 3-fold more potent than 16 and 17, respectively, likely due to the conformational constraint of the five-membered ring. Compound 21 exhibited a Kᵢ value of 2.11±0.4139 μM and was more potent than carnosic acid in the parallel assay. Both AutoDock and Glide (Friesner et al. (2006) J. Med. Chem. 49: 6177-6196) docking studies generated the same predicted binding mode for 21, as shown in FIG. 7B and FIG. 8 The substitution of the pyrrolidine ring of 21 with an aziridine ring led to a decrease in inhibitory activity. Derivative 23 has two 3,4-difluoro substituents on both benzene rings, exhibiting slightly lower potency than 20. The replacement of the 3,4-difluorophenyl group of 21 with a 2-naphthyl or methyl group drastically decreases its inhibitory potency, projecting the size of the hot spot pocket. The other three stereoisomers of 21 were also synthesized. The AlphaScreen assay of 26-28 indicated that these three stereoisomers had the similar inhibitory activities as 21. This result is consistent with that predicted from the AutoDock studies because D145 and E155 are located on the surface of the ligand binding pocket.

### 6. Isothermal Titration Calorimetry (ITC) and Site-Directed Mutagenesis Studies

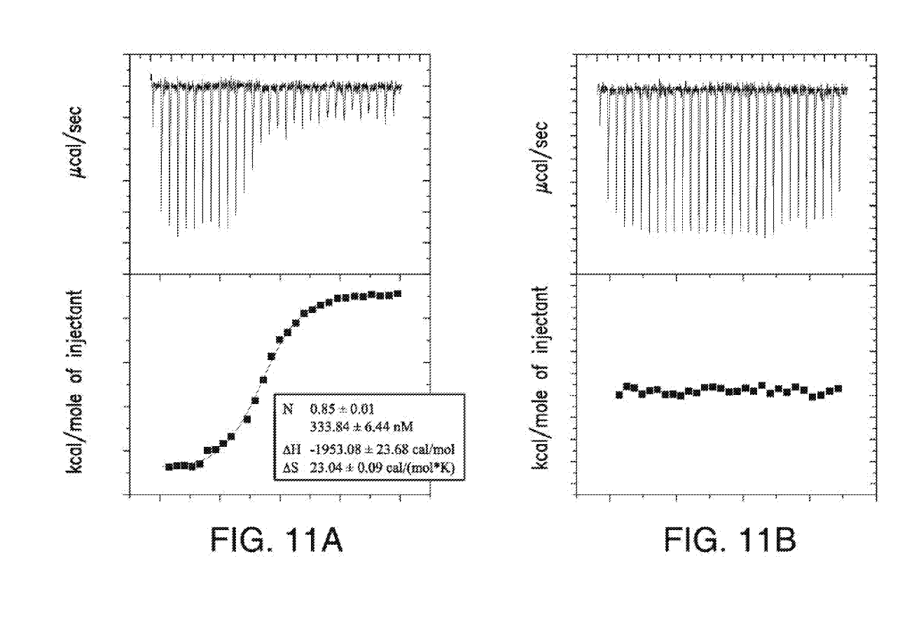

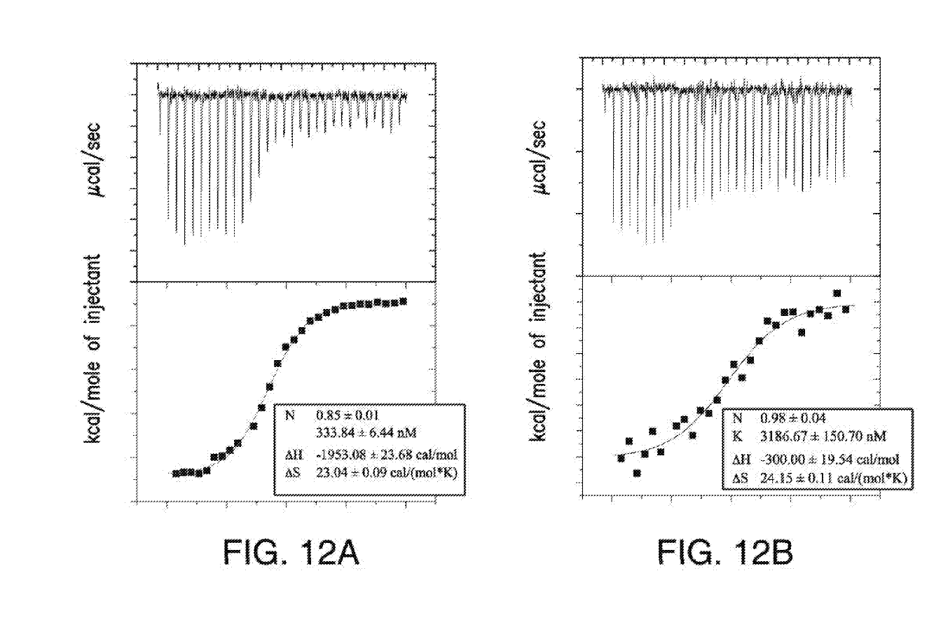

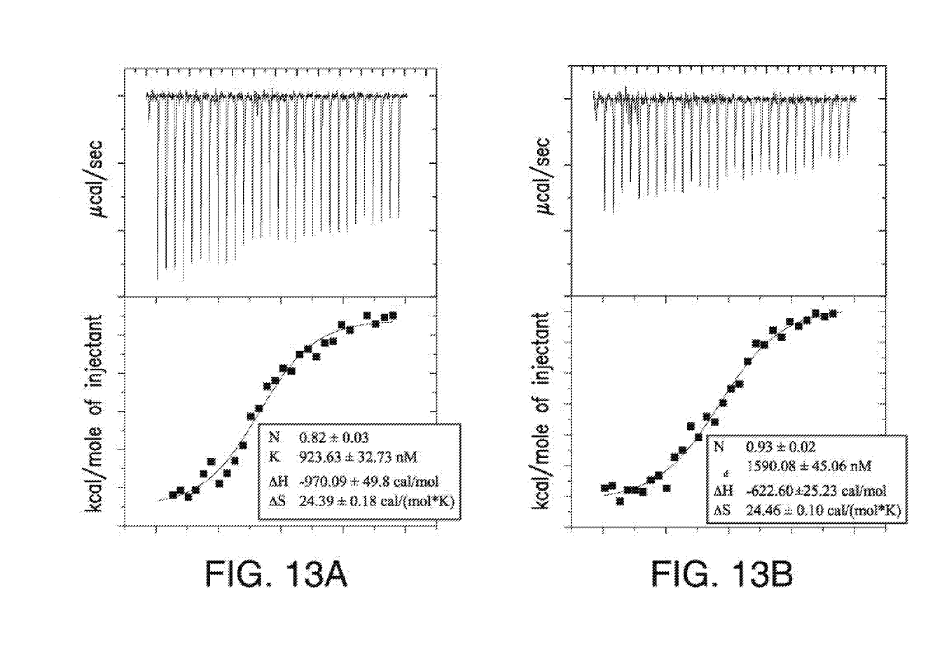

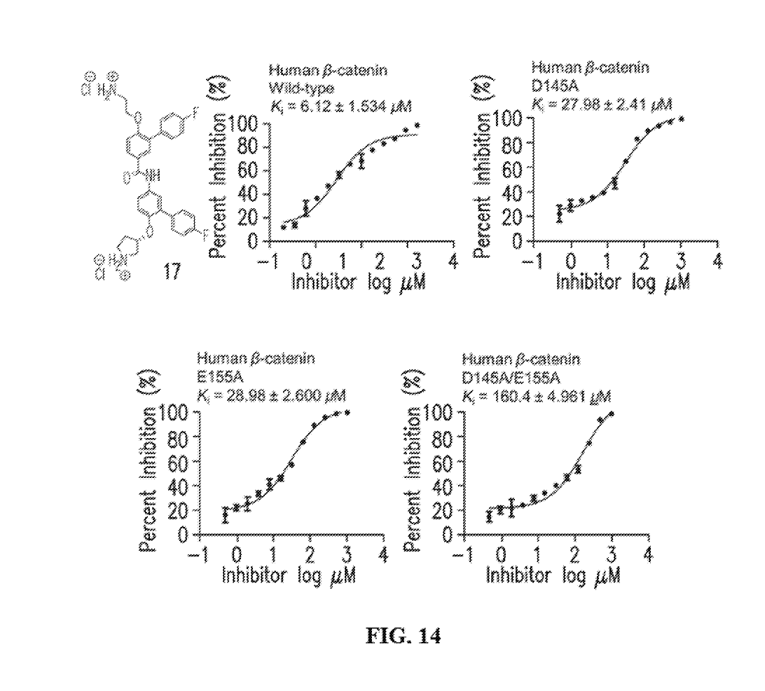

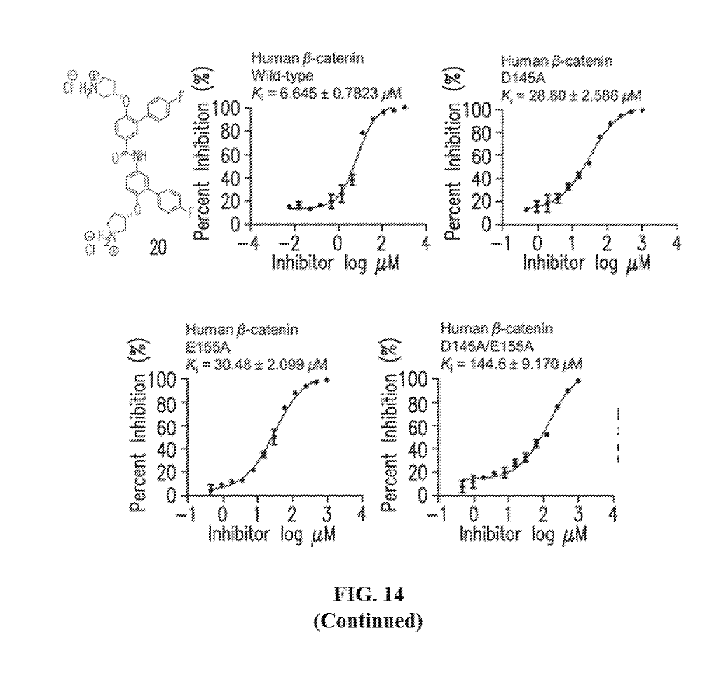

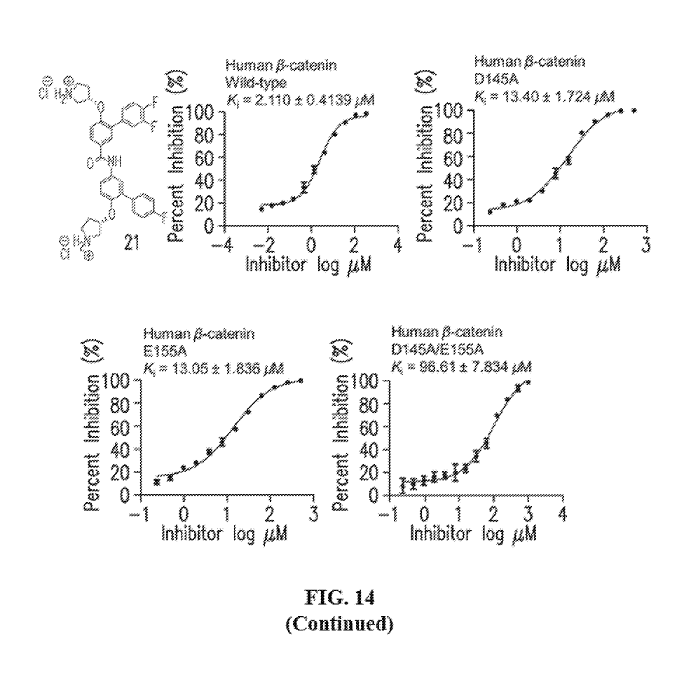

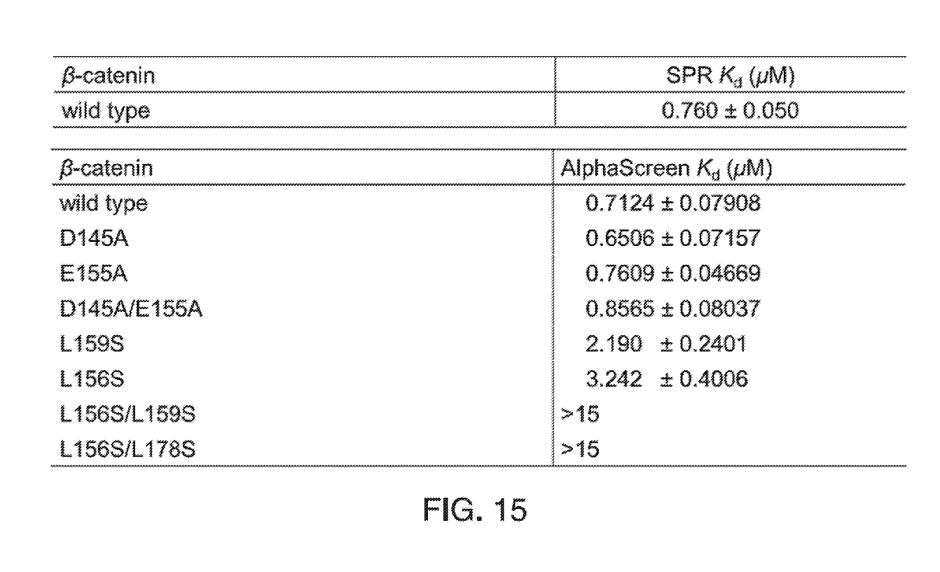

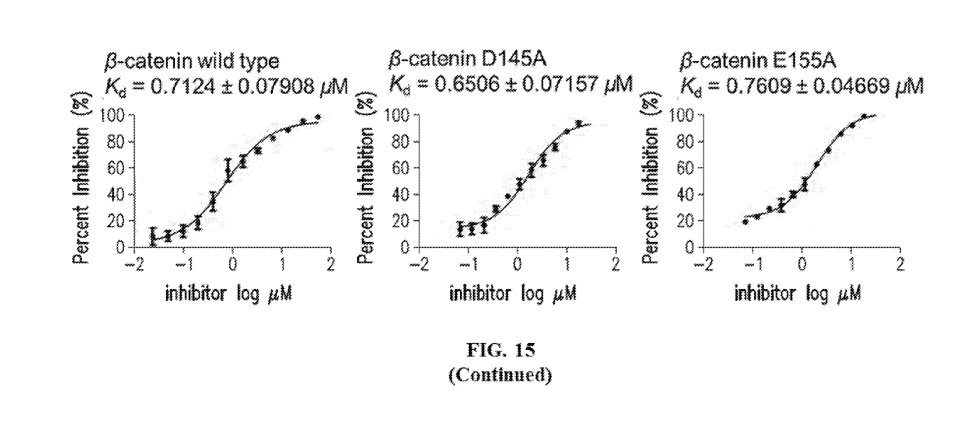

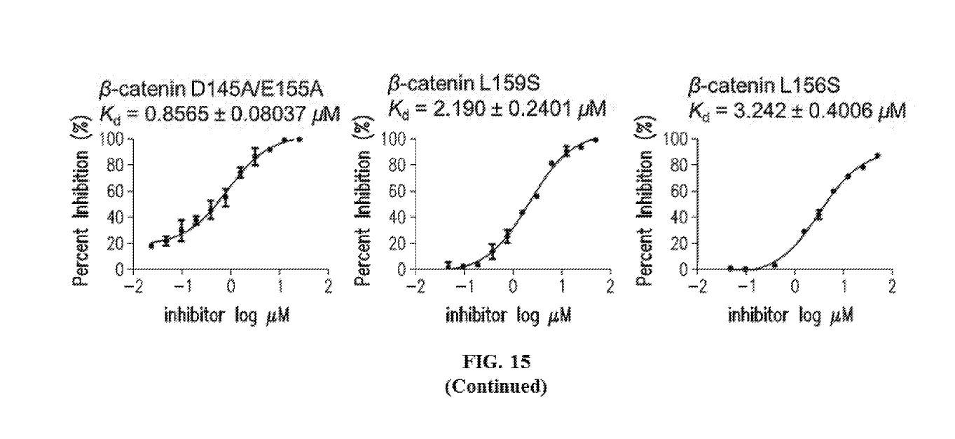

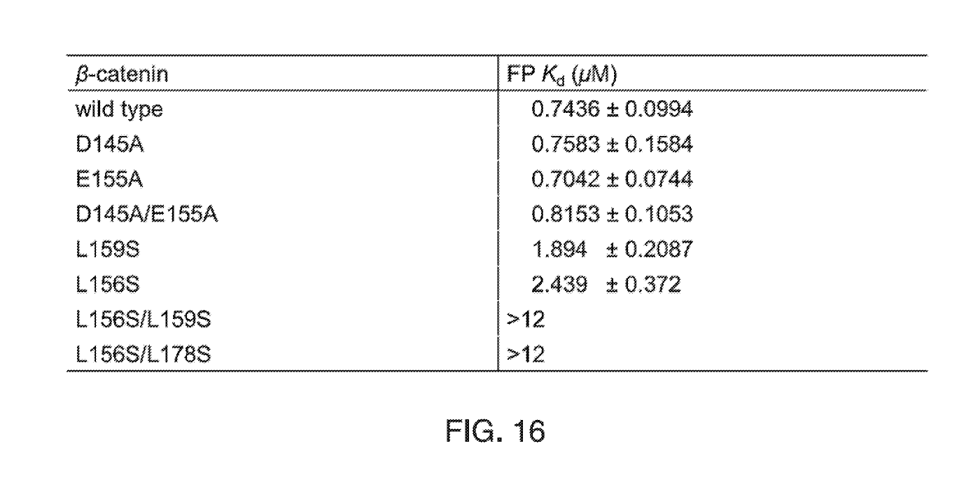

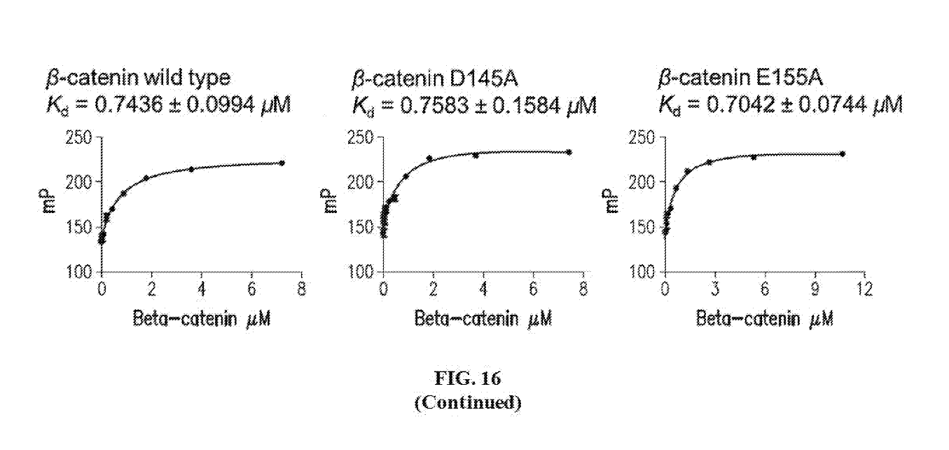

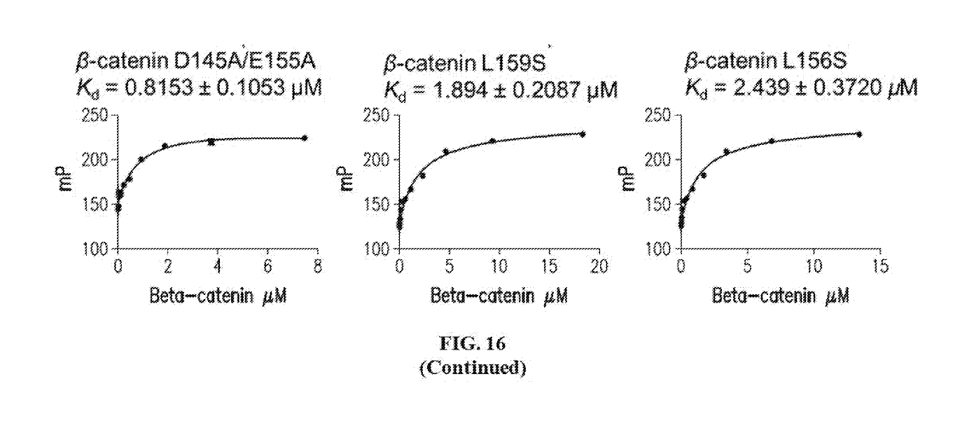

The ITC study showed that 21 bound to wild-type β-catenin with a K_{d} value of 0.333±0.006 μM (FIG. 7D, FIG. 11, FIG. 12A, FIG. 12B, FIG. 13A, and FIG. 13B). This compound did not bind to BCL9 (FIG. 11). The K_{d} value of 21 with β-catenin D145A/E155A was 9.6-fold higher than that with wild-type β-catenin (FIG. 7D), implicating the importance of the side chains of D145 and E155 for inhibitor binding. The K_{d} values of 21 with β-catenin L159S and L156S/L178S were 0.924±0.033 and 1.590±0.045 μM, respectively, suggesting the hydrophobic nature of this binding pocket. Since D145A and E155A did not affect β-catenin/BCL9 PPIs (FIG. 15 and FIG. 16), the AlphaScreen competitive inhibition assays were performed to evaluate the effects of these mutations to the Kᵢ values of 17, 20, and 21. As shown in FIG. 7E and FIG. 14, the Kᵢ values of 21 for β-catenin D145A/BCL9 and β-catenin E155A/BCL9 PPIs were 13.40±1.724 μM and 13.05±1.836 μM, respectively, indicating that the carboxylate side chains of both D145 and E155 of β-catenin were important for the inhibitory potency of 21. The deletion of both side chain carboxylates of D145 and E155 led to a drastic reduction of the inhibitory activity of 21 (Kᵢ=96.61±7.834 μM for β-catenin D145A/E155A double mutant/BCL9 PPIs). The same trend of the Kᵢ value changes was also observed for 17 and 20 (FIG. 7E).

### 7. Inhibitor Selectivity Studies

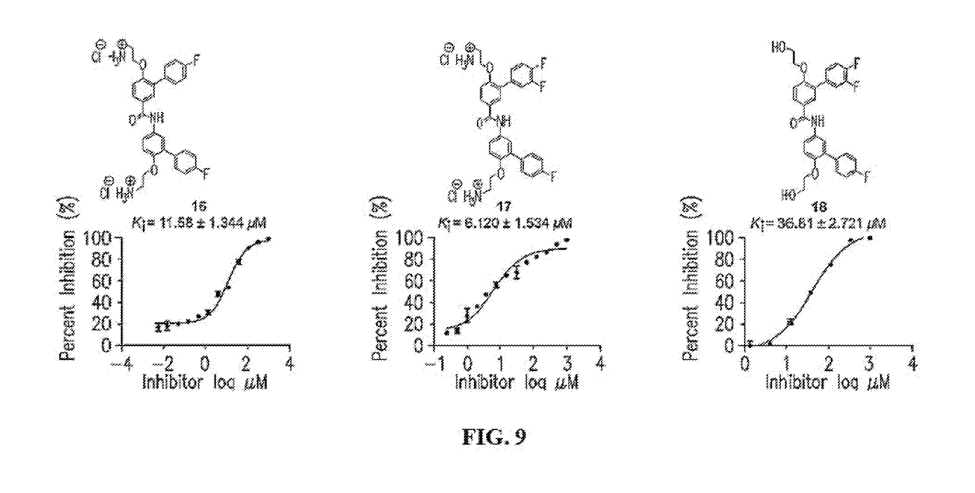

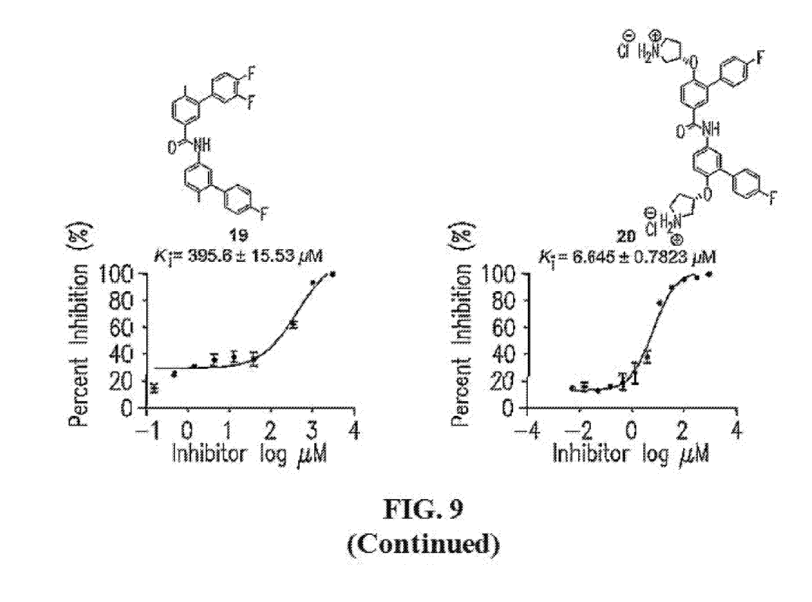

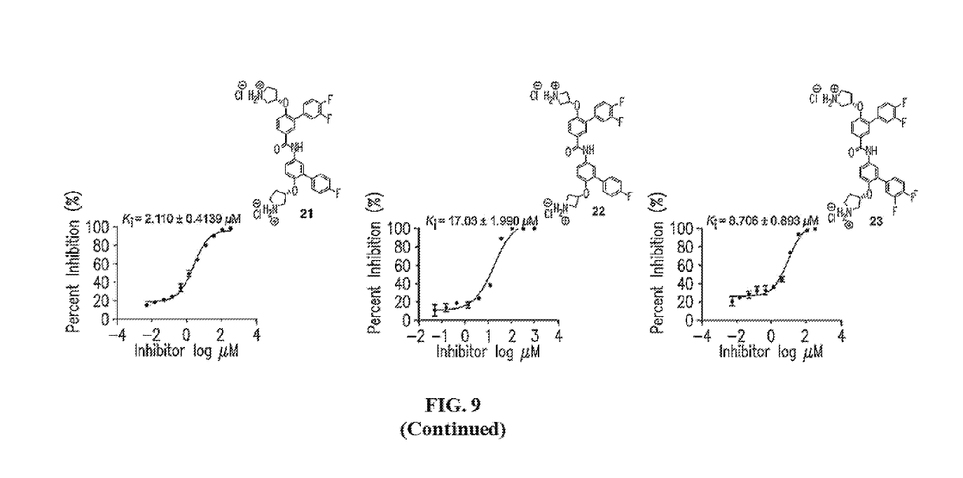

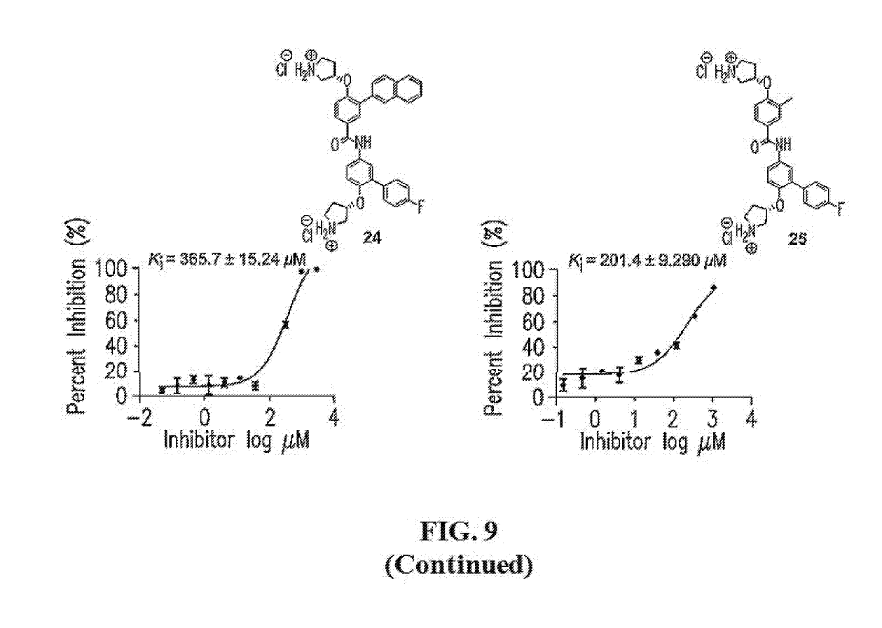

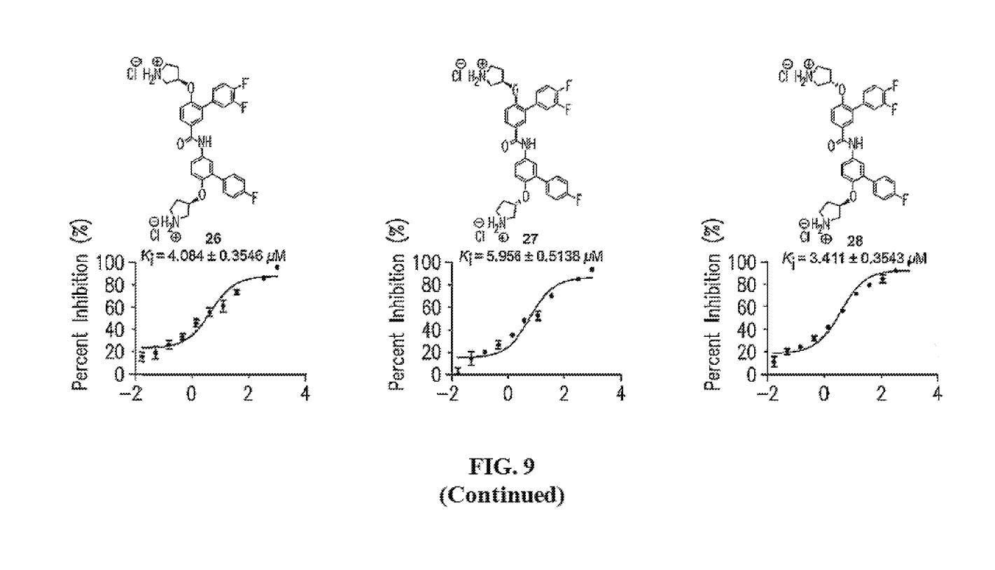

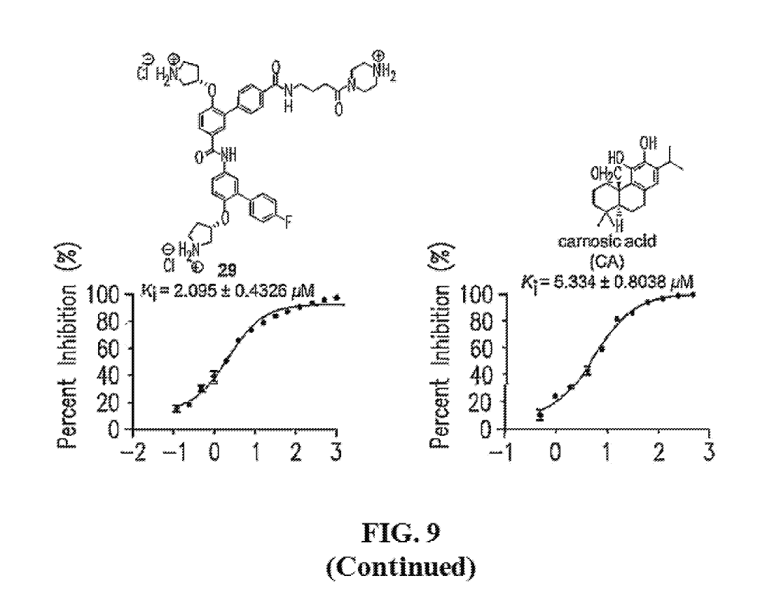

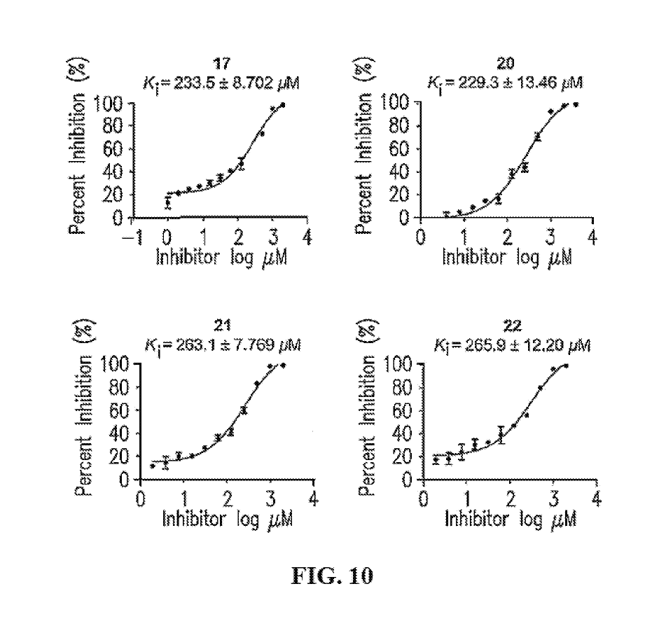

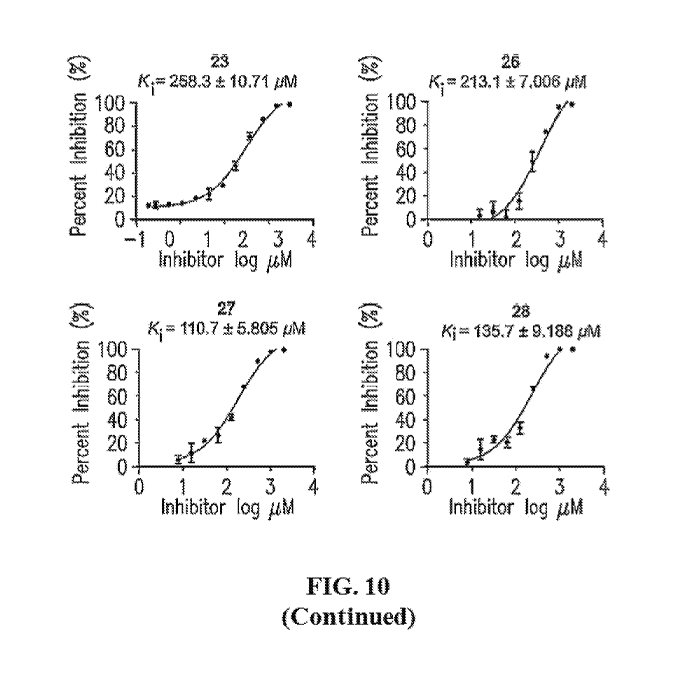

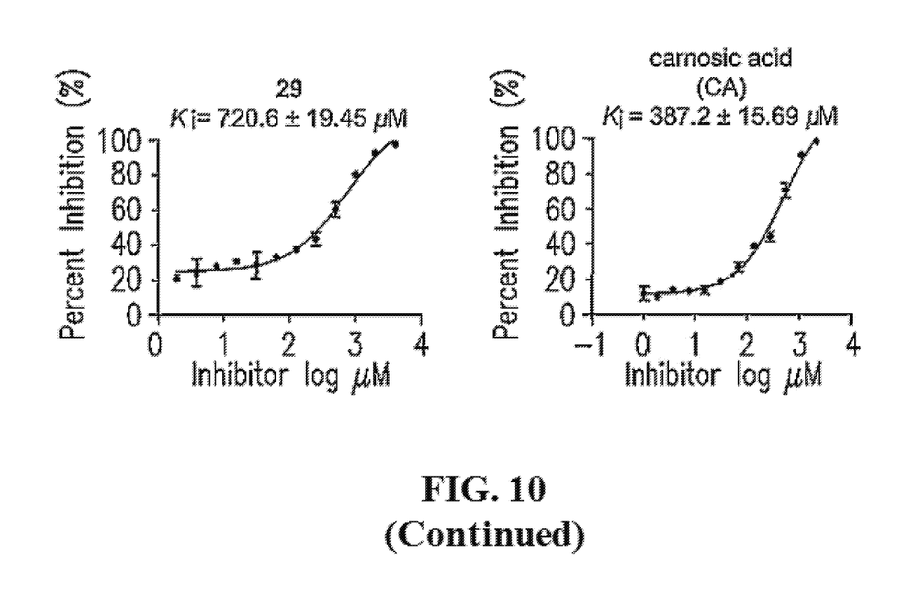

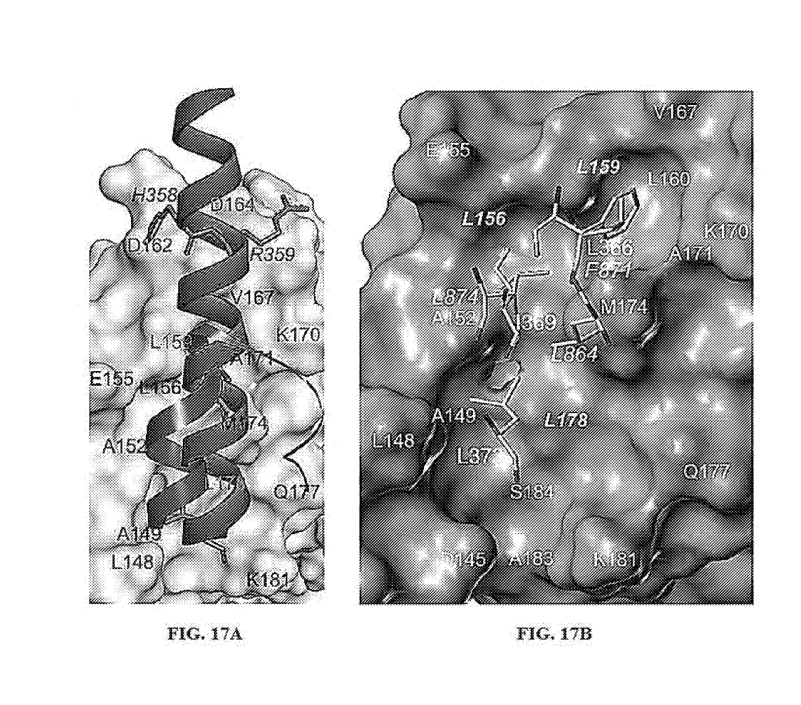

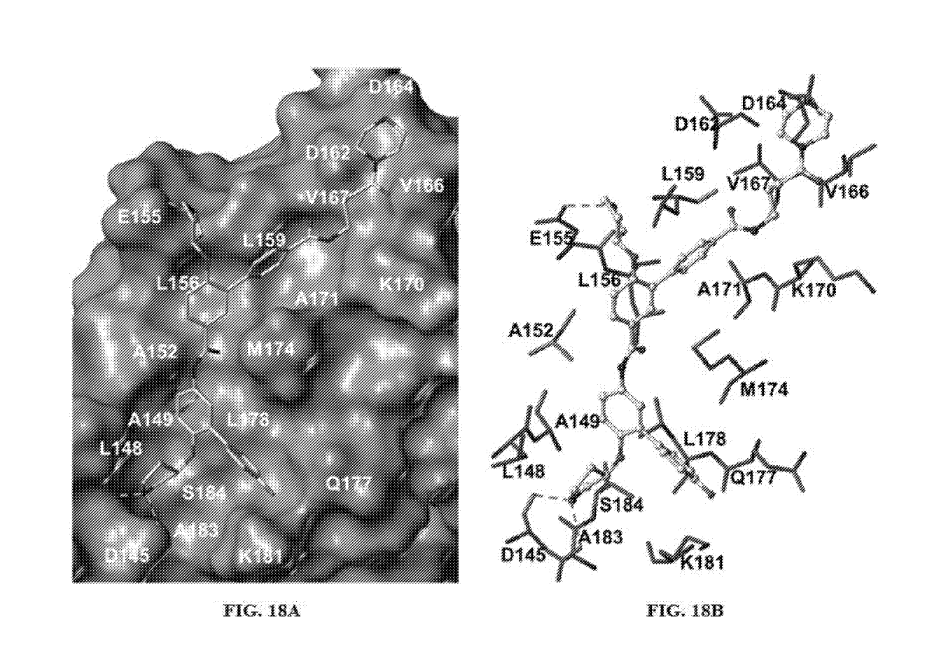

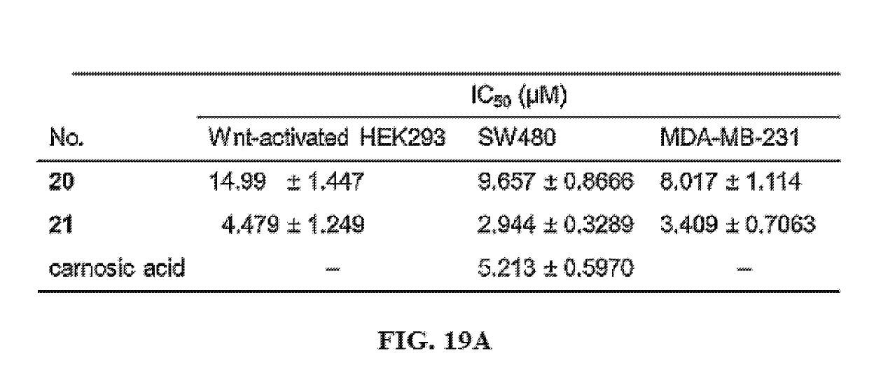

β-Catenin has two functions in cells. One is the interaction with Tcf, BCL9, etc. in the cell nucleus to culminate canonical Wnt signaling. The second is the interaction with cadherin to fulfill its function for cell-cell adhesions. The crystallographic analyses reveal that the interface of β-catenin/BCL9 PPIs is also used to bind region V of E-cadherin (FIG. 17A and FIG. 17B). Two key residues of murine E-cadherin, F871 and L874, occupy the same positions as human BCL9 L366 and 1369 and project to the same hot spot pocket of β-catenin. This overlap presents a potential risk that β-catenin/BCL9 inhibitors might disrupt β-catenin-mediated cell-cell adhesions. The AlphaScreen selectivity assay was used to quantify inhibitor selectivity between β-catenin/BCL9 and β-catenin/E-cadherin PPIs. As shown in FIG. 7C, FIG. 9, and FIG. 10, compound 21 exhibited 125-fold selectivity for β-catenin/BCL9 over β-catenin/E-cadherin PPIs and more selective than carnosic acid in the parallel assay. Noting that hot region 1 in FIG. 4A is only utilized for binding to BCL9, we designed and synthesized 29 to form charge-charge and H-bond interactions with D164 (FIG. 18A and FIG. 18B and Synthesis Scheme 12). The AlphaScreen assay showed that this compound exhibited a comparable inhibitory potency for β-catenin/BCL9 PPIs as 21. However, it exhibited much higher selectivity for β-catenin/BCL9 over β-catenin/E-cadherin PPIs, underscoring the future direction for inhibitor optimization.

### 8. Inhibition of the β-Catenin/BCL9/Tcf Transcriptional Activity and the Expression of Wnt/β-Catenin Target Genes

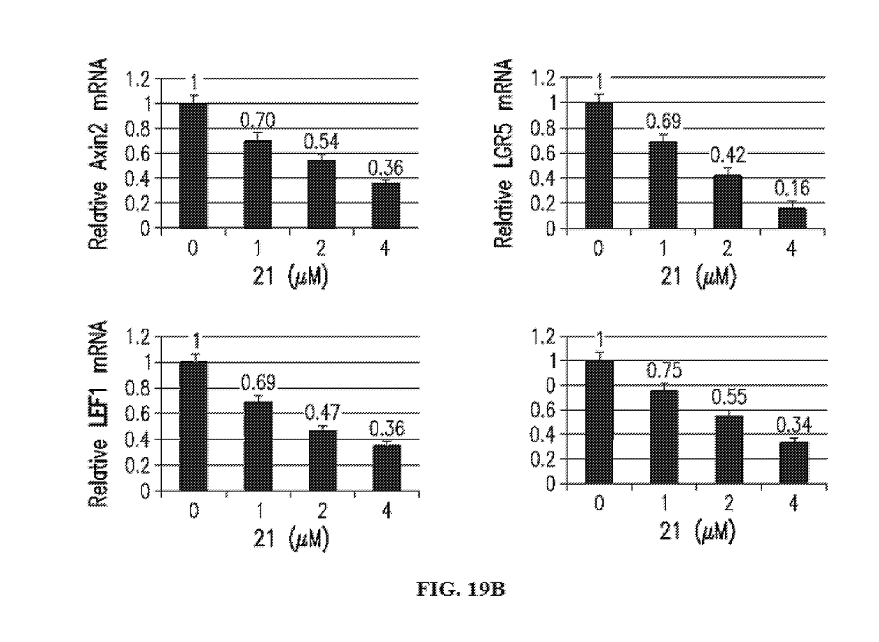

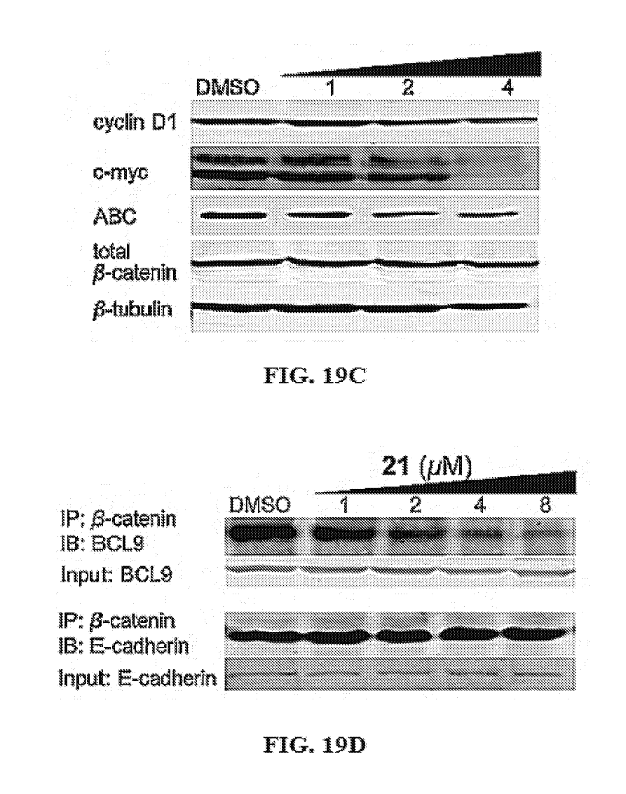

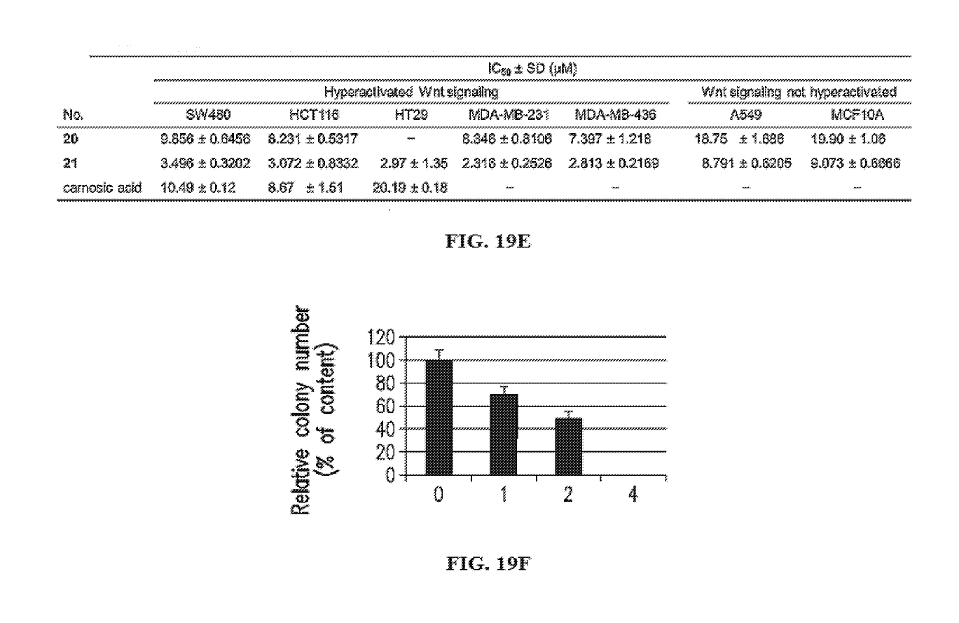

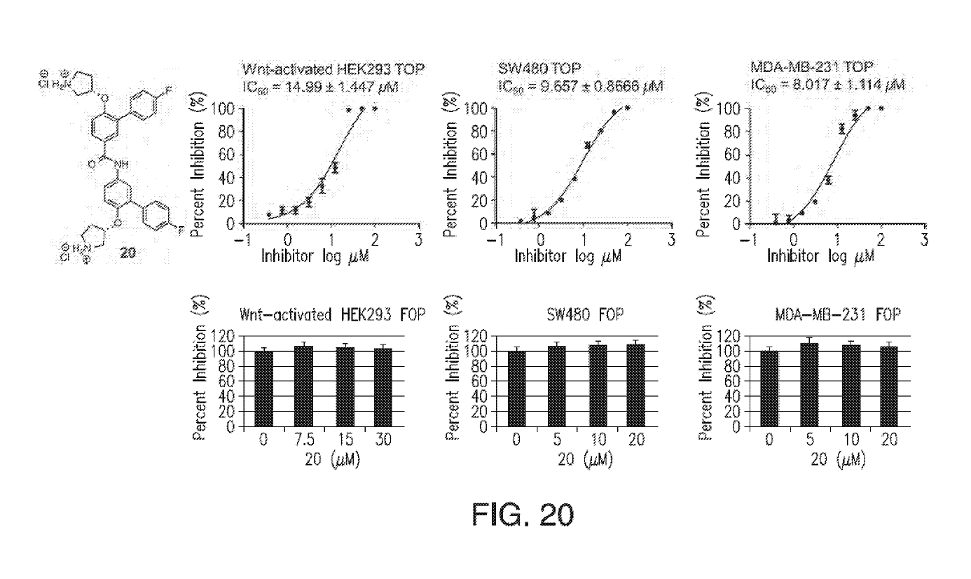

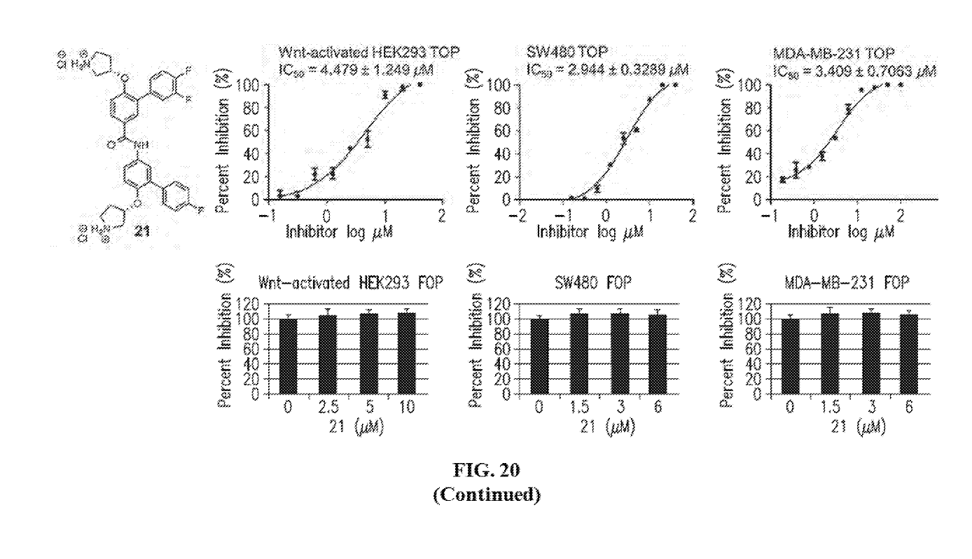

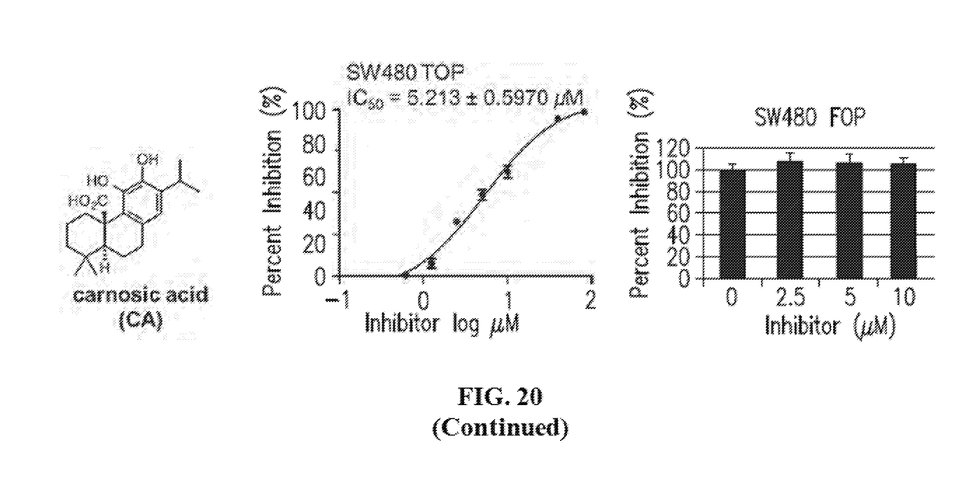

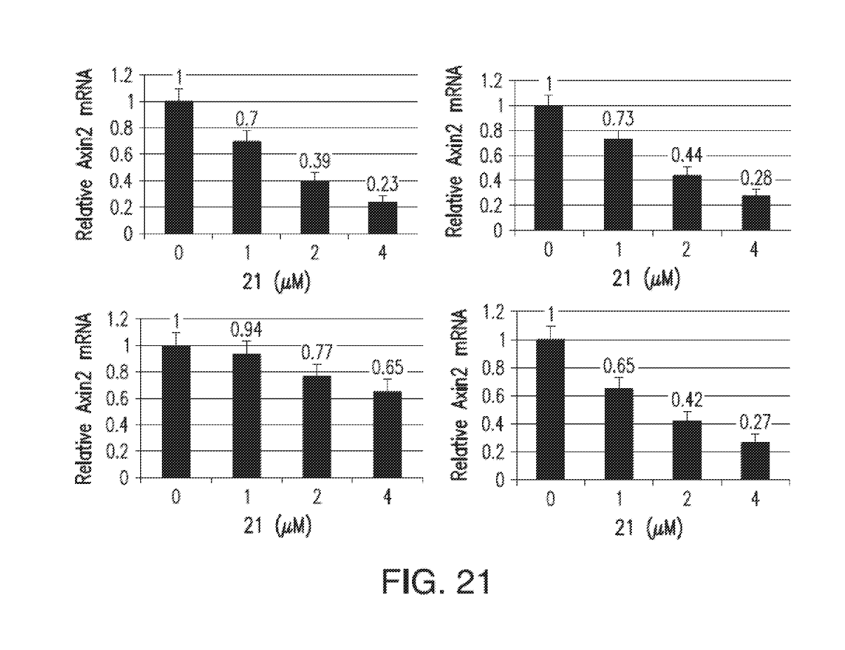

To ascertain whether new β-catenin/BCL9 inhibitors can pass the cell membrane and inhibit the transactivation of canonical Wnt signaling, the Wnt-responsive luciferase reporter assays were performed with Wnt-activated human embryonic kidney cell 293 (HEK293), colorectal cancer cell SW480, and triple-negative breast cancer cell MDA-MB-231. As shown in FIG. 19A and FIG. 20, compounds 20 and 21 can pass the cell membrane and inhibit the TOPFlash luciferase (luciferase reporter with wild-type Tcf4 binding sites) activity without reducing absolute Renilla values (internal controls) and affecting the FOPFlash luciferase (luciferase reporter with mutant Tcf4 binding sites) activity. AXIN2 and LGR5 are the specific target genes for the canonical Wnt signaling pathway. Cyclin D1 and LEF1 are two important target genes that are upregulated in many cancer cells and promote tumorigenesis. As shown in FIG. 19B, compound 21 down-regulated the transcription of AXIN2, LGR5, LEF1, and cyclin D1 in dose-dependent manners in MDA-MB-231 cells. More than 50% of mRNA expression was inhibited at the doses of 2 and 4 μM of 21. The dose-dependent inhibition of mRNA expression of Wnt target genes was also observed for SW480 cells (FIG. 21). The protein expression levels of cyclin D1, c-myc, the active form of β-catenin (ABC), and total β-catenin in SW480 cells were examined by Western blot analysis (FIG. 19C). The protein expression levels of cyclin D1 and c-myc were significantly reduced after the treatment of 21. Compound 21 reduced activated β-catenin in the cell nucleus but had no effect on E-cadherin-bound β-catenin, indicating that 21 does not inhibit the upstream sites of canonical Wnt signaling.

### 9. Cell-Based Inhibitor Selectivity and Inhibition of the Viability of Wnt/β-Catenin-Dependent Cancer Cells

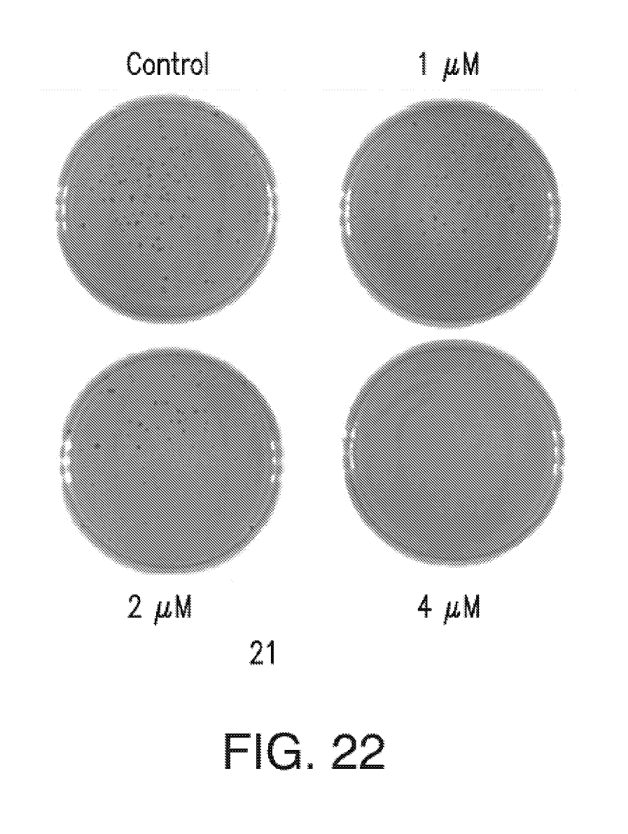

Co-immunoprecipitation experiments were performed to evaluate inhibitor selectivity in a cellular context. As shown in FIG. 19D, compound 21 inhibited β-catenin/BCL9 PPIs in a dose-dependent manner. A parallel experiment indicated that 21 had no effect on β-catenin/E-cadherin PPIs at the concentrations that were sufficient to inhibit β-catenin/BCL9 PPIs. The MTs cell viability assays were performed to assess the effect of 21 on the growth of colorectal cancer cells, SW480, HCT116, and HT29 and triple-negative breast cancer cells, MDA-MB-231 and MDA-MB-436, which have hyperactivated Wnt signaling (FIG. 19E). The MTs assay results showed that both 20 and 21 inhibited cell growth in dose-dependent manners and were more potent than carnosic acid. Compounds 20 and 21 also exhibited cell-base selectivity over Wnt signaling-latent cells, such as lung adenocarcinoma cell line A549 and normal mammary epithelial cell line MCF10A. As shown in FIG. 19F and FIG. 22, compound 21 inhibited the anchorage-independent growth of SW480 cells in a dose-dependent manner.

### G. REFERENCES

Milroy, L.-G., Grossmann, T. N., Hennig, S., Brunsveld, L. & Ottmann, C. Modulators of protein-protein interactions. Chem. Rev. 114, 4695-4748 (2014).
Clackson, T. & Wells, J. A. A hot spot of binding energy in a hormone-receptor interface. Science 267, 383-386 (1995).
Guo, W., Wisniewski, J. A. & Ji, H. Hot spot-based design of small-molecule inhibitors for protein-protein interactions. Bioorg. Med. Chem. Lett. 24, 2546-2554 (2014).
Bullock, B. N., Jochim, A. L. & Arora, P. S. Assessing helical protein interfaces for inhibitor design. J. Am. Chem. Soc. 133, 14220-14223 (2011).
Azzarito, V., Long, K., Murphy, N. S. & Wilson, A. J. Inhibition of α-helix-mediated protein-protein interactions using designed molecules. Nat. Chem. 5, 161-173 (2013).
Jayatunga, M. K. P., Thompson, S. & Hamilton, A. D. α-Helix mimetics: outwards and upwards. Bioorg. Med. Chem. Lett. 24, 717-724 (2014).
Clevers, H. & Nusse, R. Wnt/β-catenin signaling and disease. Cell 149, 1192-1205 (2012).
Anastas, J. N. & Moon, R. T. WNT signalling pathways as therapeutic targets in cancer. Nat. Rev. Cancer 13, 11-26 (2013).
Adachi, S. et al. Role of a BCL9-related β-catenin-binding protein, B9L, in tumorigenesis induced by aberrant activation of Wnt signaling. Cancer Res. 64, 8496-8501 (2004).
de la Roche, M., Worm, J. & Bienz, M. The function of BCL9 in Wnt/β-catenin signaling and colorectal cancer cells. BMC Cancer 8, 199 (2008).
Mani, M. et al. BCL9 promotes tumor progression by conferring enhanced proliferative, metastatic, and angiogenic properties to cancer cells. Cancer Res. 69, 7577-7586 (2009).
Brembeck, F. H. et al. BCL9-2 promotes early stages of intestinal tumor progression. Gastroenterology 141, 1359-1370 (2011).
Brembeck, F. H. et al. Essential role of BCL9-2 in the switch between β-catenin's adhesive and transcriptional functions. Genes Dev. 18, 2225-2230 (2004).
Zhao, J.-J. et al. miR-30-5p functions as a tumor suppressor and novel therapeutic tool by targeting the oncogenic Wnt/β-catenin/BCL9 pathway. Cancer Res. 74, 1801-1813 (2014).
Sampietro, J. et al. Crystal structure of a β-catenin/BCL9/Tcf4 complex. Mol. Cell 24, 293-300 (2006).
Kawamoto, S. A. et al. Design of triazole-stapled BCL9 α-helical peptides to target the β-catenin/B-cell CLL/lymphoma 9 (BCL9) protein-protein interaction. J. Med. Chem. 55, 1137-1146 (2012).
Takada, K. et al. Targeted disruption of the BCL9/β-catenin complex inhibits oncogenic Wnt signaling. Sci. Transl. Med. 4, 148ra117 (2012).
de la Roche, M. et al. An intrinsically labile α-helix abutting the BCL9-binding site of β-catenin is required for its inhibition by carnosic acid. Nat. Commun. 3, 680 (2012).
Hoffmans, R. & Basler, K. Identification and in vivo role of the Armadillo-Legless interaction. Development 131, 4393-4400 (2004).
Hoffmans. R. & Basler, K. BCL9-2 binds Arm/β-catenin in a Tyr142-independent manner and requires Pygopus for its function in Wg/Wnt signaling. Mech. Dev. 124, 59-67 (2007).
Valenta, T. et al. Probing transcription-specific outputs of β-catenin in vivo. Genes Dev. 25, 2631-2643 (2011).
Kawamoto, S. A. et al. Analysis of the interaction of BCL9 with β-catenin and development of fluorescence polarization and surface plasmon resonance binding assays for this interaction. Biochemistry 48, 9534-9541 (2009).
Meireles, L. M. C., Dömling, A. S. & Camacho, C. J. ANCHOR: a web server and database for analysis of protein-protein interaction binding pockets for drug discovery. Nucleic Acids Res. 38, W407-W411 (2010).
Koes, D. R. & Camacho, C. J. PocketQuery: protein-protein interaction inhibitor starting points from protein-protein interaction structure. Nucleic Acids Res. 40, W387-W392 (2012).
Halgren, T. A. Identifying and characterizing binding sites and assessing druggability. J. Chem. Inf. Model. 49, 377-389 (2009).
Ji, H. et al. Minimal pharmacophoric elements and fragment hopping, an approach directed at molecular diversity and isozyme selectivity. Design of selective neuronal nitric oxide synthase inhibitors. J. Am. Chem. Soc. 130, 3900-3914 (2008).
Ji, H. et al. Discovery of highly potent and selective inhibitors of neuronal nitric oxide synthase by fragment hopping. J. Med. Chem. 52, 779-797 (2009).
Yu, B., Huang, Z., Zhang, M., Dillard, D. R. & Ji, H. Rational design of small-molecule inhibitors for β-catenin/T-cell factor protein-protein interactions by bioisostere replacement. ACS Chem. Biol. 8 (3), 524-529 (2013).
Morris, G. M. et al. AutoDock4 and AutoDockTools4: Automated docking with selective receptor flexibility. J Comput. Chem. 30, 2785-2791 (2009).
Friesner, R. A. et al. Extra precision glide: docking and scoring incorporating a model of hydrophobic enclosure for protein-ligand complexes. J. Med. Chem. 49, 6177-6196 (2006).
Levin, K. B. et al. Following evolutionary paths to protein-protein interactions with high affinity and selectivity. Nat. Struct. Mol. Biol. 16, 1049-1055 (2009).
Meenan, N. A. G. et al. The structural and energetic basis for high selectivity in a high-affinity protein-protein interaction. Proc. Natl. Acad. Sci. U.S.A. 107, 10080-10085 (2010).
Kosloff, M., Travis, A. M., Bosch, D. E., Siderovski, D. P. & Arshavsky, V. Y. Integrating energy calculations with functional assays to decipher the specificity of G protein-RGS protein interactions. Nat. Struct. Mol. Biol. 18, 846-853 (2011).
Halgren, T. A. Identifying and characterizing binding sites and assessing druggability. J. Chem. Inf. Model. 49, 377-389 (2009).
Nikolovska-Coleska, Z. et al. Development and optimization of a binding assay for the XIAP BIR3 domain using fluorescence polarization. Anal. Biochem. 32, 261-273 (2004).
Hoffmans, R. & Basler, K. Identification and in vivo role of the Armadillo-Legless interaction. Development 131 (17), 4393-4400 (2004).
Hoffmans. R. & Basler, K. BCL9-2 binds Arm/β-catenin in a Tyr142-independent manner and requires Pygopus for its function in Wg/Wnt signaling. Mech. Dev. 124 (1), 59-67 (2007).
Valenta, T., Gay, M., Steiner, S., Draganova, K., Zemke, M., Hoffmans, R., Cinelli, P., Aguet, M., Sommer, L. & Basler, K. Probing transcription-specific outputs of 1-catenin in vivo. Genes Dev. 25 (24), 2631-2643 (2011).
Sampietro, J., Dahlberg, C. L., Cho, U. S., Hinds, T. R., Kimelman, D. & Xu, W. Crystal structure of a β-catenin/BCL9/Tcf4 complex. Mol. Cell 24 (2), 293-300 (2006).
de la Roche, M., Worm, J. & Bienz, M. The function of BCL9 in Wnt/β-catenin signaling and colorectal cancer cells. BMC Cancer 8, 199 (2008).
Kawamoto, S. A., Thompson, A. D., Coleska, A., Nikolovska-Coleska, Z., Yi, H. & Wang, S. Analysis of the interaction of BCL9 with β-catenin and development of fluorescence polarization and surface plasmon resonance binding assays for this interaction. Biochemistry 48 (40), 9534-9541 (2009).
Ji, H., Stanton, B. Z., Igarashi, J., Li, H., Martásek, P., Roman, L. J., Poulos, T. L. & Silverman, R. B. Minimal pharmacophoric elements and fragment hopping, an approach directed at molecular diversity and isozyme selectivity. Design of selective neuronal nitric oxide synthase inhibitors. J. Am. Chem. Soc. 130 (12), 3900-3914 (2008).
Ji, H., Li, H., Martásek, P., Roman, L. J., Poulos, T. L. & Silverman, R. B. Discovery of highly potent and selective inhibitors of neuronal nitric oxide synthase by fragment hopping. J. Med. Chem. 52 (3), 779-797 (2009).
Yu, B., Huang, Z., Zhang, M., Dillard, D. R. & Ji, H. Rational design of small-molecule inhibitors for β-catenin/T-cell factor protein-protein interactions by bioisostere replacement. ACS Chem. Biol. 8 (3), 524-529 (2013).
Jochim, A. L. & Arora, P. S. Assessment of helical interfaces in protein-protein interactions. Mol. Biosyst. 5 (9), 924-926 (2009).
Jochim, A. L. & Arora, P. S. Systematic analysis of helical protein interfaces reveals targets for synthetic inhibitors. ACS Chem. Biol. 5 (10), 919-923 (2010).
Bullock, B. N., Jochim, A. L. & Arora, P. S. Assessing helical protein interfaces for inhibitor design. J Am. Chem. Soc. 133 (36), 14220-14223 (2011).
Bergey, C. M., Watkins, A. M. & Arora, P. S. HippDB: a database of readily targeted helical protein-protein interactions. Bioinformatics 29 (21), 2806-2807 (2013).
Sampietro, J., Dahlberg, C. L., Cho, U. S., Hinds, T. R., Kimelman, D. & Xu, W. Crystal structure of a β-catenin/BCL9/Tcf4 complex. Mol. Cell 24 (2), 293-300 (2006).
Sharma, V., Sharma, S., Hoener zu Bentrup, K., McKinney, J. D., Russell, D. G., Jacobs, W. R. Jr. & Sacchettini, J. C. Structure of isocitrate lyase, a persistence factor of Mycobacterium tuberculosis. Nat. Struct. Biol. 7 (8), 663-668 (2000).
Czabotar, P. E., Westphal, D., Dewson, G., Ma, S., Hockings, C., Fairlie, W. D., Lee, E. F., Yao, S., Robin, A. Y., Smith, B. J., Huang, D. C. S., Kluck, R. M., Adams, J. M. & Colman, P. M. Bax crystal structures reveal how BH3 domains activate Bax and nucleate its oligomerization to induce apoptosis. Cell 152 (3), 519-531 (2013).
Zhao, X., Ghaffari, S., Lodish, H., Malashkevich, V. N. & Kim, P. S. Structure of the Bcr-Abl oncoprotein oligomerization domain. Nat. Struct. Biol. 9 (2), 117-120 (2002).
Terradot, L., Bayliss, R., Oomen, C., Leonard, G. A., Baron, C. & Waksman, G. Structures of two core subunits of the bacterial type IV secretion system, VirB8 from Brucella suis and ComB10 from Helicobacter pylori. Proc. Natl. Acad. Sci. U.S.A. 102 (12), 4596-4601 (2005).
Muto, S., Senda, M., Akai, Y., Sato, L., Suzuki, T., Nagai, R., Senda, T. & Horikoshi, M. Relationship between the structure of SET/TAF-Iβ/INHAT and its histone chaperone activity. Proc. Natl. Acad. Sci. U.S.A. 104 (11), 4285-4290 (2007).
Jiao, L., Ouyang, S., Liang, M., Niu, F., Shaw, N., Wu, W., Ding, W., Jin, C., Peng, Y., Zhu, Y., Zhang, F., Wang, T., Li, C., Zuo, X., Luan, C.-H., Li, D. & Liu, Z.-J. Structure of severe fever with thrombocytopenia syndrome virus nucleocapsid protein in complex with suramin reveals therapeutic potential. J. Virol. 87 (12), 6829-6839 (2013).
Milbum, M. V., Hassell, A. M., Lambert, M. H., Jordan, S. R., Proudfoot, A. E., Graber, P. & Wells, T. N. C. A novel dimer configuration revealed by the crystal structure at 2.4 Å resolution of human interleukin-5. Nature 363 (6425), 172-176 (1993).
Liu, X., Dai, S., Zhu, Y., Marrack, P. & Kappler, J. W. The structure of a Bcl-x_{L}/Bim fragment complex: implications for Bim function. Immunity 19 (3), 341-352 (2003).
Czabotar, P. E., Lee, E. F., van Delft, M. F., Day, C. L., Smith, B. J., Huang, D. C. S., Fairlie, W. D., Hinds, M. G. & Colman, P. M. Structural insights into the degradation of Mcl-1 induced by BH3 domains. Proc. Natl. Acad. Sci. U.S.A. 104 (15), 6217-6222 (2007).
Chen, G., Wang, C., Fuqua, C., Zhang, L.-H. & Chen, L. Crystal structure and mechanism of TraM2, a second quorum-sensing antiactivator of Agrobacterium tumefaciens strain A6. J. Bacteriol. 188 (23), 8244-8251 (2006).
Lee, E. F., Clarke, O. B., Evangelista, M., Feng, Z., Speed, T. P., Tchoubrieva, E. B., Strasser, A., Kalinna, B. H., Colman, P. M. & Fairlie, W. D. Discovery and molecular characterization of a Bcl-2-regulated cell death pathway in schistosomes. Proc. Natl. Acad. Sci. U.S.A. 108 (17), 6999-7003 (2011).
Smits, C., Czabotar, P. E., Hinds, M. G. & Day, C. L. Structural plasticity underpins promiscuous binding of the prosurvival protein A1. Structure 16 (5), 818-829 (2008).
Ku, B., Liang, C., Jung, J. U. & Oh, B.-H. Evidence that inhibition of BAX activation by BCL-2 involves its tight and preferential interaction with the BH3 domain of BAX. Cell Res. 21 (4), 627-641 (2011).
Leppänen, V.-M., Prota, A. E., Jeltsch, M., Anisimov, A., Kalkkinen, N., Strandin, T., Lankinen, H., Goldman, A., Ballmer-Hofer, K. & Alitalo, K. Structural determinants of growth factor binding and specificity by VEGF receptor 2. Proc. Natl. Acad. Sci. U.S.A. 107 (6), 2425-2430 (2010).
Quinaud, M., Plé, S., Job, V., Contreras-Martel, C., Simorre, J.-P., Attree, I. & Dessen, A. Structure of the heterotrimeric complex that regulates type III secretion needle formation. Proc. Natl. Acad. Sci. U.S.A. 104 (19), 7803-7808 (2007).
Li, Z., Zhao, B., Wang, P., Chen, F., Dong, Z., Yang, H., Guan, K.-L. & Xu, Y. Structural insights into the YAP and TEAD complex. Genes Dev. 24 (3), 235-240 (2010).
Friberg, A., Vigil, D., Zhao, B., Daniels, R. N., Burke, J. P., Garcia-Barrantes, P. M., Camper, D., Chauder, B. A., Lee, T., Olejniczak, E. T. & Fesik, S. W. Discovery of potent myeloid cell leukemia 1 (Mcl-1) inhibitors using fragment-based methods and structure-based design. J. Med. Chem. 56 (1), 15-30 (2013).
Ku, B., Woo, J.-S., Liang, C., Lee, K.-H., Hong, H.-S., E, X., Kim, K.-S., Jung, J. U. & Oh, B.-H. Structural and biochemical bases for the inhibition of autophagy and apoptosis by viral BCL-2 of murine γ-herpesvirus 68. PLoS Pathog. 4 (2), e25 (2008).
Heikkila, T., Wheatley, E., Crighton, D., Schroder, E., Boakes, A., Kaye, S. J., Mezna, M., Pang, L., Rushbrooke, M., Tumbull, A. & Olson, M. F. Co-crystal structures of inhibitors with MRCKβ, a key regulator of tumor cell invasion. PLoS One 6 (9), e24825 (2011).
Grigoriu, S., Bond, R., Cossio, P., Chen, J. A., Ly, N., Hummer, G., Page, R., Cyert, M. S. & Peti, W. The molecular mechanism of substrate engagement and immunosuppressant inhibition of calcineurin. PLoS Biol. 11 (2), e1001492 (2013).
Meireles, L. M. C., Dömling, A. S. & Camacho, C. J. ANCHOR: a web server and database for analysis of protein-protein interaction binding pockets for drug discovery. Nucleic Acids Res. 38, W407-W411 (2010).
Camacho, C. J. & Zhang, C. FastContact: rapid estimate of contact and binding free energies. Bioinformatics 21 (10), 2534-2536 (2005).
Camacho, C. J., Ma, H. & Champ, P. C. Scoring a diverse set of high-quality docked conformations: a metascore based on electrostatic and desolvation interactions. Proteins 63 (4), 868-877 (2006).
Koes, D. R. & Camacho, C. J. PocketQuery: protein-protein interaction inhibitor starting points from protein-protein interaction structure. Nucleic Acids Res. 40, W387-W392 (2012).
Kortemme, T., Kim, D. E. & Baker, D. Computational alanine scanning of protein-protein interfaces. Sci. STKE 2004 (219), pl2 (2004).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 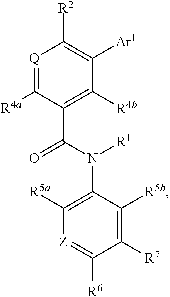
wherein Q is selected from N and CR^{4c};
wherein Z is selected from N and CR^{5c};
wherein R¹ is selected from hydrogen and C1-C4 alkyl;
wherein R² is selected from -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂—Cy¹, —NHCH₂—Cy²; —OCH₂—Cy¹, and —OCH₂—Cy²;
wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and
wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl;
wherein each of R^{4a}, R^{4b}, and R^{4c}, when present, is independently selected from hydrogen, C1-C4 alkyl, C1-C4 monohaloalkyl, and C1-C4 polyhaloalkyl;
wherein each of R^{5a}, R^{5b}, and R^{5c}, when present, is independently selected from hydrogen, C1-C4 alkyl, C1-C4 monohaloalkyl, and C1-C4 polyhaloalkyl;
wherein R⁶ is selected from hydrogen, -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂—Cy³, —NHCH₂—Cy⁴; —OCH₂—Cy³, and —OCH₂—Cy⁴;
wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and
wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl;
wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
wherein each occurrence of R²⁰, when present, is independently selected from C1-C3 alkyl, C1-C3 monohaloalkyl, C1-C3 polyhaloalkyl, and cyclopropyl;
wherein each occurrence of R^{21a} and R^{21b}, when present, is independently selected from hydrogen, C1-C3 alkyl, C1-C3 monohaloalkyl, C1-C3 polyhaloalkyl, and cyclopropyl;
wherein R⁷ is selected from Ar², -A¹-A²-Ar², and 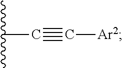
wherein each of A¹ and A², when present, is independently selected from O, NH, and CH₂, provided that each of A¹ and A² is simultaneously O; and
wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
or a pharmaceutically acceptable salt thereof.

2. The compound of claim 1, wherein R² is —O-Cy².

3. The compound of claim 1, wherein R⁶ is —O-Cy⁴.

4. The compound of claim 1, wherein Cy², when present, is an unsubstituted pyrrolidinyl.

5. The compound of claim 1, wherein Cy⁴, when present, is an unsubstituted pyrrolidinyl.

6. The compound of claim 1, wherein Ar¹ is phenyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H.

7. The compound of claim 1, wherein R⁷ is Ar².

8. The compound of claim 7, wherein Ar² is phenyl substituted with 0, 1, 2, or 3 groups independently selected from halogen, —CN, C1-C3 alkyl, C1-C3 monohaloalkyl, C1-C3 polyhaloalkyl, cyclopropyl, and —CO₂H.

9. The compound of claim 1, having a structure represented by a formula: 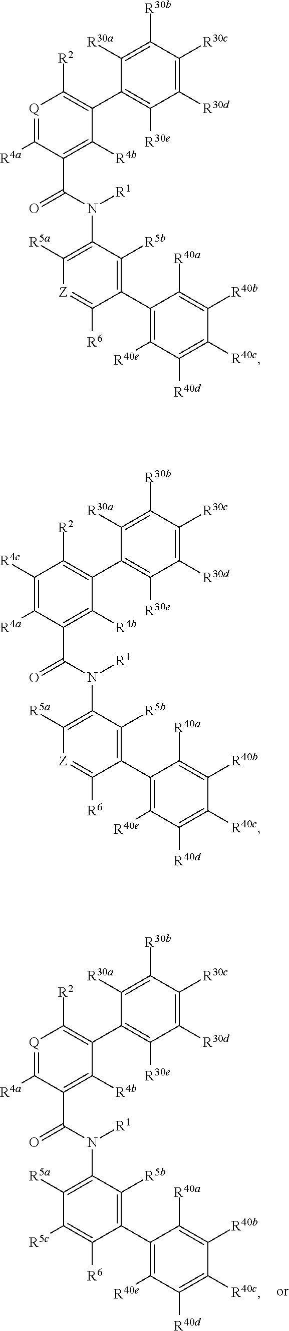 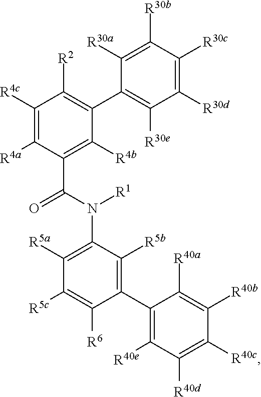
wherein each of R^{30a}, R^{30b}, R^{30c}, R^{30d} and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and
wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen.

10. The compound of claim 1, having a structure represented by a formula: 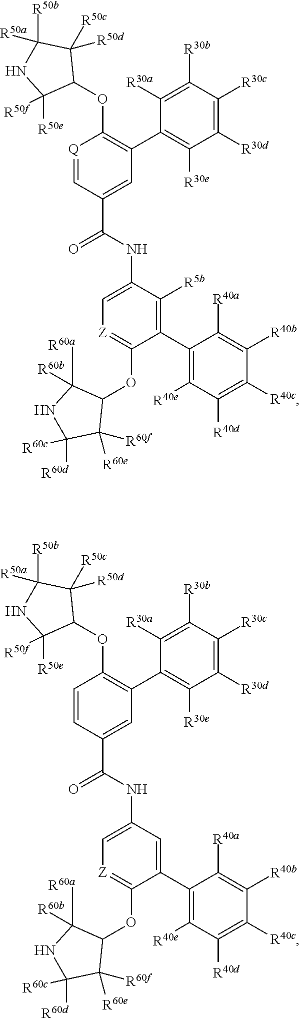 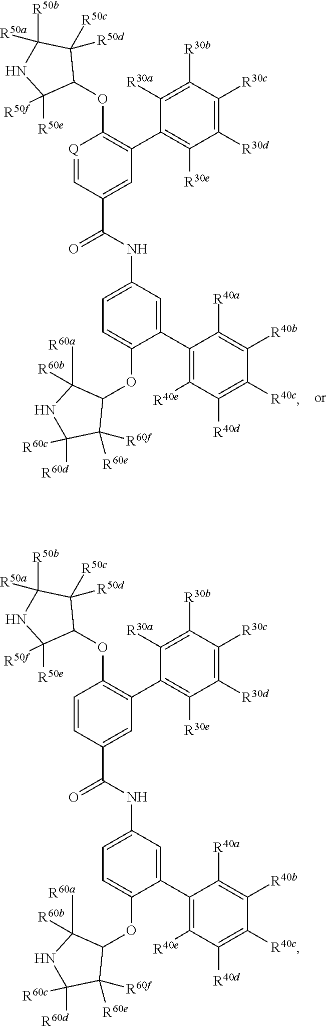
wherein each of R^{30a}, R^{30b}, R^{30c}, R^{30d}, and R^{30e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{30a}, R^{30b}, R^{30c}, R^{30d}, and R^{30e} are hydrogen; and
wherein each of R^{40a}, R^{40b}, R^{40c}, R^{40d}, and R^{40e} is independently selected from hydrogen, halogen, —CN, C1-C3 alkyl, C1-C3 monohaloalkyl, C1-C3 polyhaloalkyl, cyclopropyl, and —CO₂H; and wherein at least two of R^{40a}, R^{40b}, R^{40c}, R^{40d}, and R^{40e} are hydrogen;
wherein each of R^{50a}, R^{50b}, R^{50c}, R^{50d}, R^{50e}, and R^{50f} is independently selected from hydrogen, halogen, C1-C4 alkyl, C1-C4 monohaloalkyl, and C1-C4 polyhaloalkyl; and wherein at least three of R^{50a}, R^{50b}, R^{50c}, R^{50d}, R^{50e}, and R^{50f} are hydrogen; and
wherein each of R^{60a}, R^{60b}, R^{60c}, R^{60d}, R^{60e}, and R60^{f} is independently selected from hydrogen, halogen, C1-C4 alkyl, C1-C4 monohaloalkyl, and C1-C4 polyhaloalkyl; and wherein at least three of R^{60a}, R^{60b}, R^{60c}, R^{60d}, R^{60e}, and R^{60f} are hydrogen.

11. The compound of claim 1, present as: 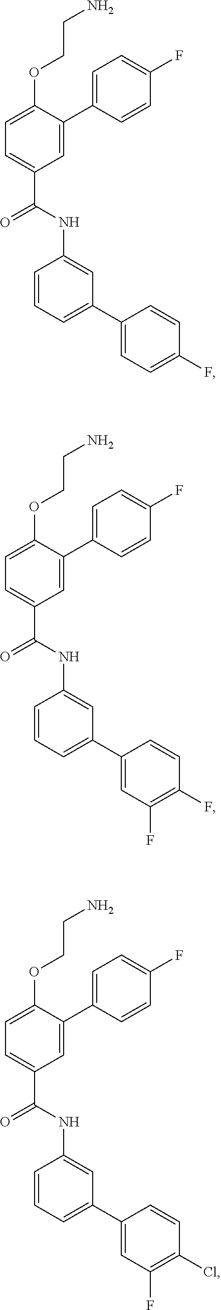 [SMILES: NCCOc1ccc(C(=O)Nc2cccc(-c3ccc(F)cc3)c2)cc1-c1ccc(F)cc1 ; NCCOc1ccc(C(=O)Nc2cccc(-c3ccc(F)c(F)c3)c2)cc1-c1ccc(F)cc1 ; NCCOc1ccc(C(=O)Nc2cccc(-c3ccc(Cl)c(F)c3)c2)cc1-c1ccc(F)cc1] 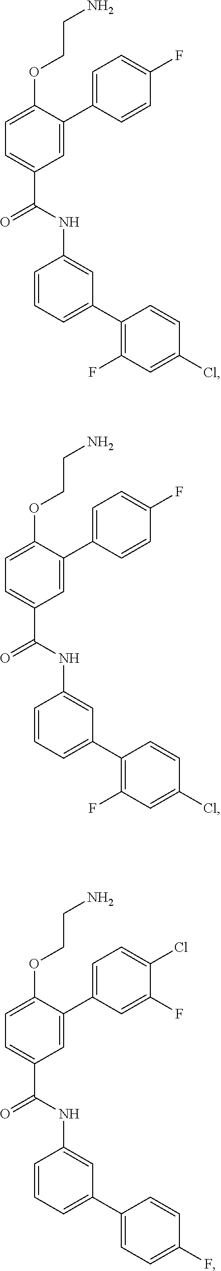 [SMILES: NCCOc1ccc(C(=O)Nc2cccc(-c3ccc(Cl)cc3F)c2)cc1-c1ccc(F)cc1 ; NCCOc1ccc(C(=O)Nc2cccc(-c3ccc(Cl)cc3F)c2)cc1-c1ccc(F)cc1 ; NCCOc1ccc(C(=O)Nc2cccc(-c3ccc(F)cc3)c2)cc1-c1ccc(Cl)c(F)c1] 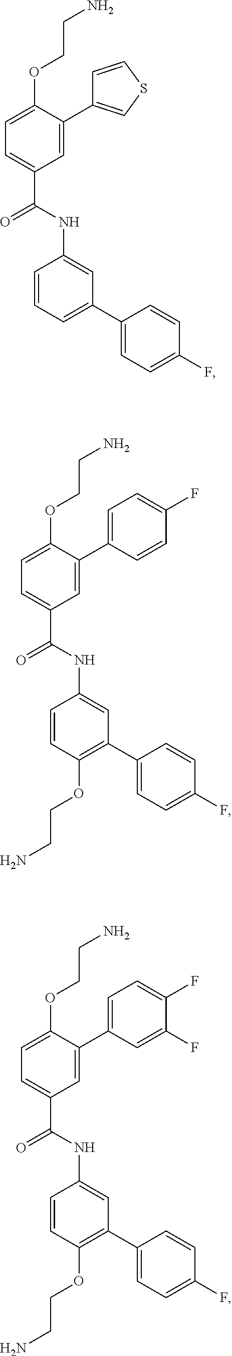 [SMILES: NCCOc1ccc(C(=O)Nc2cccc(-c3ccc(F)cc3)c2)cc1-c1ccsc1 ; NCCOc1ccc(NC(=O)c2ccc(OCCN)c(-c3ccc(F)cc3)c2)cc1-c1ccc(F)cc1 ; NCCOc1ccc(NC(=O)c2ccc(OCCN)c(-c3ccc(F)c(F)c3)c2)cc1-c1ccc(F)cc1] 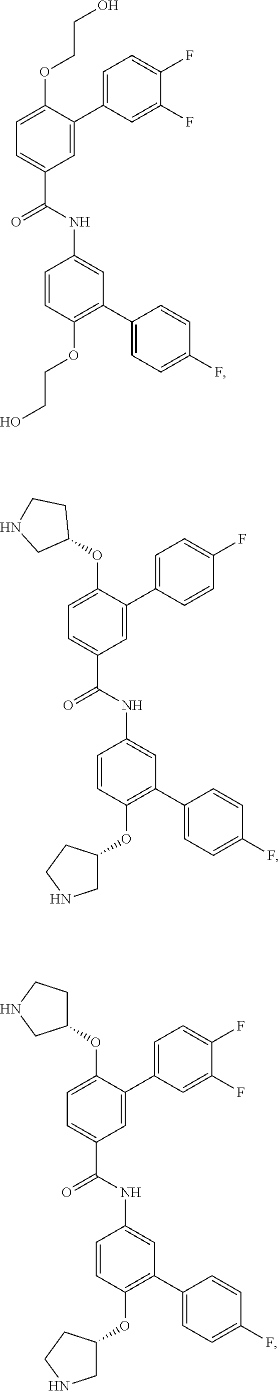 [SMILES: O=C(Nc1ccc(OCCO)c(-c2ccc(F)cc2)c1)c1ccc(OCCO)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc(F)cc2)c1 ; O=C(Nc1ccc(O[C@H]2CCNC2)c(-c2ccc(F)cc2)c1)c1ccc(O[C@H]2CCNC2)c(-c2ccc(F)c(F)c2)c1] 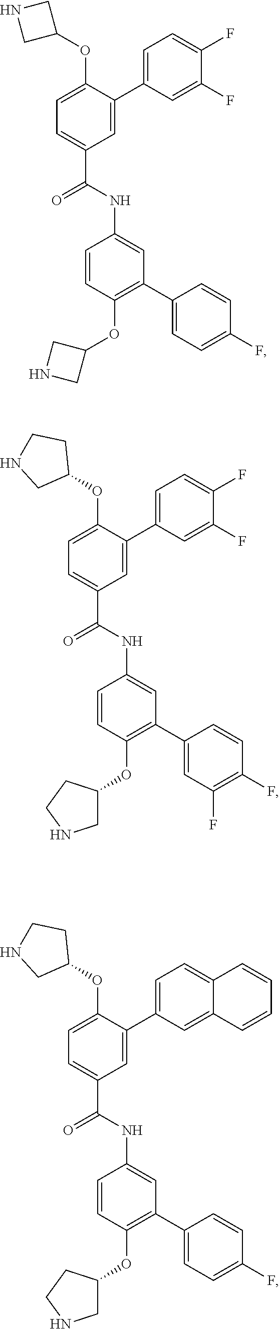 [SMILES: O=C(Nc1ccc(OC2CNC2)c(-c2ccc(F)cc2)c1)c1ccc(OC2CNC2)c(-c2ccc(F)c(F)c2)c1 ; O=C(Nc1ccc(O[C@H]2CCNC2)c(-c2ccc(F)c(F)c2)c1)c1ccc(O[C@H]2CCNC2)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc3ccccc3c2)c1] 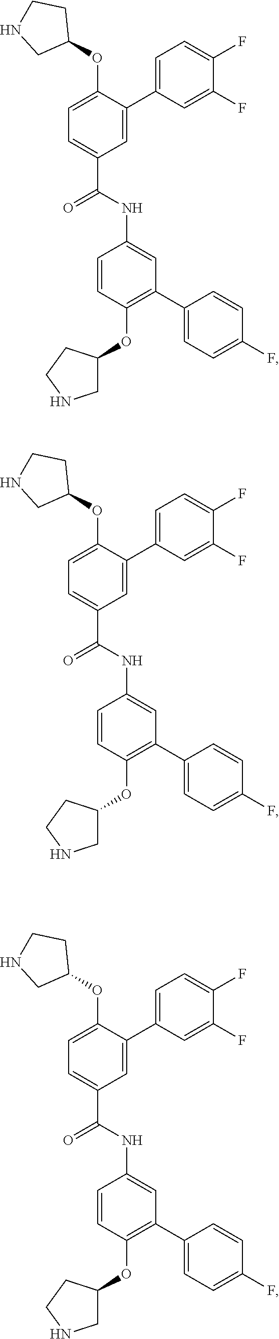 [SMILES: O=C(Nc1ccc(O[C@@H]2CCNC2)c(-c2ccc(F)cc2)c1)c1ccc(O[C@@H]2CCNC2)c(-c2ccc(F)c(F)c2)c1 ; O=C(Nc1ccc(O[C@H]2CCNC2)c(-c2ccc(F)cc2)c1)c1ccc(O[C@@H]2CCNC2)c(-c2ccc(F)c(F)c2)c1 ; O=C(Nc1ccc(O[C@@H]2CCNC2)c(-c2ccc(F)cc2)c1)c1ccc(O[C@H]2CCNC2)c(-c2ccc(F)c(F)c2)c1] 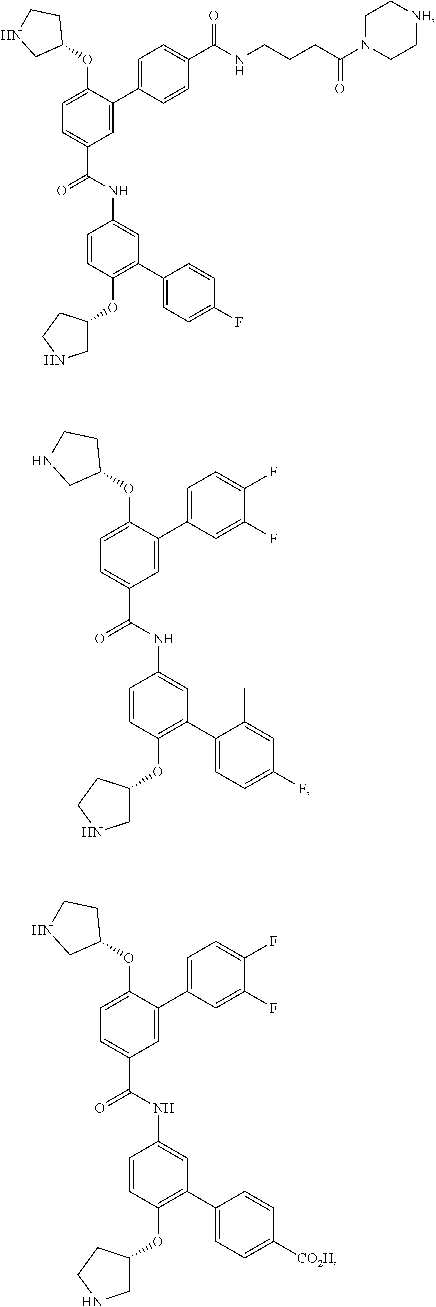 [SMILES: O=C(NCCCC(=O)N1CCNCC1)c1ccc(-c2cc(C(=O)Nc3ccc(O[C@H]4CCNC4)c(-c4ccc(F)cc4)c3)ccc2O[C@H]2CCNC2)cc1 ; Cc1cc(F)ccc1-c1cc(NC(=O)c2ccc(O[C@H]3CCNC3)c(-c3ccc(F)c(F)c3)c2)ccc1O[C@H]1CCNC1 ; O=C(O)c1ccc(-c2cc(NC(=O)c3ccc(O[C@H]4CCNC4)c(-c4ccc(F)c(F)c4)c3)ccc2O[C@H]2CCNC2)cc1] 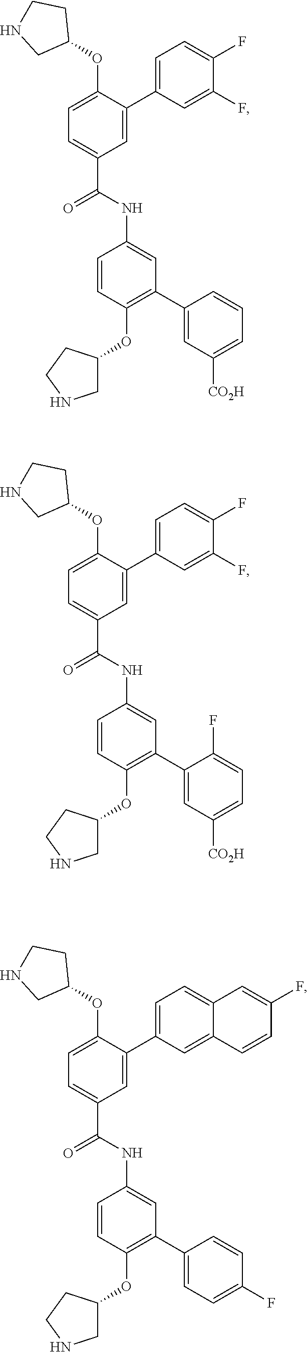 [SMILES: O=C(O)c1cccc(-c2cc(NC(=O)c3ccc(O[C@H]4CCNC4)c(-c4ccc(F)c(F)c4)c3)ccc2O[C@H]2CCNC2)c1 ; O=C(O)c1ccc(F)c(-c2cc(NC(=O)c3ccc(O[C@H]4CCNC4)c(-c4ccc(F)c(F)c4)c3)ccc2O[C@H]2CCNC2)c1 ; O=C(Nc1ccc(O[C@H]2CCNC2)c(-c2ccc(F)cc2)c1)c1ccc(O[C@H]2CCNC2)c(-c2ccc3cc(F)ccc3c2)c1] 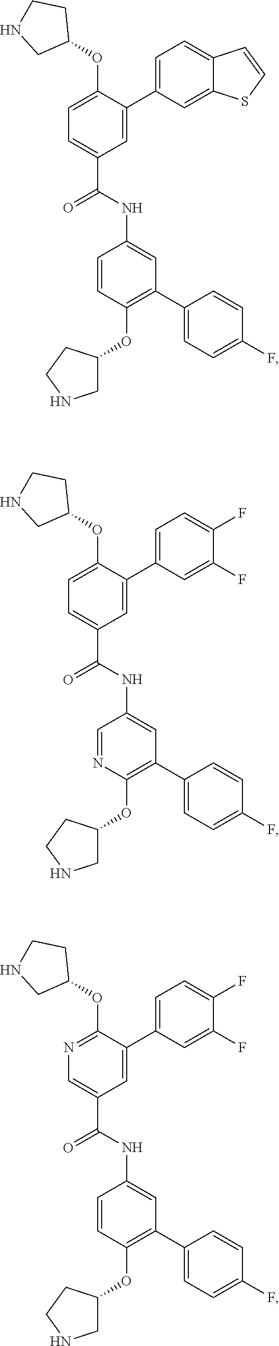 [SMILES: O=C(Nc1ccc(O[C@H]2CCNC2)c(-c2ccc(F)cc2)c1)c1ccc(O[C@H]2CCNC2)c(-c2ccc3ccsc3c2)c1 ; O=C(Nc1cnc(O[C@H]2CCNC2)c(-c2ccc(F)cc2)c1)c1ccc(O[C@H]2CCNC2)c(-c2ccc(F)c(F)c2)c1 ; O=C(Nc1ccc(O[C@H]2CCNC2)c(-c2ccc(F)cc2)c1)c1cnc(O[C@H]2CCNC2)c(-c2ccc(F)c(F)c2)c1] 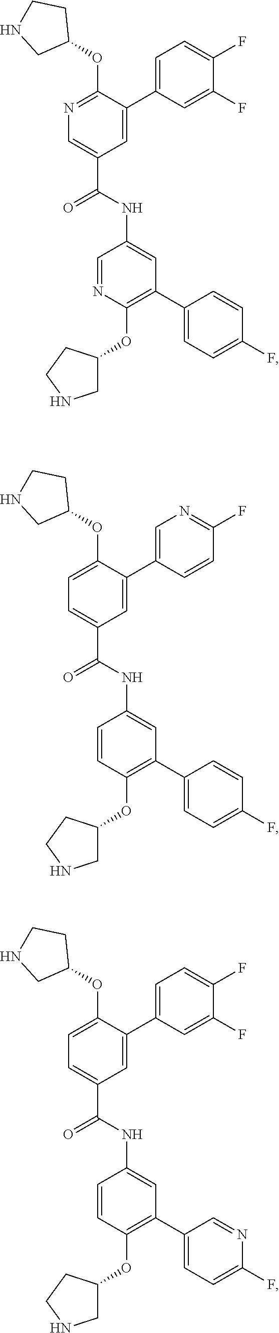 [SMILES: O=C(Nc1cnc(O[C@H]2CCNC2)c(-c2ccc(F)cc2)c1)c1cnc(O[C@H]2CCNC2)c(-c2ccc(F)c(F)c2)c1 ; O=C(Nc1ccc(O[C@H]2CCNC2)c(-c2ccc(F)cc2)c1)c1ccc(O[C@H]2CCNC2)c(-c2ccc(F)nc2)c1 ; O=C(Nc1ccc(O[C@H]2CCNC2)c(-c2ccc(F)nc2)c1)c1ccc(O[C@H]2CCNC2)c(-c2ccc(F)c(F)c2)c1] 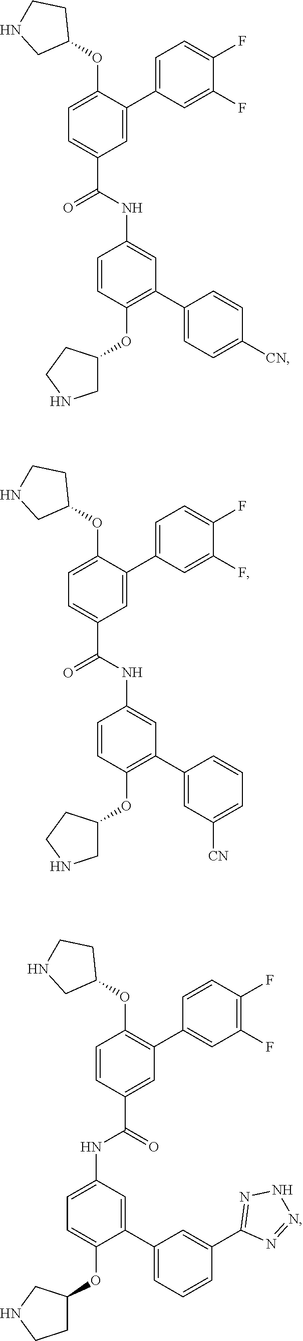 [SMILES: N#Cc1ccc(-c2cc(NC(=O)c3ccc(O[C@H]4CCNC4)c(-c4ccc(F)c(F)c4)c3)ccc2O[C@H]2CCNC2)cc1 ; N#Cc1cccc(-c2cc(NC(=O)c3ccc(O[C@H]4CCNC4)c(-c4ccc(F)c(F)c4)c3)ccc2O[C@H]2CCNC2)c1 ; O=C(Nc1ccc(O[C@H]2CCNC2)c(-c2cccc(-c3nn[nH]n3)c2)c1)c1ccc(O[C@H]2CCNC2)c(-c2ccc(F)c(F)c2)c1] 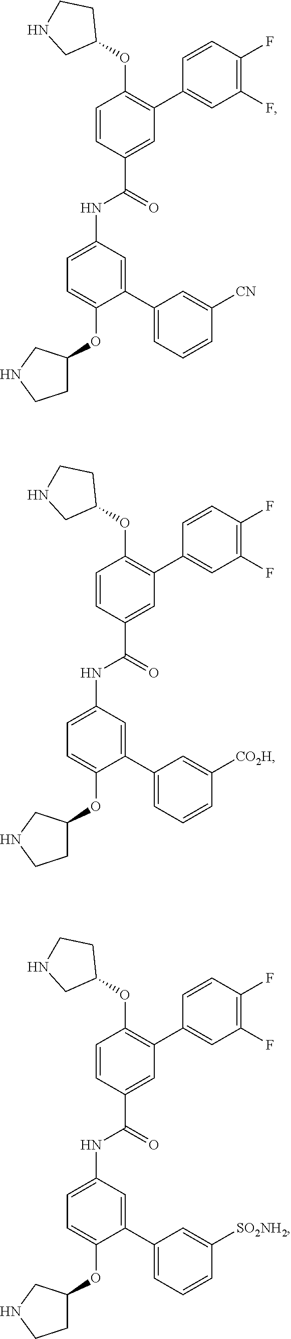 [SMILES: N#Cc1cccc(-c2cc(NC(=O)c3ccc(O[C@H]4CCNC4)c(-c4ccc(F)c(F)c4)c3)ccc2O[C@H]2CCNC2)c1 ; O=C(O)c1cccc(-c2cc(NC(=O)c3ccc(O[C@H]4CCNC4)c(-c4ccc(F)c(F)c4)c3)ccc2O[C@H]2CCNC2)c1 ; NS(=O)(=O)c1cccc(-c2cc(NC(=O)c3ccc(O[C@H]4CCNC4)c(-c4ccc(F)c(F)c4)c3)ccc2O[C@H]2CCNC2)c1] 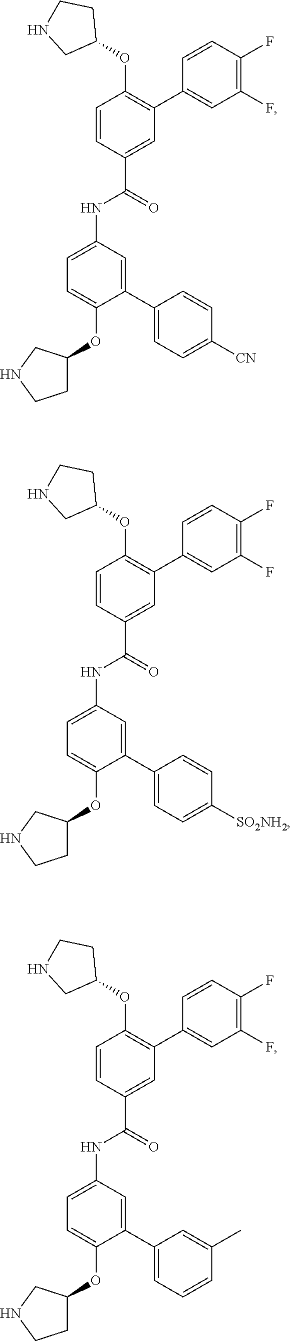 [SMILES: N#Cc1ccc(-c2cc(NC(=O)c3ccc(O[C@H]4CCNC4)c(-c4ccc(F)c(F)c4)c3)ccc2O[C@H]2CCNC2)cc1 ; NS(=O)(=O)c1ccc(-c2cc(NC(=O)c3ccc(O[C@H]4CCNC4)c(-c4ccc(F)c(F)c4)c3)ccc2O[C@H]2CCNC2)cc1 ; Cc1cccc(-c2cc(NC(=O)c3ccc(O[C@H]4CCNC4)c(-c4ccc(F)c(F)c4)c3)ccc2O[C@H]2CCNC2)c1] 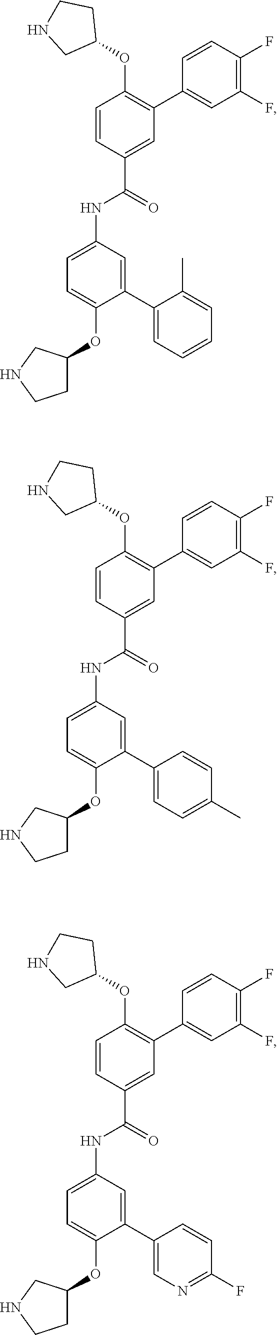 [SMILES: Cc1ccccc1-c1cc(NC(=O)c2ccc(O[C@H]3CCNC3)c(-c3ccc(F)c(F)c3)c2)ccc1O[C@H]1CCNC1 ; Cc1ccc(-c2cc(NC(=O)c3ccc(O[C@H]4CCNC4)c(-c4ccc(F)c(F)c4)c3)ccc2O[C@H]2CCNC2)cc1 ; O=C(Nc1ccc(O[C@H]2CCNC2)c(-c2ccc(F)nc2)c1)c1ccc(O[C@H]2CCNC2)c(-c2ccc(F)c(F)c2)c1] 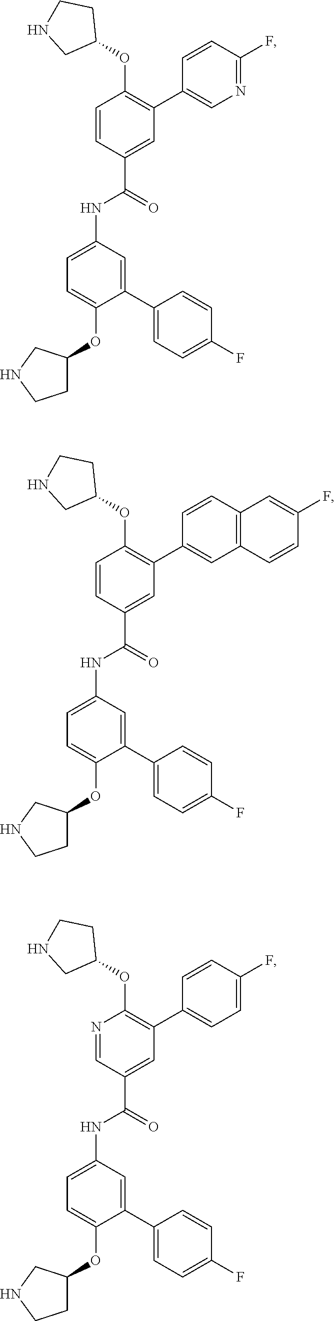 [SMILES: O=C(Nc1ccc(O[C@H]2CCNC2)c(-c2ccc(F)cc2)c1)c1ccc(O[C@H]2CCNC2)c(-c2ccc(F)nc2)c1 ; O=C(Nc1ccc(O[C@H]2CCNC2)c(-c2ccc(F)cc2)c1)c1ccc(O[C@H]2CCNC2)c(-c2ccc3cc(F)ccc3c2)c1 ; O=C(Nc1ccc(O[C@H]2CCNC2)c(-c2ccc(F)cc2)c1)c1cnc(O[C@H]2CCNC2)c(-c2ccc(F)cc2)c1] 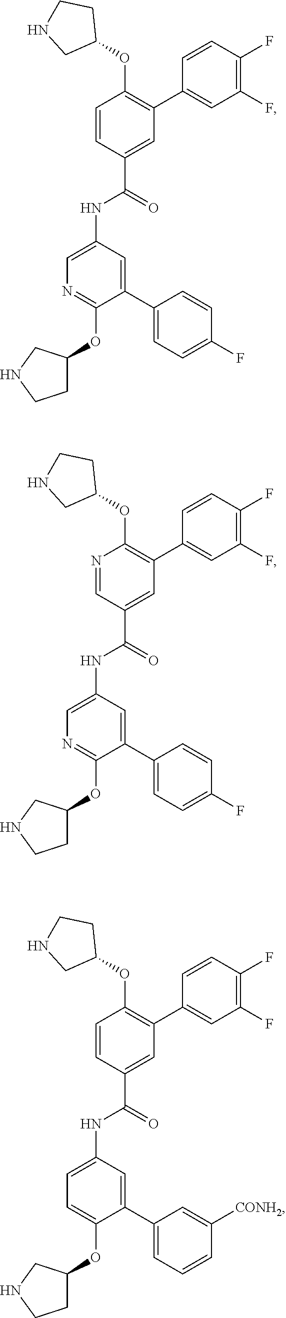 [SMILES: O=C(Nc1cnc(O[C@H]2CCNC2)c(-c2ccc(F)cc2)c1)c1ccc(O[C@H]2CCNC2)c(-c2ccc(F)c(F)c2)c1 ; O=C(Nc1cnc(O[C@H]2CCNC2)c(-c2ccc(F)cc2)c1)c1cnc(O[C@H]2CCNC2)c(-c2ccc(F)c(F)c2)c1 ; NC(=O)c1cccc(-c2cc(NC(=O)c3ccc(O[C@H]4CCNC4)c(-c4ccc(F)c(F)c4)c3)ccc2O[C@H]2CCNC2)c1] 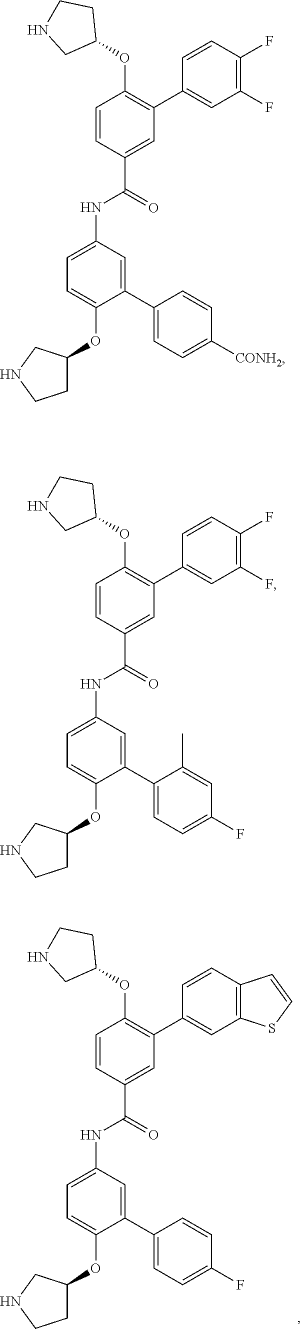 [SMILES: NC(=O)c1ccc(-c2cc(NC(=O)c3ccc(O[C@H]4CCNC4)c(-c4ccc(F)c(F)c4)c3)ccc2O[C@H]2CCNC2)cc1 ; Cc1cc(F)ccc1-c1cc(NC(=O)c2ccc(O[C@H]3CCNC3)c(-c3ccc(F)c(F)c3)c2)ccc1O[C@H]1CCNC1 ; O=C(Nc1ccc(O[C@H]2CCNC2)c(-c2ccc(F)cc2)c1)c1ccc(O[C@H]2CCNC2)c(-c2ccc3ccsc3c2)c1] 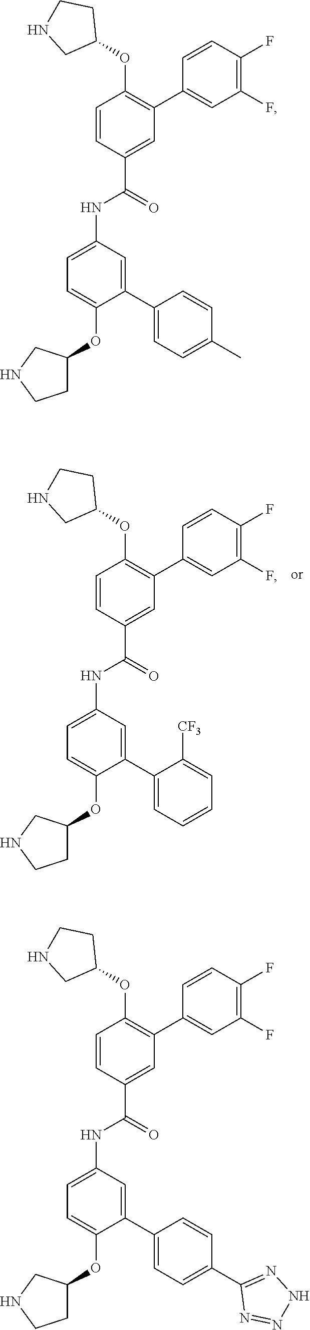 [SMILES: Cc1ccc(-c2cc(NC(=O)c3ccc(O[C@H]4CCNC4)c(-c4ccc(F)c(F)c4)c3)ccc2O[C@H]2CCNC2)cc1 ; O=C(Nc1ccc(O[C@H]2CCNC2)c(-c2ccccc2C(F)(F)F)c1)c1ccc(O[C@H]2CCNC2)c(-c2ccc(F)c(F)c2)c1 ; "or" ; O=C(Nc1ccc(O[C@H]2CCNC2)c(-c2ccc(-c3nn[nH]n3)cc2)c1)c1ccc(O[C@H]2CCNC2)c(-c2ccc(F)c(F)c2)c1]
or a subgroup thereof.

12. The compound of claim 1, wherein the compound selectively inhibits β-catenin/BCL9 interactions compared to β-catenin/cadherin interactions.

13. The compound of claim 1, wherein the compound exhibits inhibition with a Kᵢ of less than about 1.0×10⁻⁴ M when determined in competitive inhibition assay.

14. A method for the treatment of a disorder of uncontrolled cellular proliferation associated with a β-catenin/BCL9 dysfunction in a mammal comprising the step of administering to the mammal an effective amount of at least one compound having a structure represented by a formula: 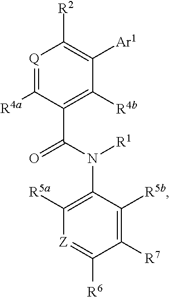
wherein Q is selected from N and CR^{4c};
wherein Z is selected from N and CR^{5c};
wherein R¹ is selected from hydrogen and C1-C4 alkyl;
wherein R² is selected from -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂—Cy¹, —NHCH₂—Cy²; —OCH₂—Cy¹, and —OCH₂—Cy²;
wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and
wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl;
wherein each of R^{4a}, R^{4b}, and R^{4c}, when present, is independently selected from hydrogen, C1-C4 alkyl, C1-C4 monohaloalkyl, and C1-C4 polyhaloalkyl;
wherein each of R^{5a}, R^{5b}, and R^{5c}, when present, is independently selected from hydrogen, C1-C4 alkyl, C1-C4 monohaloalkyl, and C1-C4 polyhaloalkyl;
wherein R⁶ is selected from hydrogen, -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂—Cy³, —NHCH₂—Cy⁴; —OCH₂—Cy³, and —OCH₂—Cy⁴;
wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and
wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl;
wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
wherein each occurrence of R²⁰, when present, is independently selected from C1-C3 alkyl, C1-C3 monohaloalkyl, C1-C3 polyhaloalkyl, and cyclopropyl;
wherein each occurrence of R^{21a} and R^{21b}, when present, is independently selected from hydrogen, C1-C3 alkyl, C1-C3 monohaloalkyl, C1-C3 polyhaloalkyl, and cyclopropyl;
wherein R⁷ is selected from Ar², -A¹-A²-Ar², and 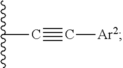
wherein each of A¹ and A², when present, is independently selected from O, NH, and CH₂, provided that each of A¹ and A² is simultaneously O; and
wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
or a pharmaceutically acceptable salt thereof.

15. The method of claim 14, wherein the mammal is human; and wherein the human has been identified to have a 1q21 chromosomal abnormality.

16. The method of claim 14, further comprising the step of identifying a mammal in need of treatment of the disorder.

17. The method of claim 16, wherein the mammal is human; and wherein the step of identifying the human in need of treatment of the disorder comprises the steps of:
(a) obtaining a sample from the human;
wherein the sample comprises cells suspected of being associated with the disorder of uncontrolled cellular proliferaton;
(b) determining if the sample comprises cells with a 1q21 chromosomal abnormality; and
(c) administering to the human the compound when the sample is positive for a 1q21 chromosomal abnormality.

18. The method of claim 14, wherein the disorder is cancer.

19. A method for inhibiting protein-protein interactions of β-catenin and BCL9 in at least one cell, comprising the step of contacting the at least one cell with an effective amount of at least one compound having a structure represented by a formula: 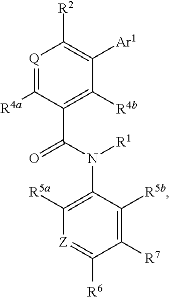
wherein Q is selected from N and CR^{4c};
wherein Z is selected from N and CR^{5c};
wherein R¹ is selected from hydrogen and C1-C4 alkyl;
wherein R² is selected from -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, —NH—(C2-C8 alkyl)-NH₂, —NH-Cy¹, —NH-Cy², —O-Cy¹, —O-Cy², —NHCH₂—Cy¹, —NHCH₂—Cy²; —OCH₂—Cy¹, and —OCH₂—Cy²;
wherein Cy¹, when present, is an amino C3-C8 cycloalkyl or hydroxy C3-C8 cycloalkyl, and wherein Cy¹ is substituted 0, 1, 2, or 3 groups independently selected from halogen, C1-C4 alkyl, C1-C4 monohaloalkyl, and C1-C4 polyhaloalkyl; and
wherein Cy², when present, is a C2-C7 heterocycloalkyl comprising at least one oxygen or nitrogen atom, and wherein Cy² is substituted with 0, 1, 2, or 3 groups independently selected from halogen, C1-C4 alkyl, C1-C4 monohaloalkyl, and C1-C4 polyhaloalkyl;
wherein each of R^{4a}, R^{4b}, and R^{4c}, when present, is independently selected from hydrogen, C1-C4 alkyl, C1-C4 monohaloalkyl, and C1-C4 polyhaloalkyl;
wherein each of R^{5a}, R^{5b}, and R^{5c}, when present, is independently selected from hydrogen, C1-C4 alkyl, C1-C4 monohaloalkyl, and C1-C4 polyhaloalkyl;
wherein R⁶ is selected from hydrogen, -(C2-C8 alkyl)-OH, -(C2-C8 alkyl)-NH₂, —O—(C2-C8 alkyl)-OH, —O—(C2-C8 alkyl)-NH₂, —NH—(C2-C8 alkyl)-OH, and —NH—(C2-C8 alkyl)-NH₂, —NH-Cy³, —NH-Cy⁴, —O-Cy³, —O-Cy⁴, —NHCH₂—Cy³, —NHCH₂—Cy⁴; —OCH₂—Cy³, and —OCH₂—Cy⁴;
wherein Cy³, when present, is an amino C3-C8 cycloalkyl or hydroxy C3-C8 cycloalkyl, and wherein Cy³ is substituted 0, 1, 2, or 3 groups independently selected from halogen, C1-C4 alkyl, C1-C4 monohaloalkyl, and C1-C4 polyhaloalkyl; and
wherein Cy⁴, when present, is a C2-C7 heterocycloalkyl comprising at least one oxygen or nitrogen atom, and wherein Cy⁴ is substituted with 0, 1, 2, or 3 groups independently selected from halogen, C1-C4 alkyl, C1-C4 monohaloalkyl, and C1-C4 polyhaloalkyl;
wherein Ar¹ is selected from aryl and heteroaryl, and wherein Ar¹ is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
wherein each occurrence of R²⁰, when present, is independently selected from C1-C3 alkyl, C1-C3 monohaloalkyl, C1-C3 polyhaloalkyl, and cyclopropyl;
wherein each occurrence of R^{21a} and R^{21b}, when present, is independently selected from hydrogen, C1-C3 alkyl, C1-C3 monohaloalkyl, C1-C3 polyhaloalkyl, and cyclopropyl;
wherein R⁷ is selected from Ar², -A¹-A²-Ar², and 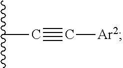
wherein each of A¹ and A², when present, is independently selected from O, NH, and CH₂, provided that each of A¹ and A² is simultaneously O; and
wherein Ar² is selected from aryl and heteroaryl, and wherein Ar² is substituted with 0, 1, 2, or 3 groups independently selected from halogen, —CN, C1-C3 alkyl, C1-C3 monohaloalkyl, C1-C3 polyhaloalkyl, cyclopropyl, —NHCOR²⁰, —NHSO₂R²⁰, —CONR^{21a}R^{21b}, —SO₂NR^{21a}R^{21b}, —CO₂H, and tetrazole;
or a pharmaceutically acceptable salt thereof.

20. The method of claim 19, wherein contacting is via administration to a mammal.


SEQUENCE LISTING (3 sequences)

SEQ ID NO: 1 - protein, 7 aa, Artificial Sequence
  Synthetic Construct
HVGGSSV

SEQ ID NO: 2 - protein, 13 aa, Artificial Sequence
  Synthetic Construct
GCGGGNHVGGSSV

SEQ ID NO: 3 - protein, 13 aa, Artificial Sequence
  Synthetic Construct
GCGGGSGVSGHNG